(19) United States
(12) Patent Application Publication
(10) Pub. No.: US 2024/0166617 A1
(43) Pub. Date: May 23, 2024

(54) PHOSPHOINOSITIDE 3 KINASE BETA INHIBITORS AND COMPOSITIONS AND METHODS THEREOF

(71) Applicants: Geode Therapeutics Inc., Brookline, MA (US); XtalPi, Inc., Shenzhen (CN)

(72) Inventors: Jean Zhao, Brookline, MA (US); Qi Wang, Brookline, MA (US); Xiang Y. Yu, Acton, MA (US); Huimin Cheng, Shenzhen (CN); Peiyu Zhang, Shenzhen (CN); Zhixiong Lin, Shenzhen (CN); Lei Fang, Shenzhen (CN); Songling Ma, Shenzhen (CN)

(21) Appl. No.: 18/273,634

(22) PCT Filed: Jan. 25, 2022
(86) PCT No.: PCT/US22/13725

§ 371 Date: Jul. 21, 2023

Related U.S. Application Data
(60) Provisional application No. 63/144,287, filed on Feb. 1, 2021.

(51) Int. Cl. C07D 311/22; C07D 405/06; C07D 405/12; C07D 405/14; C07D 413/04; C07D 413/12; C07D 471/04; C07D 495/10

(52) U.S. Cl. CPC C07D 311/22; C07D 405/06; C07D 405/12; C07D 405/14; C07D 413/04; C07D 413/12; C07D 471/04; C07D 495/10

(57) ABSTRACT

The invention provides novel phosphoinositide 3 kinase beta-selective inhibitors and pharmaceutical compositions thereof, as well as methods of their preparation and use, in therapy of various diseases and conditions, such as solid tumors.

### PRIORITY CLAIMS AND RELATED PATENT APPLICATIONS

This application claims the benefit of priority to U.S. Provisional Application Ser. No. 63/144,287, filed Feb. 1, 2021, the entire content of which is incorporated herein by reference.

### TECHNICAL FIELDS OF THE INVENTION

The invention generally relates to pharmaceuticals and therapeutic methods. More particularly, the invention provides novel phosphoinositide 3 kinase beta inhibitors and pharmaceutical compositions thereof, as well as methods of their preparation and use, in therapy of various diseases and conditions, such as multiple types of tumors.

### BACKGROUND OF THE INVENTION

Phosphoinositide 3-kinases (PI3Ks) are a family of lipid kinases that play key regulatory roles in many cellular processes including cell survival, proliferation, differentiation, motility, and metabolism (Thorpe L M et al., Nat Rev Cancer, 2015,1,7-14). There are three classes of PI3Ks: class I, class II and class III based on their characteristics and substrate specificity. Class I PI3Ks are further divided into class IA enzymes that are activated by receptor tyrosine kinases (RTKs), G protein coupled receptors (GPCRs) and oncoproteins, and class IB enzymes that are regulated exclusively by GPCRs. Class IA PI3Ks are heterodimers of a p110 catalytic subunit and a p85 regulatory subunit. Three highly homologous p110 catalytic isoforms p110α, p110β, and p110δ, encoded by genes PIK3CA, PIK3CB, and PIK3CD respectively, associated with any of five p85 regulatory isoforms, p85α (and its splicing variants p55α and p50α, encoded by PIK3R1), p85β (PIK3R2), and p55γ (PIK3R3). Class IB PI3Ks are heterodimers of a p110γ catalytic subunit (encoded by PIK3CG) coupled with regulatory isoforms p101 (PIK3R5) or p87 (p84 or p87PIKAP, encoded by PIK3R6) (Engelman J A et al. Nat Rev Genet, 2006,7, 606-19). While p110α and p110β are ubiquitously expressed, p110δ and p110γ expression is largely restricted to leukocytes (OKKenhaug K et al., Nat Rev Immunol, 2003, 3, 317-30).

In response to ligand stimulation and the subsequent activation of RTKs or GPCRs, p110 catalytic subunit is activated via interaction of its p85 regulatory subunit and uses phosphatidylinositol 4,5-biphosphate (PIP2) as a substrate to generate phosphatidylinositol 3,4,5-triphosphate (PIP3), which in turn activates AKT-dependent and -independent downstream signaling pathways. The phosphatase and tensin homolog (PTEN) lipid phosphatase removes the 3′-phosphate from PtdIns(3,4,5)P3 to counteract the activation of PI3Ks (Engelman J A et al. Nat Rev Genet, 2006, 7,606-19).

Hyperactivation of the PI3K pathway is one of the most common events in human cancers. Aberrant PI3K signaling activation is mainly caused by somatic loss of PTEN via genetic or epigenetic alterations, activation of RTKs or alterations in the isoforms of PI3Ks. These alterations are frequently detected in a range of tumor types and offer opportunities for therapeutic targeting of the pathway (Parsons R et al., Semin Cell Dev Biol, 2004,15,171-6; Janku F et al., Nat Rev Clin Oncol, 2018, 15, 273-291). Tremendous efforts have been devoted to the development of effective PI3K inhibitors. However, non-isoform-selective pan-PI3K inhibitors have not yielded exciting results due to potential severe toxicity to the immune system, which is largely dependent on p110δ and p110γ for function. Recent preclinical studies have shown that different PI3K isoforms have divergent roles in cellular signaling and cancer (Jia S et al., Curr Opin Cell Biol, 2009, 2, 199-208), suggesting that inhibitors targeting individual isoforms may achieve greater therapeutic efficacy.

PTEN deficiency, the most common mechanism leading to aberrant PI3K signaling, frequently occurs in multiple human malignant tumors such as prostate, colon, breast, thyroid, endometrial, kidney, melanoma cancers, and leukemia (Michele Milella et al., Front Oncol, 2015, 5, 24). The p110β isoform plays a dominant role in PTEN-deficient tumors through the use of pharmacological agents and genetic models. p110β ablation sufficiently inhibits the tumor formation in anterior prostates accompanied by concomitant diminution of AKT activation in a mouse prostate tumor model driven by PTEN deficiency (Jia S et al., Nature, 2008, 454, 776-9). Cell culture-based studies confirmed and extended this finding to other types of human cancer cells (prostate, brain, and breast). Knock-down of p110β inhibits downstream activation of AKT, cell transformation, and the growth of these PTEN-deficient cells and tumor xenografts (Wee S et al., Proc Natl Acad Sci USA, 2008, 105, 13057-13062). In mouse models, myeloid neoplasia driven by PTEN loss is dependent on p110β via p110β-Rac-positive-feedback Loop. Disruption of this loop may offer a new and effective therapeutic strategy for PTEN-deficient leukemia (Yuzugullu H et al. Nat Commun, 2015, 6, 8501).

It is also worth noting that PIK3CB gene amplification or mutations occur in multiple human cancer types including breast, lung, prostate, esophageal and Glioblastoma. Tumor cells expressing gain-of-function mutation, for example p110β^{E1051K}, are sensitive to p111β inhibition (Whale A D et al. Signal Transduct Target Ther, 2017, 2, 17063).

In addition to tumorigenesis, there is evidence that p110β plays essential roles in a variety of other diseases and physiological processes including thrombosis, male fertility, and Fragile X-syndrome. p110β has been proposed as an antithrombotic target based on its ability to inhibit shear activation of platelets, stable platelet aggregation and thrombus formation without causing a significant increase in bleeding (Jackson S P et al. J Thromb Haemost, 2012, 10, 2123-2126). p110β inactivation leads to a specific blockade in sperm development, without affecting the spermatogenic stem cell pool, makes it a potential drug target for a male contraceptive (Julie Guillermet-Guibert J et al., PLoS Genet, 2015,11, e1005304). Fragile X syndrome is caused by loss of function of the fragile X mental retardation protein. Preclinical results showed that p110β plays an important role in prefrontal cortex (PFC)-dependent cognitive defects in mouse models of Fragile X syndrome (Gross C et al. Cell Rep. 2015, 11, 681-688). Taken together, these findings point towards the potential of PI3Kβ-specific targeting therapies as a highly efficacious treatment modality for PTEN deficient tumors and diseases that are highly dependent on PI3Kβ, while simultaneously mitigating the severe toxicity effects of pan-PI3K inhibitors.

PI3Kβ-selective inhibitors are now emerging in the clinic, yet facing immense challenge ahead. For instance, the study of PI3Kβ-selective inhibitor SAR260301 (WO2011001114A1) was terminated permanently at Phase I clinical trial for the patients with lymphoma due to unsustained pharmacodynamic effect. One subject of AZD6482 (WO2009093972A1) was discontinued at Phase I clinical trial for the patient with thrombosis because of itching and a rash at the infusion site.

Thus, there is an urgent need for novel PI3Kβ inhibitors and treatment methods for PTEN-deficient cancers and PI3Kβ dependent diseases that provide improved clinical effectiveness with reduced side effects.

### SUMMARY OF THE INVENTION

The invention provides novel selective PI3Kβ inhibitors that are useful for treating various of diseases including cancers, e.g., breast, colon, endometrial, kidney, lung, melanoma, prostate, thyroid cancer and leukemia.

In one aspect, the invention generally relates to a compound having the structural formula (I):

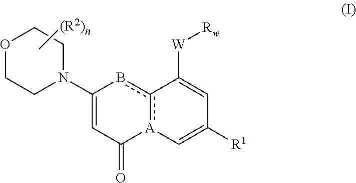

wherein
A represents carbon and B represents oxygen, or each of A and B represent nitrogen;
W represents —CHR-Q- or -Q-CHR—; wherein Q represents a bond, O, S, CH₂ or NR′; R and R′ is independently selected from hydrogen, C₁₋₄alkyl or C₁₋₄alkyl substituted with —OH or halo;
R_{w} represents Ar or a substituted or unsubstituted indoline group, wherein Ar is a group comprising a substituted or unsubstituted 6-membered aromatic or hetero-aromatic ring;
R² represents C₁₋₃ alkyl, CN, CH₂OH, CH₂F or CONH₂;
R¹ represents H, NH₂, OH, CN, CF₃, CHO, COC₁₋₄alkyl, CH₂C(═O)—NR^{1a}R^{1b}, C(═O)—NR^{1a}R^{1b}, CH₂COOR^{1c}, NR^{1d}R^{1e}, C₁₋₄ alkyl, O—C₁₋₄ alkyl, Het, Ar, NR^{1h}(C═O)R¹ⁱ, NR^{1j}(C═O)NR^{1k}R^{1l}, NR^{1j}(SO₂)R^{1m}, SO₂NR¹ⁿR^{1o}, NR^{1j}(C═O)C(CN)C(OH)R^{1p}

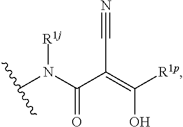

P(O)MeMe, P(O)OMeOMe; provided that, when A is carbon, B is oxygen and R_{w} is Ar, R¹ represents NH₂, CN, CHO, COC₁₋₄alkyl, CH₂C(═O)—NR^{1a}R^{1b}, CH₂COOR^{1c}, NR^{1d}R^{1e}, O—C₂₋₄ alkyl, Het, NR^{1h}(C═O)R¹ⁱ, NR^{1j}(C═O)NR^{1k}R^{1l}, NR^{1j}(SO₂)R^{1m}, SO₂NR¹ⁿR^{1o}, NR^{1j}(C═O)C(CN)C(OH)R^{1P}, P(O)MeMe, P(O)OMeOMe; when A and B is nitrogen, R_{w} is Ar, R¹ represents NH₂, CN, CHO, COC₁₋₄alkyl, CH₂C(═O)—NR^{1a}R^{1b}, CH₂COOR^{1c}, NR^{1d}R^{1e}, O—C₂₋₄ alkyl, Het, NR^{1h}(C═O)R¹ⁱ, NR^{1j}(C═O)NR^{1k}R^{1l}, NR^{1j}(SO₂)R^{1m}, SO₂NR¹ⁿR^{1o}, NR^{1j}(C═O)C(CN)C(OH)R^{1P}, P(O)MeMe, P(O)OMeOMe, C(═O)—NR^{1q}R^{1r}, wherein R^{1q} and R^{1r} together form a 3- to 8-membered nitrogen-containing heterocyclyl ring with one or more substituent selected from the group consisting of C₁₋₃ alkyl and NR^{3a}R^{3b};
wherein each of R^{1a}, R^{1b}, R^{1c}, R^{1d}, R^{1e}, R^{1h}, R¹ⁱ, R^{1j}, R^{1k}, R^{1l}, R^{1m}, R¹ⁿ, R^{1o}, R^{1p} is independently selected from H, C₁₋₄ alkyl, C₃₋₈cycloalkyl, C₃₋₈heterocycloakyl or C₁₋₄ alkyl substituted with one or more substituent selected from the group consisting of hydroxyl, OMe, CN, fluoro, NR^{3a}R^{3b}, C₃₋₈cycloalkyl, C₃₋₈heterocycloakyl; R^{1a} and R^{1b}, R^{1d} and R^{1e}, R^{1m} and R^{1o} are optionally together form a 3- to 8-membered nitrogen-containing heterocyclyl ring with one or more substituent selected from the group consisting of C₁₋₃ alkyl and NR^{3a}R^{3b}
C₁₋₄ alkyl is unsubstituted or substituted with one or more substituent selected from the group consisting of OH, F, NR^{1d}R^{1e},
R^{3a} and R^{3b} each independently are selected from the group consisting of H, and C₁₋₄ alkyl;
Het represents a 3- to 8-membered saturated or partially saturated monocyclic, bridged or spiro heterocyclyl containing at least one heteroatom each independently selected from O, S, S(═O)ₚ and N; which substituted wish one or two substituents each independently selected from the group consisting of halo, NR^{3a}R^{3b}, C₁₋₄ alkyl, OH, OMe and CN;
n represents 0, 1, 2, 3 or 4; and
p represents 1 or 2,
   or a pharmaceutically acceptable form or an isotope derivative thereof.

In another aspect, the invention generally relates to a pharmaceutical composition comprising a compound disclosed herein, effective to treat or reduce one or more diseases or disorders, in a mammal, including a human, and a pharmaceutically acceptable excipient, carrier, or diluent.

In yet another aspect, the invention generally relates to a pharmaceutical composition comprising a compound having the structural formula (I):

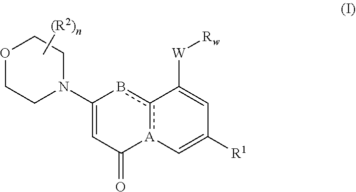

wherein
A represents carbon and B represents oxygen, or each of A and B represent nitrogen;
W represents —CHR-Q- or -Q-CHR—; wherein Q represents a bond, O, S, CH₂ or NR′; R and R′ is independently selected from hydrogen, C₁₋₄alkyl or C₁₋₄alkyl substituted with —OH or halo;
R_{w} represents Ar or a substituted or unsubstituted indoline group, wherein Ar is a group comprising a substituted or unsubstituted 6-membered aromatic or hetero-aromatic ring;
R² represents C₁₋₃ alkyl, CN, CH₂OH, CH₂F or CONH₂;
R¹ represents H, NH₂, OH, CN, CF₃, CHO, COC₁₋₄alkyl, CH₂C(═O)—NR^{1a}R^{1b}, C(═O)—NR^{1a}R^{1b}, CH₂COOR^{1c}, NR^{1d}R^{1e}, C₁₋₄ alkyl, O—C₁₋₄ alkyl, Het, Ar, NR^{1h}(C═O)R¹ⁱ, NR^{1j}(C═O)NR^{1k}R^{1l}, NR^{1j}(SO₂)R^{1m}, SO₂NR¹ⁿR^{1o}, NR^{1j}(C═O)C(CN)C(OH)R^{1p}, P(O)MeMe, P(O)OMeOMe; provided that, when A is carbon, B is oxygen and R_{w} is Ar, R¹ represents NH₂, CN, CHO, COC₁₋₄alkyl, CH₂C(═O)—NR^{1a}R^{1b}, CH₂COOR^{1c}, NR^{1d}R^{1e}, O—C₂₋₄ alkyl, Het, NR^{1h}(C═O)R¹ⁱ, NR^{1j}(C═O)NR^{1k}R^{1l}, NR^{1j}(SO₂)R^{1m}, SO₂NR¹ⁿR^{1o}, NR^{1j}(C═O)C(CN)C(OH)R^{1p}, P(O)MeMe, P(O)OMeOMe; when A and B is nitrogen, R_{w} is Ar, R¹ represents NH₂, CN, CHO, COC₁₋₄alkyl, CH₂C(═O)—NR^{1a}R^{1b}, CH₂COOR^{1c}, NR^{1d}R^{1e}, O—C₂₋₄ alkyl, Het, NR^{1h}(C═O)R¹ⁱ, NR^{1j}(C═O)NR^{1k}R^{1l}, NR^{1j}(SO₂)R^{1m}, SO₂NR¹ⁿR^{1o}, NR^{1j}(C═O)C(CN)C(OH)R^{1p}, P(O)MeMe, P(O)OMeOMe, C(═O)—NR^{1q}R^{1r}, wherein R^{1q} and R^{1r} together form a 3- to 8-membered nitrogen-containing heterocyclyl ring with one or more substituent selected from the group consisting of C₁₋₃ alkyl and NR^{3a}R^{3b};
wherein each of R^{1a}, R^{1b}, R^{1c}, R^{1d}, R^{1e}, R^{1h}, R¹ⁱ, R^{1j}, R^{1k}, R^{1l}, R^{1m}, R¹ⁿ, R^{1o}, R^{1p} is independently selected from H, C₁₋₄ alkyl, C₃₋₈cycloalkyl, C₃₋₈heterocycloakyl or C₁₋₄ alkyl substituted with one or more substituent selected from the group consisting of hydroxyl, OMe, CN, fluoro, NR^{3a}R^{3b}, C₃₋₈cycloalkyl, C₃₋₈heterocycloakyl; R^{1a} and R^{1b}, R^{1d} and R^{1e}, R^{1m} and R^{1o} are optionally together form a 3- to 8-membered nitrogen-containing heterocyclyl ring with one or more substituent selected from the group consisting of C₁₋₃ alkyl and NR^{3a}R^{3b},
C₁₋₄ alkyl is unsubstituted or substituted with one or more substituent selected from the group consisting of OH, F, NR^{1d}R^{1e},
R^{3a} and R^{3b} each independently are selected from the group consisting of H, and C₁₋₄ alkyl;
Het represents a 3- to 8-membered saturated or partially saturated monocyclic, bridged or spiro heterocyclyl containing at least one heteroatom each independently selected from O, S, S(═O)ₚ and N; which substituted wish one or two substituents each independently selected from the group consisting of halo, NR^{3a}R^{3b}, C₁₋₄ alkyl, OH, OMe and CN;
n represents 0, 1, 2, 3 or 4; and
p represents 1 or 2,
   or a pharmaceutically acceptable form or an isotope derivative thereof, effective to treat or reduce one or more diseases or disorders, in a mammal, including a human, and a pharmaceutically acceptable excipient, carrier, or diluent.

In yet another aspect, the invention generally relates to a unit dosage form comprising a pharmaceutical composition disclosed herein.

In yet another aspect, the invention generally relates to a method for treating or reducing a disease or disorder, comprising administering to a subject in need thereof a pharmaceutical composition comprising a compound having the structural formula (I):

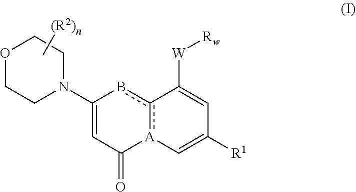

wherein
A represents carbon and B represents oxygen, or each of A and B represent nitrogen;
W represents —CHR-Q- or -Q-CHR—; wherein Q represents a bond, O, S, CH₂ or NR′; R and R′ is independently selected from hydrogen, C₁₋₄alkyl or C₁₋₄alkyl substituted with —OH or halo;
R_{w} represents Ar or a substituted or unsubstituted indoline group, wherein Ar is a group comprising a substituted or unsubstituted 6-membered aromatic or hetero-aromatic ring;
R² represents C₁₋₃ alkyl, CN, CH₂OH, CH₂F or CONH₂;
R¹ represents H, NH₂, OH, CN, CF₃, CHO, COC₁₋₄alkyl, CH₂C(═O)—NR^{1a}R^{1b}, C(═O)—NR^{1a}R^{1b}, CH₂COOR^{1c}, NR^{1d}R^{1e}, C₁₋₄ alkyl, O—C₁₋₄ alkyl, Het, Ar, NR^{1h}(C═O)R¹ⁱ, NR^{1j}(C═O)NR^{1k}R^{1l}, NR^{1j}(SO₂)R^{1m}, SO₂NR¹ⁿR^{1o}, NR^{1j}(C═O)C(CN)C(OH)R^{1p}, P(O)MeMe, P(O)OMeOMe; provided that, when A is carbon, B is oxygen and R_{w} is Ar, R¹ represents NH₂, CN, CHO, COC₁₋₄alkyl, CH₂C(═O)—NR^{1a}R^{1b}, CH₂COOR^{c}, NR^{1d}R^{1e}, O—C₂₋₄ alkyl, Het, NR^{1h}(C═O)R¹ⁱ, NR^{1j}(C═O)NR^{1k}R^{1l}, NR^{1j}(SO₂)R^{1m}, SO₂NR¹ⁿR^{1o}, NR^{1j}(C═O)C(CN)C(OH)R^{1p}, P(O)MeMe, P(O)OMeOMe; when A and B is nitrogen, R_{w} is Ar, R¹ represents NH₂, CN, CHO, COC₁₋₄alkyl, CH₂C(═O)—NR^{1a}R^{1b}, CH₂COOR^{1c}, NR^{1d}R^{1e}, O—C₂₋₄ alkyl, Het, NR^{1h}(C═O)R¹ⁱ, NR^{1j}(C═O)NR^{1k}R^{1l}, NR^{1j}(SO₂)R^{1m}, SO₂NR¹ⁿR^{1o}, NR^{1j}(C═O)C(CN)C(OH)R^{1p}, P(O)MeMe, P(O)OMeOMe, C(═O)—NR^{1q}R^{1r}, wherein R^{1q} and R^{1r} together form a 3- to 8-membered nitrogen-containing heterocyclyl ring with one or more substituent selected from the group consisting of C₁₋₃ alkyl and NR^{3a}R^{3b};
wherein each of R^{1a}, R^{1b}, R^{1c}, R^{1d}, R^{1e}, R^{1h}, R¹ⁱ, R^{1j}, R^{1k}, R^{1l}, R^{1m}, R¹ⁿ, R^{1o}, R^{1p} is independently selected from H, C₁₋₄ alkyl, C₃₋₈cycloalkyl, C₃₋₈heterocycloakyl or C₁₋₄ alkyl substituted with one or more substituent selected from the group consisting of hydroxyl, OMe, CN, fluoro, NR^{3a}R^{3b}, C₃₋₈cycloalkyl, C₃₋₈heterocycloakyl; R^{1a} and R^{1b}, R^{1d} and R^{1e}, R^{1m} and R^{1o} are optionally together form a 3- to 8-membered nitrogen-containing heterocyclyl ring with one or more substituent selected from the group consisting of C₁₋₃ alkyl and NR^{3a}R^{3b},
C₁₋₄ alkyl is unsubstituted or substituted with one or more substituent selected from the group consisting of OH, F, NR^{1d}R^{1e}
R^{3a} and R^{3b} each independently are selected from the group consisting of H, and C₁₋₄ alkyl;
Het represents a 3- to 8-membered saturated or partially saturated monocyclic, bridged or spiro heterocyclyl containing at least one heteroatom each independently selected from O, S, S(═O)ₚ and N; which substituted wish one or two substituents each independently selected from the group consisting of halo, NR^{3a}R^{3b}, C₁₋₄ alkyl, OH, OMe and CN;
n represents 0, 1, 2, 3 or 4; and
p represents 1 or 2,
   or a pharmaceutically acceptable form or an isotope derivative thereof, effective to treat cancer, or a related disease or disorder, in a mammal, including a human.

### Definitions

Unless defined otherwise, all technical and scientific terms used herein have the same meaning as commonly understood by one of ordinary skill in the art to which this invention belongs. General principles of organic chemistry, as well as specific functional moieties and reactivity, are described in “Organic Chemistry”, Thomas Sorrell, University Science Books, Sausalito: 2006.

As used herein, “at least” a specific value is understood to be that value and all values greater than that value.

The term “comprising”, when used to define compositions and methods, is intended to mean that the compositions and methods include the recited elements, but do not exclude other elements. The term “consisting essentially of”, when used to define compositions and methods, shall mean that the compositions and methods include the recited elements and exclude other elements of any essential significance to the compositions and methods. For example, “consisting essentially of” refers to administration of the pharmacologically active agents expressly recited and excludes pharmacologically active agents not expressly recited. The term consisting essentially of does not exclude pharmacologically inactive or inert agents, e.g., pharmaceutically acceptable excipients, carriers or diluents. The term “consisting of”, when used to define compositions and methods, shall mean excluding trace elements of other ingredients and substantial method steps. Embodiments defined by each of these transition terms are within the scope of this invention.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

As used herein, the term “administration” of a disclosed compound encompasses the delivery to a subject of a compound as described herein, or a prodrug or other pharmaceutically acceptable form thereof, using any suitable formulation or route of administration, as discussed herein.

The terms “disease”, “disorder” and “condition” are used interchangeably unless indicated otherwise.

As used herein, the terms “effective amount” or “therapeutically effective amount” refer to that amount of a compound or pharmaceutical composition described herein that is sufficient to effect the intended application including, but not limited to, disease treatment, as illustrated below.

In some embodiments, the amount is that effective for detectable killing or inhibition of the growth or spread of cancer cells; the size or number of tumors; or other measure of the level, stage, progression or severity of the cancer.

The therapeutically effective amount can vary depending upon the intended application, or the subject and disease condition being treated, e.g., the desired biological endpoint, the pharmacokinetics of the compound, the disease being treated, the mode of administration, and the weight and age of the patient, which can readily be determined by one of ordinary skill in the art. The term also applies to a dose that will induce a particular response in target cells, e.g., reduction of cell migration. The specific dose will vary depending on, for example, the particular compounds chosen, the species of subject and their age/existing health conditions or risk for health conditions, the dosing regimen to be followed, the severity of the disease, whether it is administered in combination with other agents, timing of administration, the tissue to which it is administered, and the physical delivery system in which it is carried.

The term “optionally substituted” is understood to mean that a given chemical moiety (e.g., an alkyl group) can (but is not required to) be bonded other substituents (e.g., heteroatoms). For instance, an alkyl group that is optionally substituted can be a fully saturated alkyl chain (i.e. a pure hydrocarbon). Alternatively, the same optionally substituted alkyl group can have substituents different from hydrogen. For instance, it can, at any point along the chain be bounded to a halogen atom, a hydroxyl group, or any other substituent described herein. Thus, the term “optionally substituted” means that a given chemical moiety has the potential to contain other functional groups, but does not necessarily have any further functional groups. Suitable substituents used in the optional substitution of the described groups include, without limitation, halogen, oxo, CN, —COOH, —CH₂CN, —O—C₁-C₆ alkyl, C₁-C₆ alkyl, —OC₁-C₆ alkenyl, —OC₁-C₆ alkynyl, —C₁-C₆ alkenyl, —C₁-C₆ alkynyl, —OH, —OP(O)(OH)₂, —OC(O)C₁-C₆ alkyl, —C(O)C₁-C₆ alkyl, —OC(O)OC₁-C₆ alkyl, NH₂, NH(C₁-C₆ alkyl), N(C₁-C₆ alkyl)₂, —NHC(O)C₁-C₆ alkyl, —C(O)NHC₁-C₆ alkyl, —S(O)₂—C₁-C₆ alkyl, —S(O)NHC₁-C₆ alkyl, and S(O)N(C₁-C₆ alkyl)₂.

As used herein, a “pharmaceutically acceptable form” of a disclosed compound includes, but is not limited to, pharmaceutically acceptable salts, esters, hydrates, solvates, isomers, prodrugs, and isotopically labeled derivatives of disclosed compounds. In one embodiment, a “pharmaceutically acceptable form” includes, but is not limited to, pharmaceutically acceptable salts, esters, isomers, prodrugs and isotopically labeled derivatives of disclosed compounds. In some embodiments, a “pharmaceutically acceptable form” includes, but is not limited to, pharmaceutically acceptable salts, esters, stereoisomers, prodrugs and isotopically labeled derivatives of disclosed compounds.

In certain embodiments, the pharmaceutically acceptable form is a pharmaceutically acceptable salt. As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, organic acids from which salts can be derived include, for example, acetic acid, propionic acid, glycolic acid, pyruvic acid, oxalic acid, lactic acid, trifluoracetic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

The salts can be prepared in situ during the isolation and purification of the disclosed compounds, or separately, such as by reacting the free base or free acid of a parent compound with a suitable base or acid, respectively. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, iron, zinc, copper, manganese, aluminum, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Organic bases from which salts can be derived include, for example, primary, secondary, and tertiary amines, substituted amines, including naturally occurring substituted amines, cyclic amines, basic ion exchange resins, and the like, such as isopropylamine, trimethylamine, diethylamine, triethylamine, tripropylamine, and ethanolamine. In some embodiments, the pharmaceutically acceptable base addition salt can be chosen from ammonium, potassium, sodium, calcium, and magnesium salts.

In certain embodiments, the pharmaceutically acceptable form is a pharmaceutically acceptable ester. As used herein, the term “pharmaceutically acceptable ester” refers to esters that hydrolyze in vivo and include those that break down readily in the human body to leave the parent compound or a salt thereof. Such esters can act as a prodrug as defined herein. Pharmaceutically acceptable esters include, but are not limited to, alkyl, alkenyl, alkynyl, aryl, aralkyl, and cycloalkyl esters of acidic groups, including, but not limited to, carboxylic acids, phosphoric acids, phosphinic acids, sulfinic acids, sulfonic acids and boronic acids. Examples of esters include formates, acetates, propionates, butyrates, acrylates and ethylsuccinates. The esters can be formed with a hydroxy or carboxylic acid group of the parent compound.

In certain embodiments, the pharmaceutically acceptable form is a “solvate” (e.g., a hydrate). As used herein, the term “solvate” refers to compounds that further include a stoichiometric or non-stoichiometric amount of solvent bound by non-covalent intermolecular forces. The solvate can be of a disclosed compound or a pharmaceutically acceptable salt thereof. Where the solvent is water, the solvate is a “hydrate”. Pharmaceutically acceptable solvates and hydrates are complexes that, for example, can include 1 to about 100, or 1 to about 10, or 1 to about 2, about 3 or about 4, solvent or water molecules. It will be understood that the term “compound” as used herein encompasses the compound and solvates of the compound, as well as mixtures thereof.

In certain embodiments, the pharmaceutically acceptable form is a prodrug. As used herein, the term “prodrug” (or “pro-drug”) refers to compounds that are transformed in vivo to yield a disclosed compound or a pharmaceutically acceptable form of the compound. A prodrug can be inactive when administered to a subject, but is converted in vivo to an active compound, for example, by hydrolysis (e.g., hydrolysis in blood). In certain cases, a prodrug has improved physical and/or delivery properties over the parent compound. Prodrugs can increase the bioavailability of the compound when administered to a subject (e.g., by permitting enhanced absorption into the blood following oral administration) or which enhance delivery to a biological compartment of interest (e.g., the brain or lymphatic system) relative to the parent compound. Exemplary prodrugs include derivatives of a disclosed compound with enhanced aqueous solubility or active transport through the gut membrane, relative to the parent compound.

The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Prodrugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. Exemplary advantages of a prodrug can include, but are not limited to, its physical properties, such as enhanced water solubility for parenteral administration at physiological pH compared to the parent compound, or it can enhance absorption from the digestive tract, or it can enhance drug stability for long-term storage.

As used herein, the term “pharmaceutically acceptable” excipient, carrier, or diluent refers to a pharmaceutically acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate, magnesium stearate, and polyethylene oxide-polypropylene oxide copolymer as well as coloring agents, release agents, coating agents, sweetening, flavoring and perfuming agents, preservatives and antioxidants can also be present in the compositions.

As used herein, the term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

As used herein, the terms “treatment” or “treating” a disease or disorder refers to a method of reducing, delaying or ameliorating such a condition before or after it has occurred. Treatment may be directed at one or more effects or symptoms of a disease and/or the underlying pathology. Treatment is aimed to obtain beneficial or desired results including, but not limited to, therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient can still be afflicted with the underlying disorder. For prophylactic benefit, the pharmaceutical compounds and/or compositions can be administered to a patient at risk of developing a particular disease, or to a patient reporting one or more of the physiological symptoms of a disease, even though a diagnosis of this disease may not have been made. The treatment can be any reduction and can be, but is not limited to, the complete ablation of the disease or the symptoms of the disease. As compared with an equivalent untreated control, such reduction or degree of prevention is at least 5%, 10%, 20%, 40%, 50%, 60%, 80%, 90%, 95%, or 100% as measured by any standard technique.

As used herein, the term “therapeutic effect” refers to a therapeutic benefit and/or a prophylactic benefit as described herein. A prophylactic effect includes delaying or eliminating the appearance of a disease or condition, delaying or eliminating the onset of symptoms of a disease or condition, slowing, halting, or reversing the progression of a disease or condition, or any combination thereof.

Compounds of the present invention are, subsequent to their preparation, preferably isolated and purified to obtain a composition containing an amount by weight equal to or greater than 95% (“substantially pure”), which is then used or formulated as described herein. In certain embodiments, the compounds of the present invention are more than 99% pure.

Solvates and polymorphs of the compounds of the invention are also contemplated herein. Solvates of the compounds of the present invention include, for example, hydrates.

As used herein, the term an “isolated” or “substantially isolated” molecule (such as a polypeptide or polynucleotide) is one that has been manipulated to exist in a higher concentration than in nature or has been removed from its native environment. For example, a subject antibody is isolated, purified, substantially isolated, or substantially purified when at least 10%, or 20%, or 40%, or 50%, or 70%, or 90% of non-subject-antibody materials with which it is associated in nature have been removed. For example, a polynucleotide or a polypeptide naturally present in a living animal is not “isolated,” but the same polynucleotide or polypeptide separated from the coexisting materials of its natural state is “isolated.” Further, recombinant DNA molecules contained in a vector are considered isolated for the purposes of the present invention. Isolated RNA molecules include in vivo or in vitro RNA replication products of DNA and RNA molecules. Isolated nucleic acid molecules further include synthetically produced molecules. Additionally, vector molecules contained in recombinant host cells are also isolated. Thus, not all “isolated” molecules need be “purified.”

As used herein, the term “purified” when used in reference to a molecule, it means that the concentration of the molecule being purified has been increased relative to molecules associated with it in its natural environment, or environment in which it was produced, found or synthesized. Naturally associated molecules include proteins, nucleic acids, lipids and sugars but generally do not include water, buffers, and reagents added to maintain the integrity or facilitate the purification of the molecule being purified. According to this definition, a substance may be 5% or more, 10% or more, 20% or more, 30% or more, 40% or more, 50% or more, 60% or more, 70% or more, 80% or more, 90% or more, 95% or more, 98% or more, 99% or more, or 100% pure when considered relative to its contaminants.

Definitions of specific functional groups and chemical terms are described in more detail below. When a range of values is listed, it is intended to encompass each value and sub-range within the range. For example, “C₁₋₄ alkyl” is intended to encompass, C₁, C₂, C₃, C₄, C₁₋₃, C₁₋₂, C₂₋₄, C₃₋₄ and C₂₋₃ alkyl groups.

As used herein, the term “alkyl” refers to a straight or branched hydrocarbon chain radical consisting solely of carbon and hydrogen atoms, containing no unsaturation, having from one to ten carbon atoms (e.g., C₁₋₁₀ alkyl). Whenever it appears herein, a numerical range such as “1 to 10” refers to each integer in the given range; e.g., “1 to 10 carbon atoms” means that the alkyl group can consist of 1 carbon atom, 2 carbon atoms, 3 carbon atoms, etc., up to and including 10 carbon atoms, although the present definition also covers the occurrence of the term “alkyl” where no numerical range is designated. In some embodiments, “alkyl” can be a C₁₋₆ alkyl group. In some embodiments, alkyl groups have 1 to 10, 1 to 8, 1 to 6, or 1 to 3 carbon atoms. Representative saturated straight chain alkyls include, but are not limited to, -methyl, -ethyl, -n-propyl, -n-butyl, -n-pentyl, and -n-hexyl; while saturated branched alkyls include, but are not limited to, -isopropyl, -sec-butyl, -isobutyl, -tert-butyl, -isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, and the like. The alkyl is attached to the parent molecule by a single bond. Unless stated otherwise in the specification, an alkyl group is optionally substituted by one or more of substituents which independently include: acyl, alkyl, alkenyl, alkynyl, alkoxy, alkylaryl, cycloalkyl, aralkyl, aryl, aryloxy, amino, amido, amidino, imino, azide, carbonate, carbamate, carbonyl, heteroalkyl, heteroaryl, heteroarylalkyl, heterocycloalkyl, hydroxy, cyano, halo, haloalkoxy, haloalkyl, ester, ether, mercapto, thio, alkylthio, arylthio, thiocarbonyl, nitro, oxo, phosphate, phosphonate, phosphinate, silyl, sulfinyl, sulfonyl, sulfonamidyl, sulfoxyl, sulfonate, urea, —Si(R^{a})₃—OR^{a}, —SR^{a}, —OC(O)—R^{a}, —N(R^{a})₂, —C(O)Rₐ, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜN(R^{a})₂ (where t is 1 or 2), —P(═O)(R^{a})(R^{a}), or —O—P(═O)(OR^{a})₂ where each R^{a} is independently hydrogen, alkyl, haloalkyl, carbocyclyl, carbocyclylalkyl, aryl, aralkyl, heterocycloalkyl, heterocycloalkylalkyl, heteroaryl or heteroarylalkyl, and each of these moieties can be optionally substituted as defined herein. In a non-limiting embodiment, a substituted alkyl can be selected from fluoromethyl, difluoromethyl, trifluoromethyl, 2-fluoroethyl, 3-fluoropropyl, hydroxymethyl, 2-hydroxyethyl, 3-hydroxypropyl, benzyl, and phenethyl.

Unless otherwise specifically defined, the term “aromatic” or “aryl” refers to cyclic, aromatic hydrocarbon groups that have 1 to 2 aromatic rings, including monocyclic or bicyclic groups such as phenyl, biphenyl or naphthyl. Where containing two aromatic rings (bicyclic, etc.), the aromatic rings of the aryl group may be joined at a single point (e.g., biphenyl), or fused (e.g., naphthyl). The aryl group may be optionally substituted by one or more substituents, e.g., 1 to 5 substituents, at any point of attachment. Exemplary substituents include, but are not limited to, H, halogen, —O—C₁-C₆ alkyl, C₁-C₆ alkyl, —C₁-C₆ alkenyl, —OC₁-C₆ alkynyl, —C₁-C₆ alkenyl, —C₁-C₆ alkynyl, —OH, —OP(O)(OH)₂, —OC(O)C₁-C₆ alkyl, —C(O)C₁-C₆ alkyl, —OC(O)OC₁-C₆alkyl, NH₂, NH(C₁-C₆ alkyl), N(C₁-C₆ alkyl)₂, —S(O)₂—C₁-C₆ alkyl, —S(O)NHC₁-C₆alkyl, and S(O)N(C₁-C₆ alkyl)₂. The substituents can themselves be optionally substituted. Furthermore, when containing two fused rings the aryl groups herein defined may have an unsaturated or partially saturated ring fused with a fully unsaturated ring. Exemplary ring systems of these aryl groups include indanyl, indenyl, tetrahydronaphthalenyl, and tetrahydrobenzoannulenyl.

The term “halogen” or “halo” refers to fluorine (F), chlorine (Cl), bromine (Br) and iodine (I).

Unless otherwise specifically defined, the terms “heteroaryl” or “hetero-aromatic” as used herein, means a monocyclic heteroaryl ring or a bicyclic heteroaryl ring. The monocyclic heteroaryl ring is a 5- or 6-membered ring. The 5-membered ring has two double bonds and contains one, two, three or four heteroatoms independently selected from the group consisting of N, O, and S. The 6-membered ring has three double bonds and contains one, two, three or four heteroatoms independently selected from the group consisting of N, O, and S. The bicyclic heteroaryl ring consists of the 5- or 6-membered heteroaryl ring fused to a phenyl group or the 5- or 6-membered heteroaryl ring fused to a cycloalkyl group or the 5- or 6-membered heteroaryl ring fused to a cycloalkenyl group or the 5- or 6-membered heteroaryl ring fused to another 5- or 6-membered heteroaryl ring. Nitrogen heteroatoms contained within the heteroaryl may be optionally oxidized to the N-oxide or optionally protected with a nitrogen protecting group known to those of skill in the art. The heteroaryl is connected to the parent molecular moiety through any carbon atom or any nitrogen atom contained within the heteroaryl. Representative examples of heteroaryl include, but are not limited to, benzothienyl, benzoxadiazolyl, cirmolinyl, 5,6-dihydroisoquinolinyl, 7,8-dihydroisoquinolinyl, 5,6-dihydroquinolinyl, 7,8-dihydroquinolinyl, furopyridinyl, furyl, imidazolyl, indazolyl, indolyl, isoxazolyl, isoquinolinyl, isothiazolyl, naphthyridinyl, oxadiazolyl, oxazolyl, pyridinyl, pyridazinyl, pyrimidinyl, pyrazinyl, pyrazolyl, pyrrolyl, pyridinium N-oxide, quinolinyl, 5,6,7,8-tetrahydroisoquinolinyl, 5,6,7,8-tetrahydroquinolinyl, tetrazolyl, thiadiazolyl, thiazolyl, thienopyridinyl, thienyl, triazolyl, and triazinyl.

The terms “heteroaryl” or “hetero-aromatic” groups of the present invention are substituted with 0, 1, 2, 3, or 4 substituents independently selected from alkenyl, alkoxy, alkoxyalkoxy, alkoxyalkyl, alkoxycarbonyl, alkoxycarbonylalkyl, alkyl, alkylcarbonyl, alkylcarbonylalkyl, alkylcarbonyloxy, alkylthio, alkylthioalkyl, alkynyl, carboxy, carboxyalkyl, cyano, cyanoalkyl, formyl, haloalkoxy, haloalkyl, halogen, hydroxy, hydroxyalkyl, mercapto, nitro, —NZ₁Z₂, and (NZ₁Z₂)carbonyl. The term “NZ₁Z₂” as used herein, means two groups, Z₁ and Z₂, which are appended to the parent molecular moiety through a nitrogen atom. Z₁ and Z₂ are each independently selected from the group consisting of hydrogen, alkyl, alkylcarbonyl, and formyl. Representative examples of NZ₁Z₂ include, but are not limited to, amino, methylamino, acetylamino, and acetylmethylamino.

As used herein, the term “alkoxy” refers to an —O-alkyl radical.

As used herein, the terms “cycloalkyl” and “carbocyclyl” each refers to a monocyclic or polycyclic radical that contains only carbon and hydrogen, and can be saturated or partially unsaturated. Unless stated otherwise in the specification, the term is intended to include both substituted and unsubstituted cycloalkyl groups. Partially unsaturated cycloalkyl groups can be termed “cycloalkenyl” if the carbocycle contains at least one double bond, or “cycloalkynyl” if the carbocycle contains at least one triple bond. Cycloalkyl groups include groups having from 3 to 13 ring atoms (i.e., C₃₋₁₃ cycloalkyl). Whenever it appears herein, a numerical range such as “3 to 10” refers to each integer in the given range; e.g., “3 to 13 carbon atoms” means that the cycloalkyl group can consist of 3 carbon atoms, 4 carbon atoms, 5 carbon atoms, etc., up to and including 13 carbon atoms. The term “cycloalkyl” also includes bridged and spiro-fused cyclic structures containing no heteroatoms. The term also includes monocyclic or fused-ring polycyclic (i.e., rings which share adjacent pairs of ring atoms) groups. Polycyclic aryl groups include bicycles, tricycles, tetracycles, and the like. In some embodiments, “cycloalkyl” can be a C₃₋₈ cycloalkyl radical. In some embodiments, “cycloalkyl” can be a C₃₋₅ cycloalkyl radical. Illustrative examples of cycloalkyl groups include, but are not limited to the following moieties: C₃₋₆ carbocyclyl groups include, without limitation, cyclopropyl (C₃), cyclobutyl (C₄), cyclopentyl (C₅), cyclopentenyl (C₅), cyclohexyl (C₆), cyclohexenyl (C₆), cyclohexadienyl (C₆) and the like. Examples of C₃₋₇ carbocyclyl groups include norbornyl (C₇). Examples of C₃₋₈ carbocyclyl groups include the aforementioned C₃₋₇ carbocyclyl groups as well as cycloheptyl (C₇), cycloheptadienyl (C₇), cycloheptatrienyl (C₇), cyclooctyl (C₈), bicyclo[2.2.1]heptanyl, bicyclo[2.2.2]octanyl, and the like. Examples of C₃₋₁₃ carbocyclyl groups include the aforementioned C₃₋₈ carbocyclyl groups as well as octahydro-1H indenyl, decahydronaphthalenyl, spiro[4.5]decanyl and the like. Unless stated otherwise in the specification, a cycloalkyl group can be optionally substituted by one or more substituents which independently include: acyl, alkyl, alkenyl, alkynyl, alkoxy, alkylaryl, cycloalkyl, aralkyl, aryl, aryloxy, amino, amido, amidino, imino, azide, carbonate, carbamate, carbonyl, heteroalkyl, heteroaryl, heteroarylalkyl, heterocycloalkyl, hydroxy, cyano, halo, haloalkoxy, haloalkyl, ester, ether, mercapto, thio, alkylthio, arylthio, thiocarbonyl, nitro, oxo, phosphate, phosphonate, phosphinate, silyl, sulfinyl, sulfonyl, sulfonamidyl, sulfoxyl, sulfonate, urea, —Si(R^{a})₃, —OR^{a}, —SR^{a}, —OC(O)—R^{a}, —N(R^{a})₂, —C(O)Rₐ, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜN(R^{a})₂ (where t is 1 or 2), —P(═O)(R^{a})(R^{a}), or —O—P(═O)(OR^{a})₂ where each R^{a} is independently hydrogen, alkyl, haloalkyl, carbocyclyl, carbocyclylalkyl, aryl, aralkyl, heterocycloalkyl, heterocycloalkylalkyl, heteroaryl or heteroarylalkyl, and each of these moieties can be optionally substituted as defined herein. The terms “cycloalkenyl” and “cycloalkynyl” mirror the above description of “cycloalkyl” wherein the prefix “alk” is replaced with “alken” or “alkyn” respectively, and the parent “alkenyl” or “alkynyl” terms are as described herein. For example, a cycloalkenyl group can have 3 to 13 ring atoms, such as 5 to 8 ring atoms. In some embodiments, a cycloalkynyl group can have 5 to 13 ring atoms.

As used herein, the term “heterocycloalkyl” refers to a cycloalkyl radical, which have one or more skeletal chain atoms selected from an atom other than carbon, e.g., O, N, S, P or combinations thereof. Unless stated otherwise in the specification, the term is intended to include both substituted and unsubstituted heterocycloalkyl groups. Illustrative examples of heterocycloalkyl include 2-hydroxy-aziridin-1-yl, 3-oxo-1-oxacyclobutan-2-yl, 2,2-dimethyl-tetrahydrofuran-3-yl, 3-carboxy-morpholin-4-yl, 1-cyclopropyl-4-methyl-piperazin-2-yl. 2-pyrrolinyl, 3-pyrrolinyl, dihydro-2H-pyranyl, 1,2,3,4-tetrahydropyridine, 3,4-dihydro-2H-[1,4]oxazine, etc.

As used herein, the terms “heterocycle”, “heterocyclic” or “heterocyclo” refer to fully saturated or partially unsaturated cyclic groups, for example, 3 to 7 membered monocyclic, 7 to 12 membered bicyclic, or 10 to 15 membered tricyclic ring systems, which have at least one heteroatom in at least one ring, wherein 0, 1, 2 or 3 atoms of each ring may be substituted by a substituent. Each ring of the heterocyclic group containing a heteroatom may have 1, 2, 3 or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. The heterocyclic group may be attached at any heteroatom or carbon atom of the ring or ring system.

### DETAILED DESCRIPTION OF THE INVENTION

The invention is based in part on the discovery of novel PI3Kβ inhibitors, pharmaceutical compositions thereof and methods of their preparation and use in therapy of various diseases and conditions, such as cancers (e.g., breast, colon, endometrial, kidney, lung, melanoma, prostate, thyroid cancer and leukemia).

In one aspect, the invention generally relates to a compound having the structural formula (I).

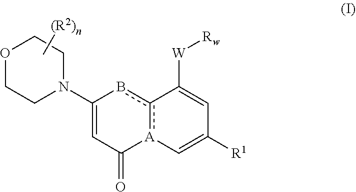

wherein
A represents carbon and B represents oxygen, or each of A and B represent nitrogen;
W represents —CHR-Q- or -Q-CHR—; wherein Q represents a bond, O, S, CH₂ or NR′; R and R′ is independently selected from hydrogen, C₁₋₄alkyl or C₁₋₄alkyl substituted with —OH or halo;
R_{w} represents Ar or a substituted or unsubstituted indoline group, wherein Ar is a group comprising a substituted or unsubstituted 6-membered aromatic or hetero-aromatic ring;
R² represents C₁₋₃ alkyl, CN, CH₂OH, CH₂F or CONH₂;
R¹ represents H, NH₂, OH, CN, CF₃, CHO, COC₁₋₄alkyl, CH₂C(═O)—NR^{1a}R^{1b}, C(═O)—NR^{1a}R^{1b}, CH₂COOR^{1c}, NR^{1d}R^{1e}, C₁₋₄ alkyl, O—C₁₋₄ alkyl, Het, Ar, NR^{1h}(C═O)R¹ⁱ, NR^{1j}(C═O)NR^{1k}R^{1l}, NR^{1j}(SO₂)R^{1m}, SO₂NR¹ⁿR^{1o}, NR^{1j}(C═O)C(CN)C(OH)R^{1p}, P(O)MeMe, P(O)OMeOMe; provided that, when A is carbon, B is oxygen and R_{w} is Ar, R¹ represents NH₂, CN, CHO, COC₁₋₄alkyl, CH₂C(═O)—NR^{1a}R^{1b}, CH₂COOR^{1c}, NR^{1d}R^{1e}, O—C₂₋₄ alkyl, Het, NR^{1h}(C═O)R¹ⁱ, NR^{1j}(C═O)NR^{1k}R^{1l}, NR^{1j}(SO₂)R^{1m}, SO₂NR¹ⁿR^{1o}, NR^{1j}(C═O)C(CN)C(OH)R^{1p}, P(O)MeMe, P(O)OMeOMe; when A and B is nitrogen, R_{w} is Ar, R¹ represents NH₂, CN, CHO, COC₁₋₄alkyl, CH₂C(═O)—NR^{1a}R^{1b}, CH₂COOR^{1c}, NR^{1d}R^{1e}, O—C₂₋₄ alkyl, Het, NR^{1h}(C═O)R¹ⁱ, NR^{1j}(C═O)NR^{1k}R^{1l}, NR^{1j}(SO₂)R^{1m}, SO₂NR¹ⁿR^{1o}, NR^{1j}(C═O)C(CN)C(OH)R^{1p}, P(O)MeMe, P(O)OMeOMe, C(═O)—NR^{1q}R^{1r}, wherein R^{1q} and R^{1r} together form a 3- to 8-membered nitrogen-containing heterocyclyl ring with one or more substituent selected from the group consisting of C₁₋₃ alkyl and NR^{3a}R^{3b};
wherein each of R^{1a}, R^{1b}, R^{1c}, R^{1d}, R^{1e}, R^{1h}, R¹ⁱ, R^{1j}, R^{1k}, R^{1l}, R^{1m}, R¹ⁿ, R^{1o}, R^{1p} is independently selected from H, C₁₋₄ alkyl, C₃₋₈cycloalkyl, C₃₋₈heterocycloakyl or C₁₋₄ alkyl substituted with one or more substituent selected from the group consisting of hydroxyl, OMe, CN, fluoro, NR^{3a}R^{3b}, C₃₋₈cycloalkyl, C₃₋₈heterocycloakyl; R^{1a} and R^{1b}, R^{1d} and R^{1e}, R^{1m} and R^{1o} are optionally together form a 3- to 8-membered nitrogen-containing heterocyclyl ring with one or more substituent selected from the group consisting of C₁₋₃ alkyl and NR^{3a}R^{3b},
C₁₋₄ alkyl is unsubstituted or substituted with one or more substituent selected from the group consisting of OH, F, NR^{1d}R^{1e},
R^{3a} and R^{3b} each independently are selected from the group consisting of H, and C₁₋₄ alkyl;
Het represents a 3- to 8-membered saturated or partially saturated monocyclic, bridged or spiro heterocyclyl containing at least one heteroatom each independently selected from O, S, S(═O)ₚ and N; which substituted wish one or two substituents each independently selected from the group consisting of halo, NR^{3a}R^{3b}, C₁₋₄ alkyl, OH, OMe and CN;
n represents 0, 1, 2, 3 or 4; and
p represents 1 or 2,
   or a pharmaceutically acceptable form or an isotope derivative thereof.

In certain embodiments, A is carbon and B is oxygen, having the structural formula (Ia):

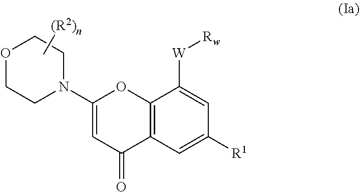

In certain embodiments of formula (Ia), R_{w} is Ar. In certain embodiments, W is —CHR⁴—NR⁵—, wherein each of R⁴ and R⁵ is independently selected from H, methyl or ethyl.

In certain embodiments of formula (Ia), R_{w} is a substituted or unsubstituted indoline group. In certain embodiments, W is —CHR⁴, wherein R⁴ is selected from H, methyl or ethyl.

In certain embodiments, each of A and B is nitrogen, having the structural formula (Ib).

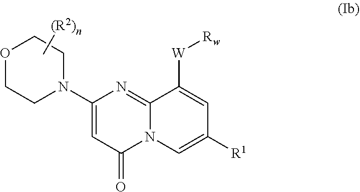

In certain embodiments of formula (Ib), R_{w} is Ar. In certain embodiments, W is —CHR⁴—NR⁵—, wherein each of R⁴ and R⁵ is independently selected from H, methyl or ethyl.

In certain embodiments of formula (Ib), R_{w} is a substituted or unsubstituted indoline group. In certain embodiments, W is —CHR⁴, wherein R⁴ is selected from H, methyl or ethyl.

In certain embodiments of formula (I), (Ia) or (Ib), each of R⁴ and R⁵ is H.

In certain embodiments of formula (I), (Ia) or (Ib), at least one of R⁴ and R⁵ is methyl or ethyl.

In certain embodiments of formula (I), (Ia) or (Ib), the Ar or the indoline group is substituted with one or more substitution groups selected from the group consisting of halogen, C₁₋₄ alkyl, OC₁₋₄ alkyl, CN, CHF₂, CF₃ and CH₂OH.

In certain embodiments of formula (I), (Ia) or (Ib), the Ar or the indoline group is substituted with at least one F atom. In certain embodiments (I), (Ia) or (Ib), the Ar or the indoline group is substituted with two one F atoms.

In certain embodiments of formula (I), (Ia) or (Ib), W—R_{w} is selected from:

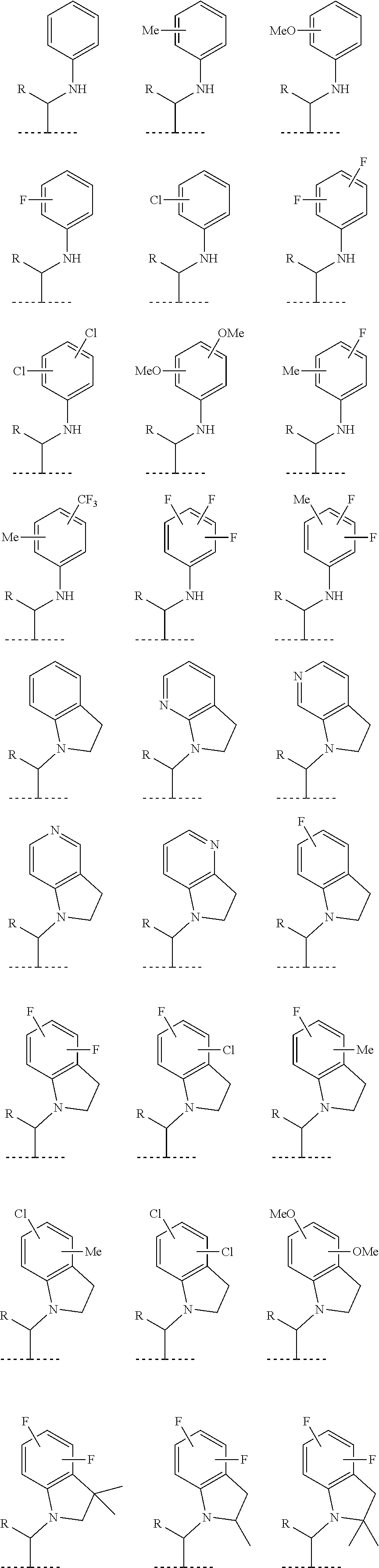

wherein R is independently selected from hydrogen, C₁₋₄alkyl or C₁₋₄alkyl substituted with —OH or halo.

In certain embodiments of formula (I), (Ia) or (Ib), n is 0.

In certain embodiments of formula (I), (Ia) or (Ib), n is 1.

In certain embodiments of formula (I), (Ia) or (Ib), R² is a C₁₋₃ alkyl. In certain embodiments (I), (Ia) or (Ib), R² is CH₂OH, CN or CONH₂.

In certain embodiments of formula (I), (Ia) or (Ib), R¹ is C(═O)—NR^{1a}R^{1b}, wherein each of R^{1a} and R^{1b} is independently selected from H and C₁₋₄ alkyl.

In certain embodiments of formula (I), (Ia) or (Ib), R¹ is C(═O)—NR^{1q}R^{1r}, wherein R^{1q} and R^{1r} together form a 3- to 8-membered nitrogen-containing heterocyclyl ring optionally substituted with one of more of C₁₋₃ alkyl and NR^{3a}R^{3b}.

In certain embodiments, R^{1q} and R^{1r} together form a 5-membered nitrogen-containing heterocyclyl ring substituted with a C₁₋₃ alkyl.

In certain embodiments, R^{1q} and R^{1r} together form a 5-membered nitrogen-containing heterocyclyl ring substituted with NR^{3a}R^{3b}.

In certain embodiments, each of R^{3a} and R^{3b} is methyl.

In certain embodiments of formula (I), (Ia) or (Ib), R¹ is C₁₋₄ alkyl, optionally substituted with hydroxy, —O—C₁₋₄ alkyl and NR^{1c}R^{1d}.

In certain embodiments of formula (I), (Ia) or (Ib), R¹ is selected from:

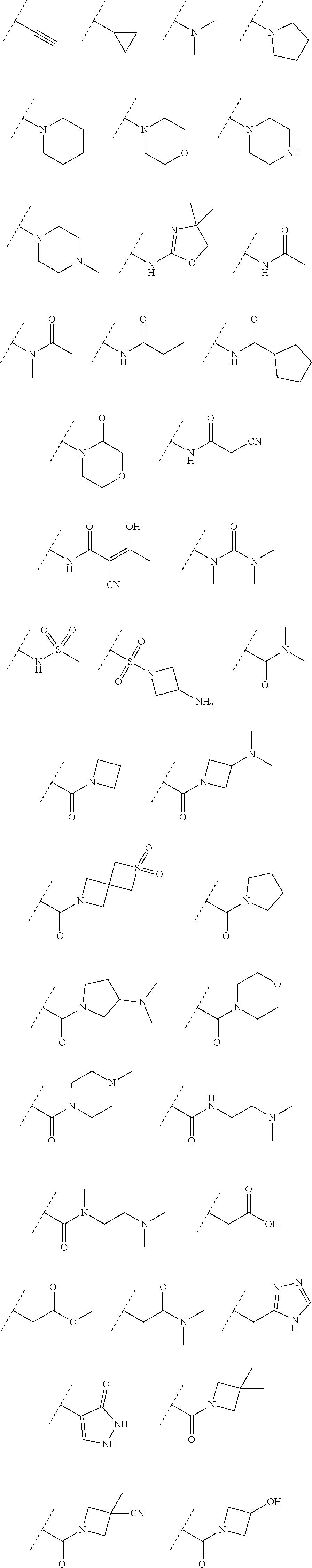

Examples of compounds according to the invention include, but are not limited to

| Entry | Compd | Structure | Name |
|---|---|---|---|
| 1 | Compound 9 | 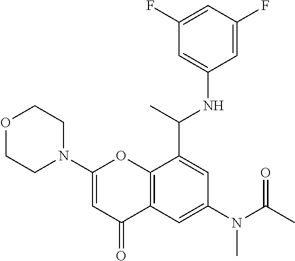 [SMILES: CC(=O)N(C)c1cc(C(C)Nc2cc(F)cc(F)c2)c2oc(N3CCOCC3)cc(=O)c2c1] | N-(8-(1-((3,5- difluorophenyl)amino)ethyl)-2- morpholino-4-oxo-4H-chromen-6- yl)-N-methylacetamide |
| 2 | Compound 10 | 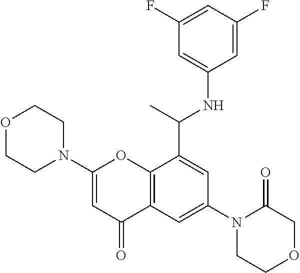 [SMILES: CC(Nc1cc(F)cc(F)c1)c1cc(N2CCOCC2=O)cc2c(=O)cc(N3CCOCC3)oc12] | 4-(8-(1-((3,5- difluorophenyl)amino)ethyl)-2- morpholino-4-oxo-4H-chromen-6- yl)morpholin-3-one |
| 3 | Compound 11 | 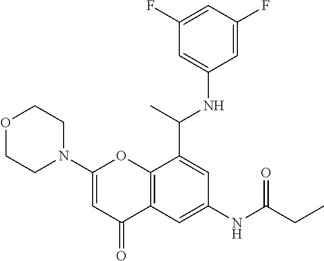 [SMILES: CCC(=O)Nc1cc(C(C)Nc2cc(F)cc(F)c2)c2oc(N3CCOCC3)cc(=O)c2c1] | N-(8-(1-((3,5- difluorophenyl)amino)ethyl)-2- morpholino-4-oxo-4H-chromen-6- yl)propionamide |
| 4 | Compound 12 | 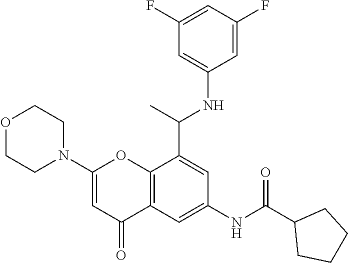 [SMILES: CC(Nc1cc(F)cc(F)c1)c1cc(NC(=O)C2CCCC2)cc2c(=O)cc(N3CCOCC3)oc12] | N-(8-(1-((3,5- difluorophenyl)amino)ethyl)-2- morpholino-4-oxo-4H-chromen-6- yl)cyclopentanecarboxamide |
| 5 | Compound 13 | 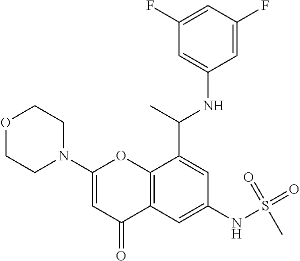 [SMILES: CC(Nc1cc(F)cc(F)c1)c1cc(NS(C)(=O)=O)cc2c(=O)cc(N3CCOCC3)oc12] | N-(8-(1-((3,5- difluorophenyl)amino)ethyl)-2- morpholino-4-oxo-4H-chromen-6- yl)methanesulfonamide |
| 6 | Compound 15 | 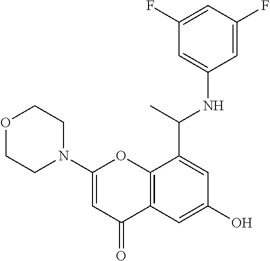 [SMILES: CC(Nc1cc(F)cc(F)c1)c1cc(O)cc2c(=O)cc(N3CCOCC3)oc12] | 8-(1-((3,5- difluorophenyl)amino)ethyl)-6- hydroxy-2-morpholino-4H- chromen-4-one |
| 7 | Compound 16 | 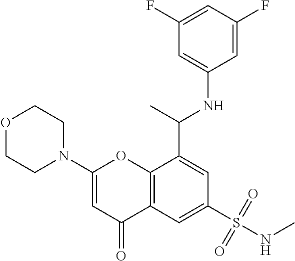 [SMILES: CNS(=O)(=O)c1cc(C(C)Nc2cc(F)cc(F)c2)c2oc(N3CCOCC3)cc(=O)c2c1] | 8-(1-((3,5- difluorophenyl)amino)ethyl)-N- methyl-2-morpholino-4-oxo-4H- chromene-6-sulfonamide |
| 8 | Compound 19 | 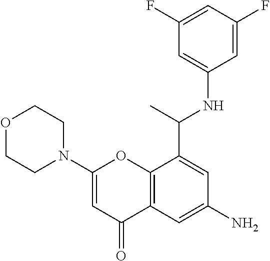 [SMILES: CC(Nc1cc(F)cc(F)c1)c1cc(N)cc2c(=O)cc(N3CCOCC3)oc12] | 8-(1-((3,5- difluorophenyl)amino)ethyl)-6- hydroxy-2-morpholino-4H- chromen-4-one |
| 9 | Compound 21 | 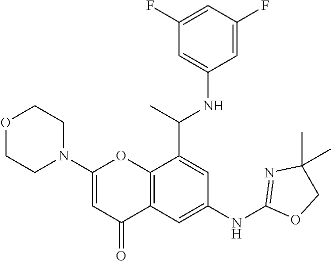 [SMILES: CC(Nc1cc(F)cc(F)c1)c1cc(NC2=NC(C)(C)CO2)cc2c(=O)cc(N3CCOCC3)oc12] | 8-(1-((3,5- difluorophenyl)amino)ethyl)-6- ((4,4-dimethyl-4,5-dihydrooxazol- 2-yl)amino)-2-morpholino-4H- chromen-4-one |
| 10 | Compound 22 | 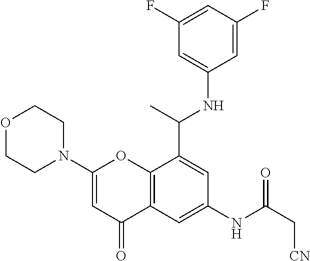 [SMILES: CC(Nc1cc(F)cc(F)c1)c1cc(NC(=O)CC#N)cc2c(=O)cc(N3CCOCC3)oc12] | 2-cyano-N-(8-(1-((3,5- difluorophenyl)amino)ethyl)-2- morpholino-4-oxo-4H-chromen-6- yl)acetamide |
| 11 | Compound 24 | 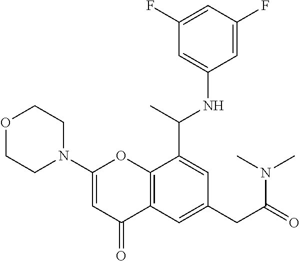 [SMILES: CC(Nc1cc(F)cc(F)c1)c1cc(CC(=O)N(C)C)cc2c(=O)cc(N3CCOCC3)oc12] | 2-(8-(1-((3,5- difluorophenyl)amino)ethyl)-2- morpholino-4-oxo-4H-chromen-6- yl)-N,N-dimethylacetamide |
| 12 | Compound 25 | 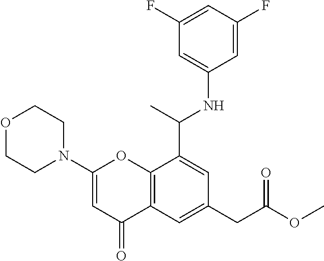 [SMILES: COC(=O)Cc1cc(C(C)Nc2cc(F)cc(F)c2)c2oc(N3CCOCC3)cc(=O)c2c1] | methyl 2-(8-(1-((3,5- difluorophenyl)amino)ethyl)-2- morpholino-4-oxo-4H-chromen-6- yl)acetate |
| 13 | Compound 27 | 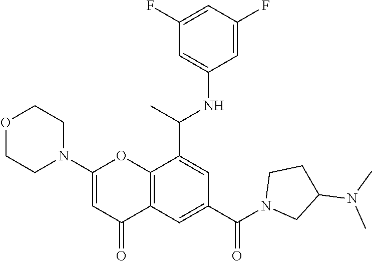 [SMILES: CC(Nc1cc(F)cc(F)c1)c1cc(C(=O)N2CCC(N(C)C)C2)cc2c(=O)cc(N3CCOCC3)oc12] | 8-(1-((3,5- difluorophenyl)amino)ethyl)-6-(3- (dimethylamino)pyrrolidine-1- carbonyl)-2-morpholino-4H- chromen-4-one |
| 14 | Compound 29 | 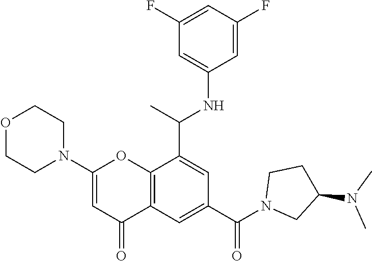 [SMILES: CC(Nc1cc(F)cc(F)c1)c1cc(C(=O)N2CC[C@@H](N(C)C)C2)cc2c(=O)cc(N3CCOCC3)oc12] | 8-(1-((3,5- difluorophenyl)amino)ethyl)-6-((R)- 3-(dimethylamino)pyrrolidine-1- carbonyl)-2-morpholino-4H- chromen-4-one |
| 15 | Compound 30 | 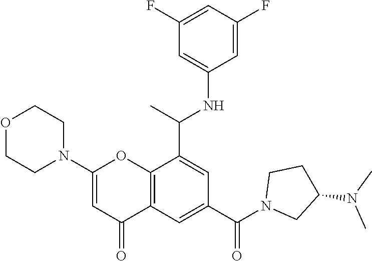 [SMILES: CC(Nc1cc(F)cc(F)c1)c1cc(C(=O)N2CC[C@H](N(C)C)C2)cc2c(=O)cc(N3CCOCC3)oc12] | 8-(1-((3,5- difluorophenyl)amino)ethyl)-6-((S)- 3-(dimethylamino)pyrrolidine-1- carbonyl)-2-morpholino-4H- chromen-4-one |
| 16 | Compound 31 | 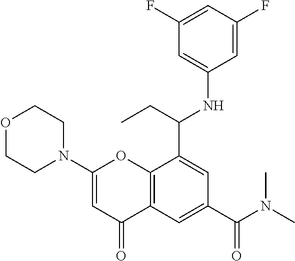 [SMILES: CCC(Nc1cc(F)cc(F)c1)c1cc(C(=O)N(C)C)cc2c(=O)cc(N3CCOCC3)oc12] | 8-(1-((3,5- difluorophenyl)amino)propyl)-N,N- dimethyl-2-morpholino-4-oxo-4H- chromene-6-carboxamide |
| 17 | Compound 32 | 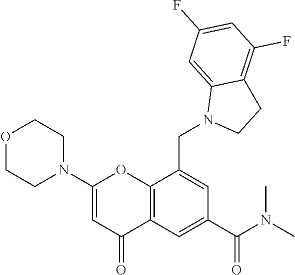 [SMILES: CN(C)C(=O)c1cc(CN2CCc3c(F)cc(F)cc32)c2oc(N3CCOCC3)cc(=O)c2c1] | 8-((4,6-difluoroindolin-1- yl)methyl)-N,N-dimethyl-2- morpholino-4-oxo-4H-chromene-6- carboxamide |
| 18 | Compound 33 | 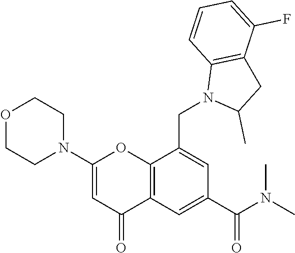 [SMILES: CC1Cc2c(F)cccc2N1Cc1cc(C(=O)N(C)C)cc2c(=O)cc(N3CCOCC3)oc12] | 8-((4-fluoro-2-methylindolin-1- yl)methyl)-N,N-dimethyl-2- morpholino-4-oxo-4H-chromene-6- carboxamide |
| 19 | Compound 34 | 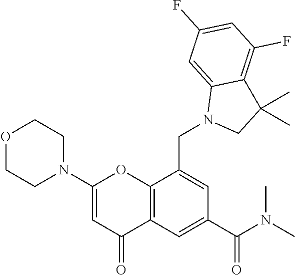 [SMILES: CN(C)C(=O)c1cc(CN2CC(C)(C)c3c(F)cc(F)cc32)c2oc(N3CCOCC3)cc(=O)c2c1] | 8-((4,6-difluoro-3,3- dimethylindolin-1-yl)methyl)-N,N- dimethyl-2-morpholino-4-oxo-4H- chromene-6-carboxamide |
| 20 | Compound 36 | 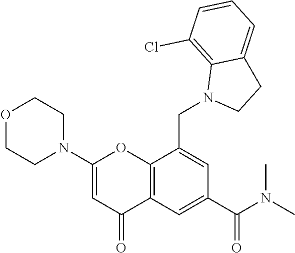 [SMILES: CN(C)C(=O)c1cc(CN2CCc3cccc(Cl)c32)c2oc(N3CCOCC3)cc(=O)c2c1] | 8-((7-chloroindolin-1-yl)methyl)- N,N-dimethyl-2-morpholino-4-oxo- 4H-chromene-6-carboxamide |
| 21 | Compound 37 | 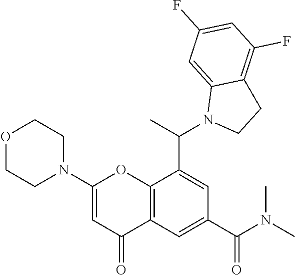 [SMILES: CC(c1cc(C(=O)N(C)C)cc2c(=O)cc(N3CCOCC3)oc12)N1CCc2c(F)cc(F)cc21] | 8-(1-(4,6-difluoroindolin-1- yl)ethyl)-N,N-dimethyl-2- morpholino-4-oxo-4H-chromene-6- carboxamide |
| 22 | Compound 38 | 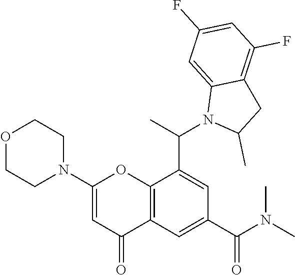 [SMILES: CC1Cc2c(F)cc(F)cc2N1C(C)c1cc(C(=O)N(C)C)cc2c(=O)cc(N3CCOCC3)oc12] | 8-(1-(4,6-difluoro-2-methylindolin- 1-yl)ethyl)-N,N-dimethyl-2- morpholino-4-oxo-4H-chromene-6- carboxamide |
| 23 | Compound 39 | 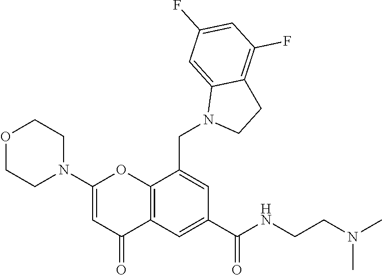 [SMILES: CN(C)CCNC(=O)c1cc(CN2CCc3c(F)cc(F)cc32)c2oc(N3CCOCC3)cc(=O)c2c1] | 8-((4,6-difluoroindolin-1- yl)methyl)-N-(2- (dimethylamino)ethyl)-2- morpholino-4-oxo-4H-chromene-6- carboxamide |
| 24 | Compound 40 | 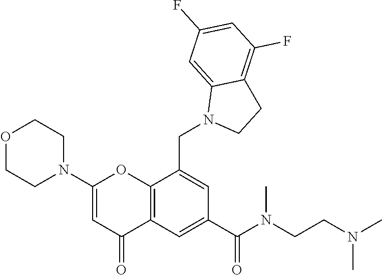 [SMILES: CN(C)CCN(C)C(=O)c1cc(CN2CCc3c(F)cc(F)cc32)c2oc(N3CCOCC3)cc(=O)c2c1] | 8-((4,6-difluoroindolin-1- yl)methyl)-N-(2- (dimethylamino)ethyl)-N-methyl-2- morpholino-4-oxo-4H-chromene-6- carboxamide |
| 25 | Compound 41 | 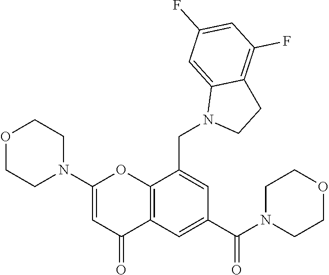 [SMILES: O=C(c1cc(CN2CCc3c(F)cc(F)cc32)c2oc(N3CCOCC3)cc(=O)c2c1)N1CCOCC1] | 8-((4,6-difluoroindolin-1- yl)methyl)-6-(morpholine-4- carbonyl)-2-morpholino-4H- chromen-4-one |
| 26 | Compound 42 | 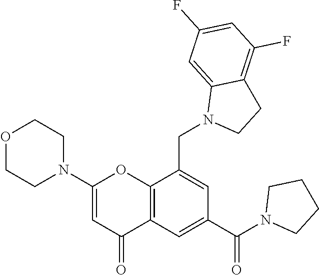 [SMILES: O=C(c1cc(CN2CCc3c(F)cc(F)cc32)c2oc(N3CCOCC3)cc(=O)c2c1)N1CCCC1] | 8-((4,6-difluoroindolin-1- yl)methyl)-2-morpholino-6- (pyrrolidine-1-carbonyl)-4H- chromen-4-one |
| 27 | Compound 43 | 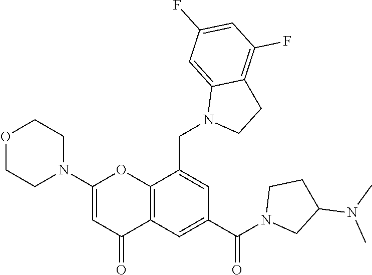 [SMILES: CN(C)C1CCN(C(=O)c2cc(CN3CCc4c(F)cc(F)cc43)c3oc(N4CCOCC4)cc(=O)c3c2)C1] | 8-((4,6-difluoroindolin-1- yl)methyl)-6-(3- (dimethylamino)pyrrolidine-1- carbonyl)-2-morpholino-4H- chromen-4-one |
| 28 | Compound 44 | 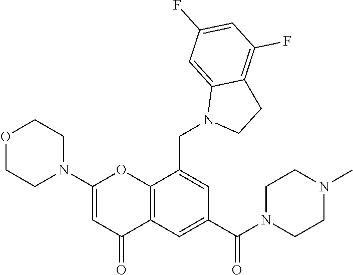 [SMILES: CN1CCN(C(=O)c2cc(CN3CCc4c(F)cc(F)cc43)c3oc(N4CCOCC4)cc(=O)c3c2)CC1] | 8-((4,6-difluoroindolin-1- yl)methyl)-6-(4-methylpiperazine- 1-carbonyl)-2-morpholino-4H- chromen-4-one |
| 29 | Compound 45 | 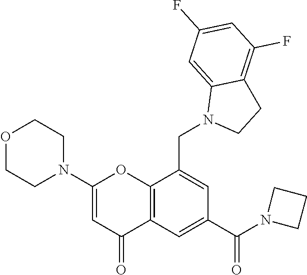 [SMILES: O=C(c1cc(CN2CCc3c(F)cc(F)cc32)c2oc(N3CCOCC3)cc(=O)c2c1)N1CCC1] | 6-(azetidine-1-carbonyl)-8-((4,6- difluoroindolin-1-yl)methyl)-2- morpholino-4H-chromen-4-one |
| 30 | Compound 46 | 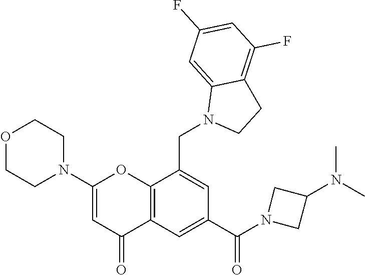 [SMILES: CN(C)C1CN(C(=O)c2cc(CN3CCc4c(F)cc(F)cc43)c3oc(N4CCOCC4)cc(=O)c3c2)C1] | 8-((4,6-difluoroindolin-1- yl)methyl)-6-(3- (dimethylamino)azetidine-1- carbonyl)-2-morpholino-4H- chromen-4-one |
| 31 | Compound 47 | 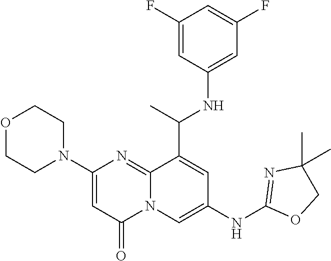 [SMILES: CC(Nc1cc(F)cc(F)c1)c1cc(NC2=NC(C)(C)CO2)cn2c(=O)cc(N3CCOCC3)nc12] | 9-(1-((3,5- difluorophenyl)amino)ethyl)-7- ((4,4-dimethyl-4,5-dihydrooxazol- 2-yl)amino)-2-morpholino-4H- pyrido[1,2-a]pyrimidin-4-one |
| 32 | Compound 48 | 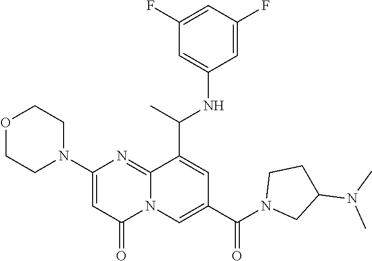 [SMILES: CC(Nc1cc(F)cc(F)c1)c1cc(C(=O)N2CCC(N(C)C)C2)cn2c(=O)cc(N3CCOCC3)nc12] | 9-(1-((3,5- difluorophenyl)amino)ethyl)-7-(3- (dimethylamino)pyrrolidine-1- carbonyl)-2-morpholino-4H- pyrido[1,2-a]pyrimidin-4-one |
| 33 | Compound 50 | 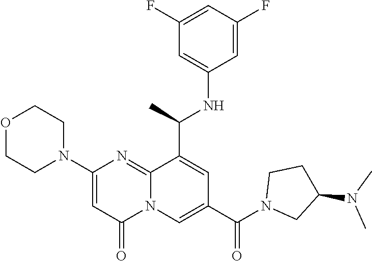 [SMILES: C[C@@H](Nc1cc(F)cc(F)c1)c1cc(C(=O)N2CC[C@@H](N(C)C)C2)cn2c(=O)cc(N3CCOCC3)nc12] | 9-((R)-1-((3,5- difluorophenyl)amino)ethyl)-7-((R)- 3-(dimethylamino)pyrrolidine-1- carbonyl)-2-morpholino-4H- pyrido[1,2-a]pyrimidin-4-one |
| 34 | Compound 51 | 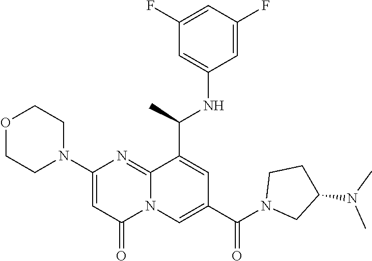 [SMILES: C[C@@H](Nc1cc(F)cc(F)c1)c1cc(C(=O)N2CC[C@H](N(C)C)C2)cn2c(=O)cc(N3CCOCC3)nc12] | 9-((R)-1-((3,5- difluorophenyl)amino)ethyl)-7-((S)- 3-(dimethylamino)pyrrolidine-1- carbonyl)-2-morpholino-4H- pyrido[1,2-a]pyrimidin-4-one |
| 35 | Compound 58 | 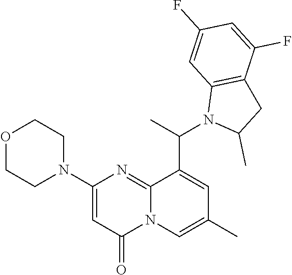 [SMILES: Cc1cc(C(C)N2c3cc(F)cc(F)c3CC2C)c2nc(N3CCOCC3)cc(=O)n2c1] | 9-(1-(4,6-difluoro-2-methylindolin- 1-yl)ethyl)-7-methyl-2-morpholino- 4H-pyrido[1,2-a]pyrimidin-4-one |
| 36 | Compound 59 | 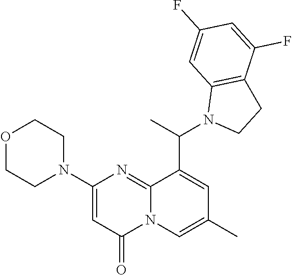 [SMILES: Cc1cc(C(C)N2CCc3c(F)cc(F)cc32)c2nc(N3CCOCC3)cc(=O)n2c1] | 9-(1-(4,6-difluoroindolin-1- yl)ethyl)-7-methyl-2-morpholino- 4H-pyrido[1,2-a]pyrimidin-4-one |
| 37 | Compound 60 | 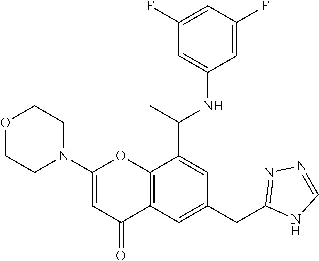 [SMILES: CC(Nc1cc(F)cc(F)c1)c1cc(Cc2nnc[nH]2)cc2c(=O)cc(N3CCOCC3)oc12] | 6-((4H-1,2,4-triazol-3-yl)methyl)-8- (1-((3,5- difluorophenyl)amino)ethyl)-2- morpholino-4H-chromen-4-one |
| 38 | Compound 62 | 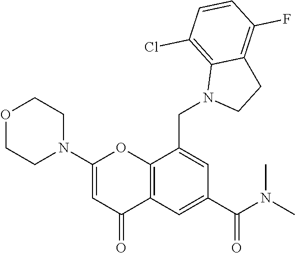 [SMILES: CN(C)C(=O)c1cc(CN2CCc3c(F)ccc(Cl)c32)c2oc(N3CCOCC3)cc(=O)c2c1] | 8-((7-chloro-4-fluoroindolin-1- yl)methyl)-N,N-dimethyl-2- morpholino-4-oxo-4H-chromene-6- carboxamide |
| 39 | Compound 63 | 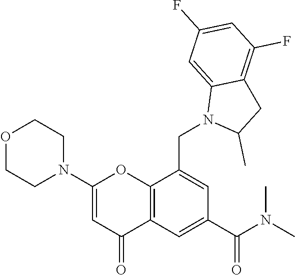 [SMILES: CC1Cc2c(F)cc(F)cc2N1Cc1cc(C(=O)N(C)C)cc2c(=O)cc(N3CCOCC3)oc12] | 8-((4,6-difluoro-2-methylindolin-1- yl)methyl)-N,N-dimethyl-2- morpholino-4-oxo-4H-chromene-6- carboxamide |
| 40 | Compound 64 | 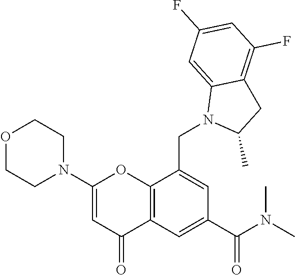 [SMILES: C[C@H]1Cc2c(F)cc(F)cc2N1Cc1cc(C(=O)N(C)C)cc2c(=O)cc(N3CCOCC3)oc12] | (S)-8-((4,6-difluoro-2- methylindolin-1-yl)methyl)-N,N- dimethyl-2-morpholino-4-oxo-4H- chromene-6-carboxamide |
| 41 | Compound 66 | 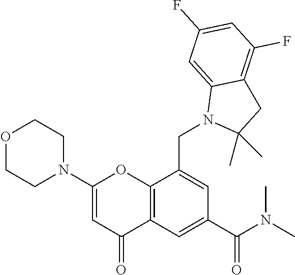 [SMILES: CN(C)C(=O)c1cc(CN2c3cc(F)cc(F)c3CC2(C)C)c2oc(N3CCOCC3)cc(=O)c2c1] | 8-((4,6-difluoro-2,2- dimethylindolin-1-yl)methyl)-N,N- dimethyl-2-morpholino-4-oxo-4H- chromene-6-carboxamide |
| 42 | Compound 68 | 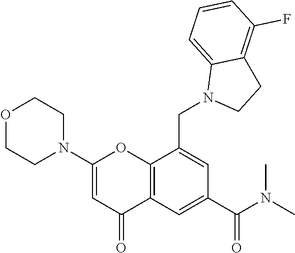 [SMILES: CN(C)C(=O)c1cc(CN2CCc3c(F)cccc32)c2oc(N3CCOCC3)cc(=O)c2c1] | 8-((4-fluoroindolin-1-yl)methyl)- N,N-dimethyl-2-morpholino-4-oxo- 4H-chromene-6-carboxamide |
| 43 | Compound 70 | 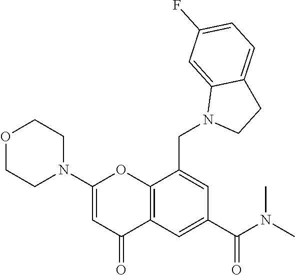 [SMILES: CN(C)C(=O)c1cc(CN2CCc3ccc(F)cc32)c2oc(N3CCOCC3)cc(=O)c2c1] | 8-((6-fluoroindolin-1-yl)methyl)- N,N-dimethyl-2-morpholino-4-oxo- 4H-chromene-6-carboxamide |
| 44 | Compound 71 | 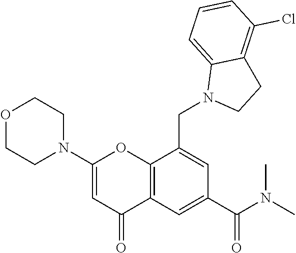 [SMILES: CN(C)C(=O)c1cc(CN2CCc3c(Cl)cccc32)c2oc(N3CCOCC3)cc(=O)c2c1] | 8-((4-chloroindolin-1-yl)methyl)- N,N-dimethyl-2-morpholino-4-oxo- 4H-chromene-6-carboxamide |
| 45 | Compound 78 | 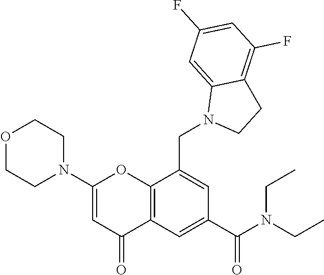 [SMILES: CCN(CC)C(=O)c1cc(CN2CCc3c(F)cc(F)cc32)c2oc(N3CCOCC3)cc(=O)c2c1] | 8-((4,6-difluoroindolin-1- yl)methyl)-N,N-diethyl-2- morpholino-4-oxo-4H-chromene-6- carboxamide |
| 46 | Compound 79 | 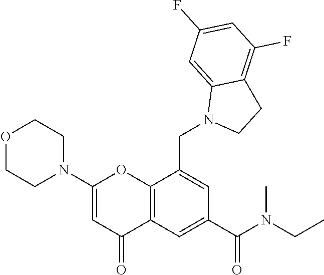 [SMILES: CCN(C)C(=O)c1cc(CN2CCc3c(F)cc(F)cc32)c2oc(N3CCOCC3)cc(=O)c2c1] | 8-((4,6-difluoroindolin-1- yl)methyl)-N-ethyl-N-methyl-2- morpholino-4-oxo-4H-chromene-6- carboxamide |
| 47 | Compound 82 | 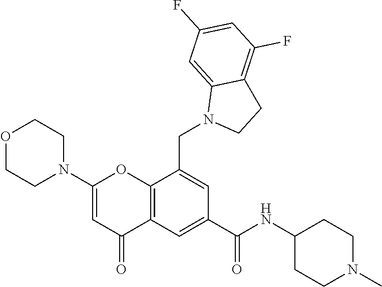 [SMILES: CN1CCC(NC(=O)c2cc(CN3CCc4c(F)cc(F)cc43)c3oc(N4CCOCC4)cc(=O)c3c2)CC1] | 8-((4,6-difluoroindolin-1- yl)methyl)-N-(1-methylpiperidin-4- yl)-2-morpholino-4-oxo-4H- chromene-6-carboxamide |
| 48 | Compound 84 | 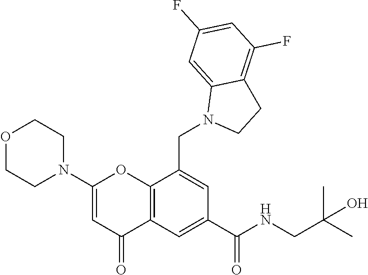 [SMILES: CC(C)(O)CNC(=O)c1cc(CN2CCc3c(F)cc(F)cc32)c2oc(N3CCOCC3)cc(=O)c2c1] | 8-((4,6-difluoroindolin-1- yl)methyl)-N-(2-hydroxy-2- methylpropyl)-2-morpholino-4-oxo- 4H-chromene-6-carboxamide |
| 49 | Compound 86 | 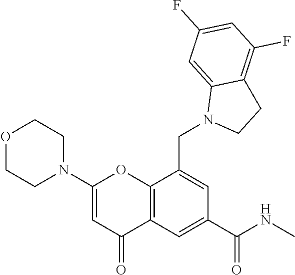 [SMILES: CNC(=O)c1cc(CN2CCc3c(F)cc(F)cc32)c2oc(N3CCOCC3)cc(=O)c2c1] | 8-((4,6-difluoroindolin-1- yl)methyl)-N-methyl-2-morpholino- 4-oxo-4H-chromene-6-carboxamide |
| 50 | Compound 90 | 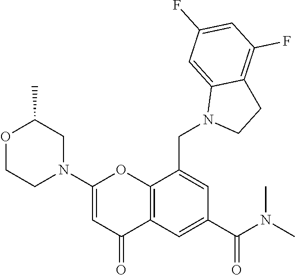 [SMILES: C[C@@H]1CN(c2cc(=O)c3cc(C(=O)N(C)C)cc(CN4CCc5c(F)cc(F)cc54)c3o2)CCO1] | (R)-8-((4,6-difluoroindolin-1- yl)methyl)-N,N-dimethyl-2-(2- methylmorpholino)-4-oxo-4H- chromene-6-carboxamide |
| 51 | Compound 91 | 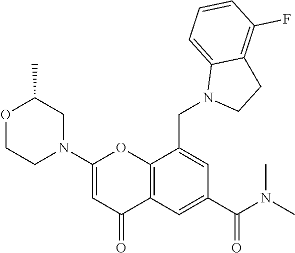 [SMILES: C[C@@H]1CN(c2cc(=O)c3cc(C(=O)N(C)C)cc(CN4CCc5c(F)cccc54)c3o2)CCO1] | (R)-8-((4-fluoroindolin-1- yl)methyl)-N,N-dimethyl-2-(2- methylmorpholino)-4-oxo-4H- chromene-6-carboxamide |
| 52 | Compound 93 | 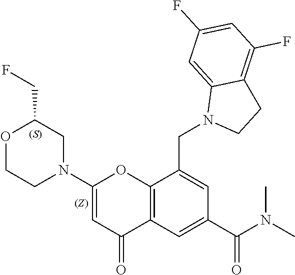 | (S)-8-((4,6-difluoroindolin-1- yl)methyl)-2-(2- (fluoromethyl)morpholino)-N,N- dimethyl-4-oxo-4H-chromene-6- carboxamide |
| 53 | Compound 96 | 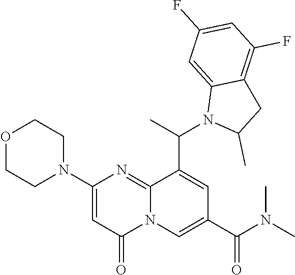 [SMILES: CC1Cc2c(F)cc(F)cc2N1C(C)c1cc(C(=O)N(C)C)cn2c(=O)cc(N3CCOCC3)nc12] | 9-(1-(4,6-difluoro-2-methylindolin- 1-yl)ethyl)-N,N-dimethyl-2- morpholino-4-oxo-4H-pyrido[1,2- a]pyrimidine-7-carboxamide |
| 54 | Compound 98 | 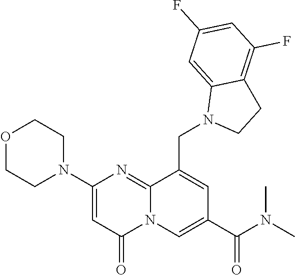 [SMILES: CN(C)C(=O)c1cc(CN2CCc3c(F)cc(F)cc32)c2nc(N3CCOCC3)cc(=O)n2c1] | 9-((4,6-difluoroindolin-1- yl)methyl)-N,N-dimethyl-2- morpholino-4-oxo-4H-pyrido[1,2- a]pyrimidine-7-carboxamide |
| 55 | Compound 99 | 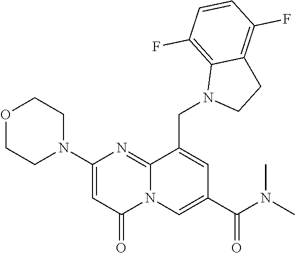 [SMILES: CN(C)C(=O)c1cc(CN2CCc3c(F)ccc(F)c32)c2nc(N3CCOCC3)cc(=O)n2c1] | 9-((4,7-difluoroindolin-1- yl)methyl)-N,N-dimethyl-2- morpholino-4-oxo-4H-pyrido[1,2- a]pyrimidine-7-carboxamide |
| 56 | Compound 100 | 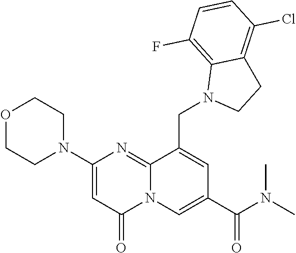 [SMILES: CN(C)C(=O)c1cc(CN2CCc3c(Cl)ccc(F)c32)c2nc(N3CCOCC3)cc(=O)n2c1] | 9-((4-chloro-7-fluoroindolin-1- yl)methyl)-N,N-dimethyl-2- morpholino-4-oxo-4H-pyrido[1,2- a]pyrimidine-7-carboxamide |
| 57 | Compound 101 | 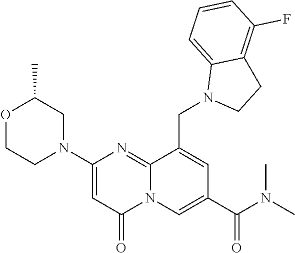 [SMILES: C[C@@H]1CN(c2cc(=O)n3cc(C(=O)N(C)C)cc(CN4CCc5c(F)cccc54)c3n2)CCO1] | (R)-9-((4-fluoroindolin-1- yl)methyl)-N,N-dimethyl-2-(2- methylmorpholino)-4-oxo-4H- pyrido[1,2-a]pyrimidine-7- carboxamide |
| 58 | Compound 102 | 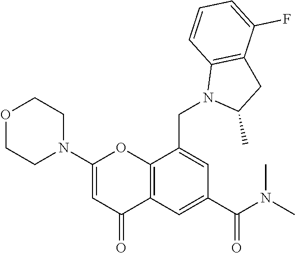 [SMILES: C[C@H]1Cc2c(F)cccc2N1Cc1cc(C(=O)N(C)C)cc2c(=O)cc(N3CCOCC3)oc12] | (S)-8-((4-fluoro-2-methylindolin-1- yl)methyl)-N,N-dimethyl-2- morpholino-4-oxo-4H-chromene-6- carboxamide |

or a pharmaceutically acceptable form or an isotope derivative thereof.

In certain embodiments, a compound of the invention has one or more (e.g., 1, 2, 3) deuterium atoms replacing one or more (e.g., 1, 2, 3) hydrogen atoms. In certain embodiments, a compound of the invention has one deuterium atom replacing one hydrogen atom.

In another aspect, the invention generally relates to a pharmaceutical composition comprising a compound disclosed herein, effective to treat or reduce one or more diseases or disorders, in a mammal, including a human, and a pharmaceutically acceptable excipient, carrier, or diluent.

In yet another aspect, the invention generally relates to a pharmaceutical composition comprising a compound having the structural formula (I):

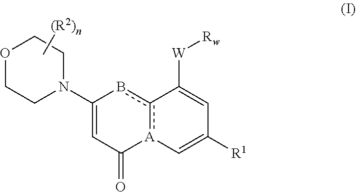

wherein
A represents carbon and B represents oxygen, or each of A and B represent nitrogen;
W represents —CHR-Q- or -Q-CHR—; wherein Q represents a bond, O, S, CH₂ or NR′; R and R′ is independently selected from hydrogen, C₁₋₄alkyl or C₁₋₄alkyl substituted with —OH or halo;
R_{w} represents Ar or a substituted or unsubstituted indoline group, wherein Ar is a group comprising a substituted or unsubstituted 6-membered aromatic or hetero-aromatic ring;
R² represents C₁₋₃ alkyl, CN, CH₂OH, CH₂F or CONH₂;
R¹ represents H, NH₂, OH, CN, CF₃, CHO, COC₁₋₄alkyl, CH₂C(═O)—NR^{1a}R^{1b}, C(═O)—NR^{1a}R^{1b}, CH₂COOR^{1c}, NR^{1d}R^{1e}, C₁₋₄ alkyl, O—C₁₋₄ alkyl, Het, Ar, NR^{1h}(C═O)R¹ⁱ, NR^{1j}(C═O)NR^{1k}R^{1l}, NR^{1j}(SO₂)R^{1m}, SO₂NR¹ⁿR^{1o}, NR^{1j}(C═O)C(CN)C(OH)R^{1p}, P(O)MeMe, P(O)OMeOMe; provided that, when A is carbon, B is oxygen and R_{w} is Ar, R¹ represents NH₂, CN, CHO, COC₁₋₄alkyl, CH₂C(═O)—NR^{1a}R^{1b}, CH₂COOR^{1c}, NR^{1d}R^{1e}, O—C₂₋₄ alkyl, Het, NR^{1h}(C═O)R¹ⁱ, NR^{1j}(C═O)NR^{1k}R^{1l}, NR^{1j}(SO₂)R^{1m}, SO₂NR¹ⁿR^{1o}, NR^{1j}(C═O)C(CN)C(OH)R^{1p}, P(O)MeMe, P(O)OMeOMe; when A and B is nitrogen, R_{w} is Ar, R¹ represents NH₂, CN, CHO, COC₁₋₄alkyl, CH₂C(═O)—NR^{1a}R^{1b}, CH₂COOR^{1c}, NR^{1d}R^{1e}, O—C₂₋₄ alkyl, Het, NR^{1h}(C═O)R¹ⁱ, NR^{1j}(C═O)NR^{1k}R^{1l}, NR^{1j}(SO₂)R^{1m}, SO₂NR¹ⁿR^{1o}, NR^{1j}(C═O)C(CN)C(OH)R^{1p}, P(O)MeMe, P(O)OMeOMe, C(═O)—NR^{1q}R^{1r}, wherein R^{1q} and R^{1r} together form a 3- to 8-membered nitrogen-containing heterocyclyl ring with one or more substituent selected from the group consisting of C₁₋₃ alkyl and NR^{3a}R^{3b};
wherein each of R^{1a}, R^{1b}, R^{1c}, R^{1d}, R^{1e}, R^{1h}, R¹ⁱ, R^{1j}, R^{1k}, R^{1l}, R^{1m}, R¹ⁿ, R^{1o}, R^{1p} is independently selected from H, C₁₋₄ alkyl, C₃₋₈cycloalkyl, C₃₋₈heterocycloakyl or C₁₋₄ alkyl substituted with one or more substituent selected from the group consisting of hydroxyl, OMe, CN, fluoro, NR^{3a}R^{3b}, C₃₋₈cycloalkyl, C₃₋₈heterocycloakyl; R^{1a} and R^{1b}, R^{1d} and R^{1e}, R¹ⁿ and R^{1o} are optionally together form a 3- to 8-membered nitrogen-containing heterocyclyl ring with one or more substituent selected from the group consisting of C₁₋₃ alkyl and NR^{3a}R^{3b}
C₁₋₄ alkyl is unsubstituted or substituted with one or more substituent selected from the group consisting of OH, F, NR^{1d}R^{1e},
R^{3a} and R^{3b} each independently are selected from the group consisting of H, and C₁₋₄ alkyl;
Het represents a 3- to 8-membered saturated or partially saturated monocyclic, bridged or spiro heterocyclyl containing at least one heteroatom each independently selected from O, S, S(═O)ₚ and N; which substituted wish one or two substituents each independently selected from the group consisting of halo, NR^{3a}R^{3b}, C₁₋₄ alkyl, OH, OMe and CN;
n represents 0, 1, 2, 3 or 4; and
p represents 1 or 2, or a pharmaceutically acceptable form or an isotope derivative thereof, effective to treat or reduce one or more diseases or disorders, in a mammal, including a human, and a pharmaceutically acceptable excipient, carrier, or diluent.

In certain embodiments, the pharmaceutical composition is suitable for oral administration.

In certain embodiments, the pharmaceutical composition is useful to treat or reduce cancer.

In certain embodiments, the pharmaceutical composition is useful to treat or reduce breast, colon, endometrial, kidney, lung, melanoma, prostate, thyroid cancer or leukemia.

In yet another aspect, the invention generally relates to a unit dosage form comprising a pharmaceutical composition disclosed herein.

In certain embodiments, the unit dosage form is suitable for oral administration.

In certain embodiments, the unit dosage form is a tablet or a capsule.

In certain embodiments, the unit dosage form is suitable for intravenous administration.

In certain embodiments, the unit dosage form is in the form of a liquid formulation.

In yet another aspect, the invention generally relates to a method for treating or reducing a disease or disorder, comprising administering to a subject in need thereof a pharmaceutical composition comprising a compound having the structural formula (I):

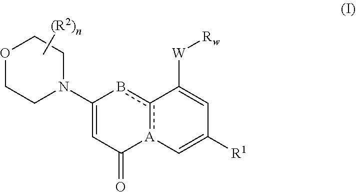

wherein
A represents carbon and B represents oxygen, or each of A and B represent nitrogen;
W represents —CHR-Q- or -Q-CHR—; wherein Q represents a bond, O, S, CH₂ or NR′; R and R′ is independently selected from hydrogen, C₁₋₄alkyl or C₁₋₄alkyl substituted with —OH or halo;
R_{w} represents Ar or a substituted or unsubstituted indoline group, wherein Ar is a group comprising a substituted or unsubstituted 6-membered aromatic or hetero-aromatic ring;
R² represents C₁₋₃ alkyl, CN, CH₂OH, CH₂F or CONH₂;
R¹ represents H, NH₂, OH, CN, CF₃, CHO, COC₁₋₄alkyl, CH₂C(═O)—NR^{1a}R^{1b}, C(═O)—NR^{1a}R^{1b}, CH₂COOR^{1c}, NR^{1d}R^{1e}, C₁₋₄ alkyl, O—C₁₋₄ alkyl, Het, Ar, NR^{1h}(C═O)R¹ⁱ, NR^{1j}(C═O)NR^{1k}R^{1l}, NR^{1j}(SO₂)R^{1m}, SO₂NR¹ⁿR^{1o}, NR^{1j}(C═O)C(CN)C(OH)R^{1p}, P(O)MeMe, P(O)OMeOMe; provided that, when A is carbon, B is oxygen and R_{w} is Ar, R¹ represents NH₂, CN, CHO, COC₁₋₄alkyl, CH₂C(═O)—NR^{1a}R^{1b}, CH₂COOR^{1c}, NR^{1d}R^{1e}, O—C₂₋₄ alkyl, Het, NR^{1h}(C═O)R¹ⁱ, NR^{1j}(C═O)NR^{1k}R^{1l}, NR^{1j}(SO₂)R^{1m}, SO₂NR¹ⁿR^{1o}, NR^{1j}(C═O)C(CN)C(OH)R^{1p}, P(O)MeMe, P(O)OMeOMe; when A and B is nitrogen, R_{w} is Ar, R¹ represents NH₂, CN, CHO, COC₁₋₄alkyl, CH₂C(═O)—NR^{1a}R^{1b}, CH₂COOR^{1c}, NR^{1d}R^{1e}, O—C₂₋₄ alkyl, Het, NR^{1h}(C═O)R¹ⁱ, NR^{1j}(C═O)NR^{1k}R^{1l}, NR^{1j}(SO₂)R^{1m}, SO₂NR¹ⁿR^{1o}, NR^{1j}(C═O)C(CN)C(OH)R^{1p}, P(O)MeMe, P(O)OMeOMe, C(═O)—NR^{1q}R^{1r}, wherein R^{1q} and R^{1r} together form a 3- to 8-membered nitrogen-containing heterocyclyl ring with one or more substituent selected from the group consisting of C₁₋₃ alkyl and NR^{3a}R^{3b};
wherein each of R^{1a}, R^{1b}, R^{1c}, R^{1d}, R^{1e}, R^{1h}, R¹ⁱ, R^{1j}, R^{1k}, R^{1l}, R^{1m}, R¹ⁿ, R^{1o}, R^{1p} is independently selected from H, C₁₋₄ alkyl, C₃₋₈cycloalkyl, C₃₋₈heterocycloakyl or C₁₋₄ alkyl substituted with one or more substituent selected from the group consisting of hydroxyl, OMe, CN, fluoro, NR^{3a}R^{3b}, C₃₋₈cycloalkyl, C₃₋₈heterocycloakyl; R^{1a} and R^{1b}, R^{1d} and R^{1e}, R¹ⁿ and R^{1o} are optionally together form a 3- to 8-membered nitrogen-containing heterocyclyl ring with one or more substituent selected from the group consisting of C₁₋₃ alkyl and NR^{3a}R^{3b}
C₁₋₄ alkyl is unsubstituted or substituted with one or more substituent selected from the group consisting of OH, F, NR^{1d}R^{1e},
R^{3a} and R^{3b} each independently are selected from the group consisting of H, and C₁₋₄ alkyl;
Het represents a 3- to 8-membered saturated or partially saturated monocyclic, bridged or spiro heterocyclyl containing at least one heteroatom each independently selected from O, S, S(═O)ₚ and N; which substituted wish one or two substituents each independently selected from the group consisting of halo, NR^{3a}R^{3b}, C₁₋₄ alkyl, OH, OMe and CN;
n represents 0, 1, 2, 3 or 4; and
p represents 1 or 2,
   or a pharmaceutically acceptable form or an isotope derivative thereof, effective to treat cancer, or a related disease or disorder, in a mammal, including a human.

In certain embodiments, the cancer that may be treated is selected from breast, colon, endometrial, kidney, lung, melanoma, prostate, thyroid cancer and leukemias. Other PTEN-deficient neoplasm may also be treated with the compounds and pharmaceutical compositions of the invention, for example, brain (gliomsa), glioblastomas, Bannayan-Zonana syndrome, Cowden disease, Lhermitte-Duclos disease, colorectal cancer, Wilm's tumor, Ewing's sarcoma, Rhabdomyosarcoma, ependymoma, medulloblastoma, head and neck cancer, liver cancer, squamous cell carcinoma, ovarian cancer, pancreatic cancer, sarcoma cancer, osteosarcoma, giant cell tumor of bone, lymphoblastic T cell, malignant lymphoma, hodykins lymphoma, non-hodgkins lymphoma, lymphoblastic T cell lymphoma, Burkitt's lymphoma, follicylar lymphoma, neuroblastoma, bladder cancer, urothelial cancer, vulval cancer, cervical cancer, renal cancer, mesothelioma, esophageal cancer, salivary gland cancer, hepatocellular cancer, gastric cancer, nasopharangeal cancer, buccal cancer, cancer of the mouth, gastrointestinal stromal tumor and testicular cancer.

In yet another aspect, the invention generally relates to use of a compound disclosed herein, and a pharmaceutically acceptable excipient, carrier, or diluent, in preparation of a medicament for treating a disease or disorder.

In certain embodiments, the disease or disorder is cancer, or a related disease or disorder.

In certain embodiments, the cancer that may be treated is selected from breast, colon, endometrial, kidney, lung, melanoma, prostate, thyroid cancer and leukemia.

In certain embodiments, the medicament is for oral administration.

In certain embodiments, the medicament is for intravenous administration.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are contemplated by the present invention. Those of ordinary skill in the art will readily appreciate that analogous ratios are contemplated for more complex isomer mixtures.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic methods well known in the art, and subsequent recovery of the pure enantiomers.

Isotopically-labeled compounds are also within the scope of the present disclosure. As used herein, an “isotopically-labeled compound” refers to a presently disclosed compound including pharmaceutical salts and prodrugs thereof, each as described herein, in which one or more atoms are replaced by an atom having an atomic mass or mass number different from the atomic mass or mass number usually found in nature. Examples of isotopes that can be incorporated into compounds presently disclosed include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

By isotopically-labeling the presently disclosed compounds, the compounds may be useful in drug and/or substrate tissue distribution assays. Tritiated (3H) and carbon-14 (¹⁴C) labeled compounds are particularly preferred for their ease of preparation and detectability. Further, substitution with heavier isotopes such as deuterium (2H) can afford certain therapeutic advantages resulting from greater metabolic stability, for example increased in vivo half-life or reduced dosage requirements and, hence, may be preferred in some circumstances. Isotopically labeled compounds presently disclosed, including pharmaceutical salts, esters, and prodrugs thereof, can be prepared by any means known in the art.

Further, substitution of normally abundant hydrogen (¹H) with heavier isotopes such as deuterium can afford certain therapeutic advantages, e.g., resulting from improved absorption, distribution, metabolism and/or excretion (ADME) properties, creating drugs with improved efficacy, safety, and/or tolerability. Benefits may also be obtained from replacement of normally abundant C with ¹³C. (See, WO 2007/005643, WO 2007/005644, WO 2007/016361, and WO 2007/016431.)

Stereoisomers (e.g., cis and trans isomers) and all optical isomers of a presently disclosed compound (e.g., R and S enantiomers), as well as racemic, diastereomeric and other mixtures of such isomers are within the scope of the present disclosure.

Compounds of the present invention are, subsequent to their preparation, preferably isolated and purified to obtain a composition containing an amount by weight equal to or greater than 95% (“substantially pure”), which is then used or formulated as described herein. In certain embodiments, the compounds of the present invention are more than 99% pure.

Solvates and polymorphs of the compounds of the invention are also contemplated herein. Solvates of the compounds of the present invention include, for example, hydrates.

Any appropriate route of administration can be employed, for example, parenteral, intravenous, subcutaneous, intramuscular, intraventricular, intracorporeal, intraperitoneal, rectal, or oral administration. Most suitable means of administration for a particular patient will depend on the nature and severity of the disease or condition being treated or the nature of the therapy being used and on the nature of the active compound.

Compositions for parenteral injection comprise pharmaceutically-acceptable sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate. Proper fluidity may be maintained, for example, by the use of coating materials such as lecithin, by the maintenance of the required particle size in the case of dispersions, and by the use of surfactants.

These compositions can also contain adjuvants such as preservative, wetting agents, emulsifying agents, and dispersing agents. Prevention of the action of microorganisms may be ensured by the inclusion of various antibacterial and antifungal agents, for example, paragen, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption, such as aluminum monostearate and gelatin.

Compounds of the present invention may also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically-acceptable and metabolizable lipid capable of forming liposomes can be used. The present compositions in liposome form can contain, in addition to a compound of the present invention, stabilizers, preservatives, excipients, and the like. The preferred lipids are the phospholipids and the phosphatidyl cholines (lecithins), both natural and synthetic. Methods to form liposomes are known in the art. See, for example, Prescott, Ed., Methods in Cell Biology, Volume XIV, Academic Press, New York, N.Y. (1976), p. 33 et seq.

Total daily dose of the compositions of the invention to be administered to a human or other mammal host in single or divided doses may be in amounts, for example, from 0.0001 to 300 mg/kg body weight daily and more usually 1 to 300 mg/kg body weight. The dose, from 0.0001 to 300 mg/kg body, may be given twice a day.

Solid dosage forms for oral administration include capsules, tablets, pills, powders, and granules. In such solid dosage forms, the compounds described herein or derivatives thereof are admixed with at least one inert customary excipient (or carrier) such as sodium citrate or dicalcium phosphate or (i) fillers or extenders, as for example, starches, lactose, sucrose, glucose, mannitol, and silicic acid, (ii) binders, as for example, carboxymethylcellulose, alginates, gelatin, polyvinylpyrrolidone, sucrose, and acacia, (iii) humectants, as for example, glycerol, (iv) disintegrating agents, as for example, agar-agar, calcium carbonate, potato or tapioca starch, alginic acid, certain complex silicates, and sodium carbonate, (v) solution retarders, as for example, paraffin, (vi) absorption accelerators, as for example, quaternary ammonium compounds, (vii) wetting agents, as for example, cetyl alcohol, and glycerol monostearate, (viii) adsorbents, as for example, kaolin and bentonite, and (ix) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents. Solid compositions of a similar type may also be employed as fillers in soft and hard-filled gelatin capsules using such excipients as lactose or milk sugar as well as high molecular weight polyethyleneglycols, and the like. Solid dosage forms such as tablets, dragees, capsules, pills, and granules can be prepared with coatings and shells, such as enteric coatings and others known in the art.

Liquid dosage forms for oral administration include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, such as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like. Besides such inert diluents, the composition can also include additional agents, such as wetting, emulsifying, suspending, sweetening, flavoring, or perfuming agents.

Materials, compositions, and components disclosed herein can be used for, can be used in conjunction with, can be used in preparation for, or are products of the disclosed methods and compositions. It is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

### Examples

The following examples are given for the purpose of illustrating the invention, but not for limiting the scope or spirit of the invention.

Compounds of the invention, including those specifically disclosed herein above and herein below, may be prepared as described in the following schemes. For example, the compounds of Formula (I) may be prepared as described in Schemes below, which are known to those of skill in the art for making fragments and combinations thereof. Although the present invention has been described in detail with preferred embodiments, those of ordinary skill in the art should understand that modifications, variations, and equivalent replacements made to the present invention within the scope of the present invention belong to the protection of the present invention.

### Abbreviations

| Abbreviation | Name |
|---|---|
| DCM | Dichloromethane |
| DMA | Dimethylacetamide |
| DMF | N,N-dimethyl formamide |
| DMSO | Dimethyl sulfoxide |
| HPLC | High-performance liquid chromatography |
| LCMS | Liquid chromatography-mass spectrometry |
| MS | Mass spectrometry |
| NIS | N-Iodosuccinimide |
| TLC | Thin-layer chromatography |
| Pd₂(dba)₃ | Tri (dibenzylidene acetone) dipalladium |
| Pd(dppf)Cl₂ | 1,1′-Bis(diphenylphosphino)ferrocene]palladium(II) |
| | dichloride |

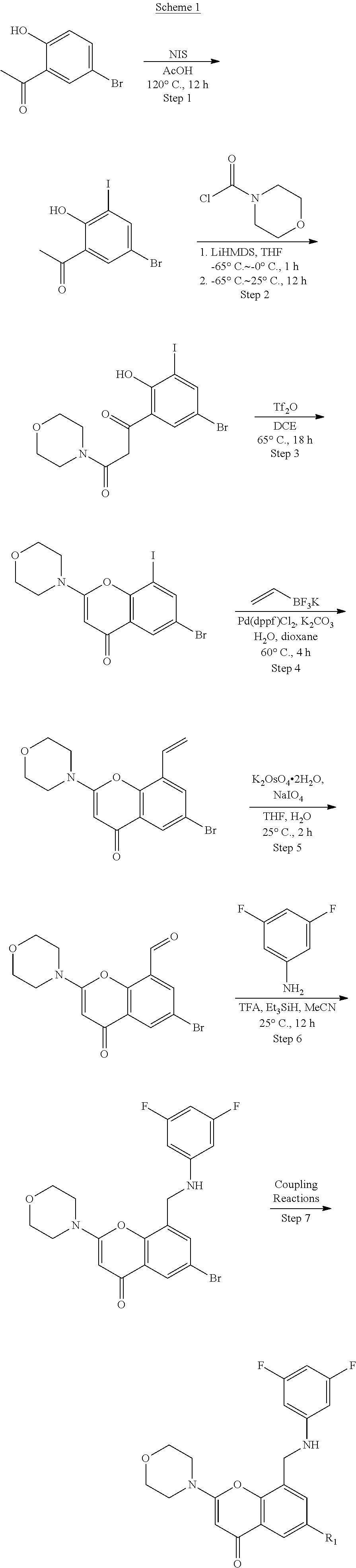

### General Procedures for Preparing Compounds in Scheme 1

### Preparation of 1-(5-bromo-2-hydroxy-3-iodophenyl)ethanone (Step 1 in Scheme 1)

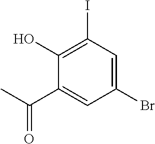

To a solution of 1-(5-bromo-2-hydroxy-phenyl)ethanone (40 g, 186.01 mmol, 1 eq) in AcOH (400 mL) was added NIS (50.22 g, 223.21 mmol, 1.2 eq) at 25° C. and the reaction mixture was stirred at 120° C. for 12 hours. HPLC and LC-MS showed 1-(5-bromo-2-hydroxy-phenyl)ethanone was consumed completely. The mixture was cooled to room temperature and poured into ice water (400 mL). There was brown precipitate formed. The collected solid was triturated with EtOH (100 mL) at 25° C. for half an hour. Compound 1-(5-bromo-2-hydroxy-3-iodo-phenyl)ethanone (45 g, 131.99 mmol, 70.96% yield) was obtained as yellow solid. ¹H NMR (CDCl₃, 400 MHz) δ 13.08 (s, 1H), 8.07 (d, J=2.4 Hz, 1H), 7.86 (d, J=2.4 Hz, 1H), 2.66 (s, 3H).

### Preparation of 1-(5-bromo-2-hydroxy-3-iodophenyl)-3-morpholinopropane-1,3-dione (Step 2 in Scheme 1)

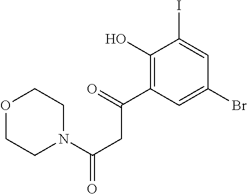 [SMILES: O=C(CC(=O)N1CCOCC1)c1cc(Br)cc(I)c1O]

To a solution of 1-(5-bromo-2-hydroxy-3-iodo-phenyl)ethanone (45 g, 131.99 mmol, 1 eq) in THF (400 mL) was added LiHMIDS (1 M, 422.36 mL, 3.2 eq) dropwise at −65° C. under N₂ and then the reaction mixture was stirred at 0° C. for an hour. The mixture was re-cooled to −65° C. and morpholine-4-carbonyl chloride (21.72 g, 145.19 mmol, 16.97 mL, 1.1 eq) was added dropwise at −65° C. The mixture was stirred at 25° C. for 12 hours. LC-MS showed 1-(5-bromo-2-hydroxy-3-iodo-phenyl)ethanone was consumed completely. The mixture was cooled to 0° C. and adjusted to pH=7 with 2M HCl. The aqueous phase was extracted with ethyl acetate (200 mL×3). The combined organic phase was washed with brine (150 mL), dried over anhydrous Na₂SO₄, filtered and concentrated in vacuum. The crude material was triturated with MTBE (200 mL) at 25° C. for half an hour. 1-(5-bromo-2-hydroxy-3-iodo-phenyl)-3-morpholino-propane-1,3-dione (57 g, 125.54 mmol, 95.11% yield) was obtained as yellow solid. ¹H NMR (DMSO-d₆, 400 MHz) δ 8.06 (s, 1H), 7.96 (s, 1H), 4.32 (s, 2H), 3.59˜3.55 (m, 4H), 3.45˜3.37 (m, 4H).

### Preparation of 6-bromo-8-iodo-2-morpholino-4H-chromen-4-one (Step 3 in Scheme 1)

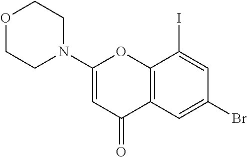 [SMILES: O=c1cc(N2CCOCC2)oc2c(I)cc(Br)cc12]

To a solution of 1-(5-bromo-2-hydroxy-3-iodo-phenyl)-3-morpholino-propane-1,3-dione (44 g, 96.90 mmol, 1 eq) in DCM (440 mL) was added Tf₂O (109.36 g, 387.62 mmol, 63.95 mL, 4 eq) dropwise at 0° C. The mixture was stirred at 25° C. for 12 hours under N₂. TLC (Petroleum ether:Ethyl acetate=0:1, R_{f}=0.4) showed 1-(5-bromo-2-hydroxy-3-iodo-phenyl)-3-morpholino-propane-1,3-dione was consumed completely. The reaction mixture was cooled to 0° C. and MeOH (200 mL) was added and the mixture was stirred at 25° C. for an hour. The mixture was concentrated and the residue was adjusted to pH=7 with sat. NaHCO₃. The aqueous phase was extracted with DCM (200 mL×3). The combined organic phase was washed with brine (200 mL), dried over anhydrous Na₂SO₄, filtered and concentrated in vacuum. The residue was purified by column chromatography (SiO₂, Petroleum ether/Ethyl acetate˜Ethyl acetate:MeOH=50/1˜4:1). 6-bromo-8-iodo-2-morpholino-chromen-4-one (11.6 g, 26.60 mmol, 27.45% yield) was obtained as yellow solid. ¹H NMR (DMSO-d₆, 400 MHz) δ 8.29 (d, J=2.4 Hz, 1H), 7.95 (d, J=2.4 Hz, 1H), 5.59 (s, 1H), 3.81-3.72 (m, 4H), 3.62˜3.57 (m, 4H).

### Preparation of 6-bromo-2-morpholino-8-vinyl-4H-chromen-4-one (Step 4 in Scheme 1)

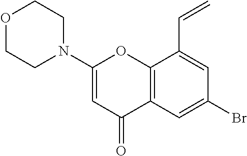 [SMILES: C=Cc1cc(Br)cc2c(=O)cc(N3CCOCC3)oc12]

To a mixture of 6-bromo-8-iodo-2-morpholino-chromen-4-one (8.4 g, 19.26 mmol, 1 eq) and potassium trifluoro(vinyl) boranuide (2.84 g, 21.19 mmol, 1.1 eq) in dioxane (80 mL) was added Pd(dppf)Cl₂ (704.79 mg, 963.22 umol, 0.05 eq) and a solution of K₂CO₃ (5.32 g, 38.53 mmol, 2 eq) in H₂O (10 mL) at 25° C. under N₂. The mixture was stirred at 60° C. for 4 hours. LC-MS showed 6-bromo-8-iodo-2-morpholino-chromen-4-one was consumed completely. After filtration, the filtrate was concentrated. The residue was dissolved in EtOAc (50 mL) and H₂O (80 mL). The organic phase was separated and the aqueous phase was extracted with EtOAc (50 mL×2). The combined organic phase was washed with brine (20 mL), dried over anhydrous Na₂SO₄, filtered and concentrated in vacuum. The crude product was triturated with MTBE (30 mL) at 25° C. for half an hour. Compound 6-bromo-2-morpholino-8-vinyl-chromen-4-one (6.9 g, crude) was obtained as yellow solid. ¹H NMR (DMSO-d₆, 400 MHz) δ 8.09 (d, J=2.4 Hz, 1H), 7.90 (d, J=2.4 Hz, 1H), 7.12 (q, J=11.2, 1H), 6.13 (d, J=17.6 Hz, 1H), 5.60-5.55 (m, 2H), 3.77-3.69 (m, 4H), 3.55-3.49 (m, 4H).

### Preparation of 6-bromo-2-morpholino-4-oxo-4H-chromene-8-carbaldehyde (Step 5 in Scheme 1)

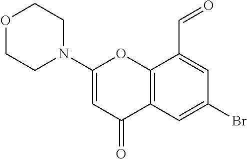 [SMILES: O=Cc1cc(Br)cc2c(=O)cc(N3CCOCC3)oc12]

To a solution of 6-bromo-2-morpholino-8-vinyl-chromen-4-one (6.3 g, 18.74 mmol, 1 eq) in THF (50 mL) and H₂O (10 mL) was added K₂OsO₄·2H₂O (345.25 mg, 937.00 umol, 0.05 eq) at 25° C. and the reaction mixture was stirred at 25° C. for half an hour. NaIO₄ (12.02 g, 56.22 mmol, 3.12 mL, 3 eq) was added to the mixture at 0° C. in portions. The reaction mixture was stirred at 0° C. for half an hour. TLC (petroleum ether:EtOAc=0:1, R_{f}=0.23) indicated 6-bromo-2-morpholino-8-vinyl-chromen-4-one was consumed completely. The mixture was quenched with water (100 mL) and extracted with ethyl acetate (80 mL×3). The combined organic phase was washed with brine (50 mL), dried with anhydrous Na₂SO₄, filtered and concentrated in vacuum. The residue was purified by column chromatography (SiO₂, Petroleum ether/Ethyl acetate˜EtOAc:MeOH=30:1˜4:1). Compound 6-bromo-2-morpholino-4-oxo-chromene-8-carbaldehyde (5.2 g, 15.38 mmol, 82.06% yield) was obtained as yellow solid. ¹H NMR (DMSO-d₆, 400 MHz) δ 10.33 (s, 1H), 8.24˜8.21 (m, 2H), 5.66 (s, 1H), 3.72˜3.75 (m, 4H), 3.61-3.63 (m, 4H).

### Preparation of 6-bromo-8-(((3,5-difluorophenyl)amino)methyl)-2-morpholino-4H-chromen-4-one (Step 6 in Scheme 1)

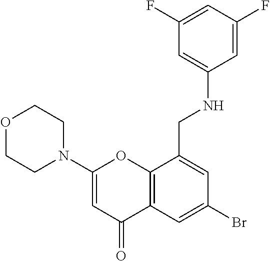 [SMILES: O=c1cc(N2CCOCC2)oc2c(CNc3cc(F)cc(F)c3)cc(Br)cc12]

To a mixture of 6-bromo-2-morpholino-4-oxo-chromene-8-carbaldehyde (5.2 g, 15.38 mmol, 1 eq) and 3,5-difluoroaniline (3.97 g, 30.76 mmol, 2 eq) in CH₃CN (2 mL) was added Et₃SiH (10.73 g, 92.27 mmol, 14.74 mL, 6 eq) and TFA (7.01 g, 61.51 mmol, 4.55 mL, 4 eq) at 25° C. The reaction mixture was stirred at 25° C. for 12 hours. LC-MS showed 6-bromo-2-morpholino-4-oxo-chromene-8-carbaldehyde was consumed completely. The mixture was adjusted to pH=7 with sat. NaHCO₃ at 0° C. The mixture was extracted with ethyl acetate (80 mL×3). The combined organic phase was washed with brine (80 mL), dried over anhydrous Na₂SO₄, filtered and concentrated in vacuum. The residue was purified by column chromatography (SiO₂, Petroleum ether/Ethyl acetate=50/1 to 0:1). Compound 6-bromo-8-[(3,5-difluoroanilino)methyl]-2-morpholino-chromen-4-one (1.8 g, 3.99 mmol, 25.94% yield) was obtained as yellow solid. ¹H NMR (DMSO-d₆, 400 MHz) δ 7.89 (d, J=2.4 Hz, 1H), 7.64 (d, J=2.4 Hz, 1H), 6.34˜6.28 (m, 3H), 5.59 (s, 1H), 4.53 (d, J=6.0 Hz, 2H), 3.72˜3.67 (m, 4H), 3.53˜3.50 (m, 4H).

### Preparation of Final Compounds in Scheme 1 (Step 7 in Scheme 1)

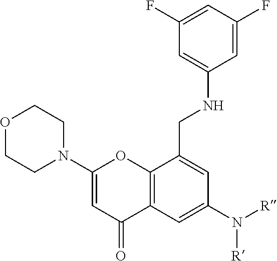

A mixture of 6-bromo-8-(((3,5-difluorophenyl)amino)methyl)-2-morpholino-4H-chromen-4-one (332.40 umol, 1 eq), R′NHR″ (997.21 umol˜1.662 mmol, 3 eq˜5 eq), Pd₂(dba)₃ (33.24 umol˜66.48 umol, 0.1 eq˜0.2 eq), XPhos (49.86 umol˜99.72 umol, 0.15 eq˜0.3 eq) and Cs₂CO₃ (997.21 umol˜2.66 mmol, 3 eq˜8 eq) in dioxane (4.5 mL/mmol˜9.0 mL/mmol) was stirred at 100° C. for 10 hours˜12 hours under N₂. HPLC and LC-MS showed the reaction was complete. After filtration, the filtrate was concentrated under reduced pressure. The residue was dissolved in EtOAc and washed with water. The combined organic layer was washed with brine, dried over Na₂SO₄, filtered and concentrated under reduced pressure. The residue was purified by prep-HPLC (column: Phenomenex Gemini-NX 80*30 mm*3 um or Phenomenex luna C18 80*40 mm*3 um; mobile phase: [water (10 mM NH₄HCO₃)-MeCN]; B %: 30%-45%, 10 mins or [water (0.04% HCl)-MeCN]; B %: 20%-50%, 7 mins). The aqueous solution was lyophilized to give the desired product.

### Compound 1

### Preparation of 8-(((3,5-difluorophenyl)amino)methyl)-2,6-dimorpholino-4H-chromen-4-one (Step 7 in Scheme 1)

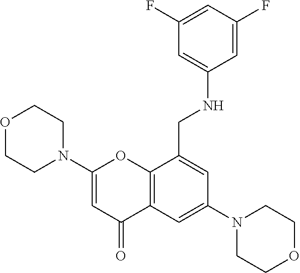 [SMILES: O=c1cc(N2CCOCC2)oc2c(CNc3cc(F)cc(F)c3)cc(N3CCOCC3)cc12]

A mixture of 6-bromo-8-(((3,5-difluorophenyl)amino)methyl)-2-morpholino-4H-chromen-4-one (150 mg, 332.40 umol, 1 eq), morpholine (86.88 mg, 997.21 umol, 87.75 uL, 3 eq), Pd₂(dba)₃ (30.44 mg, 33.24 umol, 0.1 eq), XPhos (23.77 mg, 49.86 umol, 0.15 eq) and Cs₂CO₃ (324.91 mg, 997.21 umol, 3 eq) in dioxane (3 mL) was stirred at 100° C. for 10 hours under N₂. TLC (petroleum ether:EtOAc=0:1, R_{f}=0.2) and LC-MS showed the reaction was complete. After filtration, the filtrate was concentrated under reduced pressure. The residue was dissolved in EtOAc (5 mL) and washed with water (2 mL×2). The combined organic layer was washed with brine (2 mL×1), dried over Na₂SO₄, filtered and concentrated under reduced pressure. The residue was purified by prep-HPLC (column: Phenomenex Gemini-NX 80*30 mm*3 um; mobile phase: [water (10 mM NH₄HCO₃)-MeCN]; B %: 30%-45%, 10 mins). The eluent was removed under freeze drying. Compound 8-(((3,5-difluorophenyl)amino)methyl)-2,6-dimorpholino-4H-chromen-4-one (26.3 mg, 57.49 umol, 17.30% yield) was obtained as white solid. ¹H NMR (DMSO-d₆, 400 MHz) δ 7.30 (d, J=2.8 Hz, 1H), 7.23 (d, J=2.8 Hz, 1H), 6.75 (t, J=2.8 Hz, 1H), 6.31-6.25 (m, 3H), 5.50 (s, 1H), 4.45 (t, J=5.2 Hz, 2H), 3.74 (t, J=4.4 Hz, 4H), 3.66 (t, J=4.4 Hz, 4H), 3.46 (t, J=4.4 Hz, 4H), 3.09 (t, J=4.4 Hz, 4H). HPLC: 97.61% (220 nm), 99.42% (254 nm). MS (ESI): mass calcd. For C₂₄H₂₅F₂N₃O₄ 457.18 m/z found 458.2 [M+H]⁺.

### Compound 2

### 8-(((3,5-difluorophenyl)amino)methyl)-6-(dimethylamino)-2-morpholino-4H-chromen-4-one was prepared according to the procedure described herein for Step 7 in Scheme 1

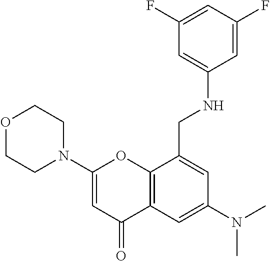 [SMILES: CN(C)c1cc(CNc2cc(F)cc(F)c2)c2oc(N3CCOCC3)cc(=O)c2c1]

The desired compound (22.2 mg, 48.48 umol, 10.94% yield, 98.69% purity, HCl) was obtained as yellow solid. ¹H NMR (DMSO-d₆, 400 MHz) δ 7.20˜7.09 (m, 2H), 6.33˜6.21 (m, 3H), 5.65 (s, 1H), 4.47 (s, 2H), 3.71˜3.59 (m, 8H), 2.92 (s, 6H). HPLC: 98.69% (220 nm), 98.47% (254 nm). MS (ESI): mass calcd. For C₂₂H₂₃F₂N₃O₃ 415.17 m/z found 416.2 [M+H]⁺.

### Compound 3

### Preparation of 8-(((3,5-difluorophenyl)amino)methyl)-2-morpholino-4-oxo-4H-chromene-6-carbonitrile (Step 7 in Scheme 1)

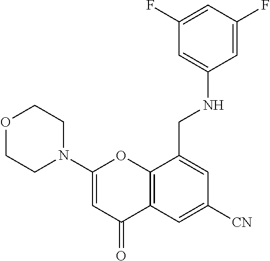 [SMILES: N#Cc1cc(CNc2cc(F)cc(F)c2)c2oc(N3CCOCC3)cc(=O)c2c1]

To a solution of 6-bromo-8-[(3,5-difluoroanilino)methyl]-2-morpholino-chromen-4-one (0.1 g, 221.60 umol, 1 eq) in DMA (1 mL) was added Zn(CN)₂ (78.07 mg, 664.80 umol, 42.20 uL, 3.0 eq), DPPF (19.66 mg, 35.46 umol, 0.16 eq) and Pd₂(dba)₃ (16.23 mg, 17.73 umol, 0.08 eq) at 25° C. under N₂. The mixture was stirred at 170° C. for 2 hours. LC-MS and HPLC showed 6-bromo-8-[(3,5-difluoroanilino)methyl]-2-morpholino-chromen-4-one was consumed completely. The reaction mixture was filtered. The filtrate was purified by prep-HPLC (column: Welch Xtimate C18 150*25 mm*5 um; mobile phase: [water (0.04% HCl)-MeCN]; B %: 25%-45%, 8 mins). The eluent was removed under freeze drying. Compound 8-[(3,5-difluoroanilino)methyl]-2-morpholino-4-oxo-chromene-6-carbonitrile (27.5 mg, 69.20 umol, 31.23% yield) was obtained as white solid. ¹H NMR (DMSO-d₆, 400 MHz) δ 8.20 (d, J=2.4 Hz, 1H), 7.86 (d, J=2.4 Hz, 1H), 6.94˜6.79 (m, 1H), 6.37˜6.22 (m, 3H), 5.65 (s, 1H), 4.56 (s, 2H), 3.75˜3.67 (m, 4H), 3.56˜3.49 (m, 4H). HPLC: 96.81% (220 nm), 99.10% (254 nm). MS (ESI): mass calcd. For C₂₁H₁₇F₂N₃O₃ 397.12 m/z found 398.1 [M+H]⁺.

### Compound 4

### Preparation of 8-(((3,5-difluorophenyl)amino)methyl)-6-ethoxy-2-morpholino-4H-chromen-4-one (Step 7 in Scheme 1)

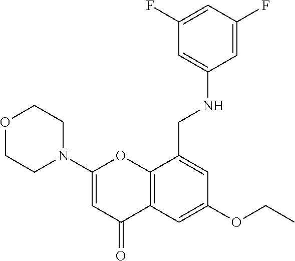 [SMILES: CCOc1cc(CNc2cc(F)cc(F)c2)c2oc(N3CCOCC3)cc(=O)c2c1]

To a mixture of 6-bromo-8-[(3,5-difluoroanilino)methyl]-2-morpholino-chromen-4-one (0.15 g, 332.40 umol, 1 eq) and EtONa (67.86 mg, 997.21 umol, 3 eq) in EtOH (0.3 mL) and toluene (0.2 mL) was added ditert-butyl-[2-(2,4,6-triisopropylphenyl)phenyl]phosphane (28.23 mg, 66.48 umol, 0.2 eq), Pd(OAc)₂ (7.46 mg, 33.24 umol, 0.1 eq) and Cs₂CO₃ (162.45 mg, 498.60 umol, 1.5 eq) at 25° C. under N₂. The mixture was stirred at 100° C. for 12 hours. LC-MS and HPLC showed 6-bromo-8-[(3,5-difluoroanilino)methyl]-2-morpholino-chromen-4-one was consumed completely. The reaction mixture was filtered and the filtrate was concentrated. The residue was purified by prep-HPLC (column: Xtimate C18 100*30 mm*3 um; mobile phase: [water (0.04% HCl)-MeCN]; B %: 30%-60%, 8 mins). The solvent was removed under freeze drying. Compound 8-[(3,5-difluoroanilino) methyl]-6-ethoxy-2-morpholino-chromen-4-one (4.9 mg, 11.77 umol, 3.54% yield) was obtained as white solid. ¹H NMR (DMSO-d₆, 400 MHz) δ 7.24 (d, J=2.4 Hz, 1H), 7.09 (d, J=2.4 Hz, 1H), 6.40˜6.21 (m, 3H), 5.61 (s, 1H), 4.51 (s, 2H), 4.04 (q, J=8.0 Hz, 2H), 3.69˜3.67 (m, 4H), 3.52˜3.50 (m, 4H), 1.31 (t, J=6.8 Hz, 3H). HPLC: 93.72% (220 nm), 96.88% (254 nm). MS (ESI): mass calcd. For C₂₂H₂₂F₂O₄N₂ 416.15 m/z found 417.1 [M+H]⁺.

### Compound 5

### Preparation of 8-(((3,5-difluorophenyl)amino)methyl)-6-ethynyl-2-morpholino-4H-chromen-4-one (Step 7 in Scheme 1)

### Preparation of 8-(((3,5-difluorophenyl)amino)methyl)-2-morpholino-6-((trimethylsilyl)ethynyl)-4H-chromen-4-one

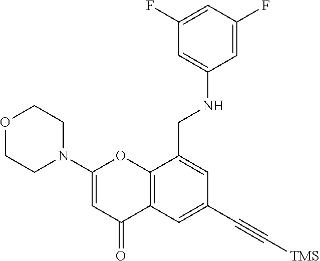 [SMILES: C[Si](C)(C)C#Cc1cc(CNc2cc(F)cc(F)c2)c2oc(N3CCOCC3)cc(=O)c2c1]

To a mixture of 6-bromo-8-[(3,5-difluoroanilino)methyl]-2-morpholino-chromen-4-one (0.18 g, 398.88 umol, 1 eq) and ethynyl(trimethyl)silane (195.89 mg, 1.99 mmol, 276.29 uL, 5 eq) in MeCN (3 mL) was added [2-(2-aminophenyl)phenyl]-chloro-palladiumtritert-butylphosphane (20.44 mg, 39.89 umol, 0.1 eq) and N-cyclohexyl-N-methyl-cyclohexanamine (233.75 mg, 1.20 mmol, 253.80 uL, 3 eq) at 25° C. under N₂. The reaction mixture was stirred at 80° C. for 12 hours. LC-MS showed 6-bromo-8-[(3,5-difluoroanilino)methyl]-2-morpholino-chromen-4-one was consumed completely. The reaction mixture was filtered and the filtrate was concentrated under reduced pressure. Compound 8-[(3,5-difluoroanilino)methyl]-2-morpholino-6-(2-trimethylsilyl ethynyl)chromen-4-one (0.18 g, crude) was obtained as yellow solid. MS (ESI): mass calcd. For C₂₅H₂₆F₂O₃N₂Si 468.57 m/z found 469.0 [M+H]⁺.

### Preparation of 8-(((3,5-difluorophenyl)amino)methyl)-6-ethynyl-2-morpholino-4H-chromen-4-one

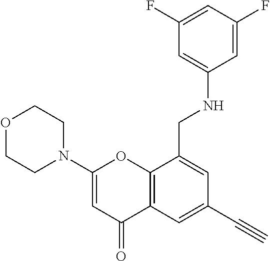 [SMILES: C#Cc1cc(CNc2cc(F)cc(F)c2)c2oc(N3CCOCC3)cc(=O)c2c1]

To a solution of 8-[(3,5-difluoroanilino)methyl]-2-morpholino-6-(2-trimethylsilylethynyl) chromen-4-one (0.17 g, 362.81 umol, 1 eq) in MeOH (2 mL) was added K₂CO₃ (100.28 mg, 725.62 umol, 2 eq) at 25° C. and the mixture was stirred at 25° C. for an hour. LC-MS and HPLC showed 8-[(3,5-difluoroanilino)methyl]-2-morpholino-6-(2-trimethylsilylethynyl) chromen-4-one was consumed completely. The mixture was cooled to 0° C. and adjusted to pH=7 with 2M HCl. The aqueous phase was extracted with ethyl acetate (15 mL×3). The combined organic phase was washed with brine (10 mL), dried over anhydrous Na₂SO₄, filtered and concentrated in vacuum. The residue was purified by prep-HPLC (column: Xtimate C18 100*30 mm*3 um; mobile phase: [water (0.04% HCl)-MeCN]; B %: 30%-60%, 6 mins). The eluent was removed under freeze drying. Compound 8-[(3,5-difluoroanilino)methyl]-6-ethynyl-2-morpholino-chromen-4-one (8.3 mg, 18.78 umol, 5.18% yield, 97.94% purity, HCl) was obtained as white solid. ¹H NMR (DMSO-d₆, 400 MHz) δ 7.85 (d, J=2.0 Hz, 1H), 7.56 (d, J=2.0 Hz, 1H), 6.88 (s, 1H), 6.35˜6.21 (m, 3H), 5.59 (s, 1H), 4.52 (s, 2H), 4.23 (s, 1H), 3.71˜3.66 (m, 4H), 3.53˜3.51 (m, 4H). HPLC: 97.94% (220 nm), 99.62% (254 nm). MS (ESI): mass calcd. For C₂₂H₁₈F₂O₃N₂ 396.13 m/z found 397.1 [M+H]⁺.

### Compound 6

### Preparation of 6-cyclopropyl-8-(((3,5-difluorophenyl)amino)methyl)-2-morpholino-4H-chromen-4-one (Step 7 in Scheme 1)

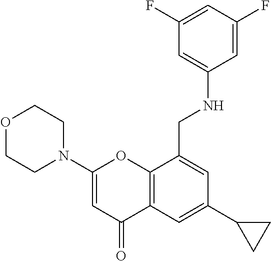 [SMILES: O=c1cc(N2CCOCC2)oc2c(CNc3cc(F)cc(F)c3)cc(C3CC3)cc12]

To a mixture of 6-bromo-8-[(3,5-difluoroanilino)methyl]-2-morpholino-chromen-4-one (0.2 g, 443.20 umol, 1 eq) and cyclopropylboronic acid (304.56 mg, 3.55 mmol, 8 eq) in dioxane (10 mL) and H₂O (2 mL) was added PCy₃ (12.43 mg, 44.32 umol, 14.37 uL, 0.1 eq), K₃PO₄ (188.15 mg, 886.41 umol, 2 eq) and Pd(OAc)₂ (4.98 mg, 22.16 umol, 0.05 eq) at 25° C. under N₂. The mixture was stirred at 90° C. for 12 hours. LC-MS showed 6-bromo-8-[(3,5-difluoroanilino) methyl]-2-morpholino-chromen-4-one was consumed completely. The reaction mixture was filtered and the filtrate was concentrated in vacuum. The residue was purified by prep-HPLC (column: Phenomenex Gemini-NX C18 75*30 mm*3 um; mobile phase: [water (0.05% NH₃H₂O+10 mM NH₄HCO₃)-MeCN]; B %: 35%-60%, 8 mins). The solvent was concentrated and the obtained was further purified by prep-HPLC (column: Phenomenex luna C18 80*40 mm*3 um; mobile phase: [water (0.04% HCl)-MeCN]; B %: 42%-52%, 7 mins). The solvent was removed under freeze drying. Compound 6-cyclopropyl-8-[(3,5-difluoroanilino)methyl]-2-morpholino-chromen-4-one (7.2 mg, 16.84 umol, 3.80% yield, 96.49% purity) was obtained as white solid. ¹H NMR (DMSO-d₆, 400 MHz) δ 7.51 (d, J=2.0 Hz, 1H), 7.31 (d, J=2.0 Hz, 1H), 6.36˜6.22 (m, 3H), 5.60 (s, 1H), 4.47 (s, 2H), 3.68˜3.66 (m, 4H), 3.53˜3.48 (m, 4H), 2.03˜1.96 (m, 1H), 0.98˜0.96 (m, 2H), 0.66˜0.58 (m, 2H). HPLC: 98.24% (220 nm), 100% (254 nm). MS (ESI): mass calcd. For C₂₃H₂₂F₂O₃N₂ 412.16 m/z found 413.1 [M+H]⁺.

### Compound 7

### Preparation of 3-(8-(((3,5-difluorophenyl)amino)methyl)-2-morpholino-4-oxo-4H-chromen-6-yl)-1,1-dimethylurea (Step 7 in Scheme 1)

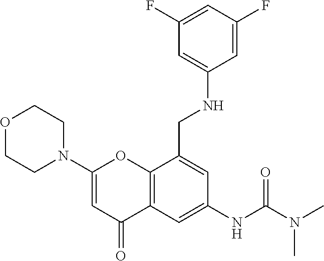 [SMILES: CN(C)C(=O)Nc1cc(CNc2cc(F)cc(F)c2)c2oc(N3CCOCC3)cc(=O)c2c1]

To a mixture of 6-bromo-8-[(3,5-difluoroanilino)methyl]-2-morpholino-chromen-4-one (0.2 g, 443.20 umol, 1 eq) and 1,1-dimethylurea (78.10 mg, 886.41 umol, 2 eq) in dioxane (3 mL) was added Cs₂CO₃ (288.81 mg, 886.41 umol, 2 eq) and BrettPhos Pd G3 (40.18 mg, 44.32 umol, 0.1 eq) at 25° C. under N₂. The mixture was stirred at 100° C. for 12 hours. LC-MS and HPLC showed 6-bromo-8-[(3,5-difluoroanilino)methyl]-2-morpholino-chromen-4-one was consumed completely. The reaction mixture was filtered and the filtrate was concentrated in vacuum. The residue was purified by prep-HPLC (column: Welch Xtimate C18 150*25 mm*5 um; mobile phase: [water (0.04% HCl)-MeCN]; B %: 20%-40%, 8 mins). Compound 3-[8-[(3,5-difluoroanilino)methyl]-2-morpholino-4-oxo-chromen-6-yl]-1,1-dimethyl-urea (8.7 mg, 17.13 umol, 3.86% yield, 97.43% purity, HCl) was obtained as white solid. ¹H NMR (DMSO-d₆, 400 MHz) δ 8.50 (s, 1H), 7.93 (d, J=2.4 Hz, 1H), 7.76 (d, J=2.4 Hz, 1H), 6.86 (s, 1H), 6.26˜6.24 (m, 3H), 5.50 (s, 1H), 4.46 (s, 2H), 3.69˜3.67 (m, 4H), 3.50˜3.48 (m, 4H), 2.91 (s, 6H). HPLC: 97.42% (220 nm), 99.13% (254 nm). MS (ESI): mass calcd. For C₂₂H₂₄F₂O₄N₄ 458.18 m/z found 459.2 [M+H]⁺.

### Compound 8

### Preparation of N-(8-(((3,5-difluorophenyl)amino)methyl)-2-morpholino-4-oxo-4H-chromen-6-yl)acetamide (Step 7 in Scheme 1)

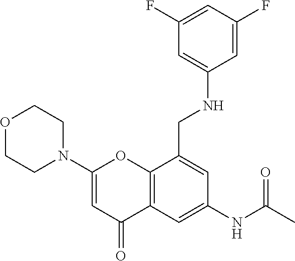 [SMILES: CC(=O)Nc1cc(CNc2cc(F)cc(F)c2)c2oc(N3CCOCC3)cc(=O)c2c1]

A mixture of 6-bromo-8-(((3,5-difluorophenyl)amino)methyl)-2-morpholino-4H-chromen-4-one (0.15 g, 332.40 umol, 1 eq), acetamide (58.90 mg, 997.21 umol, 3 eq), CuI (12.66 mg, 66.48 umol, 0.2 eq), N1,N2-dimethylethane-1,2-diamine (5.86 mg, 66.48 umol, 7.16 uL, 0.2 eq) and Cs₂CO₃ (324.91 mg, 997.21 umol, 3 eq) in dioxane (3 mL) was stirred at 100° C. for 10 hours under N₂. LC-MS showed the reaction was complete. After filtration, the filtrate was concentrated under reduced pressure. The residue was purified by prep-HPLC (column: Phenomenex luna C18 80*40 mm*3 um; mobile phase: [water (0.04% HCl)-MeCN]; B %: 19%-49%, 7 mins). The eluent was removed under freeze drying. Compound N-(8-(((3,5-difluorophenyl)amino)methyl)-2-morpholino-4-oxo-4H-chromen-6-yl)acetamide (42.8 mg, 91.87 umol, 27.64% yield, HCl) was obtained as white solid. ¹H NMR (DMSO-d₆, 400 MHz) δ 10.13 (s, 1H), 8.20 (d, J=2.4 Hz, 1H), 7.65 (d, J=2.0 Hz, 1H), 6.93 (s, 1H), 6.26˜6.23 (m, 3H), 5.61 (s, 1H), 4.51 (s, 2H), 3.70 (t, J=4.4 Hz, 4H), 3.52 (t, J=4.4 Hz, 4H), 2.02 (s, 3H). HPLC: 94.83% (220 nm), 97.01% (254 nm). MS (ESI): mass calcd. For C₂₂H₂₁F₂N₃O₄ 429.15 m/z found 430.2 [M+H]⁺.

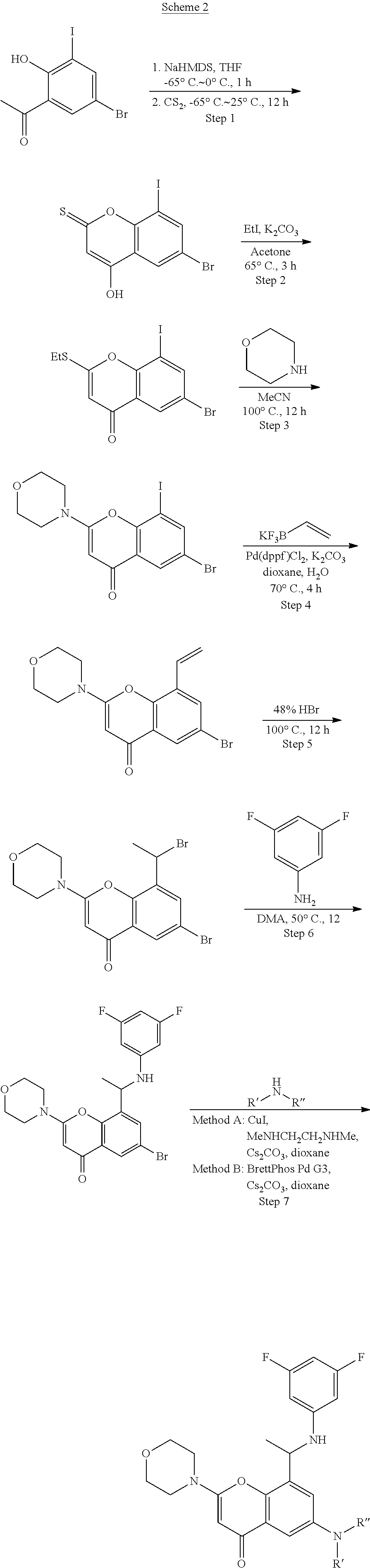

### General Procedures for Preparing Compounds in Scheme 2

### Preparation of 6-bromo-4-hydroxy-8-iodo-2H-chromene-2-thione (Step 1 in Scheme 2)

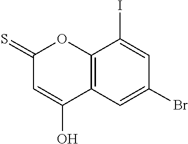 [SMILES: Oc1cc(=S)oc2c(I)cc(Br)cc12]

To a solution of 1-(5-bromo-2-hydroxy-3-iodo-phenyl)ethanone (42 g, 123.19 mmol, 1.0 eq) in THF (500 mL) was added NaHMDS (1 M, 431.16 mL, 3.5 eq) at −65° C. under N₂. The mixture was stirred at 0° C. for an hour, then cooled to −65° C. and CS₂ (15.01 g, 197.10 mmol, 11.91 mL, 1.6 eq) was added dropwise. The mixture was stirred at 25° C. for 12 hours. LCMS showed the reaction was complete. The reaction mixture was poured into ice water (1000 mL) and pH was adjusted to ˜4 with con. HCl slowly. The organic layer was separated and the aqueous phase was extracted with EtOAc (500 mL×3). The combined organic layer was washed with brine (300 mL×2), dried over Na₂SO₄, filtered and concentrated under reduced pressure. The crude product was triturated with DCM (300 mL) at 25° C. for half an hour. Compound 6-bromo-4-hydroxy-8-iodo-chromene-2-thione (80 g, 208.88 mmol, 56.52% yield) was obtained as yellow solid. ¹H NMR (DMSO-d₆, 400 MHz) δ 8.33 (d, J=2.4 Hz, 1H), 7.94 (d, J=2.4 Hz, 1H), 6.55 (s, 1H).

### Preparation of 6-bromo-2-(ethylthio)-8-iodo-4H-chromen-4-one (Step 2 in Scheme 2)

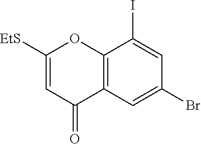

To a solution of 6-bromo-4-hydroxy-8-iodo-chromene-2-thione (70 g, 182.77 mmol, 1.0 eq) in acetone (100 mL) was added K₂CO₃ (30.31 g, 219.32 mmol, 1.2 eq) and EtI (99.77 g, 639.69 mmol, 51.16 mL, 3.5 eq) at 25° C. The mixture was stirred at 65° C. for 3 hours. LC-MS showed the reaction was consumed completely. The solvent was removed under reduced pressure. The residue was dissolved in EtOAc (300 mL) and washed with 0.05N HCl until pH=7. The combined organic layer was washed with brine (100 mL×2), dried over Na₂SO₄, filtered and concentrated under reduced pressure. The crude product was triturated with EtOAc (120 mL) at 25° C. for 30 min. Compound 6-bromo-2-ethylsulfanyl-8-iodo-chromen-4-one (60 g, 145.97 mmol, 79.86% yield) was obtained as yellow solid. ¹H NMR (DMSO-d₆, 400 MHz) δ 8.40 (d, J=2.4 Hz, 1H), 8.01 (d, J=2.4 Hz, 1H), 6.45 (s, 1H), 3.29˜3.26 (m, 2H), 1.39 (t, J=7.2 Hz, 3H).

### Preparation of 6-bromo-8-iodo-2-morpholino-4H-chromen-4-one (Step 3 in Scheme 2)

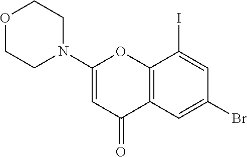 [SMILES: O=c1cc(N2CCOCC2)oc2c(I)cc(Br)cc12]

To a solution of 6-bromo-2-ethylsulfanyl-8-iodo-chromen-4-one (50 g, 121.64 mmol, 1.0 eq) in CH₃CN (300 mL) was added morpholine (31.79 g, 364.92 mmol, 32.11 mL, 3 eq) at 25° C. and the reaction mixture was stirred at 100° C. for 12 hours. LC-MS showed the reaction was consumed completely. The mixture was concentrated under reduced pressure at 45° C. The crude product was triturated with petroleum ether (200 mL×2) at 25° C. for half an hour. Compound 6-bromo-8-iodo-2-morpholino-chromen-4-one (49 g, 112.38 mmol, 92.38% yield) was obtained as yellow solid. ¹H NMR (DMSO-d₆, 400 MHz) δ 8.29 (d, J=2.4 Hz, 1H), 7.95 (d, J=2.4 Hz, 1H), 5.59 (s, 1H), 3.78˜3.73 (m, 4H), 3.63˜3.60 (m, 4H).

### Preparation of 6-bromo-2-morpholino-8-vinyl-4H-chromen-4-one (Step 4 in Scheme 2)

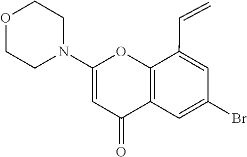 [SMILES: C=Cc1cc(Br)cc2c(=O)cc(N3CCOCC3)oc12]

To a mixture of 6-bromo-8-iodo-2-morpholino-chromen-4-one (5 g, 11.47 mmol, 1.0 eq) and potassium trifluoro(vinyl)boranuide (1.69 g, 12.61 mmol, 1.1 eq) in dioxane (60 mL) and H₂O (10 mL) was added K₂CO₃ (3.17 g, 22.93 mmol, 2.0 eq) and Pd(dppf)Cl₂ (419.52 mg, 573.34 umol, 0.05 eq) at 25° C. under N₂. The mixture was stirred at 70° C. for 4 hours. LC-MS showed the reaction was complete. The reaction mixture was filtered and the filtrate was concentrated. The residue was dissolved in EtOAc (80 mL) and H₂O (80 mL). The organic phase was separated and the aqueous phase was extracted with ethyl acetate (50 mL×2). The combined organic phase was washed with brine (50 mL), dried over anhydrous Na₂SO₄, filtered and concentrated in vacuum. The crude product was triturated with MTBE (50 mL) at 25° C. for half an hour. Compound 6-bromo-2-morpholino-8-vinyl-chromen-4-one (3.8 g, 11.30 mmol, 98.58% yield) was obtained as yellow solid. ¹H NMR (DMSO-d₆, 400 MHz) δ 8.08 (d, J=2.4 Hz, 1H), 7.90 (d, J=2.4 Hz, 1H), 7.11 (dd, J=11.2 Hz, 17.6 Hz, 1H), 6.12 (d, J=17.6 Hz, 1H), 5.60˜5.55 (m, 2H), 3.73˜3.71 (m, 4H), 3.55˜3.51 (m, 4H).

### Preparation of 6-bromo-8-(1-bromoethyl)-2-morpholino-4H-chromen-4-one (Step 5 in Scheme 2)

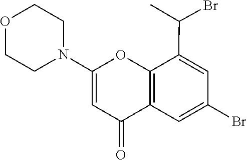 [SMILES: CC(Br)c1cc(Br)cc2c(=O)cc(N3CCOCC3)oc12]

A solution of 6-bromo-2-morpholino-8-vinyl-chromen-4-one (3.8 g, 11.30 mmol, 1.0 eq) in HBr (40 mL, 48%) was stirred at 100° C. for 12 hours. LCMS showed the reaction was complete. There was some yellow solid was formed. After filtration, compound 6-bromo-8-(1-bromoethyl)-2-morpholino-chromen-4-one (3.8 g, 9.11 mmol, 80.60% yield) was obtained as yellow solid. ¹H NMR (DMSO-d₆, 400 MHz) δ 7.99 (d, J=2.4 Hz, 1H), 7.85 (d, J=2.4 Hz, 1H), 5.85 (s, 1H), 5.63˜5.61 (m, 1H), 3.76˜3.71 (m, 8H), 1.39 (d, J=6.4 Hz, 3H).

### Preparation of 6-bromo-8-(1-((3,5-difluorophenyl)amino)ethyl)-2-morpholino-4H-chromen-4-one (Step 6 in Scheme 2)

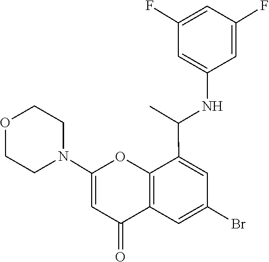 [SMILES: CC(Nc1cc(F)cc(F)c1)c1cc(Br)cc2c(=O)cc(N3CCOCC3)oc12]

A mixture of 6-bromo-8-(1-bromoethyl)-2-morpholino-chromen-4-one (3.8 g, 9.11 mmol, 1.0 eq) and 3,5-difluoroaniline (4.71 g, 36.44 mmol, 4.0 eq) in DMA (40 mL) was stirred at 50° C. for 12 hours. LC-MS showed the reaction was complete. The mixture was poured into water (100 mL) and extracted with ethyl acetate (50 mL×3). The combined organic phase was washed with brine (40 mL), dried with anhydrous Na₂SO₄, filtered and concentrated in vacuum. The residue was purified by flash chromatography (Biotage; 40 g SepaFlash® Silica Flash Column, Eluent of Petroleum ether/Ethyl acetate˜ Ethyl acetate:MeOH=40:1˜4:1). Compound 6-bromo-8-[1-(3,5-difluoroanilino) ethyl]-2-morpholino-chromen 4-one (1.6 g, 3.44 mmol, 37.74% yield) was obtained as yellow solid. ¹H NMR (DMSO-d₆, 400 MHz) δ 7.87 (d, J=2.4 Hz, 1H), 7.64 (d, J=2.4 Hz, 1H), 6.94 (d, J=6.8 Hz, 1H), 6.26 (t, J=9.6 Hz, 1H), 6.16 (d, J=2.0 Hz, 2H), 5.60 (s, 1H), 5.03˜4.92 (m, 1H), 3.76˜3.70 (m, 4H), 3.58˜3.51 (m, 4H), 1.49 (d, J=6.8 Hz, 3H).

### Preparation of Final Compounds in Scheme 2 (Step 7 in Scheme 2)

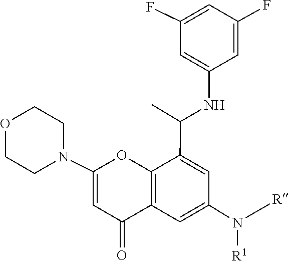

Method A: To a mixture of 6-bromo-8-[1-(3,5-difluoroanilino)ethyl]-2-morpholino-chromen-4-one (279.40 umol, 1.0 eq) and R′N′R″ (838.20 umol˜1.40 mmol, 3 eq˜5 eq), in dioxane (5 mL/mmol˜14 mL/mmol) was added CuI (111.76 umol˜558.80 umol, 0.4 eq˜2.0 eq), N,N′-dimethylethane-1,2-diamine (55.88 umol˜139.70 umol, 0.2 eq˜0.5 eq) and Cs₂CO₃ (838.19 umol, 3.0 eq) at 25° C. under N₂. The mixture was stirred at 100° C. for 10 hours˜48 hours.

Method B: To a mixture of 6-bromo-8-[1-(3,5-difluoroanilino)ethyl]-2-morpholino-chromen-4-one (279.40 umol, 1.0 eq) and R′N′R″ (838.20 umol˜1.40 mmol, 3 eq˜5 eq), in dioxane (5 mL/mmol˜14 mL/mmol) was added [2-(2-aminophenyl) phenylmethylsulfonyloxy-palladium dicyclohexyl-[3,6-dimethoxy-2-(2,4,6-triisopropylphenyl) phenyl]phosphane (27.94 umol, 0.1 eq) and Cs₂CO₃ (838.19 umol, 3.0 eq) at 25° C. under N₂. The mixture was stirred at 100° C. for 10 hours˜48 hours.

Work up: LC-MS showed the reaction was complete. The reaction mixture was filtered and the filtrate was concentrated. The residue was purified by prep-HPLC (column: Welch Xtimate C18 100*25 mm*3 um or Waters Xbridge BEH C18 100*30 mm*10 um; mobile phase: [water (0.05% HCl)-MeCN]; B %: 25%-55%, 8 mins or [water (10 mM NH₄HCO₃)-MeCN]; B %: 35%-45%, 8 mins). The eluent was lyophilized to give the desired product.

### Compound 9

### Preparation of N-(8-(1-((3,5-difluorophenyl)amino)ethyl)-2-morpholino-4-oxo-4H-chromen-6-yl)-N-methylacetamide (Step 7 in Scheme 2) using Method B

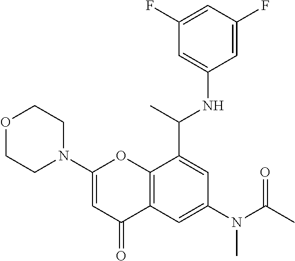 [SMILES: CC(=O)N(C)c1cc(C(C)Nc2cc(F)cc(F)c2)c2oc(N3CCOCC3)cc(=O)c2c1]

To a mixture of 6-bromo-8-[1-(3,5-difluoroanilino)ethyl]-2-morpholino-chromen-4-one (150 mg, 322.38 umol, 1.0 eq) and N-methylacetamide (117.82 mg, 1.61 mmol, 123.11 uL, 5.0 eq) in dioxane (3 mL) was added [2-(2-aminophenyl)phenylmethylsulfonyloxy-palladium dicyclohexyl-[3,6-dimethoxy-2-(2,4,6-triisopropylphenyl)phenyl]phosphane (29.22 mg, 32.24 umol, 0.1 eq) and Cs₂CO₃ (210.08 mg, 644.76 umol, 2.0 eq) at 25° C. under N₂. The mixture was stirred at 100° C. for 12 hours. LC-MS showed the reaction was complete. The reaction mixture was filtered and the filtrate was concentrated. The residue was purified by prep-HPLC (column: Welch Xtimate C18 100*25 mm*3 um; mobile phase: [water (0.05% HCl)-MeCN]; B %: 25%-55%, 8 mins). The eluent was removed under freeze drying. The obtained product was further purified by prep-HPLC (column: Waters Xbridge BEH C18 100*30 mm*10 um; mobile phase: [water (NH₄HCO₃)-MeCN]; B %: 15%-40%, 8 mins). The eluent was removed under freeze drying. Compound N-[8-[1-(3,5-difluoroanilino)ethyl]-2-morpholino-4-oxo-chromen-6-yl]-N-methyl-acetamide (26.4 mg, 57.44 umol, 17.82% yield, 99.54% purity) was obtained as white solid. ¹H NMR (DMSO-d₆, 400 MHz) δ 7.67 (d, J=2.4 Hz, 1H), 7.41˜7.39 (m, 1H), 6.87 (d, J=7.2 Hz, 1H), 6.28˜6.13 (m, 3H), 5.59 (s, 1H), 4.99˜4.97 (m, 1H), 3.74˜3.72 (m, 4H), 3.57˜3.64 (m, 4H), 3.16 (s, 3H), 2.52 (s, 3H), 1.53 (d, J=6.4 Hz, 3H). HPLC: 99.55% (220 nm), 100% (254 nm). MS (ESI): mass calcd. For C₂₄H₂₅F₂O₄N₃ 457.18 m/z found 458.2 [M+H]⁺.

### Compound 10

### 4-(8-(1-((3,5-difluorophenyl)amino)ethyl)-2-morpholino-4-oxo-4H-chromen-6-yl)morpholin-3-one was prepared according to the procedure described herein for Step 7 in Scheme 2 using Method A

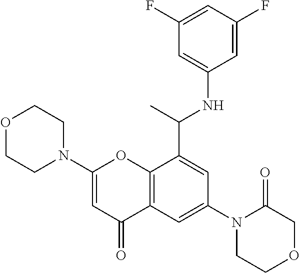 [SMILES: CC(Nc1cc(F)cc(F)c1)c1cc(N2CCOCC2=O)cc2c(=O)cc(N3CCOCC3)oc12]

The desired compound (52.5 mg, 102.01 umol, 36.51% yield, 94.33% purity) was obtained as white solid using Method A. ¹H NMR (DMSO-d₆, 400 MHz) δ 7.78 (d, J=2.4 Hz, 1H), 7.65 (d, J=2.4 Hz, 1H), 6.89 (s, 1H), 6.28˜6.06 (m, 3H), 5.58 (s, 1H), 4.98˜4.96 (m, 1H), 4.18 (s, 2H), 3.98˜3.93 (m, 2H), 3.76˜3.64 (m, 6H), 3.57˜3.51 (m, 4H), 1.49 (d, J=6.8 Hz, 3H). HPLC: 94.33% (220 nm), 99.63% (254 nm). MS (ESI): mass calcd. For C₂₅H₂₅F₂N₅O₃ 485.18 m/z found 486.1 [M+H]⁺.

### Compound 11

### N-(8-(1-((3,5-difluorophenyl)amino)ethyl)-2-morpholino-4-oxo-4H-chromen-6-yl)propionamide was prepared according to the procedure described herein for Step 7 in Scheme 2 using Method A

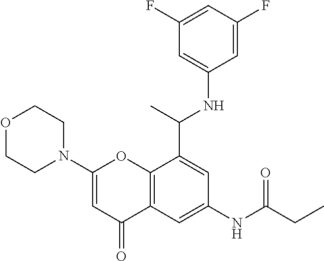 [SMILES: CCC(=O)Nc1cc(C(C)Nc2cc(F)cc(F)c2)c2oc(N3CCOCC3)cc(=O)c2c1]

The desired compound (3.2 mg, 7.00 umol, 2.50% yield) was obtained as white solid using Method A. ¹H NMR (400 MHz, DMSO-d6) δ 10.02 (s, 1H), 8.13 (d, J=2.8 Hz, 1H), 7.76 (d, J=2.8 Hz, 1H), 7.01˜6.99 (m, 1H), 6.23˜6.07 (m, 3H), 5.54 (s, 1H), 4.94˜4.92 (m, 1H), 3.73˜3.71 (m, 4H), 3.54˜3.51 (m, 4H), 2.50˜2.27 (m, 2H), 1.49 (d, J=6.8 Hz, 3H), 1.06 (t, J=7.6 Hz, 3H). LCMS: 94.64% (220 nm), 98.87% (254 nm). MS (ESI): mass calcd. For C₂₄H₂₅F₂N₃O₄ 457.18, m/z found 458.1 [M+H]⁺.

### Compound 12

### N-(8-(1-((3,5-difluorophenyl)amino)ethyl)-2-morpholino-4-oxo-4H-chromen-6-yl)cyclopentanecarboxamide was prepared according to the procedure described herein for Step 7 in Scheme 2 using Method A

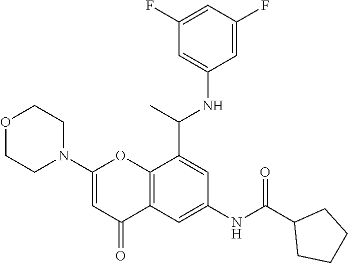 [SMILES: CC(Nc1cc(F)cc(F)c1)c1cc(NC(=O)C2CCCC2)cc2c(=O)cc(N3CCOCC3)oc12]

The desired compound (30.6 mg, 59.09 umol, 21.15% yield, 96.08% purity) was obtained as pale yellow solid using Method A. ¹H NMR (DMSO-d₆, 400 MHz) δ 10.01 (s, 1H), 8.12 (d, J=2.4 Hz, 1H), 7.78 (d, J=2.4 Hz, 1H), 6.97 (d, J=5.6 Hz, 1H), 6.20 (t, J=9.6 Hz, 1H), 6.11-6.08 (m, 2H), 5.50 (s, 1H), 4.93˜4.90 (m, 1H), 3.71 (t, J=4.8 Hz, 4H), 3.53˜3.49 (m, 4H), 2.74˜2.70 (m, 1H), 1.80˜2.07 (m, 2H), 1.70˜1.66 (m, 4H), 1.52 (s, 2H), 1.47 (d, J=6.8 Hz, 3H). LCMS: 96.08% (220 nm), 99.64% (254 nm). MS (ESI): mass calcd. For C₂₇H₂₉F₂N₃O₄ 497.21 m/z found 498.2 [M+H]⁺.

### Compound 13

### N-(8-(1-((3,5-difluorophenyl)amino)ethyl)-2-morpholino-4-oxo-4H-chromen-6-yl)methanesulfonamide was prepared according to the procedure described herein for Step 7 in Scheme 2 using Method A

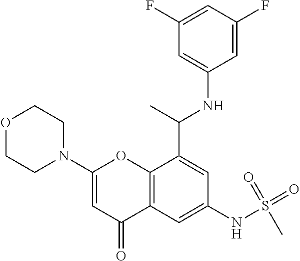 [SMILES: CC(Nc1cc(F)cc(F)c1)c1cc(NS(C)(=O)=O)cc2c(=O)cc(N3CCOCC3)oc12]

The desired compound (12.5 mg, 29.49 umol, 20.15% yield, 97.30% purity) was obtained as white solid using Method A. ¹H NMR (DMSO-d₆, 400 MHz) δ 9.86 (s, 1H), 7.62 (d, J=2.4 Hz, 1H), 7.43 (d, J=2.8 Hz, 1H), 6.98 (d, J=6.4 Hz, 1H), 6.24˜6.19 (m, 1H), 6.12˜6.09 (m, 2H), 5.56 (s, 1H), 4.94 (t, J=6.4 Hz, 1H), 3.71 (t, J=4.8 Hz, 4H), 3.55˜3.30 (m, 4H), 2.85 (s, 3H), 1.49 (d, J=6.8 Hz, 3H). LCMS: 98.01% (220 nm), 100.00% (254 nm). MS (ESI): mass calcd. For C₂₂H₂₃F₂N₃O₅S 479.13 m/z found 480.1 [M+H]⁺.

### Compound 14

### 2-(8-(1-((3,5-difluorophenyl)amino)ethyl)-2-morpholino-4-oxo-4H-chromen-6-yl)guanidine was prepared according to the procedure described herein for Step 7 in Scheme 2 using Method A

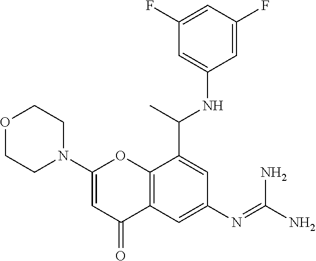 [SMILES: CC(Nc1cc(F)cc(F)c1)c1cc(N=C(N)N)cc2c(=O)cc(N3CCOCC3)oc12]

The desired compound (48.3 mg, 106.45 umol, 24.76% yield, 97.73% purity) was obtained as yellow solid using Method A. ¹H NMR (DMSO-d₆, 400 MHz) δ 9.99 (s, 1H), 7.62 (d, J=2.4 Hz, 1H), 7.57 (s, 4H), 7.38 (s, 1H), 6.25˜6.13 (m, 3H), 5.71 (s, 1H), 4.98˜4.95 (m, 1H), 3.78˜3.73 (m, 4H), 3.69˜3.63 (m, 4H), 1.50 (d, J=6.8 Hz, 3H). LCMS: 97.73% (220 nm), 98.13% (254 nm). MS (ESI): mass calcd. For C₂₂H₂₃F₂N₅O₃ 443.18 m/z found 444.1 [M+H]⁺.

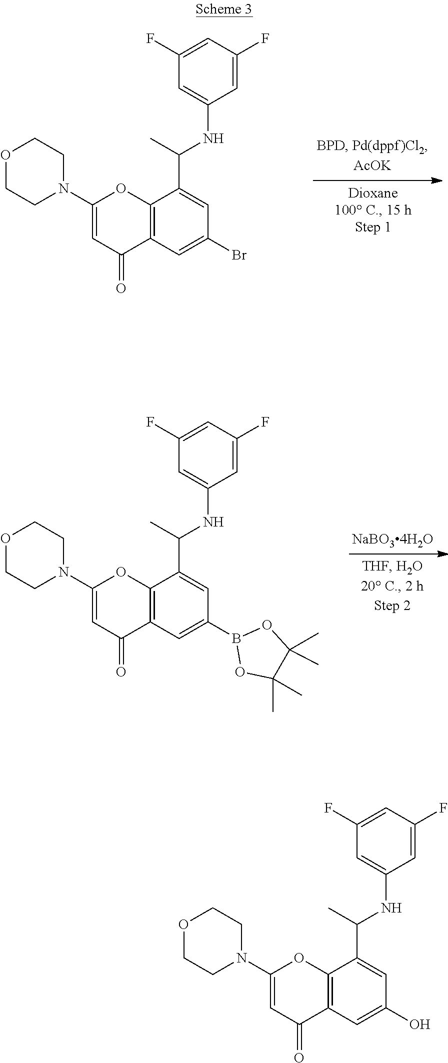 [SMILES: CC(Nc1cc(F)cc(F)c1)c1cc(Br)cc2c(=O)cc(N3CCOCC3)oc12 ; CC(Nc1cc(F)cc(F)c1)c1cc(B2OC(C)(C)C(C)(C)O2)cc2c(=O)cc(N3CCOCC3)oc12 ; CC(Nc1cc(F)cc(F)c1)c1cc(O)cc2c(=O)cc(N3CCOCC3)oc12]

### Compound 15

### Preparation of 8-(1-((3,5-difluorophenyl)amino)ethyl)-6-hydroxy-2-morpholino-4H-chromen-4-one (Steps 1-2 in Scheme 3)

### Preparation of 8-(1-((3,5-difluorophenyl)amino)ethyl)-2-morpholino-6-(4,4,5,5-tetramethyl-1,3,2-dioxaborolan-2-yl)-4H-chromen-4-one (Step 1 in Scheme 3)

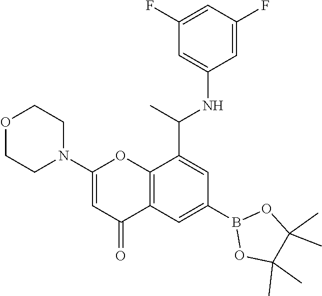 [SMILES: CC(Nc1cc(F)cc(F)c1)c1cc(B2OC(C)(C)C(C)(C)O2)cc2c(=O)cc(N3CCOCC3)oc12]

A mixture of 6-bromo-8-[1-(3,5-difluoroanilino)ethyl]-2-morpholino-chromen-4-one (0.3 g, 644.76 umol, 1 eq), BPD (491.19 mg, 1.93 mmol, 3 eq), Pd(dppf)Cl₂ (47.18 mg, 64.48 umol, 0.1 eq) and AcOK (189.83 mg, 1.93 mmol, 3 eq) in dioxane (3 mL) was degassed and purged with N₂ for 3 times and then the mixture was stirred at 100° C. for 15 hours under N₂ atmosphere. LC-MS showed the reaction was complete. The reaction mixture was filtered and the filtrate was concentrated in vacuum. Compound 8-[1-(3,5-difluoroanilino)ethyl]-2-morpholino-6-(4,4,5,5-tetramethyl-1,3,2-dioxaborolan-2-yl)chromen-4-one (1.170 g, crude) was obtained as brown oil. ¹H NMR (DMSO-d₆, 400 MHz) δ 8.18 (s, 1H), 7.85 (s, 1H), 7.04 (d, J=6.8 Hz, 1H), 6.24 (t, J=9.6 Hz, 1H), 6.13 (d, J=10.4 Hz, 2H), 5.56 (s, 1H), 4.99 (t, J=6.4 Hz, 1H), 3.74 (t, J=4.4 Hz, 4H), 3.55 (t, J=5.2 Hz, 4H), 1.48 (d, J=6.8 Hz, 3H), 1.29 (s, 12H).

### Preparation of 8-(1-((3,5-difluorophenyl)amino)ethyl)-6-hydroxy-2-morpholino-4H-chromen-4-one (Step 2 in Scheme 3)

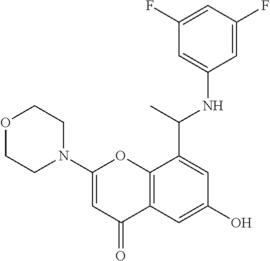 [SMILES: CC(Nc1cc(F)cc(F)c1)c1cc(O)cc2c(=O)cc(N3CCOCC3)oc12]

To a solution of 8-[1-(3,5-difluoroanilino)ethyl]-2-morpholino-6-(4,4,5,5-tetramethyl-1,3,2-dioxaborolan-2-yl)chromen-4-one (1.1 g, 2.15 mmol, 1 eq) in THF (20 mL) and H₂O (20 mL) was added sodium 3-oxidodioxaborirane tetrahydrate (5.82 g, 37.84 mmol, 7.28 mL, 17.62 eq). The mixture was stirred at 20° C. for 2 hours. LC-MS and HPLC showed 8-[1-(3,5-difluoroanilino)ethyl]-2-morpholino-6-(4,4,5,5-tetramethyl-1,3,2-dioxaborolan-2-yl)chromen-4-one was consumed completely. The reaction mixture was partitioned between EtOAc (45 mL) and H₂O (15 mL). The aqueous phase was separated and then extracted with EtOAc (10 mL×3). The organic phase was washed with brine (10 mL), dried over with Na₂SO₄, filtered and concentrated under reduced pressure. The residue was purified by prep-HPLC (column: Waters Xbridge BEH C18 100*30 mm*10 um; mobile phase: [water (10 mM NH₄HCO₃)-MeCN]; B %: 25%-50%, 8 mins). The eluent was removed under freeze drying. Compound 8-[1-(3,5-difluoroanilino)ethyl]-6-hydroxy-2-morpholino-chromen-4-one (56.5 mg, 140.41 umol, 40.36% yield, 100.00% purity) was obtained as white solid. ¹H NMR (DMSO-d₆, 400 MHz) δ 9.74 (s, 1H), 7.13 (d, J=2.8 Hz, 1H), 7.00 (d, J=2.8 Hz, 1H), 6.95 (s, 1H), 6.24 (t, J=6.8 Hz, 1H), 6.12 (d, J=9.2 Hz, 2H), 5.60 (s, 1H), 4.92 (d, J=6.4 Hz, 1H), 3.74 (t, J=4.4 Hz, 4H), 3.58˜3.53 (m, 4H), 1.48 (d, J=6.4 Hz, 3H). LCMS: 100.00% (220 nm), 100.00% (254 nm). MS (ESI): mass calcd. For C₂₁H₂₀F₂N₂O₄ 402.14 m/z found 403.1 [M+H]⁺.

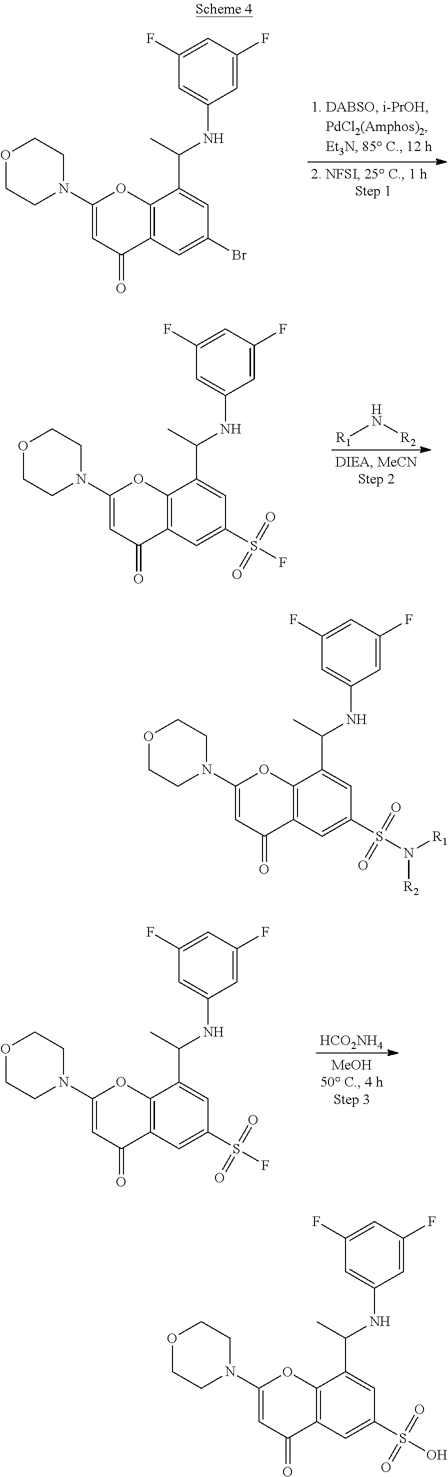

### General Procedures for Preparing Compounds in Scheme 4

### Preparation of 8-(1-((3,5-difluorophenyl)amino)ethyl)-2-morpholino-4-oxo-4H-chromene-6-sulfonyl fluoride (Step 1 in Scheme 4)

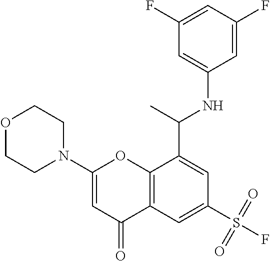 [SMILES: CC(Nc1cc(F)cc(F)c1)c1cc(S(=O)(=O)F)cc2c(=O)cc(N3CCOCC3)oc12]

To a mixture of 6-bromo-8-[1-(3,5-difluoroanilino)ethyl]-2-morpholino-chromen-4-one (1 g, 2.15 mmol, 1.0 eq) and 1,4-diazoniabicyclo[2.2.2]octane-1,4-disulfinate (568.10 mg, 2.36 mmol, 1.1 eq) in i-PrOH (15 mL) was added Et₃N (652.43 mg, 6.45 mmol, 897.43 uL, 3.0 eq) and 4-ditert-butylphosphanyl-N,N-dimethyl-aniline dichloropalladium (152.18 mg, 214.92 umol, 152.18 uL, 0.1 eq) at 25° C. under N₂. The mixture was stirred at 85° C. for 12 hours and then cooled to 25° C. To the above reaction mixture was added NFSI (1.02 g, 3.22 mmol, 1.5 eq) and the mixture was stirred at 25° C. for an hour. LC-MS showed ˜20% of 8-(1-((3,5-difluorophenyl)amino)ethyl)-2-morpholino-4H-chromen-4-one and ˜65% of desired compound was detected. The reaction mixture was filtered and the filtrate was concentrated. The residue was purified by column chromatography (SiO₂, Petroleum ether/Ethyl acetate˜EtOAc:MeOH=40:1˜4:1). The solvent was concentrated. Compound 8-[1-(3,5-difluoroanilino)ethyl]-2-morpholino-4-oxo-chromene-6-sulfonyl fluoride (0.48 g, 1.02 mmol, 11.92% yield) was obtained as yellow solid. ¹H NMR (DMSO-d₆, 400 MHz) δ 8.38 (d, J=2.4 Hz, 1H), 8.09 (d, J=2.4 Hz, 1H), 7.04 (d, J=6.8 Hz, 1H), 6.33˜6.11 (m, 3H), 5.75 (s, 1H), 5.11-4.98 (m, 1H), 3.77˜3.72 (m, 4H), 3.65˜3.54 (m, 4H), 1.54 (d, J=6.8 Hz, 3H).

### Preparation of Compounds in Scheme 4 (Step 2 in Scheme 4)

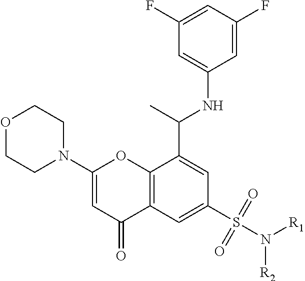

To a mixture of 8-[1-(3,5-difluoroanilino)ethyl]-2-morpholino-4-oxo-chromene-6-sulfonyl fluoride (156.90 umol, 1.0 eq) and R₁NHR₂ (0.31 mmol, 2.0 eq, HCl or free base) in MeCN (6 mL/mmol˜12 mL/mmol) was added DIEA (0.47 mmol, 390.42 uL, 3.0 eq) dropwise at room temperature. The mixture was stirred at 80° C. for 12 hours. HPLC and LC-MS showed 8-[1-(3,5-difluoroanilino)ethyl]-2-morpholino-4-oxo-chromene-6-sulfonyl fluoride was consumed completely. The mixture was quenched with water (5 mL) and extracted with ethyl acetate (5 mL×3). The combined organic phase was washed with brine (5 mL), dried with anhydrous Na₂SO₄, filtered and concentrated in vacuum. The residue was purified by prep-HPLC (column: Phenomenex luna C18 80*40 mm*3 um or Phenomenex Gemini-NX C18 75*30 mm*3 um or Waters Xbridge BEH C18 10*30 mm*10 um; mobile phase: [water (0.04% HCl)-MeCN]; B %: 32%-54%, 7 mins or [water (10 mM NH₄HCO₃)-MeCN]; B %: 25%-45%, 8 mins or [water (10 mM NH₄HCO₃)-MeCN]; B %: 30%-60%, 10 mins). The aqueous solution was lyophilized to give the desired product.

### Compound 16

### Preparation of 8-(1-((3,5-difluorophenyl)amino)ethyl)-N-methyl-2-morpholino-4-oxo-4H-chromene-6-sulfonamide (Step 2 in Scheme 4)

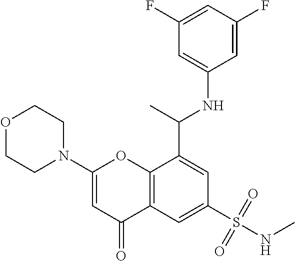 [SMILES: CNS(=O)(=O)c1cc(C(C)Nc2cc(F)cc(F)c2)c2oc(N3CCOCC3)cc(=O)c2c1]

To a mixture of 8-[1-(3,5-difluoroanilino)ethyl]-2-morpholino-4-oxo-chromene-6-sulfonyl fluoride (0.31 g, 661.76 umol, 1 eq) and methanamine (89.36 mg, 1.32 mmol, 2 eq, HCl) in CH₃CN (5 mL) was added DIEA (256.58 mg, 1.99 mmol, 345.80 uL, 3 eq) at 25° C. The mixture was stirred at 80° C. for 12 hours. HPLC and LC-MS showed 8-[1-(3,5-difluoroanilino)ethyl]-2-morpholino-4-oxo-chromene-6-sulfonyl fluoride was consumed completely. The mixture was quenched with water (20 mL) and the aqueous phase was extracted with ethyl acetate (15 mL×3). The combined organic phase was washed with brine (10 mL), dried with anhydrous Na₂SO₄, filtered and concentrated in vacuum. The residue was purified by prep-HPLC (column: Phenomenex Gemini-NX 80*40 mm*3 um; mobile phase: [water (10 mM NH₄HCO₃)-MeCN]; B %: 20%-50%, 8 mins). The eluent was removed under freeze drying. Compound 8-[1-(3,5-difluoroanilino)ethyl]-N-methyl-2-morpholino-4-oxo-chromene-6-sulfonamide (118.9 mg, 247.97 umol, 37.47% yield, 100% purity) was obtained as white solid. ¹H NMR (400 MHz, DMSO-d₆) δ 8.20 (d, J=2.4 Hz, 1H), 7.86 (d, J=2.4 Hz, 1H), 7.49˜7.51 (m, 1H), 7.05 (d, J=6.8 Hz, 1H), 6.25˜6.14 (m, 3H), 5.66 (s, 1H), 5.03˜5.00 (m, 1H), 3.74˜3.72 (m, 4H), 3.60˜3.53 (m, 4H), 2.22 (d, J=4.8 Hz, 3H), 1.53 (d, J=6.4 Hz, 3H). LCMS: 100.00% (220 nm), 100.00% (254 nm). MS (ESI): mass calcd. For C₂₂H₂₃F₂N₃O₅S 479.13 m/z found 480.1 [M+H]⁺.

### Compound 17

### 6-((3-aminoazetidin-1-yl)sulfonyl)-8-(1-((3,5-difluorophenyl)amino)ethyl)-2-morpholino-4H-chromen-4-one was prepared according to the procedure described herein for Step 2 in Scheme 4)

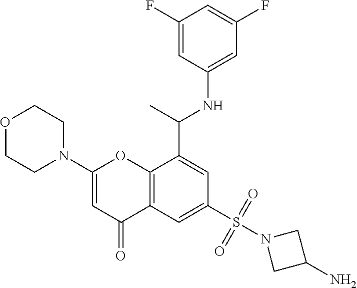 [SMILES: CC(Nc1cc(F)cc(F)c1)c1cc(S(=O)(=O)N2CC(N)C2)cc2c(=O)cc(N3CCOCC3)oc12]

The desired compound (18.8 mg, 33.96 umol, 11.71% yield, 94.02% purity) was obtained as white solid. ¹H NMR (400 MHz, DMSO-d₆) δ 8.10 (d, J=2.4 Hz, 1H), 7.80 (d, J=2.0 Hz, 1H), 7.08 (d, J=6.8 Hz, 1H), 6.24˜6.18 (m, 3H), 5.70 (s, 1H), 5.09˜5.02 (m, 1H), 3.78˜3.72 (m, 4H), 3.63˜3.56 (m, 5H), 3.31-3.24 (m, 2H), 3.05˜3.02 (m, 1H), 2.90˜2.87 (m, 1H), 1.55 (d, J=6.8 Hz, 3H). LCMS: 94.02% (220 nm), 94.50% (254 nm). MS (ESI): mass calcd. For C₂₄H₂₆F₂N₄O₅S 520.16 m/z found 521.2 [M+H]⁺.

### Compound 18

### Preparation of 8-(1-((3,5-difluorophenyl)amino)ethyl)-2-morpholino-4-oxo-4H-chromene-6-sulfonic acid (Step 3 in Scheme 4)

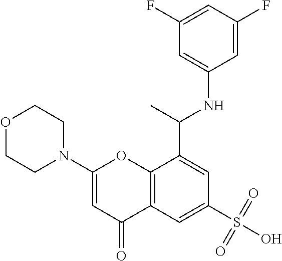 [SMILES: CC(Nc1cc(F)cc(F)c1)c1cc(S(=O)(=O)O)cc2c(=O)cc(N3CCOCC3)oc12]

To a solution of 8-[1-(3,5-difluoroanilino)ethyl]-2-morpholino-4-oxo-chromene-6-sulfonyl fluoride (120 mg, 256.17 umol, 1.0 eq) in MeOH (10 mL) was added ammonia formic acid (484.58 mg, 7.68 mmol, 30 eq). The mixture was stirred at 50° C. for 4 hours. LC-MS and HPLC showed 8-[1-(3,5-difluoroanilino)ethyl]-2-morpholino-4-oxo-chromene-6-sulfonyl fluoride was consumed completely. The mixture was concentrated in vacuum. The residue was purified by prep-HPLC (column: Waters Xbridge BEH C18 100*25 mm*5 um; mobile phase: [water (10 mM NH₄HCO₃)-MeCN]; B %: 15%-45%, 10 mins). The eluent was removed under freeze drying. Compound 8-[1-(3,5-difluoroanilino)ethyl]-2-morpholino-4-oxo-chromene-6-sulfonic acid (30 mg, 64.58 umol, 24.71% yield, 99.58% purity) was obtained as a white solid. ¹H NMR (400 MHz, D₂O) δ 8.20 (d, J=2.0 Hz, 1H), 7.95 (d, J=2.0 Hz, 1H), 6.09˜6.03 (m, 3H), 5.63 (s, 1H), 4.85˜4.83 (m, 1H), 3.79˜3.78 (m, 4H), 3.58˜3.57 (m, 4H), 1.48 (d, J=6.8 Hz, 3H). LCMS: 99.58% (220 nm), 100.00% (254 nm). MS (ESI): mass calcd. For C₂₁H₂₀F₂N₂O₆S 466.10 m/z found 467.1 [M+H]⁺.

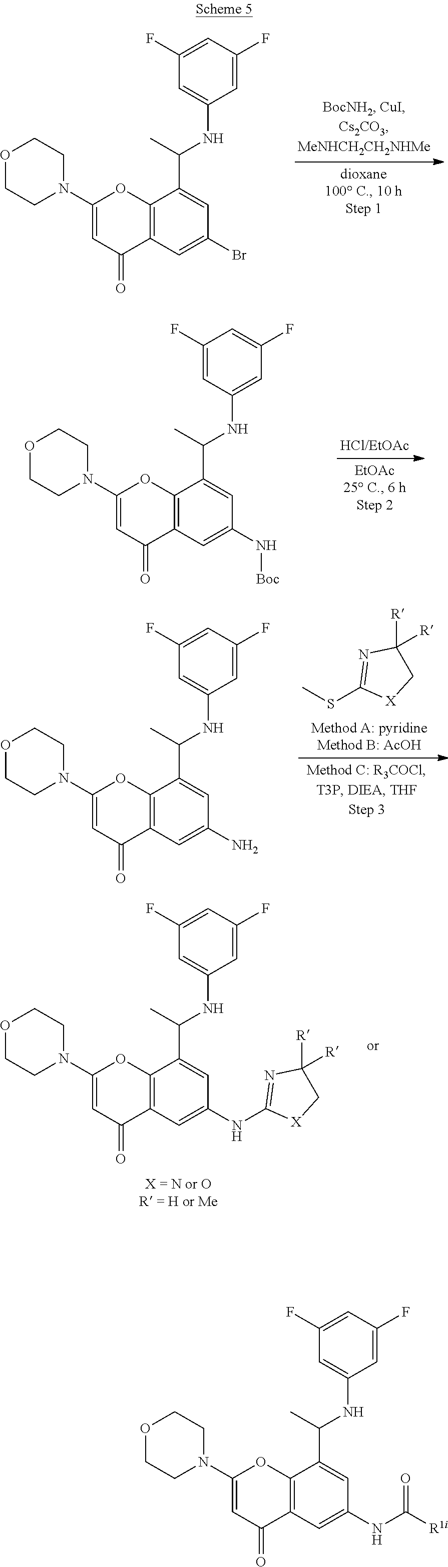

### General Procedures for Preparing Compounds in Scheme 5

### Preparation of tert-butyl (8-(1-((3,5-difluorophenyl)amino)ethyl)-2-morpholino-4-oxo-4H-chromen-6-yl)carbamate (Step 1 in Scheme 5)

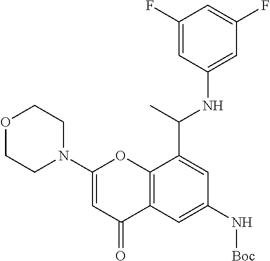 [SMILES: CC(Nc1cc(F)cc(F)c1)c1cc(NC(=O)OC(C)(C)C)cc2c(=O)cc(N3CCOCC3)oc12]

A mixture of 6-bromo-8-[1-(3,5-difluoroanilino)ethyl]-2-morpholino-chromen-4-one (1 g, 2.15 mmol, 1 eq), tert-butyl carbamate (755.31 mg, 6.45 mmol, 5.76 uL, 3 eq), CuI (409.32 mg, 2.15 mmol, 1 eq), N,N′-dimethylethane-1,2-diamine (56.84 mg, 644.76 umol, 69.40 uL, 0.3 eq) and Cs₂CO₃ (2.10 g, 6.45 mmol, 3 eq) in dioxane (10 mL) was degassed and purged with N₂ for 3 times. Then the mixture was stirred at 100° C. for 10 hours under N₂ atmosphere. TLC (Petroleum ether:Ethyl acetate=1:1, R_{f}=0.25), LC-MS and HPLC showed the reaction was complete. The mixture was quenched with water (10 mL) and the aqueous phase was extracted with ethyl acetate (15 mL×3). The combined organic phase was washed with brine (10 mL), dried with anhydrous Na₂SO₄, filtered and concentrated in vacuum. The residue was purified by flash silica gel chromatography (Biotage; 12 g SepaFlash® Silica Flash Column, Eluent of 0˜100% Ethyl acetate gradient @ 60 mL/min). The eluent was removed under reduced pressure. Compound tert-butylN-[8-[1-(3,5-difluoroanilino)thyl]-2-morpholino-4-oxo-chro men-6-yl]carbamate (0.55 g, 1.10 mmol, 51.03% yield) was obtained as pale yellow solid. ¹H NMR (DMSO-d₆, 400 MHz) δ 9.52 (s, 1H), 7.95 (s, 1H), 7.65 (d, J=2.8 Hz, 1H), 6.96 (d, J=6.4 Hz, 1H), 6.23˜6.08 (m, 3H), 5.51 (s, 1H), 4.90 (t, J=6.0 Hz, 1H), 3.71 (t, J=4.8 Hz, 4H), 3.50 (t, J=4.8 Hz, 4H), 1.45˜1.49 (m, 12H).

### Compound 19

### Preparation of 6-amino-8-(1-((3,5-difluorophenyl)amino)ethyl)-2-morpholino-4H-chromen-4-one (Step 2 in Scheme 5)

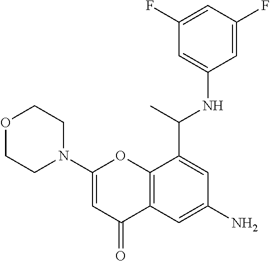 [SMILES: CC(Nc1cc(F)cc(F)c1)c1cc(N)cc2c(=O)cc(N3CCOCC3)oc12]

To a solution of tert-butyl N-[8-[1-(3,5-difluoroanilino)ethyl]-2-morpholino-4-oxo-chromen-6-yl]carbamate (0.66 g, 1.28 mm ol, 1 eq) in EtOAc (2 mL) was added HCl/EtOAc (4 M, 6.69 mL, 24.39 eq). The mixture was stirred at 25° C. for 6 hours. LC-MS showed the reaction was complete. There was pale yellow precipitate formed. After filtration, the solid was collected and dried. Compound 6-amino-8-[1-(3,5-difluoroanilino)ethyl]-2-morpholino-chromen-4-one (471 mg, 1.08 mmol, 98.09% yield, HCl) was obtained as pale yellow solid. ¹H NMR (400 MHz, DMSO-d₆) δ 7.62 (s, 1H), 7.38 (s, 1H), 7.08 (s, 1H), 6.30˜5.97 (m, 3H), 5.71 (s, 1H), 4.96˜4.94 (m, 1H), 3.78˜3.76 (m, 4H), 3.57˜3.54 (m, 4H), 1.49 (d, J=9.6 Hz, 3H). HPLC: 95.08% (220 nm), 96.94% (254 nm). MS (ESI): mass calcd. For C₂₁H₂₁F₂N₃O₃ 401.16 m/z found 402.1 [M+H]⁺.

### Preparation of Compounds in Scheme 5 (Step 3 in Scheme 5)

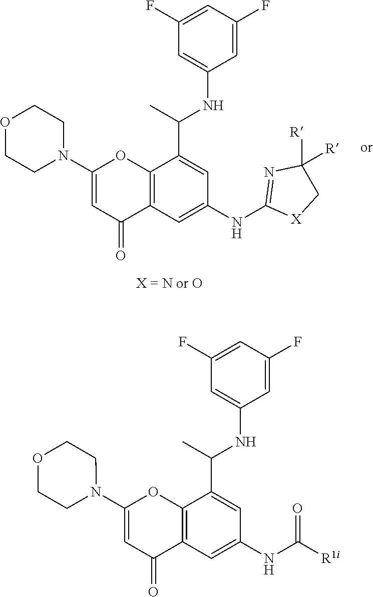

To a solution of 6-amino-8-[1-(3,5-difluoroanilino)ethyl]-2-morpholino-chromen-4-one (274.06 umol, 1 eq, HCl) in pyridine (5 mL/mmol) or AcOH (10 mL/mmol) was added MeS-amine (1.37 mmol, 5 eq). Or to a mixture of 6-amino-8-[1-(3,5-difluoroanilino)ethyl]-2-morpholino-chromen-4-one (124.56 umol, 1.0 eq), 2-cyanoacetic acid (373.69 umol, 3.0 eq) and DIEA (622.81 umol, 108.48 uL, 5.0 eq) in THF (15 mL/mmol) was added T3P (448.42 umol, 266.69 uL, 50% purity, 3.6 eq) dropwise at 0° C. Then the mixture was stirred at 25° C.˜150° C. for 4 hours˜15 hours. HPLC and LC-MS showed the reaction was complete. The mixture was filtered and the filtrate was concentrated in vacuum or the mixture was basified with sat. NaOH and NaHCO₃ to pH=7. There was precipitate formed. The precipitate was collected by filtration. The residue or the filter cake was purified by prep-HPLC (column: Welch Xtimate C18 100*25 mm*3 um or Phenomenex luna C18 80*40 mm*3 um; mobile phase: [water (0.05% HCl)-MeCN]; B %: 15%-30%, 8 mins or [water (0.04% HCl)-MeCN]; B %: 27%-42%, 5.5 mins or [water (0.04% HCl)-MeCN]; B %: 23%-47%, 7 mins). The eluent was removed under freeze drying.

### Compound 20

### Preparation of 8-(1-((3,5-difluorophenyl)amino)ethyl)-6-((4,5-dihydro-1H-imidazol-2-yl)amino)-2-morpholino-4H-chromen-4-one (Step 3 in Scheme 5)

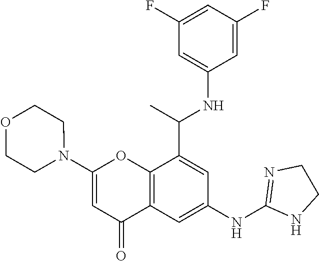 [SMILES: CC(Nc1cc(F)cc(F)c1)c1cc(NC2=NCCN2)cc2c(=O)cc(N3CCOCC3)oc12]

To a solution of 6-amino-8-[1-(3,5-difluoroanilino)ethyl]-2-morpholino-chromen-4-one (120 mg, 274.06 umol, 1 eq, HCl) in pyridine (1.5 mL) was added 2-methylsulfanyl-4,5-dihydro-1H-imidazole-hydroiodide (334.48 mg, 1.37 mmol, 5 eq). The mixture was stirred at 150° C. for 15 hours. HPLC and LC-MS showed the reaction was complete. The mixture was filtered and the filtrate was concentrated in vacuum. The residue was purified by prep-HPLC (column: Welch Xtimate C18 100*25 mm*3 um; mobile phase: [water (0.05% HCl)-MeCN]; B %: 15%-30%, 8 mins). The eluent was removed under freeze drying. Compound 8-(1-((3,5-difluorophenyl)amino)ethyl)-6-((4,5-dihydro-1H-imidazol-2-yl)amino)-2-morpholino-4H-chromen-4-one (13.7 mg, 26.34 umol, 9.61% yield, 97.28% purity, HCl) was obtained as yellow solid. ¹H NMR (DMSO-d₆, 400 MHz) δ 10.64 (s, 1H), 8.40 (s, 2H), 7.67 (d, J=2.8 Hz, 1H), 7.39 (d, J=2.4 Hz, 1H), 6.23 (t, J=9.6 Hz, 1H), 6.15˜6.13 (m, 2H), 5.66 (s, 1H), 5.0˜4.95 (m, 1H), 3.75˜3.72 (m, 8H), 3.58˜3.56 (m, 4H), 1.51 (d, J=6.8 Hz, 3H). LCMS: 97.28% (220 nm), 98.00% (254 nm). MS (ESI): mass calcd. For C₂₄H₂₅O₃N₅F₂ 469.19 m/z found 470.2 [M+H]⁺.

### Compound 21

### 8-(1-((3,5-difluorophenyl)amino)ethyl)-6-((4,4-dimethyl-4,5-dihydrooxazol-2-yl)amino)-2-morpholino-4H-chromen-4-one was prepared according to the procedure described herein for Step 3 in Scheme 5

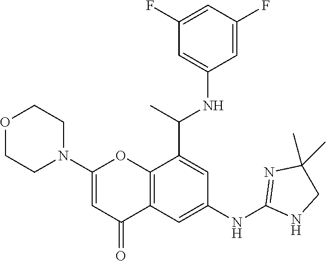 [SMILES: CC(Nc1cc(F)cc(F)c1)c1cc(NC2=NC(C)(C)CN2)cc2c(=O)cc(N3CCOCC3)oc12]

The desired compound (4.7 mg, 9.43 umol, 4.13% yield, 90.56% purity) was obtained as yellow solid. ¹H NMR (DMSO-d₆, 400 MHz) δ 7.82 (s, 1H), 7.49 (s, 1H), 6.23 (s, 1H), 6.16 (d, J=9.2 Hz, 2H), 5.67 (s, 1H), 4.98 (s, 1H), 4.56 (s, 2H), 3.73 (s, 4H), 3.60˜3.57 (m, 4H), 1.51 (d, J=4.8 Hz, 3H), 1.41 (s, 6H). LCMS: 90.56% (220 nm), 88.11% (254 nm). MS (ESI): mass calcd. For C₂₆H₂₈F₂N₄O₄ 498.21 m/z found 499.2 [M+H]⁺.

### Compound 22

### 2-cyano-N-(8-(1-((3,5-difluorophenyl)amino)ethyl)-2-morpholino-4-oxo-4H-chromen-6-yl)acetamide was prepared according to the procedure described herein for Step 3 in Scheme 5

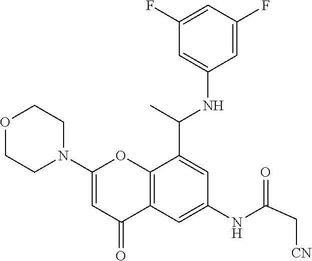 [SMILES: CC(Nc1cc(F)cc(F)c1)c1cc(NC(=O)CC#N)cc2c(=O)cc(N3CCOCC3)oc12]

The desired compound (14.0 mg, 29.42 umol, 23.62% yield, 98.45% purity) was obtained as white solid. ¹H NMR (400 MHz, DMSO-d6) δ 10.46 (s, 1H), 8.11 (d, J=2.8 Hz, 1H), 7.66 (d, J=2.8 Hz, 1H), 7.00 (s, 1H), 6.21 (t, J=9.6 Hz, 1H), 6.10 (d, J=9.6 Hz, 2H), 5.58 (s, 1H), 4.93 (d, J=6.8 Hz, 1H), 3.86 (s, 2H), 3.73˜3.71 (m, 4H), 3.64˜3.53 (m, 4H), 1.49 (d, J=6.5 Hz, 3H). LCMS: 98.45% (220 nm), 98.82% (254 nm). MS (ESI): mass calcd. For C₂₄H₂₂F₂N₄O₄ 468.16 m/z found 469.1 [M+H]⁺.

### Compound 23

### (Z)-2-cyano-N-(8-(1-((3,5-difluorophenyl)amino)ethyl)-2-morpholino-4-oxo-4H-chromen-6-yl)-3-hydroxybut-2-enamide was prepared according to the procedure described herein for Step 3 in

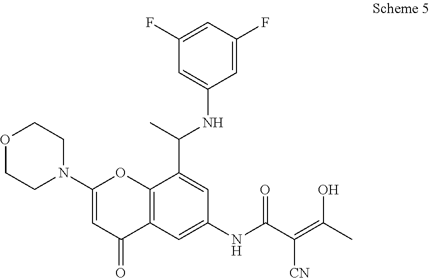 [SMILES: C/C(O)=C(\C#N)C(=O)Nc1cc(C(C)Nc2cc(F)cc(F)c2)c2oc(N3CCOCC3)cc(=O)c2c1]

The desired compound (5.8 mg, 10.03 umol, 6.71% yield, 94.59% purity, HCl) was obtained as yellow solid. ¹H NMR (DMSO-d₆, 400 MHz) δ 8.12 (d, J=2.4 Hz, 1H), 7.67 (d, J=2.4 Hz, 1H), 6.27˜6.07 (m, 3H), 5.73 (s, 1H), 5.02˜4.84 (m, 1H), 3.77˜3.70 (m, 4H), 3.67˜3.62 (m, 4H), 2.19 (s, 3H), 1.49 (d, J=6.8 Hz, 3H). HPLC: 94.60% (220 nm), 92.11% (254 nm). MS (ESI): mass calcd. For C₂₆H₂₄F₂N₄O₅ 510.17 m/z found 511.1 [M+H]⁺.

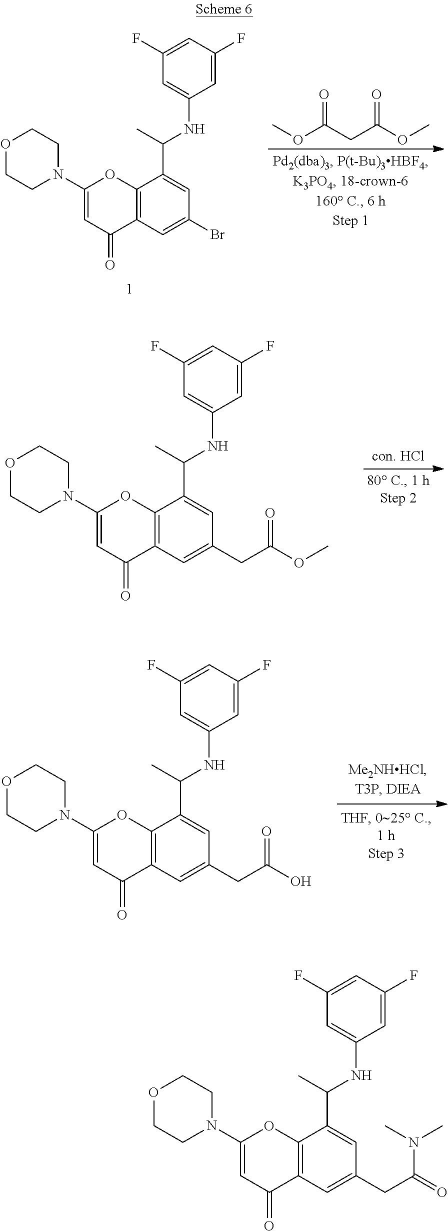

### Compound 24

### Preparation of 2-(8-(1-((3,5-difluorophenyl)amino)ethyl)-2-morpholino-4-oxo-4H-chromen-6-yl)-N,N-dimethylacetamide (Steps 1-3 in Scheme 6)

### Compound 25

### Preparation of methyl 2-(8-(1-((3,5-difluorophenyl)amino)ethyl)-2-morpholino-4-oxo-4H-chromen-6-yl)acetate (Step 1 in Scheme 6)

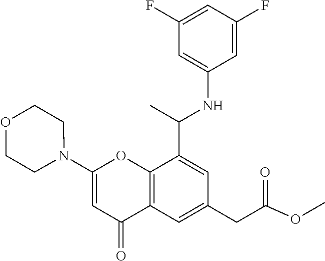 [SMILES: COC(=O)Cc1cc(C(C)Nc2cc(F)cc(F)c2)c2oc(N3CCOCC3)cc(=O)c2c1]

A mixture of 6-bromo-8-[1-(3,5-difluoroanilino)ethyl]-2-morpholino-chromen-4-one (1 g, 2.15 mmol, 1 eq), dimethylpropanedioate (1.87 g, 14.18 mmol, 1.63 mL, 6.6 eq), tritert-butylphosphonium tetrafluoroborate (124.71 mg, 429.84 umol, 0.2 eq), K₃PO₄ (1.28 g, 6.02 mmol, 2.8 eq), 18-crown-6 (284.03 mg, 1.07 mmol, 0.5 eq) and Pd₂(dba)₃ (196.81 mg, 214.92 umol, 0.1 eq) was degassed and purged with N₂ for 3 times and then the mixture was stirred at 160° C. for 6 hours under N₂ atmosphere. TLC (Petroleum ether:Ethyl acetate=0:1, R_{f}=0.44), LC-MS and HPLC indicated the reaction was complete. The reaction mixture was filtered and the filter cake was washed with ethyl acetate (10 mL×6). The combined filtrate was concentrated in vacuum. The residue was quenched with water (100 mL) and then extracted with ethyl acetate (15 mL×3). The combined organic phase was washed with brine (10 mL×3), dried with anhydrous Na₂SO₄, filtered and concentrated in vacuum. The residue was purified by flash silica gel chromatography (Biotage; 20 g SepaFlash® Silica Flash Column, Eluent of 0˜100% Ethyl acetate, 0˜40% MeOH/Ethyl acetate gradient @ 60 mL/min). The eluent was removed under reduced pressure to give 804 mg of methyl 2-[8-[1-(3,5-difluoroanilino)ethyl]-2-morpholino-4-oxo-chromen-6-yl]acetate. 30 mg of crude product was further purified by prep-HPLC (column: Welch Xtimate C18 100*25 mm*3 um; mobile phase: [water (0.05% HCl)-MeCN]; B %: 30%-45%, 8 mins). Compound methyl 2-[8-[1-(3,5-difluoroanilino)ethyl]-2-morpholino-4-oxo-chromen-6-yl]acetate (10.7 mg, 22.37 umol, 1.04% yield, 95.83% purity) was obtained as yellow solid. ¹H NMR (DMSO-d₆, 400 MHz) δ 7.74 (d, J=2.0 Hz, 1H), 7.49 (d, J=2.0 Hz, 1H), 6.23˜6.18 (m, 1H), 6.14 (t, J=1.6 Hz, 2H), 5.73 (s, 1H), 4.98˜4.93 (m, 1H), 3.75˜3.71 (m, 6H), 3.70˜3.59 (m, 7H), 1.50 (d, J=6.8 Hz, 3H). LCMS: 95.83% (220 nm), 96.03% (254 nm). MS (ESI): mass calcd. For C₂₄H₂₄F₂N₂O₅ 458.17 m/z found 459.1 [M+H]⁺.

### Compound 26

### Preparation of 2-(8-(1-((3,5-difluorophenyl)amino)ethyl)-2-morpholino-4-oxo-4H-chromen-6-yl)acetic acid (Step 2 in Scheme 6)

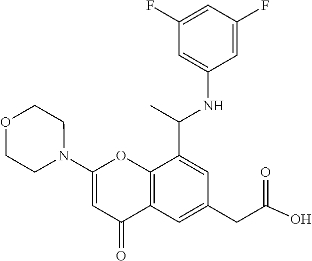 [SMILES: CC(Nc1cc(F)cc(F)c1)c1cc(CC(=O)O)cc2c(=O)cc(N3CCOCC3)oc12]

A solution of methyl 2-[8-[1-(3,5-difluoroanilino)ethyl]-2-morpholino-4-oxo-chromen-6-yl] acetate (0.1 g, 218.12 umol, 1 eq) in HCl (12 M, 0.15 mL, 8.25 eq) was stirred at 80° C. for an hour. LC-MS showed methyl 2-[8-[1-(3,5-difluoroanilino)ethyl]-2-morpholino-4-oxo-chromen-6-yl]acetate was consumed completely. The mixture was basified with sat. NaOH (0.5 mL) to pH=5. The resulting precipitate (150 mg) was collected by filtration. 20 mg of the precipitate was further purified by prep-HPLC (column: Welch Xtimate C18 100*25 mm*3 um; mobile phase: [water (0.05% HCl)-MeCN]; B %: 25%-40%, 8 mins). The eluent was removed under freeze drying. Compound 2-[8-[1-(3,5-difluoroanilino)ethyl]-2-morpholino-4-oxo-chromen-6-yl]acetic acid (2.4 mg, 5.33 umol, 2.44% yield, 98.64% purity) was obtained as white solid. ¹H NMR (DMSO-d₆, 400 MHz) δ 7.72 (d, J=2.0 Hz, 1H), 7.48 (d, J=2.0 Hz, 1H), 6.92 (s, 1H), 6.22˜6.18 (m, 1H), 6.14˜6.08 (m, 2H), 5.64 (s, 1H), 4.96˜4.95 (m, 1H), 3.74 (t, J=4.4 Hz, 4H), 3.63 (d, J=3.2 Hz, 2H), 3.56˜3.53 (m, 4H), 1.50 (d, J=6.8 Hz, 3H). LCMS: 98.64% (220 nm), 99.38% (254 nm). MS (ESI): mass calcd. For C₂₃H₂₂F₂N₂O₅ 444.15 m/z found 445.1 [M+H]⁺.

### Preparation of 2-(8-(1-((3,5-difluorophenyl)amino)ethyl)-2-morpholino-4-oxo-4H-chromen-6-yl)-N,N-dimethylacetamide (Step 3 in Scheme 6)

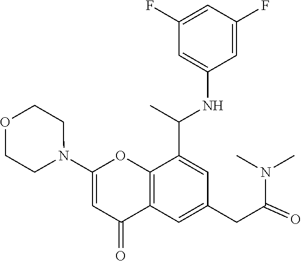 [SMILES: CC(Nc1cc(F)cc(F)c1)c1cc(CC(=O)N(C)C)cc2c(=O)cc(N3CCOCC3)oc12]

To a solution of 2-[8-[1-(3,5-difluoroanilino)ethyl]-2-morpholino-4-oxo-chromen-6-yl]acetic acid (130 mg, 270.33 umol, 1 eq, HCl), DIEA (279.51 mg, 2.16 mmol, 376.70 uL, 8 eq) and N-methylmethanamine hydrochloride (132.27 mg, 1.62 mmol, 6 eq) in THF (1.5 mL) was added T3P (344.06 mg, 540.67 umol, 321.55 uL, 50% purity, 2 eq) dropwise at 0° C. After addition, the mixture was stirred at 25° C. for 2 hours. LC-MS and HPLC showed 2-[8-[1-(3,5-difluoroanilino)ethyl]-2-morpholino-4-oxo-chromen-6-yl]acetic acid was consumed completely. The reaction mixture was quenched by addition water (0.5 mL). The mixture was directly purified by prep-HPLC (column: Waters Xbridge BEH C18 100*25 mm*5 um; mobile phase: [water (10 mM NH₄HCO₃)-MeCN]; B %: 25%-50%, 10 mins). The eluent was removed under freeze drying. Compound 2-[8-[1-(3,5-difluoroanilino)ethyl]-2-morpholino-4-oxo-chromen-6-yl]-N,N-dimethyl-acetamide (10.4 mg, 21.88 umol, 8.09% yield, 99.18% purity) was obtained as white solid. ¹H NMR (DMSO-d₆, 400 MHz) δ 7.66 (d, J=2.0 Hz, 1H), 7.36 (d, J=2.0 Hz, 1H), 6.94 (d, J=6.4 Hz, 1H), 6.23˜6.18 (m, 1H), 6.11 (dd, J=2.0 Hz, 10.4 Hz, 2H), 5.54 (s, 1H), 4.97˜4.91 (m, 1H), 3.76 (s, 4H), 3.72 (s, 2H), 3.57˜3.51 (m, 4H), 2.84 (s, 3H), 2.74 (s, 3H), 1.50 (d, J=6.8 Hz, 3H). LCMS: 99.18% (220 nm), 100.00% (254 nm). MS (ESI): mass calcd. For C₂₅H₂₇F₂N₃O₄ 471.20 m/z found 472.2 [M+H]⁺.

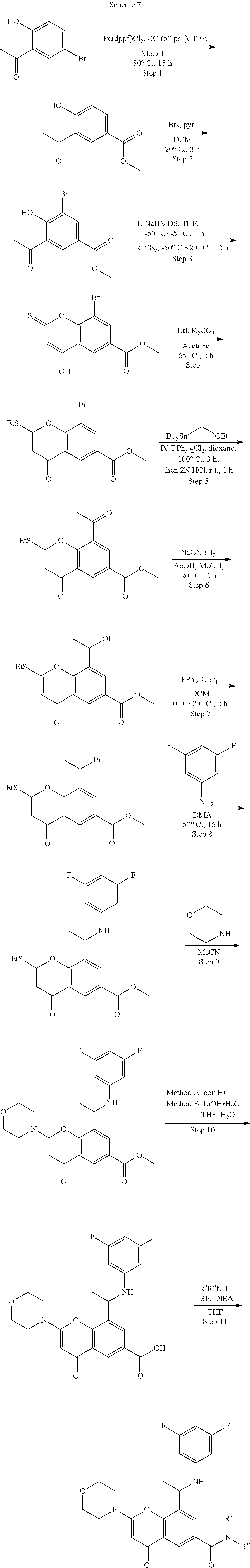

### General Procedures for Preparing Compounds in Scheme 7

### Preparation of methyl 3-acetyl-4-hydroxybenzoate (Step 1 in Scheme 7)

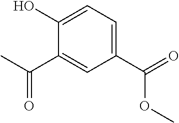

A mixture of 1-(5-bromo-2-hydroxy-phenyl)ethanone (10 g, 46.50 mmol, 1 eq), Pd(dppf)Cl₂ (1.70 g, 2.33 mmol, 0.05 eq) and TEA (9.41 g, 93.00 mmol, 12.95 mL, 2 eq) in MeOH (300 mL) was stirred at 80° C. for 15 hours under CO (50 psi.). TLC (petroleum ether:EtOAc=5:1, R_{f}=0.45) and LCMS showed the reaction was complete. After filtration, the filtrate was removed under reduced pressure. The residue was dissolved in EtOAc (100 mL) and washed with 0.5N HCl to pH=7 and brine (50 mL×3). The organic layer was dried over Na₂SO₄, filtered and concentrated under reduced pressure. The residue was purified by flash silica gel chromatography (Biotage®; 40 g SepaFlash® Silica Flash Column, Eluent of 0˜16% Ethyl acetate/petroleum ether gradient @80 mL/min). The eluent was removed under reduced pressure. Compound methyl 3-acetyl-4-hydroxy-benzoate (9 g, 46.35 mmol, 99.67% yield) was obtained as brown needle solid. ¹H NMR (DMSO-d₆, 400 MHz) δ 12.20 (s, 1H), 8.36 (s, 1H), 8.03 (d, J=8.4 Hz, 1H), 7.07 (d, J=8.8 Hz, 1H), 3.84 (s, 3H), 2.67 (s, 3H).

### Preparation of methyl 3-acetyl-5-bromo-4-hydroxybenzoate (Step 2 in Scheme 7)

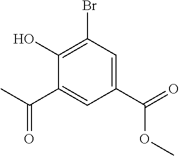

To a mixture of methyl 3-acetyl-4-hydroxy-benzoate (37 g, 190.54 mmol, 1 eq) and pyridine (60.29 g, 762.17 mmol, 61.52 mL, 4 eq) in DCM (400 mL) was added Br₂ (33.50 g, 209.60 mmol, 10.80 mL, 1.1 eq) dropwise at 0° C. Then the mixture was stirred at 20° C. for 3 hours under N₂. TLC (petroleum ether:EtOAc=5:1, R_{f}=0.45) showed the reaction was complete. The mixture was poured into ice water (300 mL) and made pH=4 with 2N HCl slowly. The organic layer was separated and the aqueous was extracted with DCM (300 mL×2). The combined organic layer was washed with brine (300 mL×5), dried over Na₂SO₄, filtered and concentrated under reduced pressure. The residue was triturated with petroleum ether (200 mL). The solid was collected after filtration. Compound methyl 3-acetyl-5-bromo-4-hydroxy-benzoate (48.5 g, 177.60 mmol, 93.21% yield) was obtained as brown solid. ¹H NMR (DMSO-d₆, 400 MHz) δ 13.28 (s, 1H), 8.44 (d, J=2.0 Hz, 1H), 8.31 (d, J=2.0 Hz, 1H), 3.87 (s, 3H), 2.76 (s, 3H).

### Preparation of methyl 8-bromo-4-hydroxy-2-thioxo-2H-chromene-6-carboxylate (Step 3 in Scheme 7

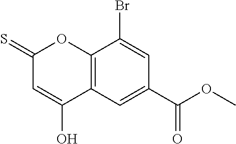 [SMILES: COC(=O)c1cc(Br)c2oc(=S)cc(O)c2c1]

To a solution of methyl 3-acetyl-5-bromo-4-hydroxy-benzoate (40 g, 146.48 mmol, 1 eq) in THF (200 mL) was added NaHMDS (1 M, 512.67 mL, 3.5 eq) dropwise at −50° C. The mixture was then stirred at −5° C.˜0° C. for an hour under N₂. Then CS₂ (17.84 g, 234.36 mmol, 14.16 mL, 1.6 eq) was added dropwise at −50° C. and the mixture was stirred at 20° C. for 12 hours under N₂. TLC (petroleum ether:EtOAc=0:1, R_{f}=0.2) and LCMS showed the reaction was nearly complete. The mixture was poured into ice water (1 L) and made pH=4 with con. HCl slowly. The organic layer was separated and the aqueous was extracted with EtOAc (400 mL×3). The combined organic layer was washed with brine (200 mL×5), dried over Na₂SO₄, filtered and concentrated under reduced pressure. The residue was triturated with DCM (100 mL). The solid was collected after filtration. Compound methyl 8-bromo-4-hydroxy-2-thioxo-chromene-6-carboxylate (28.5 g, 67.73 mmol, 46.24% yield, 74.89% purity) was obtained as yellow solid. ¹H NMR (DMSO-d₆, 400 MHz) δ 8.36 (d, J=2.0 Hz, 1H), 8.32 (d, J=2.0 Hz, 1H), 6.63 (s, 1H), 3.91 (s, 3H).

### Preparation of methyl 8-bromo-2-(ethylthio)-4-oxo-4H-chromene-6-carboxylate (Step 4 in Scheme 7

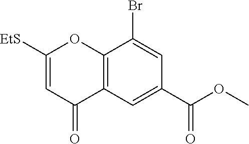

To a solution of methyl 8-bromo-4-hydroxy-2-thioxo-chromene-6-carboxylate (28.5 g, 90.44 mmol, 1 eq) and EtI (49.37 g, 316.53 mmol, 25.32 mL, 3.5 eq) in acetone (600 mL) was added K₂CO₃ (15.00 g, 108.52 mmol, 25.55 L, 1.20 eq). The mixture was stirred at 65° C. for 2 hours. TLC (petroleum ether:EtOAc=5:1, R_{f}=0.2) and LC-MS showed the reaction was nearly complete. The solvent was removed under reduced pressure. The residue was dissolved in EtOAc (300 mL) and washed with 0.05N HCl to pH=7. The combined organic layer was washed with brine (100 mL×2), dried over Na₂SO₄, filtered and concentrated under reduced pressure. The residue was triturated with EtOAc (20 mL). The solid was collected after filtration. Compound methyl 8-bromo-2-ethylsulfanyl-4-oxo-chromene-6-carboxylate (25 g, 72.85 mmol, 80.55% yield) was obtained as brown solid. ¹H NMR (DMSO-d₆, 400 MHz) δ 8.44 (d, J=2.0 Hz, 1H), 8.42 (d, J=2.0 Hz, 1H), 6.51 (s, 1H), 3.92 (s, 3H), 3.26 (q, J=7.2 Hz, 2H), 1.40 (t, J=7.2 Hz, 3H).

### Preparation of methyl 8-acetyl-2-(ethylthio)-4-oxo-4H-chromene-6-carboxylate (Step 5 in Scheme 7)

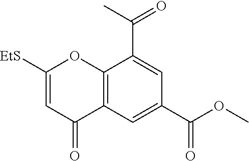

A mixture of methyl 8-bromo-2-ethylsulfanyl-4-oxo-chromene-6-carboxylate (10 g, 29.14 mmol, 1 eq), tributyl(1-ethoxyvinyl)stannane (13.33 g, 36.91 mmol, 12.46 mL, 1.27 eq) and Pd(PPh₃)₂Cl₂ (1.02 g, 1.46 mmol, 0.05 eq) in dioxane (150 mL) was stirred at 100° C. for 3 hours under N₂. TLC (petroleum ether:EtOAc=4:1, R_{f}=0.2) and LC-MS showed the reaction was complete. The mixture was cooled to r.t. and 17.48 mL of 2N HCl was added and the mixture was stirred at 20° C. for an hour. The organic solvent was removed under reduced pressure. The aqueous was made pH=7 with sat. NaHCO₃ and extracted with DCM (100 mL×3). The combined organic layer was washed with brine (100 mL×3), dried over Na₂SO₄, filtered and concentrated under reduced pressure. The residue was triturated with petroleum ether and MTBE (v:v=10:1, 110 mL×2). The solid was collected after filtration. Compound methyl 8-acetyl-2-ethylsulfanyl-4-oxo-chromene-6-carboxylate (8 g, 26.12 mmol, 89.63% yield) was obtained as brown solid. ¹H NMR (DMSO-d₆, 400 MHz) δ 8.64 (d, J=2.0 Hz, 1H), 8.53 (d, J=2.4 Hz, 1H), 6.52 (s, 1H), 3.93 (s, 3H), 3.22 (q, J=7.2 Hz, 2H), 2.72 (s, 3H), 1.36 (t, J=7.2 Hz, 3H).

### Preparation of methyl 2-(ethylthio)-8-(1-hydroxyethyl)-4-oxo-4H-chromene-6-carboxylate (Step 6 in Scheme 7)

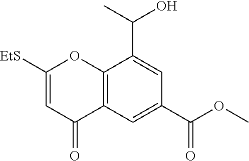

To a solution of methyl 8-acetyl-2-ethylsulfanyl-4-oxo-chromene-6-carboxylate (7 g, 22.85 mmol, 1 eq) in MeOH (70 mL) and AcOH (41.17 g, 685.53 mmol, 39.21 mL, 30 eq) was added NaBH₃CN (2.87 g, 45.70 mmol, 2 eq) in portion at 20° C. and then the mixture was stirred at 20° C. for two hours. TLC (petroleum ether:EtOAc=2:1, R_{f}=0.2) and LC-MS showed the reaction was complete. The mixture was quenched with ice water (100 mL) at 0° C. and the organic solvent was removed under reduced pressure. There was some yellow solid formed. After filtration, the solid (2 g) was collected. The filtrate was extracted with EtOAc (100 mL×3). The combined organic layer was washed with sat.NaHCO₃ to pH=7 and brine (50 mL×2), dried over Na₂SO₄, filtered and concentrated under reduced pressure. The residue was triturated with EtOAc (30 mL). The solid (3.2 g) was collected after filtration. The filtrate was concentrated under reduced pressure and the residue was purified by flash silica gel chromatography (Biotage®; 20 g SepaFlash® Silica Flash Column, Eluent of 0˜7% DCM and EtOAc gradient @ 80 mL/min). The eluent was removed under reduced pressure. Compound methyl 2-ethylsulfanyl-8-(1-hydroxyethyl)-4-oxo-chromene-6-carboxylate (5.2 g, 16.86 mmol, 73.80% yield) was obtained as brown solid. ¹H NMR (DMSO-d₆, 400 MHz) δ 8.44 (d, J=2.4 Hz, 1H), 8.40 (d, J=2.0 Hz, 1H), 6.45 (s, 1H), 5.64 (d, J=4.8 Hz, 1H), 5.24 (t, J=6.0 Hz, 1H), 3.92 (s, 3H), 3.23 (q, J=7.2 Hz, 2H), 1.40 (d, J=6.0 Hz, 3H), 1.37 (t, J=7.2 Hz, 3H).

### Preparation of methyl 8-(1-bromoethyl)-2-(ethylthio)-4-oxo-4H-chromene-6-carboxylate (Step 7 in Scheme 7)

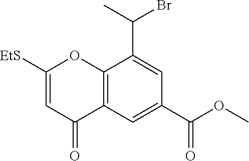

To a solution of methyl 2-ethylsulfanyl-8-(1-hydroxyethyl)-4-oxo-chromene-6-carboxylate (4 g, 12.97 mmol, 1 eq) in DCM (100 mL) was added PPh₃ (5.10 g, 19.46 mmol, 1.5 eq) and CBr₄ (6.45 g, 19.46 mmol, 1.5 eq) in portions with stirring at 0° C. Then the mixture was stirred at 20° C. for 2 hours under N₂. TLC (Petroleum ether:Ethyl acetate=1:1, R_{f}=0.8) indicated one major new spot with lower polarity was detected which was desired. The mixture was concentrated under reduced pressure at 20° C. The residue was purified by flash silica gel chromatography (Biotage®; 120 g SepaFlash® Silica Flash Column, Eluent of 0˜20% Ethyl acetate/Petroleum ether gradient @100 mL/min). The eluent was removed under reduced pressure. Methyl 8-(1-bromoethyl)-2-ethylsulfanyl-4-oxo-chromene-6-carboxylate (3.5 g, 9.43 mmol, 72.68% yield) was obtained as yellow solid. ¹H NMR (DMSO-d₆, 400 MHz) δ 8.49 (d, J=2.0 Hz, 1H), 8.40 (d, J=1.2 Hz, 1H), 6.51 (s, 1H), 5.88 (t, J=6.8 Hz, 1H), 3.92 (s, 3H), 3.27 (q, J=7.2 Hz, 2H), 2.12 (d, J=7.2 Hz, 3H), 1.39 (t, J=7.2 Hz, 3H).

### Preparation of methyl 8-(1-((3,5-difluorophenyl)amino)ethyl)-2-(ethylthio)-4-oxo-4H-chromene-6-carboxylate (Step 8 in Scheme 7)

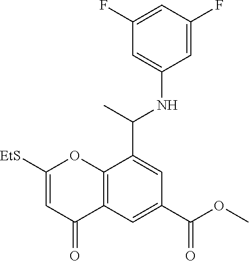

A mixture of methyl 8-(1-bromoethyl)-2-ethylsulfanyl-4-oxo-chromene-6-carboxylate (2 g, 5.39 mmol, 1 eq) and 3,5-difluoroaniline (1.25 g, 9.70 mmol, 1.8 eq) in DMA (20 mL) was stirred at 50° C. for 16 hours. TLC (Petroleum ether:Ethyl acetate=2:1, R_{f}=0.3) indicated one major new spot with larger polarity was detected. The mixture was cooled to 20° C. The residue was poured into ice-water (w/w=1/1, 100 mL) and stirred for 5 mins. The aqueous phase was extracted with ethyl acetate (50 mL×3). The combined organic phase was washed with brine (30 mL×2), dried with anhydrous Na₂SO₄, filtered and concentrated in vacuum. The residue was purified by flash silica gel chromatography (Biotage®; 40 g SepaFlash® Silica Flash Column, Eluent of 0˜20% Ethyl acetate/Petroleum ether gradient @ 100 mL/min). Methyl 8-[1-(3,5-difluoroanilino)ethyl]-2-ethylsulfanyl-4-oxo-chromene-6-carboxylate (1.4 g, 3.34 mmol, 61.96% yield) was obtained as yellow solid. ¹H NMR (DMSO-d₆, 400 MHz) δ 8.43 (d, J=2.0 Hz, 1H), 8.19 (d, J=2.0 Hz, 1H), 7.09 (d, J=6.8 Hz, 1H), 6.49 (s, 1H), 6.23 (t, J=9.6 Hz, 1H), 6.14 (dd, J=2.0 Hz, J=14.0 Hz, 2H), 4.98 (t, J=6.4 Hz, 1H), 3.86 (s, 3H), 3.25 (q, J=7.2 Hz, 2H), 1.52 (d, J=6.8 Hz, 3H), 1.38 (t, J=7.2 Hz, 3H).

### Preparation of methyl 8-(1-((3,5-difluorophenyl)amino)ethyl)-2-morpholino-4-oxo-4H-chromene-6-carboxylate (Step 9 in Scheme 7)

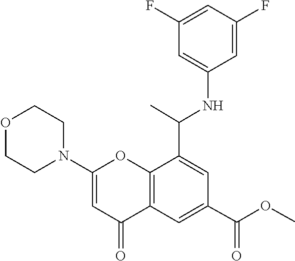 [SMILES: COC(=O)c1cc(C(C)Nc2cc(F)cc(F)c2)c2oc(N3CCOCC3)cc(=O)c2c1]

A solution of methyl 8-[1-(3,5-difluoroanilino)ethyl]-2-ethylsulfanyl-4-oxo-chromene-6-carboxylate (0.6 g, 1.43 mmol, 1 eq) and morpholine (498.49 mg, 5.72 mmol, 503.53 uL, 4 eq) in MeCN (5 mL) was stirred at 80° C. for 10 hours. Morpholine (249.25 mg, 2.86 mmol, 251.76 uL, 4 eq) was added and the mixture was stirred at 80° C. for 10 hours. TLC (petroleum ether:EtOAc=0:1, R_{f}=0.15) and LC-MS showed the reaction was nearly complete. The solvent was removed under reduced pressure. The residue was triturated with EtOAc (10 mL). The solid was collected. Compound methyl 8-[1-(3,5-difluoroanilino)ethyl]-2-morpholino-4-oxo-chromene-6-carboxylate (0.47 g, 1.06 mmol, 73.93% yield) was obtained as yellow solid. ¹H NMR (DMSO-d₆, 400 MHz) δ 8.39 (d, J=2.4 Hz, 1H), 8.10 (d, J=2.0 Hz, 1H), 7.07 (d, J=6.8 Hz, 1H), 6.22 (t, J=4.0 Hz, 1H), 6.14 (dd, J=2.0 Hz, J=10.4 Hz, 2H), 5.63 (s, 1H), 5.00 (t, J=6.8 Hz, 1H), 3.86 (s, 3H), 3.75 (t, J=4.4 Hz, 4H), 3.58 (t, J=4.8 Hz, 4H), 1.50 (d, J=6.4 Hz, 3H).

### Preparation of 8-(1-((3,5-difluorophenyl)amino)ethyl)-2-morpholino-4-oxo-4H-chromene-6-carboxylic acid (Step 10 in Scheme 7)

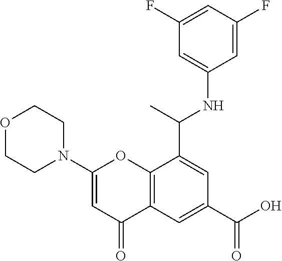 [SMILES: CC(Nc1cc(F)cc(F)c1)c1cc(C(=O)O)cc2c(=O)cc(N3CCOCC3)oc12]

A solution of methyl 8-[1-(3,5-difluoroanilino)ethyl]-2-morpholino-4-oxo-chromene-6-carboxylate (0.24 g, 540.02 umol, 1 eq) in HCl (12 M, 4.50 mL, 100 eq) was stirred at 80° C. for an hour. TLC (petroleum ether:EtOAc=0:1, R_{f}=0.05) and LC-MS showed the reaction was complete. There was some white solid formed. The solid was collected after filtration and concentrated under reduced pressure. The solid was used to the next step without further purification. Compound 8-[1-(3,5-difluoroanilino)ethyl]-2-morpholino-4-oxo-chromene-6-carboxylic acid (0.2 g, 464.68 umol, 86.05% yield) was obtained as pink solid. ¹H NMR (DMSO-d₆, 400 MHz) δ 8.37 (d, J=2.4 Hz, 1H), 8.11 (d, J=2.4 Hz, 1H), 6.21 (t, J=4.4 Hz, 1H), 6.15 (dd, J=2.4 Hz, J=10.4 Hz, 2H), 5.77 (s, 1H), 5.00 (q, J=6.4 Hz, 1H), 3.75 (t, J=4.8 Hz, 4H), 3.61 (t, J=3.6 Hz, 4H), 1.50 (d, J=6.8 Hz, 3H).

### Preparation of Compounds in (Step 11 in Scheme 7)

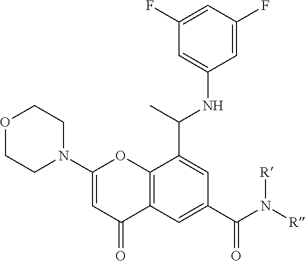

To a mixture of 8-[1-(3,5-difluoroanilino)ethyl]-2-morpholino-4-oxo-chromene-6-carboxylic acid, R′N′R″ (255.57 umol˜697.02 umol, 1.1 eq˜3 eq) and DIEA (1.16 mmol, 202.35 uL, 5 eq) in THF (6 mL/mmol˜12 mL/mmol) and DMF (2 mL/mmol) was added T3P (302.04 umol, 50% purity, 1.3 eq) dropwise at 0° C. and the mixture was stirred at 20° C. for 2 hours˜3 hours. HPLC and LC-MS showed the reaction was complete. The mixture was extracted with EtOAc. The combined organic layer was washed with brine, dried over Na₂SO₄, filtered and concentrated under reduced pressure. The residue was purified by prep-HPLC (column: Phenomenex Gemini-NX 80*40 mm*3 um or Phenomenex Gemini-NX 80*30 mm*3 um or Phenomenex Gemini-NX C18 80*30 mm*5 um or Waters Xbridge BEH C18 100*25 mm*5 um; mobile phase: [water (10 mM NH₄HCO₃)-MeCN]; B %: 15%-45%, 8 mins or mobile phase: [water (10 mM NH₄HCO₃)-MeCN]; B %: 25%-50%, 10 mins or; [water (10 mM NH₄HCO₃)-MeCN]; B %: 25%-55%, 8 mins). The aqueous solution was lyophilized to give desired product.

### Compound 27

### Preparation of 8-(1-((3,5-difluorophenyl)amino)ethyl)-6-(3-(dimethylamino)pyrrolidine-1-carbonyl)-2-morpholino-4H-chromen-4-one (Step 11 in Scheme 7)

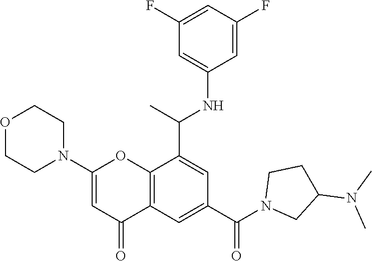 [SMILES: CC(Nc1cc(F)cc(F)c1)c1cc(C(=O)N2CCC(N(C)C)C2)cc2c(=O)cc(N3CCOCC3)oc12]

To a mixture of 8-[1-(3,5-difluoroanilino)ethyl]-2-morpholino-4-oxo-chromene-6-carboxylic acid (0.1 g, 232.34 umol, 1 eq), N,N-dimethylpyrrolidin-3-amine (34.49 mg, 302.04 umol, 1.3 eq) and DIEA (150.14 mg, 1.16 mmol, 202.35 uL, 5 eq) in THF (3 mL) and DMF (0.5 mL) was added T3P (192.21 mg, 302.04 umol, 179.63 uL, 50% purity, 1.3 eq) dropwise at 0° C. and the mixture was stirred at 20° C. for 3 hours. HPLC and LCMS showed the reaction was complete. The mixture was extracted with EtOAc (20 mL×3). The combined organic layer was washed with brine (20 mL×3), dried over Na₂SO₄, filtered and concentrated under reduced pressure. The residue was purified by prep-HPLC (column: Phenomenex Gemini-NX 80*40 mm*3 um; mobile phase: [water (10 mM NH₄HCO₃)-MeCN]; B %: 15%-45%, 8 mins). The eluent was dried under freeze drying. Compound 8-[1-(3,5-difluoroanilino)ethyl]-6-[3-(dimethylamino)pyrrolidine-1-carbonyl]-2-morpholino-chromen-4-one (46.1 mg, 84.44 umol, 36.34% yield, 96.45% purity) was obtained as white solid. ¹H NMR (DMSO-d₆, 400 MHz) δ 7.91 (d, J=4.8 Hz, 1H), 7.63 (d, J=12.0 Hz, 1H), 6.99˜6.94 (m, 1H), 6.22˜6.14 (m, 3H), 5.59 (s, 1H), 5.01 (t, J=2.4 Hz, 1H), 3.74 (t, J=4.4 Hz, 4H), 3.58˜3.50 (m, 5H), 3.38˜3.10 (m, 3H), 2.68˜2.60 (m, 1H), 2.30˜1.80 (m, 7H), 1.68˜1.52 (m, 1H), 1.49 (d, J=5.6 Hz, 3H). HPLC: 96.45% (220 nm), 100% (254 nm). MS (ESI): mass calcd. For C₂₈H₃₂F₂N₄O₄ 526.24 m/z found 527.3 [M+H]⁺.

### Compound 28

### 8-(1-((3,5-difluorophenyl)amino)ethyl)-6-(2,2-dioxido-2-thia-6-azaspiro[3.3]heptane-6-carbonyl)-2-morpholino-4H-chromen-4-one was prepared according to the procedure described herein for Step 11 in Scheme 7

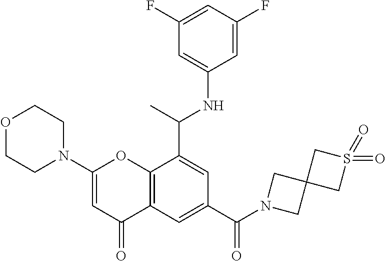 [SMILES: CC(Nc1cc(F)cc(F)c1)c1cc(C(=O)N2CC3(C2)CS(=O)(=O)C3)cc2c(=O)cc(N3CCOCC3)oc12]

The desired compound (52.3 mg, 89.94 umol, 48.39% yield, 96.23% purity) was obtained as yellow solid. ¹H NMR (DMSO-d₆, 400 MHz) δ 8.02 (d, J=2.4 Hz, 1H), 8.84 (d, J=2.0 Hz, 1H), 7.03 (d, J=6.8 Hz, 1H), 6.22 (t, J=7.2 Hz, 1H), 6.16 (dd, J=2.0 Hz, J=7.2 Hz, 2H), 5.63 (s, 1H), 5.01 (t, J=6.4 Hz, 1H), 4.53˜4.26 (m, 8H), 3.74 (t, J=4.8 Hz, 4H), 3.59 (t, J=4.8 Hz, 4H), 1.50 (d, J=6.4 Hz, 3H). HPLC: 96.23% (220 nm), 99.02% (254 nm). MS (ESI): mass calcd. For C₂₇H₂₇F₂N₃O₆S 559.16 m/z found 560.2 [M+H]⁺.

### Compound 29

### 8-(1-((3,5-difluorophenyl)amino)ethyl)-6-((R)-3-(dimethylamino)pyrrolidine-1-carbonyl)-2-morpholino-4H-chromen-4-one was prepared according to the procedure described herein for Step 11 in Scheme 7

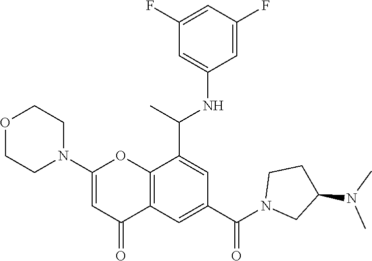 [SMILES: CC(Nc1cc(F)cc(F)c1)c1cc(C(=O)N2CC[C@@H](N(C)C)C2)cc2c(=O)cc(N3CCOCC3)oc12]

The desired compound (103.1 mg, 191.84 umol, 48.57% yield, 97.98% purity) was obtained as white solid. ¹H NMR (DMSO-d₆, 400 MHz) δ 7.91 (s, 1H), 7.66 (d, J=11.6 Hz, 1H), 6.99˜6.92 (m, 1H), 6.16˜6.14 (m, 3H), 5.59 (s, 1H), 5.05˜4.98 (m, 1H), 3.73 (t, J=4.4 Hz, 4H), 3.58˜3.52 (m, 5H), 3.19˜2.99 (m, 3H), 2.68˜2.60 (m, 1H), 2.16 (s, 3H), 2.01˜1.93 (m, 4H), 1.71˜1.66 (m, 1H), 1.51 (d, J=6.4 Hz, 3H). HPLC: 97.98% (220 nm), 100% (254 nm). MS (ESI): mass calcd. For C₂₈H₃₂F₂N₄O₄ 526.24 m/z found 527.3 [M+H]⁺.

### Compound 30

### 8-(1-((3,5-difluorophenyl)amino)ethyl)-6-((S)-3-(dimethylamino)pyrrolidine-1-carbonyl)-2-morpholino-4H-chromen-4-one was prepared according to the procedure described herein for Step 11 in Scheme 7

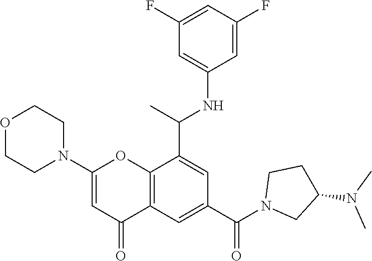 [SMILES: CC(Nc1cc(F)cc(F)c1)c1cc(C(=O)N2CC[C@H](N(C)C)C2)cc2c(=O)cc(N3CCOCC3)oc12]

The desired compound (116.3 mg, 215.45 umol, 54.55% yield, 97.55% purity) was obtained as white solid. ¹H NMR (DMSO-d₆, 400 MHz) δ 7.91 (d, J=4.8 Hz, 1H), 7.65 (d, J=11.6 Hz, 1H), 6.98˜6.96 (m, 1H), 6.22˜6.14 (m, 3H), 5.59 (s, 1H), 5.00 (t, J=3.6 Hz, 1H), 3.73 (t, J=4.4 Hz, 4H), 3.58˜3.52 (m, 5H), 3.28˜2.92 (m, 3H), 2.69˜2.60 (m, 1H), 2.15 (s, 3H), 1.93˜1.80 (m, 4H), 1.68˜1.63 (m, 1H), 1.49 (d, J=5.6 Hz, 3H). HPLC: 97.55% (220 nm), 99.72% (254 nm). MS (ESI): mass calcd. For C₂₈H₃₂F₂N₄O₄ 526.24 m/z found 527.3 [M+H]⁺.

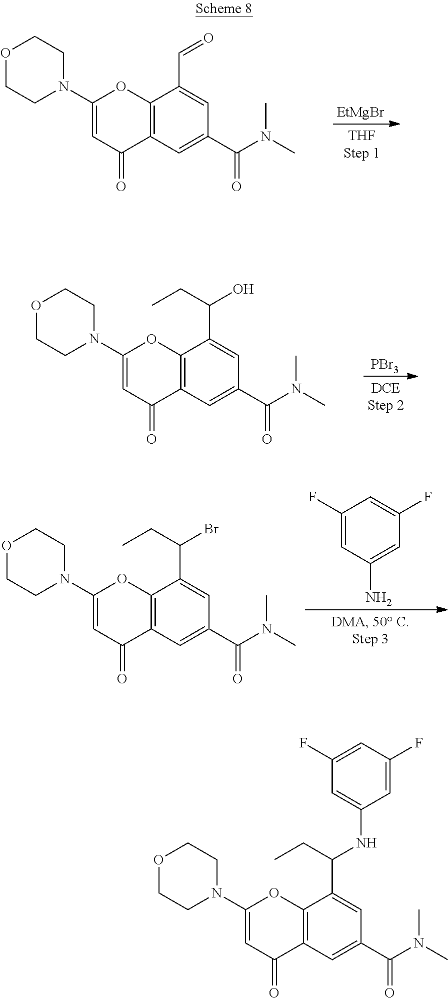

### Compound 31

### Preparation of 8-(1-((3,5-difluorophenyl)amino)propyl)-N,N-dimethyl-2-morpholino-4-oxo-4H-chromene-6-carboxamide (Steps 1-3 in Scheme 8)

### Preparation of 8-(1-hydroxypropyl)-N,N-dimethyl-2-morpholino-4-oxo-4H-chromene-6-carboxamide (Step 1 in Scheme 8)

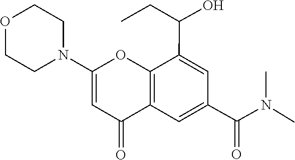 [SMILES: CCC(O)c1cc(C(=O)N(C)C)cc2c(=O)cc(N3CCOCC3)oc12]

To a mixture of 8-formyl-N,N-dimethyl-2-morpholino-4-oxo-chromene-6-carboxamide (0.3 g, 908.17 umol, 1 eq) in THF (4 mL) was added EtMgBr (3 M, 605.45 uL, 2 eq) dropwise at 0° C. under N₂ and the mixture was stirred at 0° C. for 2 hours. Then the mixture was stirred at 20° C. for 3 hours. Then EtMgBr (3 M, 302.72 uL, 1 eq) was added dropwise and the mixture was stirred at 0° C. for 2 hours. TLC (EtOAc:MeOH=20:1, R_{f}=0.15), HPLC and LCMS showed the reaction was nearly complete. The mixture was quenched with sat. NH₄Cl (5 mL) and made pH=7 with 1N HCl at 0° C. The aqueous was extracted with DCM:i-PrOH (v:v=3:1, 40 mL×3). The combined organic layer was washed with brine (5 mL×1), dried over Na₂SO₄, filtered and concentrated under reduced pressure. The residue was purified by flash silica gel chromatography (Biotage®; 4 g SepaFlash® Silica Flash Column, Eluent of 0˜20% MeOH/Ethylacetate gradient @30 mL/min). The eluent was removed under reduced pressure. A mixture of compound 8-(1-hydroxypropyl)-N,N-dimethyl-2-morpholino-4-oxo-chromene-6-carboxamide (0.1 g, crude) and 8-(hydroxymethyl)-N,N-dimethyl-2-morpholino-4-oxo-4H-chromene-6-carboxamide was obtained as yellow solid. ¹H NMR (DMSO-d₆, 400 MHz) δ 7.79 (s, 1H), 7.72 (s, 1H), 5.56 (s, 1H), 5.44 (d, J=4.4 Hz, 1H), 5.04˜5.00 (m, 1H), 3.74 (t, J=4.8 Hz, 4H), 3.54 (t, J=8.4 Hz, 4H), 2.99 (s, 3H), 2.94 (s, 3H), 1.80˜1.64 (m, 2H), 0.87 (t, J=7.2 Hz, 3H). MS (ESI): mass calcd. For C₁₉H₂₄N₂O₅ 360.17 m/z found 360.2 [M+H]⁺.

### Preparation of 8-(1-bromopropyl)-N,N-dimethyl-2-morpholino-4-oxo-4H-chromene-6-carboxamide (Step 2 in Scheme 8)

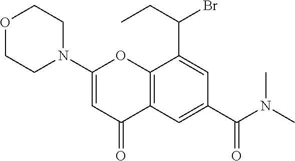 [SMILES: CCC(Br)c1cc(C(=O)N(C)C)cc2c(=O)cc(N3CCOCC3)oc12]

To a mixture of 8-(1-hydroxypropyl)-N,N-dimethyl-2-morpholino-4-oxo-chromene-6-carboxamide (0.1 g, 277.47 umol, 1 eq) and 8-(hydroxymethyl)-N,N-dimethyl-2-morpholino-4-oxo-4H-chromene-6-carboxamide (277.47 umol, 1 eq) in DCE (1 mL) was added PBr₃ (150.22 mg, 554.93 umol, 52.71 uL, 2 eq) dropwise at 0° C. and then the mixture was stirred at 50° C. for 2 hours. TLC (Ethyl acetate:MeOH=15:1, R_{f}=0.56), HPLC and LC-MS showed the reaction was complete. The mixture was quenched with ice water (2 mL) at 0° C. and made pH=7 with sat.Na₂CO₃. The aqueous was extracted with DCM (15 mL×3). The combined organic layer was washed with brine (10 mL×1), dried over Na₂SO₄, filtered and concentrated under reduced pressure. A mixture of compound 8-(1-bromopropyl)-N,N-dimethyl-2-morpholino-4-oxo-chromene-6-carboxamide (90 mg, 212.62 umol, 76.63% yield) and 8-(bromomethyl)-N,N-dimethyl-2-morpholino-4-oxo-4H-chromene-6-carboxamide was obtained as brown solid. ¹H NMR (DMSO-d₆, 400 MHz) δ 7.88 (s, 1H), 7.86 (s, 1H), 5.75 (s, 1H), 5.64˜5.60 (m, 1H), 3.75 (t, J=4.8 Hz, 4H), 3.60 (t, J=8.4 Hz, 4H), 3.00 (s, 3H), 2.93 (s, 3H), 2.36˜2.32 (m, 2H), 0.99 (t, J=7.2 Hz, 3H). MS (ESI): mass calcd. For C₁₉H₂₃BrN₂O₄ 422.08 m/z found 423.1 [M+H]⁺.

### Preparation of 8-(1-((3,5-difluorophenyl)amino)propyl)-N,N-dimethyl-2-morpholino-4-oxo-4H-chromene-6-carboxamide (Step 3 in Scheme 8)

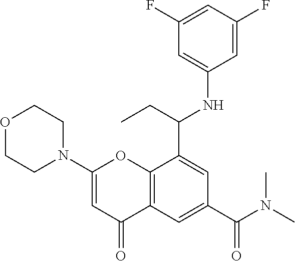 [SMILES: CCC(Nc1cc(F)cc(F)c1)c1cc(C(=O)N(C)C)cc2c(=O)cc(N3CCOCC3)oc12]

A mixture of 8-(bromomethyl)-N,N-dimethyl-2-morpholino-4-oxo-chromene-6-carboxamide (84.04 mg, 212.62 umol, 1 eq) and 8-(1-bromopropyl)-N,N-dimethyl-2-morpholino-4-oxo-chromene-6-carboxamide (90.00 mg, 212.62 umol, 1 eq) and 3,5-difluoroaniline (109.80 mg, 850.46 umol, 4 eq) in DMA (1 mL) was stirred at 50° C. for 10 hours. LCMS and HPLC showed the reaction was nearly complete. The reaction mixture was purified by prep-HPLC (column: Welch Xtimate C18 100*25 mm*3 um; mobile phase: [water (0.05% HCl)-MeCN]; B %: 20%-40%, 8 mins). The solvent was removed under freeze drying. Compound 8-[1-(3,5-difluoroanilino)propyl]-N,N-dimethyl-2-morpholino-4-oxo-chromene-6-carboxamide (13.8 mg, 29.20 umol, 13.73% yield, 99.76% purity) was obtained as yellow solid. ¹H NMR (DMSO-d₆, 400 MHz) δ 7.80 (d, J=2.0 Hz, 1H), 7.55 (d, J=2.0 Hz, 1H), 6.93˜6.90 (m, 1H), 6.23˜6.15 (m, 3H), 5.66 (s, 1H), 4.82 (t, J=6.0 Hz, 1H), 3.76 (t, J=4.8 Hz, 4H), 3.61 (t, J=8.4 Hz, 4H), 2.95 (s, 3H), 2.74 (s, 3H), 1.89-1.78 (m, 2H), 0.96 (t, J=7.2 Hz, 3H). HPLC: 99.76% (220 nm), 99.78% (254 nm). MS (ESI): mass calcd. For C₂₅H₂₇F₂N₃O₄ 471.20 m/z found 472.2 [M+H]⁺.

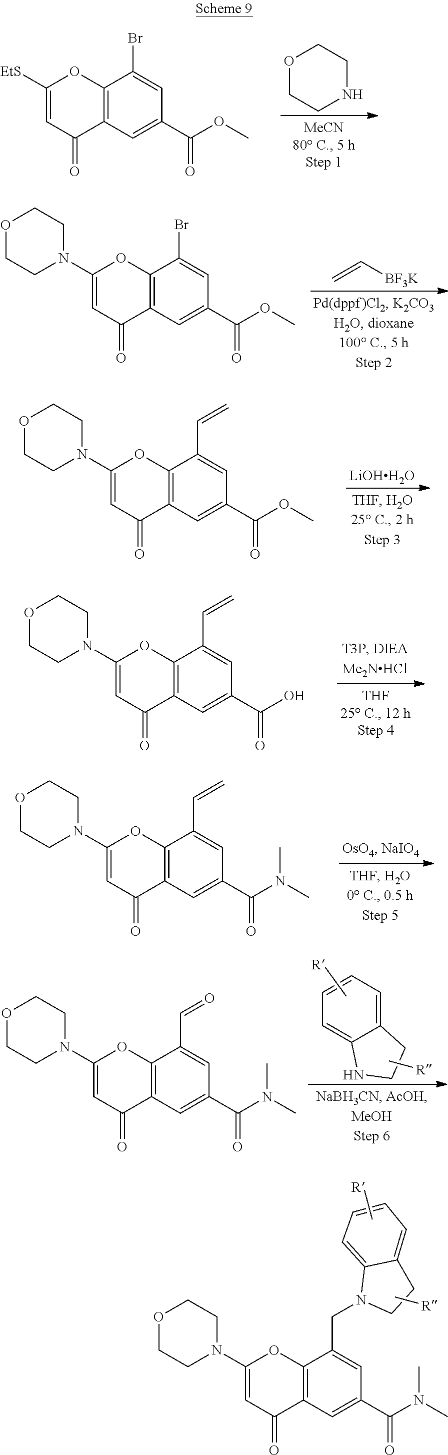

### General Procedures for Preparing Compounds in Scheme 9

### Preparation of methyl 8-bromo-2-morpholino-4-oxo-4H-chromene-6-carboxylate (Step 1 in Scheme 9)

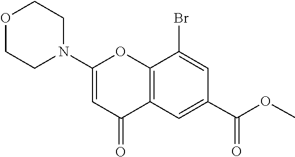 [SMILES: COC(=O)c1cc(Br)c2oc(N3CCOCC3)cc(=O)c2c1]

A solution of methyl 8-bromo-2-ethylsulfanyl-4-oxo-chromene-6-carboxylate (2 g, 5.83 mmol, 1 eq) and morpholine (2.03 g, 23.31 mmol, 2.05 mL, 4 eq) in MeCN (30 mL) was stirred at 80° C. for 5 hours. TLC (petroleum ether:EtOAc=1:1, R_{f}=0.15) and LC-MS showed the reaction was nearly complete. The solvent was removed under reduced pressure. The residue was triturated with EtOAc (30 mL). The solid was collected. Compound methyl 8-bromo-2-morpholino-4-oxo-chromene-6-carboxylate (1.5 g, 4.07 mmol, 69.91% yield) was obtained as off-white solid. ¹H NMR (DMSO-d₆, 400 MHz) δ 8.41 (s, 1H), 8.33 (s, 1H), 5.63 (s, 1H), 3.90 (s, 3H), 3.75 (t, J=5.2 Hz, 4H), 3.59 (t, J=4.8 Hz, 4H).

### Preparation of methyl 2-morpholino-4-oxo-8-vinyl-4H-chromene-6-carboxylate (Step 2 in Scheme 9)

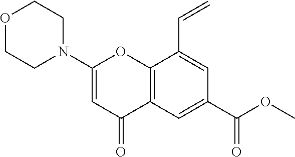 [SMILES: C=Cc1cc(C(=O)OC)cc2c(=O)cc(N3CCOCC3)oc12]

A mixture of methyl 8-bromo-2-morpholino-4-oxo-chromene-6-carboxylate (3.9 g, 10.59 mmol, 1 eq), Pd(dppf)Cl₂ (775.08 mg, 1.06 mmol, 0.1 eq), K₂CO₃ (2.93 g, 21.19 mmol, 2 eq) and potassium hydride trifluoro(vinyl)boron (2.13 g, 15.89 mmol, 1.5 eq) in dioxane (50 mL) and H₂O (5 mL) was stirred at 100° C. for 5 hours under N₂. TLC (petroleum ether:EtOAc=0:1, R_{f}=0.18) and LC-MS showed the reaction was nearly complete. After filtration, the filtrate was concentrated under reduced pressure. The residue was dissolved in EtOAc (100 mL) and water (100 mL). There was some off-white solid. The solid (2.2 g) was collected after filtration. The filtrate was extracted with EtOAc (30 mL×2). The combined organic layer was washed with brine (20 mL×1), dried over Na₂SO₄, filtered and concentrated under reduced pressure. The residue was triturated with EtOAc (30 mL) to afford 0.3 g of product. Compound methyl 2-morpholino-4-oxo-8-vinyl-chromene-6-carboxylate (2.5 g, 7.93 mmol, 74.85% yield) was obtained as white solid. ¹H NMR (DMSO-d₆, 400 MHz) δ 8.41 (d, J=2.0 Hz, 1H), 8.33 (d, J=2.4 Hz, 1H), 7.18 (dd, J=6.0 Hz, J=11.6 Hz, 1H), 6.08 (d, J=17.2 Hz, 1H), 5.61-5.58 (m, 2H), 3.91 (s, 3H), 3.74 (t, J=4.4 Hz, 4H), 3.55 (t, J=4.8 Hz, 4H).

### Preparation of 2-morpholino-4-oxo-8-vinyl-4H-chromene-6-carboxylic acid (Step 3 in Scheme 9)

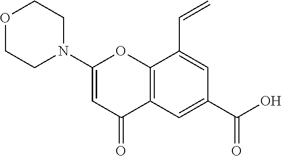 [SMILES: C=Cc1cc(C(=O)O)cc2c(=O)cc(N3CCOCC3)oc12]

A solution of methyl 2-morpholino-4-oxo-8-vinyl-chromene-6-carboxylate (4 g, 12.69 mmol, 1.0 eq) and LiOH·H₂O (1.60 g, 38.06 mmol, 3.0 eq) in THF (20 mL) and H₂O (20 mL) was stirred at 25° C. for 2 hours. LC-MS showed the reaction was complete. The mixture was adjusted to pH=4 with 2M HCl. There was yellow precipitate formed. The filter cake was collected after filtration. Compound 2-morpholino-4-oxo-8-vinyl-chromene-6-carboxylic acid (3.49 g, 11.58 mmol, 91.31% yield) was obtained as yellow solid. ¹H NMR (DMSO-d₆, 400 MHz) δ 8.40 (d, J=1.8 Hz, 1H), 8.33 (d, J=1.8 Hz, 1H), 7.19 (dd, J=11.2 Hz, 17.6 Hz, 1H), 6.07 (d, J=17.6 Hz, 1H), 5.61˜5.55 (m, 2H), 3.72˜3.75 (m, 4H), 3.57˜3.53 (m, 4H).

### Preparation of N,N-dimethyl-2-morpholino-4-oxo-8-vinyl-4H-chromene-6-carboxamide (Step 4 in Scheme 9)

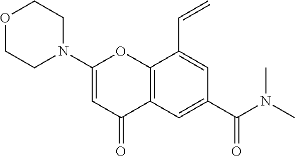 [SMILES: C=Cc1cc(C(=O)N(C)C)cc2c(=O)cc(N3CCOCC3)oc12]

To a mixture of 2-morpholino-4-oxo-8-vinyl-chromene-6-carboxylic acid (1.4 g, 4.65 mmol, 1.0 eq) and N-methylmethanamine (1.14 g, 13.95 mmol, 1.28 mL, 3.0 eq, HCl) in THF (30 mL) was added DIEA (3.00 g, 23.25 mmol, 4.05 mL, 5.0 eq) and T3P (2.22 g, 6.98 mmol, 2.07 mL, 1.5 eq) dropwise at 0° C. The mixture was stirred at 25° C. for 12 hours. The mixture was adjusted to pH=6 with 2M HCl. The mixture was extracted with ethyl acetate (50 mL×3). The combined organic phase was washed with brine (20 mL), dried with anhydrous Na₂SO₄, filtered and concentrated in vacuum. Compound N,N-dimethyl-2-morpholino-4-oxo-8-vinyl-chromene-6-carboxamide (1.7 g, crude) was obtained as yellow solid. ¹H NMR (DMSO-d₆, 400 MHz) δ 7.93 (d, J=2.0 Hz, 1H), 7.82 (d, J=2.0 Hz, 1H), 7.18 (dd, J=11.2 Hz, 17.6 Hz, 1H), 6.09 (d, J=17.6 Hz, 1H), 5.62˜5.50 (m, 2H), 3.77˜3.72 (m, 4H), 3.58˜3.51 (m, 4H), 3.06˜2.92 (m, 6H).

### Preparation of 8-formyl-N,N-dimethyl-2-morpholino-4-oxo-4H-chromene-6-carboxamide (Step 5 in Scheme 9)

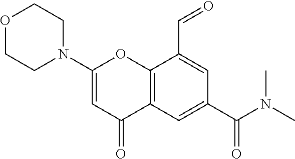 [SMILES: CN(C)C(=O)c1cc(C=O)c2oc(N3CCOCC3)cc(=O)c2c1]

To a solution of N,N-dimethyl-2-morpholino-4-oxo-8-vinyl-chromene-6-carboxamide (1.7 g, 5.18 mmol, 1.0 eq) in THF (15 mL) and H₂O (10 mL) was added OSO₄ (0.25 g, 983.37 umol, 51.02 uL, 0.19 eq) at 25° C. Then NaIO₄ (3.32 g, 15.53 mmol, 860.64 uL, 3.0 eq) was added in portions at 0° C. and then the mixture was stirred at 0° C. for half an hour. The mixture was quenched with water (50 mL) and then extracted with ethyl acetate (50 mL×3). The combined organic phase was washed with H₂O (30 mL×2) and brine (30 mL×2), dried with anhydrous Na₂SO₄, filtered and concentrated in vacuum. Compound 8-formyl-N,N-dimethyl-2-morpholino-4-oxo-chromene-6-carboxamide (1.2 g, 3.63 mmol, 70.17% yield) was obtained as yellow solid. ¹H NMR (DMSO-d₆, 400 MHz) δ10.41 (s, 1H), 8.16 (dd, J=2.0 Hz, 14.8 Hz, 2H), 5.66 (s, 1H), 3.77˜3.72 (m, 4H), 3.66˜3.60 (m, 4H), 3.08˜2.96 (m, 6H).

### Preparation of Compounds in Scheme 9 (Step 6 in Scheme 9)

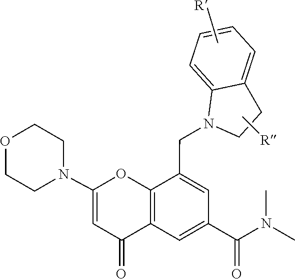

To a mixture of 8-formyl-N,N-dimethyl-2-morpholino-4-oxo-chromene-6-carboxamide (3.63 mmol, 1.0 eq) and indoline (3.63 mmol˜7.27 mmol, 1.0 eq˜2.0 eq) in MeOH (5 mL/mmol 7 mL/mmol) was added AcOH (108.98 mmol, 30 eq) at 25° C. and then the mixture was stirred at 25° C. for an hour. Then NaBH₃CN (3.63 mmol˜7.27 mmol, 1.0 eq˜2.0 eq) was added at 0° C. and the reaction mixture was stirred at 25° C. for an hour˜2 hours. The mixture was cooled to 0° C. and adjusted to pH=6 with sat. NaHCO₃. The mixture was extracted with ethyl acetate. The combined organic phase was washed with brine, dried with anhydrous Na₂SO₄, filtered and concentrated in vacuum. The residue was purified by flash silica gel chromatography (Biotage; 20 g SepaFlash® Silica Flash Column, Eluent of 0˜20% MeOH/Ethyl acetate gradient @ 50 mL/min) or by prep-HPLC (column: Phenomenex luna C18 80*40 mm*3 um or Waters Xbridge BEH C18 100*30 mm*10 um; mobile phase: [water (0.04% HCl)-MeCN]; B %: 34%-54%, 7 mins or [water (0.04% HCl)-MeCN]; B %: 38%-52%, 7 mins or [water (10 mM NH₄HCO₃)-MeCN]; B %: 20%-50%, 10 mins or [water (10 mM NH₄HCO₃)-MeCN]; B %: 30%-50%, 8 mins). The solvent was concentrated under reduced pressure or the aqueous solution was lyophilized to give desired product.

### Compound 32

### Preparation of 8-((4,6-difluoroindolin-1-yl)methyl)-N,N-dimethyl-2-morpholino-4-oxo-4H-chromene-6-carboxamide (Step 6 in Scheme 9)

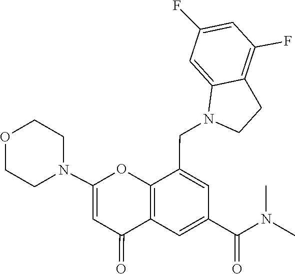 [SMILES: CN(C)C(=O)c1cc(CN2CCc3c(F)cc(F)cc32)c2oc(N3CCOCC3)cc(=O)c2c1]

To a mixture of 8-formyl-N,N-dimethyl-2-morpholino-4-oxo-chromene-6-carboxamide (1.2 g, 3.63 mmol, 1.0 eq) and 4,6-difluoroindoline (1.13 g, 7.27 mmol, 2.0 eq) in MeOH (20 mL) was added AcOH (6.54 g, 108.98 mmol, 6.23 mL, 30 eq) at 25° C. and then the mixture was stirred at 25° C. for an hour. Then NaBH₃CN (456.57 mg, 7.27 mmol, 2 eq) was added at 0° C. and the reaction mixture was stirred at 25° C. for an hour. The mixture was cooled to 0° C. and adjusted to pH=6 with sat. NaHCO₃. The mixture was extracted with ethyl acetate (80 mL×3). The combined organic phase was washed with brine (50 mL), dried with anhydrous Na₂SO₄, filtered and concentrated in vacuum. The residue was purified by flash silica gel chromatography (Biotage; 20 g SepaFlash® Silica Flash Column, Eluent of 0˜20% MeOH/Ethyl acetate gradient @ 50 mL/min). The solvent was concentrated. The obtained was triturated with MTBE and CH₃CN (v:v=20:1, 30 mL) at 25° C. for half an hour. Compound 8-[(4,6-difluoroindolin-1-yl)methyl]-N,N-dimethyl-2-morpholino-4-oxo-chromene-6-carboxamide (1.2 g, 2.52 mmol, 69.38% yield, 98.6% purity) was obtained as white solid. ¹H NMR (DMSO-d₆, 400 MHz) δ 7.84 (d, J=1.6 Hz, 1H), 7.59 (d, J=1.6 Hz, 1H), 6.47˜6.23 (m, 2H), 5.59 (s, 1H), 4.63 (s, 2H), 3.72˜3.66 (m, 4H), 3.56˜3.51 (m, 4H), 3.48˜3.45 (m, 2H), 3.01˜2.94 (m, 5H), 2.89 (s, 3H). HPLC: 98.66% (220 nm), 98.16% (254 nm). MS (ESI): mass calcd. For C₂₅H₂₅F₂O₄N₃ 469.18 m/z found 470.2 [M+H]⁺.

### Compound 33

### 8-((4-fluoro-2-methylindolin-1-yl)methyl)-N,N-dimethyl-2-morpholino-4-oxo-4H-chromene-6-carboxamide was prepared according to the procedure described herein for Step 6 in Scheme 9

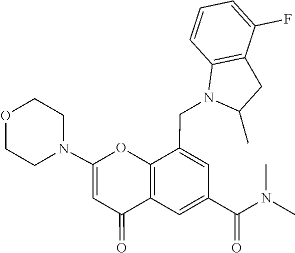 [SMILES: CC1Cc2c(F)cccc2N1Cc1cc(C(=O)N(C)C)cc2c(=O)cc(N3CCOCC3)oc12]

The desired compound (28.3 mg, 58.07 umol, 19.18% yield, 95.52% purity) was obtained as white solid. ¹H NMR (DMSO-d6, 400 MHz) δ 7.82 (d, J=2.0 Hz, 1H), 7.52 (d, J=2.0 Hz, 1H), 6.98˜6.94 (m, 1H), 6.38 (t, J=8.4 Hz, 1H), 6.10 (d, J=8.0 Hz, 1H), 5.64 (s, 1H), 4.59 (d, J=6.8 Hz, 2H), 3.90˜3.87 (m, 1H), 3.72˜3.69 (m, 4H), 3.54 (d, J=4.4 Hz, 4H), 3.31-3.27 (m, 2H), 2.96˜2.82 (m, 6H), 1.25 (d, J=6.0 Hz, 3H). MS: 95.52% (220 nm), 95.90% (254 nm). MS (ESI): mass calcd. For C₂₆H₂₈FN₃O₄ 465.21 m/z found 466.2 [M+H]⁺.

### Compound 34

### Preparation of 8-((4,6-difluoro-3,3-dimethylindolin-1-yl)methyl)-N,N-dimethyl-2-morpholino-4-oxo-4H-chromene-6-carboxamide was prepared according to the procedure described herein for Step 6 in Scheme 9

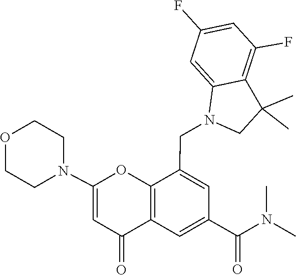 [SMILES: CN(C)C(=O)c1cc(CN2CC(C)(C)c3c(F)cc(F)cc32)c2oc(N3CCOCC3)cc(=O)c2c1]

The desired compound (33.1 mg, 65.63 umol, 24.09% yield, 98.65% purity) was obtained as white solid. ¹H NMR (DMSO-d₆, 400 MHz) δ 7.86 (d, J=2.0 Hz, 1H), 7.56 (s, 1H), 6.41 (d, J=10.8 Hz, 1H), 6.31 (t, J=10.4 Hz, 1H), 5.59 (s, 1H), 4.64 (s, 2H), 3.67 (d, J=5.2 Hz, 4H), 3.53 (d, J=5.2 Hz, 4H), 3.21 (s, 2H), 2.97 (s, 3H), 2.91 (s, 3H), 1.32 (s, 6H). HPLC: 98.65% (220 nm), 99.03% (254 nm). MS (ESI): mass calcd. For C₂₇H₂₉F₂N₃O₄ 497.21 m/z found 498.2 [M+H]⁺.

### Compound 35

### 8-((4-hydroxyindolin-1-yl)methyl)-N,N-dimethyl-2-morpholino-4-oxo-4H-chromene-6-carboxamide was prepared according to the procedure described herein for Step 6 in Scheme 9

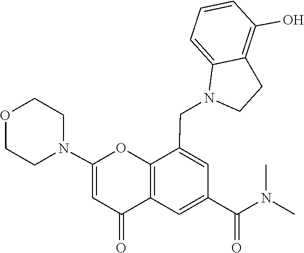 [SMILES: CN(C)C(=O)c1cc(CN2CCc3c(O)cccc32)c2oc(N3CCOCC3)cc(=O)c2c1]

The desired compound (36.4 mg, 80.98 umol, 26.75% yield, 100% purity) was obtained as gray solid. ¹H NMR (DMSO-d6, 400 MHz) δ 9.10 (s, 1H), 7.84 (d, J=2.0 Hz, 1H), 7.63 (d, J=2.0 Hz, 1H), 6.84 (t, J=8.0 Hz, 1H), 6.15 (dd, J=8.0 Hz, 10.0 Hz, 2H), 5.60 (s, 1H), 4.50 (s, 2H), 3.69 (t, J=4.4 Hz, 4H), 3.54 (t, J=4.8 Hz, 4H), 3.31 (s, 2H), 2.98 (s, 3H), 2.90 (s, 3H), 2.84 (t, J=8.0 Hz, 2H). MS: 100.00% (220 nm), 98.43% (254 nm). MS (ESI): mass calcd. For C₂₅H₂₇N₃O₅ 449.20 m/z found 450.2 [M+H]⁺.

### Compound 36

### 8-((7-chloroindolin-1-yl)methyl)-N,N-dimethyl-2-morpholino-4-oxo-4H-chromene-6-carboxamide was prepared according to the procedure described herein for Step 6 in Scheme 9

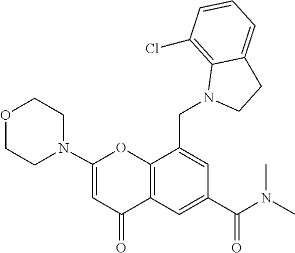 [SMILES: CN(C)C(=O)c1cc(CN2CCc3cccc(Cl)c32)c2oc(N3CCOCC3)cc(=O)c2c1]

The desired compound (2.1 mg, 4.31 umol, 1.42% yield, 96.01% purity) was obtained as white solid. ¹H NMR (400 MHz, DMSO-d₆) δ 7.83 (d, J=2 Hz, 1H), 7.63 (d, J=2 Hz, 1H), 7.10˜6.97 (m, 2H), 6.66 (t, J=7.6 Hz, 1H), 5.58 (s, 1H), 4.97 (s, 2H), 3.71˜3.69 (m, 4H), 3.55˜3.51 (m, 4H), 3.50˜3.46 (m, 2H), 3.03 (t, J=8.6 Hz, 2H), 2.98˜2.82 (m, 6H). LCMS: 96.01% (220 nm), 95.47% (254 nm). MS (ESI): mass calcd. For C₂₅H₂₆ClN₃O₄ 467.1 m/z found 468.1 [M+H]⁺.

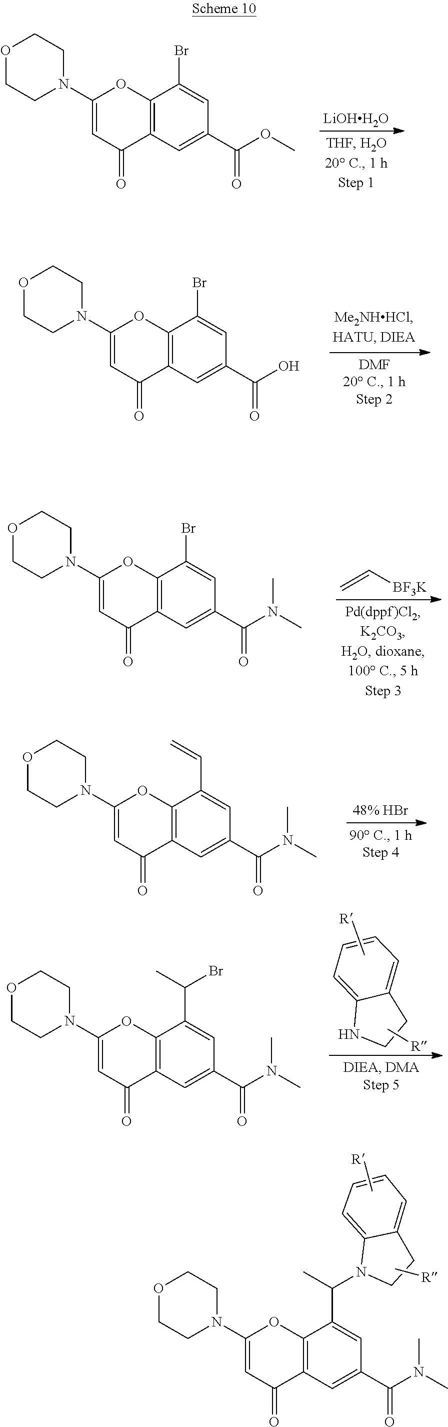

### General Procedures for Preparing Compounds in Scheme 10

### Preparation of 8-bromo-2-morpholino-4-oxo-4H-chromene-6-carboxylic acid (Step 1 in Scheme 10)

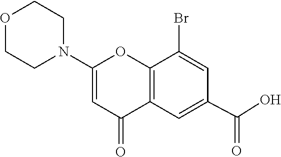 [SMILES: O=C(O)c1cc(Br)c2oc(N3CCOCC3)cc(=O)c2c1]

A solution of methyl 8-bromo-2-morpholino-4-oxo-chromene-6-carboxylate (2 g, 5.43 mmol, 1 eq) and LiOH·H₂O (683.80 mg, 16.30 mmol, 3 eq) in H₂O (10 mL) and THF (5 mL) was stirred at 20° C. for an hour. TLC (petroleum ether:EtOAc=0:1, R_{f}=0) and LC-MS showed the reaction was complete. The mixture was immediately made pH=4 with 2N HCl at 0° C. and the organic solvent was removed under reduced pressure. There was some off white solid formed. The solid was collected after filtration and dried under reduced pressure. Compound 8-bromo-2-morpholino-4-oxo-chromene-6-carboxylic acid (1.82 g, 5.14 mmol, 94.60% yield) was obtained as off white solid. ¹H NMR (DMSO-d₆, 400 MHz) δ 8.42 (d, J=2.0 Hz, 1H), 8.33 (d, J=2.0 Hz, 1H), 5.64 (s, 1H), 3.75 (t, J=4.4 Hz, 4H), 3.60 (t, J=5.6 Hz, 4H).

### Preparation of 8-bromo-N,N-dimethyl-2-morpholino-4-oxo-4H-chromene-6-carboxamide (Step 2 in Scheme 10)

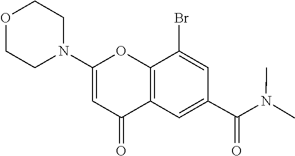 [SMILES: CN(C)C(=O)c1cc(Br)c2oc(N3CCOCC3)cc(=O)c2c1]

To a mixture of 8-bromo-2-morpholino-4-oxo-chromene-6-carboxylic acid (1.8 g, 5.08 mmol, 1 eq), DIEA (3.94 g, 30.50 mmol, 5.31 mL, 6 eq) and N-methylmethanamine (1.24 g, 15.25 mmol, 1.40 mL, 3 eq, HCl) in DMF (10 mL) was added HATU (2.51 g, 6.61 mmol, 1.3 eq) in portions. The mixture was stirred at 20° C. for an hour. TLC (petroleum ether:EtOAc=0:1, R_{f}=0.12), HPLC and LC-MS showed the reaction was nearly complete. The mixture was poured into ice water (30 mL) and there was some off white solid formed. The solid (0.5 g, off white solid) was collected after filtration. The aqueous was extracted with EtOAc (10 mL×5). The combined organic layer was washed with brine (5 mL×3), dried over Na₂SO₄, filtered and concentrated under reduced pressure. The solid was used to the next step without further purification. The residue was purified by flash silica gel chromatography (Biotage®; 20 g SepaFlash® Silica Flash Column, Eluent of 0˜15% methanol/Ethyl acetate @ 50 mL/min). The eluent was removed under reduced pressure (0.7 g, brown solid). Compound 8-bromo-N,N-dimethyl-2-morpholino-4-oxo-chromene-6-carboxamide (1.2 g, 3.15 mmol, 61.93% yield) was obtained as brown solid. ¹H NMR (DMSO-d₆, 400 MHz) δ 7.99 (d, J=2.0 Hz, 1H), 7.86 (d, J=2.0 Hz, 1H), 5.62 (s, 1H), 3.75 (t, J=4.8 Hz, 4H), 3.59 (t, J=4.4 Hz, 4H), 2.99 (s, 3H), 2.93 (s, 3H).

### Preparation of N,N-dimethyl-2-morpholino-4-oxo-8-vinyl-4H-chromene-6-carboxamide (Step 3 in Scheme 10)

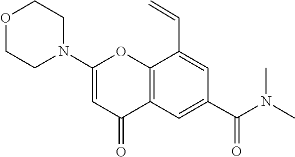 [SMILES: C=Cc1cc(C(=O)N(C)C)cc2c(=O)cc(N3CCOCC3)oc12]

A mixture of potassium hydride trifluoro(vinyl)boron (843.28 mg, 6.30 mmol, 1.5 eq), Pd(dppf)Cl₂ (307.10 mg, 419.70 umol, 0.1 eq), K₂CO₃ (1.16 g, 8.39 mmol, 2 eq) and 9-bromo-N,N-dimethyl-2-morpholino-4-oxo-pyrido[1,2-a]pyrimidine-7-carboxamide (1.6 g, 4.20 mmol, 1 eq) in dioxane (15 mL) and H₂O (1.5 mL) was stirred at 100° C. for 5 hours under N₂. TLC (EtOAc:MeOH=20:1, R_{f}=0.13) and LC-MS showed the reaction was complete. DCM (20 mL) was added and filtered. The organic layer was separated and the aqueous was extracted with DCM (10 mL×3). The combined organic layer was washed with brine (5 mL×1), dried over Na₂SO₄, filtered and concentrated under reduced pressure. The residue was triturated with petroleum ether (50 mL) and EtOAc (5 mL), then dried under reduced pressure. Compound N,N-dimethyl-2-morpholino-4-oxo-9-vinyl-pyrido[1,2-a]pyrimidine-7-carboxamide (1.3 g, 3.96 mmol, 94.33% yield) was obtained as brown solid. ¹H NMR (DMSO-d₆, 400 MHz) δ 7.94 (s, 1H), 7.82 (d, J=4.4 Hz, 1H), 7.18 (dd, J=6.4 Hz, 11.2 Hz, 1H), 6.10 (d, J=17.2 Hz, 1H), 5.59 (s, 1H), 5.56 (d, J=10.2 Hz, 1H), 4.04 (t, J=6.8 Hz, 4H), 3.56 (t, J=4.8 Hz, 4H), 3.01 (s, 3H), 2.94 (s, 3H).

### Preparation of 8-(1-bromoethyl)-N,N-dimethyl-2-morpholino-4-oxo-4H-chromene-6-carboxamide (Step 4 in Scheme 10)

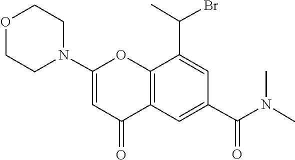 [SMILES: CC(Br)c1cc(C(=O)N(C)C)cc2c(=O)cc(N3CCOCC3)oc12]

A mixture of N,N-dimethyl-2-morpholino-4-oxo-8-vinyl-chromene-6-carboxamide (0.3 g, 913.63 umol, 1 eq) in HBr (7.70 g, 45.68 mmol, 5.17 mL, 48% purity, 50 eq) was stirred at 90° C. for an hour. TLC (petroleum ether:EtOAc=0:1, R_{f}=0.5) and LCMS showed the reaction was complete. There was some brown solid formed. The solid was collected after filtration. The filtrate was made pH=7 with sat. Na₂CO₃ and then extracted with DCM (10 mL×3). The combined organic layer was washed with brine (5 mL×2), dried over Na₂SO₄, filtered and concentrated under reduced pressure. Compound 8-(1-bromoethyl)-N,N-dimethyl-2-morpholino-4-oxo-chromene-6-carboxamide (0.27 g, 659.71 umol, 72.21% yield) was obtained as brown solid. ¹H NMR (DMSO-d₆, 400 MHz) δ 7.89 (d, J=4.4 Hz, 2H), 5.75 (q, J=6.4 Hz, 1H), 5.63 (s, 1H), 3.75 (t, J=4.8 Hz, 4H), 3.62 (t, J=4.4 Hz, 4H), 3.00 (s, 3H), 2.94 (s, 3H), 2.10 (d, J=6.8 Hz, 3H).

### Preparation of Compounds in Scheme 10 (Step 5 in Scheme 10)

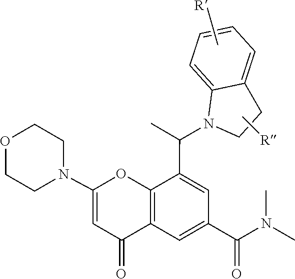

A solution of 8-(1-bromoethyl)-N,N-dimethyl-2-morpholino-4-oxo-chromene-6-carboxamide (293.20 umol, 1 eq), indoline (439.80 umol, 1.5 eq) and DIEA (0.88 mmol˜1.47 mmol, 3 eq˜5 eq) in DMA (7 mL/mmol) was stirred at 50° C. for 10 hours. LC-MS and HPLC showed the reaction was complete. The mixture was quenched with ice water and extracted with EtOAc. The combined organic layer was washed with brine, dried over Na₂SO₄, filtered and concentrated under reduced pressure. The residue was purified by prep-HPLC (column: Phenomenex Luna C18 100*30 mm*5 um or Phenomenex luna C18 80*40 mm*3 um; mobile phase: [water (0.04% HCl)-MeCN]; B %: 30%-60%, 10 mins or [water (0.04% HCl)-MeCN]; B %: 40%-60%, 7 mins). The eluent was made pH=7 with sat. NaHCO₃ and extracted with EtOAc (15 mL×3). The combined organic layer was washed with brine (10 mL×1), dried over Na₂SO₄, filtered and concentrated under reduced pressure. The residue was further purified by prep-TLC (EtOAc:MeOH=20:1). The obtained was dried under freeze drying to obtain the desired product.

### Compound 37

### Preparation of 8-(1-(4,6-difluoroindolin-1-yl)ethyl)-N,N-dimethyl-2-morpholino-4-oxo-4H-chromene-6-carboxamide (Step 5 in Scheme 10)

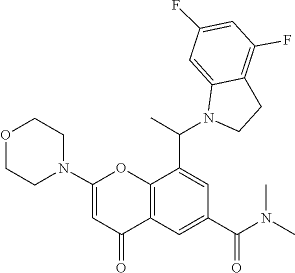 [SMILES: CC(c1cc(C(=O)N(C)C)cc2c(=O)cc(N3CCOCC3)oc12)N1CCc2c(F)cc(F)cc21]

A solution of 8-(1-bromoethyl)-N,N-dimethyl-2-morpholino-4-oxo-chromene-6-carboxamide (0.12 g, 293.20 umol, 1 eq), 4,6-difluoroindoline (68.23 mg, 439.80 umol, 1.5 eq) and DIEA (189.47 mg, 1.47 mmol, 255.35 uL, 5 eq) in DMA (2 mL) was stirred at 50° C. for 10 hours. LC-MS and HPLC showed the reaction was complete. The mixture was quenched with ice water (5 mL) and extracted with EtOAc (5 mL×3). The combined organic layer was washed with brine (2 mL×1), dried over Na₂SO₄, filtered and concentrated under reduced pressure. The residue was purified by prep-HPLC (column: Phenomenex Luna C18 100*30 mm*5 um; mobile phase: [water (0.04% HCl)-MeCN]; B %: 30%-60%, 10 mins). The eluent was made pH=7 with sat. NaHCO₃ and extracted with EtOAc (15 mL×3). The combined organic layer was washed with brine (10 mL×1), dried over Na₂SO₄, filtered and concentrated under reduced pressure. The residue was further purified by prep-TLC (EtOAc:MeOH=20:1). The obtained was dried under freeze drying. Compound 8-[1-(4,6-difluoroindolin-1-yl)ethyl]-N,N-dimethyl-2-morpholino-4-oxo-chromene-6-carboxamide (3.1 mg, 6.11 umol, 2.08% yield, 95.29% purity) was obtained as white solid. ¹H NMR (DMSO-d₆, 400 MHz) δ 8.07 (d, J=1.6 Hz, 1H), 7.80 (s, 1H), 6.17˜6.08 (m, 2H), 5.65 (s, 1H), 5.38 (q, J=6.0 Hz, 1H), 3.66 (t, J=5.6 Hz, 4H), 3.55 (t, J=4.4 Hz, 4H), 3.30˜3.24 (m, 2H), 3.13 (s, 3H), 3.01 (s, 3H), 3.01˜2.88 (m, 2H), 1.31 (d, J=7.2 Hz, 3H). HPLC: 95.29% (220 nm), 94.27% (254 nm). MS (ESI): mass calcd. For C₂₆H₂₇F₂N₃O₄ 483.20 m/z found 484.1 [M+H]⁺.

### Compound 38

### 8-(1-(4,6-difluoro-2-methylindolin-1-yl)ethyl)-N,N-dimethyl-2-morpholino-4-oxo-4H-chromene-6-carboxamide was prepared according to the procedure described herein for Step 5 in Scheme 10

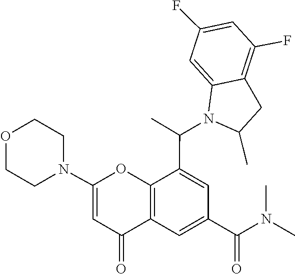 [SMILES: CC1Cc2c(F)cc(F)cc2N1C(C)c1cc(C(=O)N(C)C)cc2c(=O)cc(N3CCOCC3)oc12]

The desired compound (5.8 mg, 10.95 umol, 3.73% yield, 93.91% purity) was obtained as white solid. ¹H NMR (DMSO-d₆, 400 MHz) δ 8.09 and 8.08 (d, J=2.0 Hz, 2H), 7.86 and 7.84 (d, J=1.6 Hz, 2H), 6.31-6.07 (m, 4H), 5.65 and 5.62 (s, 2H), 5.45˜5.21 (m, 2H), 4.12˜4.07 (m, 1H), 3.68˜3.34 (m, 10H), 3.30˜3.13 (m, 1H), 3.13 (d, J=8.8 Hz, 6H), 3.03 (d, J=8.8 Hz, 6H), 2.56˜2.15 (m, 2H), 1.73 (d, J=7.2 Hz, 3H), 1.63 (d, J=7.2 Hz, 3H), 1.14 (d, J=6.4 Hz, 3H), 0.75 (d, J=6.0 Hz, 3H). HPLC: 93.91% (220 nm), 92.44% (254 nm). MS (ESI): mass calcd. For C₂₇H₂₉F₂N₃O₄ 497.21 m/z found 498.2 [M+H]⁺.

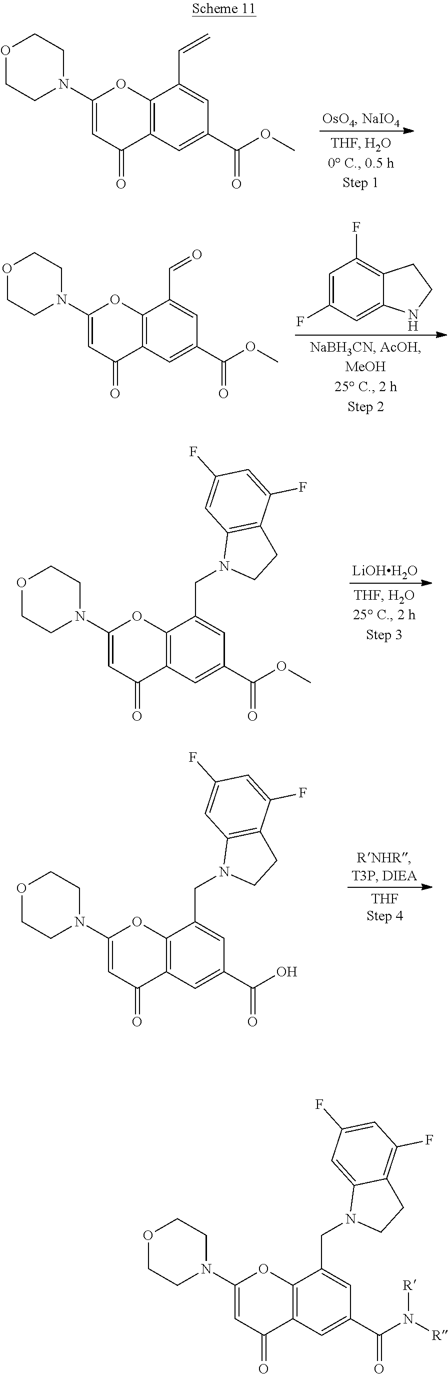

### General Procedures for Preparing Compounds in Scheme 11

### Preparation of methyl 8-formyl-2-morpholino-4-oxo-4H-chromene-6-carboxylate (Step 1 in Scheme 11)

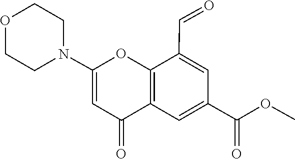 [SMILES: COC(=O)c1cc(C=O)c2oc(N3CCOCC3)cc(=O)c2c1]

To a solution of methyl 2-morpholino-4-oxo-8-vinyl-chromene-6-carboxylate (10 g, 31.71 mmol, 1.0 eq) in THF (80 mL) and H₂O (20 mL) was added OSO₄ (0.25 g, 983.37 umol, 51.02 uL, 3.10e-2 eq) at 25° C. Then NaIO₄ (20.35 g, 95.14 mmol, 5.27 mL, 3.0 eq) was added to the mixture in portions at 0° C. and the reaction mixture was stirred 0° C. for 0.5 hour. LC-MS showed the reaction was complete. The mixture was quenched with water (80 mL) and extracted with ethyl acetate (60 mL×3). The combined organic phase was washed with H₂O (30 mL×2) and brine (20 mL), dried with anhydrous Na₂SO₄, filtered and concentrated in vacuum. Compound methyl 8-formyl-2-morpholino-4-oxo-chromene-6-carboxylate (9 g, 28.36 mmol, 89.44% yield) was obtained as yellow solid. ¹H NMR (DMSO-d₆, 400 MHz) δ 10.39 (s, 1H), 8.63 (d, J=2.0 Hz, 1H), 8.55 (d, J=2.0 Hz, 1H), 5.64 (s, 1H), 3.84 (s, 3H), 3.75˜3.73 (m, 4H), 3.64˜3.62 (m, 4H).

### Preparation of methyl 8-((4,6-difluoroindolin-1-yl)methyl)-2-morpholino-4-oxo-4H-chromene-6-carboxylate (Step 2 in Scheme 11)

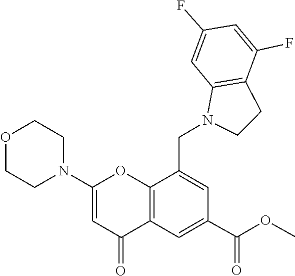 [SMILES: COC(=O)c1cc(CN2CCc3c(F)cc(F)cc32)c2oc(N3CCOCC3)cc(=O)c2c1]

To a mixture of methyl 8-formyl-2-morpholino-4-oxo-chromene-6-carboxylate (5 g, 15.76 mmol, 1.0 eq) and 4,6-difluoroindoline (3.42 g, 22.06 mmol, 1.4 eq) in MeOH (40 mL) was added AcOH (28.39 g, 472.75 mmol, 27.04 mL, 30 eq) at 25° C. and the mixture was stirred at 25° C. for an hour. Then NaBH₃CN (1.98 g, 31.52 mmol, 2.0 eq) was added in portions at 0° C. The mixture was then stirred at 25° C. for an hour. LC-MS showed the reaction was complete. The mixture was cooled to 0° C. and adjusted to pH=7 with sat. NaHCO₃. The organic solvent was removed under reduced pressure at 45° C. The aqueous was extracted with ethyl acetate (50 mL×3). The combined organic phase was washed with brine (30 mL), dried with anhydrous Na₂SO₄, filtered and concentrated in vacuum. Compound methyl 8-[(4,6-difluoroindolin-1-yl)methyl]-2-morpholino-4-oxo-chromene-6-carboxylate (5.7 g, 12.49 mmol, 79.25% yield) was obtained as yellow solid. ¹H NMR (DMSO-d₆, 400 MHz) δ 8.41 (s, 1H), 8.07 (s, 1H), 6.44˜6.42 (m, 1H), 6.32˜6.30 (m, 1H), 5.59 (s, 1H), 4.62 (s, 2H), 3.85 (s, 3H), 3.70˜3.66 (m, 4H), 3.53˜3.49 (m, 4H), 3.46˜3.44 (m, 2H), 2.96˜2.94 (m, 2H).

### Preparation of 8-((4,6-difluoroindolin-1-yl)methyl)-2-morpholino-4-oxo-4H-chromene-6-carboxylic acid (Step 3 in Scheme 11)

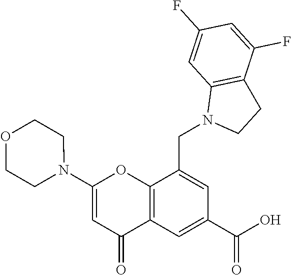 [SMILES: O=C(O)c1cc(CN2CCc3c(F)cc(F)cc32)c2oc(N3CCOCC3)cc(=O)c2c1]

To a solution of methyl 8-[(4,6-difluoroindolin-1-yl)methyl]-2-morpholino-4-oxo-chromene-6-carboxylate (5.5 g, 12.05 mmol, 1.0 eq) in THF (40 mL) was added a solution of LiOH·H₂O (2.02 g, 48.20 mmol, 4.0 eq) in H₂O (40 mL). The reaction mixture was stirred at 25° C. for 2 hours. LC-MS showed the reaction was complete. The mixture was adjusted to pH=4 with 2M HCl and there was some off-white precipitate formed. The filter cake was collected after filtration. The filtrate was concentrated to remove the organic solvent under reduced pressure at 45° C. The aqueous was extracted with ethyl acetate (50 mL×3). The combined organic phase was washed with brine (30 mL), dried with anhydrous Na₂SO₄, filtered and concentrated in vacuum. Compound 8-[(4,6-difluoroindolin-1-yl)methyl]-2-morpholino-4-oxo-chromene-6-carboxylic acid (2 g, 4.52 mmol, 37.51% yield) was obtained as off-white solid. ¹H NMR (DMSO-d₆, 400 MHz) δ 8.42 (d, J=2.0 Hz, 1H), 8.08 (d, J=2.0 Hz, 1H), 6.48˜6.28 (m, 2H), 5.60 (s, 1H), 4.64 (s, 2H), 3.71-3.65 (m, 4H), 3.56˜3.53 (m, 4H), 3.47 (t, J=8.4 Hz, 2H), 2.97 (t, J=8.4 Hz, 2H).

### Preparation of Compounds in Scheme 11 (Step 4 in Scheme 11)

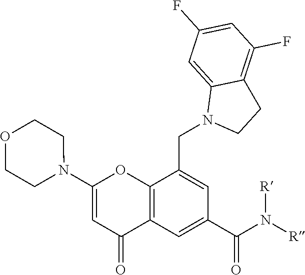

To a mixture of 8-[(4,6-difluoroindolin-1-yl)methyl]-2-morpholino-4-oxo-chromene-6-carboxylic acid (4.48 mmol, 1.0 eq) and R′N′R″ (5.82 mmol˜13.44 mmol, 1.3 eq˜3.0 eq) in THF (5 mL/mmol˜10 mL/mmol) was added DIEA (22.40 mmol˜38.56 mmol, 5.0 eq˜7.5 eq) and T3P (6.72 mmol˜10.07 mmol, 50% purity, 1.5 eq˜2.25 eq) dropwise at 0° C. under N₂. The mixture was then stirred at 25° C. for 2 hours˜72 hours. LCMS showed 8-[(4,6-difluoroindolin-1-yl)methyl]-2-morpholino-4-oxo-chromene-6-carboxylic acid was consumed completely. The mixture was adjusted to pH=7 with 2M HCl. There was white precipitate formed. After filtration, the filter cake was triturated with MTBE and CH₃CN (v:v=20:1) for two times at 25° C. for half an hour. Or the reaction mixture was quenched by addition ice-water. The mixture was filtered and the filtrate was concentrated in vacuum. The residue was purified by prep-HPLC (column: Welch Xtimate C18 100*25 mm*3 um or Waters Xbridge Prep OBD C18 150*40 mm*10 um or Phenomenex luna C18 80*40 mm*3 um; mobile phase: [water (0.05% HCl)-MeCN]; B %: 15%-35%, 8 mins or [water (0.04% HCl)-MeCN]; B %: 45%-55%, 7 mins or [water (0.04% HCl)-MeCN]; B %: 18%-55%, 7 mins or [water (10 mM NH₄HCO₃)-MeCN]; B %: 25%-55%, 8 mins). The aqueous solution was lyophilized to give desired product.

### Compound 39

### Preparation of 8-((4,6-difluoroindolin-1-yl)methyl)-N-(2-(dimethylamino)ethyl)-2-morpholino-4-oxo-4H-chromene-6-carboxamide was prepared according to the procedure described herein for Step 4 in Scheme 11

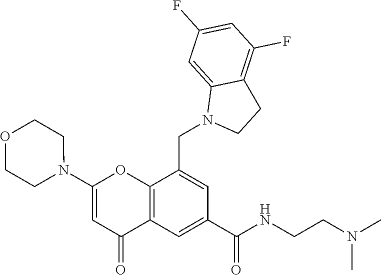 [SMILES: CN(C)CCNC(=O)c1cc(CN2CCc3c(F)cc(F)cc32)c2oc(N3CCOCC3)cc(=O)c2c1]

The desired compound (23.7 mg, 45.38 umol, 28.68% yield, 98.15% purity) was obtained as pale yellow solid. ¹H NMR (DMSO-d6, 400 MHz) δ 9.09 (s, 1H), 8.45 (s, 1H), 8.15 (s, 1H), 6.44˜6.31 (m, 2H), 5.73 (s, 1H), 4.64 (s, 2H), 3.69˜3.65 (m, 4H), 3.64˜3.55 (m, 2H), 3.56˜3.49 (m, 4H), 3.48 (m, 2H), 3.26 (s, 2H), 2.97 (s, 2H), 2.81 (s, 6H). MS: 98.15% (220 nm), 100.00% (254 nm). MS (ESI): mass calcd. For C₂₇H₃₀F₂N₄O₄ 512.22 m/z found 513.2 [M+H]⁺.

### Compound 40

### Preparation of 8-((4,6-difluoroindolin-1-yl)methyl)-N-(2-(dimethylamino)ethyl)-N-methyl-2-morpholino-4-oxo-4H-chromene-6-carboxamide was prepared according to the procedure described herein for Step 4 in Scheme 11

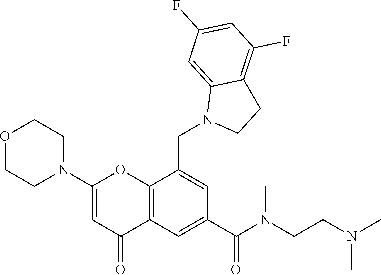 [SMILES: CN(C)CCN(C)C(=O)c1cc(CN2CCc3c(F)cc(F)cc32)c2oc(N3CCOCC3)cc(=O)c2c1]

The desired compound (16.7 mg, 31.48 umol, 19.90% yield, 99.26% purity) was obtained as yellow solid. ¹H NMR (400 MHz, DMSO-d₆) δ 8.01 (s, 1H), 7.82 (s, 1H), 6.43 (d, J=9.6 Hz, 1H), 6.32 (t, J=8.0 Hz, 1H), 5.79 (s, 1H), 4.63 (s, 2H), 3.81-3.79 (m, 2H), 3.69˜3.66 (m, 4H), 3.58˜3.55 (m, 4H), 3.51 (t, J=8.4 Hz, 2H), 3.35˜2.33 (m, 2H), 2.98˜2.93 (m, 5H), 2.85˜2.83 (m, 6H). LCMS: 99.26% (220 nm), 99.76% (254 nm). MS (ESI): mass calcd. For C₂₈H₃₂F₂N₄O₄ 526.24 m/z found 527.2 [M+H]⁺.

### Compound 41

### Preparation of 8-((4,6-difluoroindolin-1-yl)methyl)-6-(morpholine-4-carbonyl)-2-morpholino-4H-chromen-4-one was prepared according to the procedure described herein for Step 4 in Scheme 11

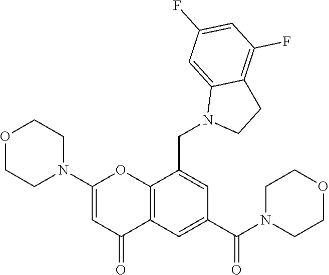 [SMILES: O=C(c1cc(CN2CCc3c(F)cc(F)cc32)c2oc(N3CCOCC3)cc(=O)c2c1)N1CCOCC1]

The desired compound (18.2 mg, 32.47 umol, 20.52% yield, 91.26% purity) was obtained as yellow solid. ¹H NMR (DMSO-d₆, 400 MHz) δ7.85 (s, 1H), 7.57 (s, 1H), 6.50˜6.20 (m, 2H), 5.60 (s, 1H), 4.64 (s, 2H), 3.70˜3.66 (m, 4H), 3.57˜3.40 (m, 12H), 3.30˜3.26 (m, 2H), 2.98 (t, J=8.4 Hz, 2H). HPLC: 91.27% (220 nm), 100% (254 nm). MS (ESI): mass calcd. For C₂₇H₂₇F₂O₅N₃ 511.19 m/z found 512.2 [M+H]⁺.

### Compound 42

### 8-((4,6-difluoroindolin-1-yl)methyl)-2-morpholino-6-(pyrrolidine-1-carbonyl)-4H-chromen-4-one was prepared according to the procedure described herein for Step 4 in Scheme 11)

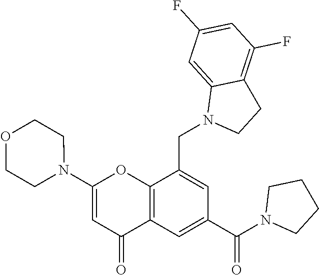 [SMILES: O=C(c1cc(CN2CCc3c(F)cc(F)cc32)c2oc(N3CCOCC3)cc(=O)c2c1)N1CCCC1]

The desired compound (22.6 mg, 44.90 umol, 24.83% yield, 98.45% purity) was obtained as a white solid. ¹H NMR (400 MHz, DMSO-d₆) δ 7.97 (d, J=2.0 Hz, 1H), 7.70 (d, J=2.0 Hz, 1H), 6.44 (d, J=2.0 Hz, 1H), 6.35 (t, J=2.0 Hz, 1H), 5.61 (s, 1H), 4.64 (s, 2H), 3.71-3.65 (m, 4H), 3.54˜3.51 (m, 4H), 3.34˜3.32 (m, 2H), 2.97˜2.94 (m, 2H), 1.89˜1.76 (m, 4H). LCMS: 98.45% (220 nm), 100.00% (254 nm). MS (ESI): mass calcd. For C₂₇H₂₇F₂N₃O₄ 495.20 m/z found 496.20 [M+H]⁺.

### Compound 43

### 8-((4,6-difluoroindolin-1-yl)methyl)-6-(3-(dimethylamino)pyrrolidine-1-carbonyl)-2-morpholino-4H-chromen-4-one was prepared according to the procedure described herein for Step 4 in Scheme 11

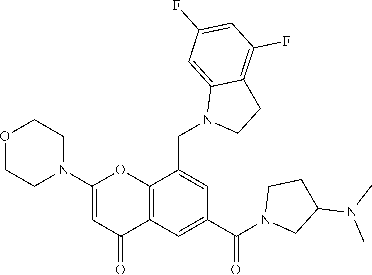 [SMILES: CN(C)C1CCN(C(=O)c2cc(CN3CCc4c(F)cc(F)cc43)c3oc(N4CCOCC4)cc(=O)c3c2)C1]

The desired compound (44 mg, 80.75 umol, 44.65% yield, 98.84% purity) was obtained as a pale yellow solid. ¹H NMR (400 MHz, DMSO-d₆) δ 10.86 (s, 1H), 8.01 (s, 1H), 7.73 (s, 1H), 6.44 (d, J=10.4 Hz, 1H), 6.31 (t, J=10.0 Hz, 1H), 5.63 (s, 1H), 4.65 (s, 2H), 3.69˜3.68 (m, 2H), 3.67˜3.65 (m, 4H), 3.56˜3.53 (m, 4H), 3.49˜3.47 (m, 2H), 2.99˜2.97 (m, 2H), 2.88˜2.66 (m, 6H), 2.64˜2.62 (m, 2H), 2.18˜2.16 (m, 1H), 1.72˜1.46 (m, 2H). HPLC: 98.85% (220 nm), 99.79% (254 nm). MS (ESI): mass calcd. For C₂₉H₃₂F₂N₄O₄ 538.24 m/z found 539.2 [M+H]⁺.

### Compound 44

### Preparation of 8-((4,6-difluoroindolin-1-yl)methyl)-6-(4-methylpiperazine-1-carbonyl)-2-morpholino-4H-chromen-4-one was prepared according to the procedure described herein for Step 4 in Scheme 11

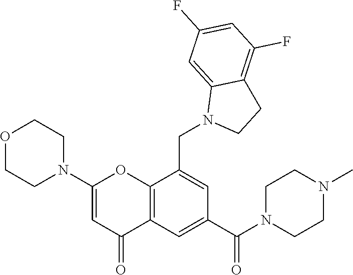 [SMILES: CN1CCN(C(=O)c2cc(CN3CCc4c(F)cc(F)cc43)c3oc(N4CCOCC4)cc(=O)c3c2)CC1]

The desired compound (23.2 mg, 43.01 umol, 23.78% yield, 97.24% purity) was obtained as pale yellow solid. ¹H NMR (400 MHz, DMSO-d₆) δ 11.09 (s, 1H), 7.93 (d, J=2.0 Hz, 1H), 7.64 (d, J=2.0 Hz, 1H), 6.44 (d, J=1.6 Hz, 1H), 6.36 (t, J=1.6 Hz, 1H), 5.65 (s, 1H), 4.64 (s, 2H), 4.04˜4.00 (m, 4H), 3.71-3.67 (m, 4H), 3.57˜3.53 (m, 4H), 3.48˜3.46 (m, 2H), 3.37 (s, 2H), 2.99˜2.95 (m, 2H), 2.97 (t, J=8.4 Hz, 2H), 2.74 (d, J=4.4 Hz, 3H). LCMS: 97.25% (220 nm), 100.00% (254 nm). MS (ESI): mass calcd. For C₂₈H₃₀F₂N₄O₄ 524.22 m/z found 525.2 [M+H]⁺.

### Compound 45

### Preparation of 6-(azetidine-1-carbonyl)-8-((4,6-difluoroindolin-1-yl)methyl)-2-morpholino-4H-chromen-4-one was prepared according to the procedure described herein for Step 4 in Scheme 11

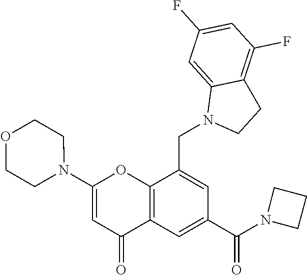 [SMILES: O=C(c1cc(CN2CCc3c(F)cc(F)cc32)c2oc(N3CCOCC3)cc(=O)c2c1)N1CCC1]

The desired compound (12.4 mg, 25.49 umol, 14.09% yield, 98.96% purity) was obtained as white solid. ¹H NMR (DMSO-d₆, 400 MHz) δ 8.07 (d, J=2.0 Hz, 1H), 7.77 (d, J=2.0 Hz, 1H), 6.44 (d, J=9.6 Hz, 1H), 6.32 (t, J=9.6 Hz, 1H), 5.60 (s, 1H), 4.65 (s, 2H), 4.24 (t, J=7.2 Hz, 2H), 4.04 (t, J=7.6 Hz, 2H), 3.72-3.64 (m, 4H), 3.56-3.51 (m, 4H), 3.49-3.44 (m, 2H), 2.97 (t, J=8.4 Hz, 2H), 2.25 (t, J=7.8 Hz, 2H). HPLC: 98.96% (220 nm), 100% (254 nm). MS (ESI): mass calcd. For C₂₆H₂₅F₂O₄N₃ 481.18 m/z found 482.1 [M+H]⁺.

### Compound 46

### 8-((4,6-difluoroindolin-1-yl)methyl)-6-(3-(dimethylamino)azetidine-1-carbonyl)-2-morpholino-4H-chromen-4-one was prepared according to the procedure described herein for Step 4 in Scheme 11

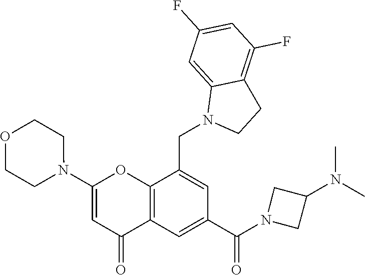 [SMILES: CN(C)C1CN(C(=O)c2cc(CN3CCc4c(F)cc(F)cc43)c3oc(N4CCOCC4)cc(=O)c3c2)C1]

The desired compound (18.1 mg, 34.40 umol, 19.02% yield, 99.70% purity) was obtained as white solid. ¹H NMR (DMSO-d6, 400 MHz) δ 8.09 (s, 1H), 7.73 (s, 1H), 6.43 (d, J=10.0 Hz, 1H), 6.31 (t, J=9.2 Hz, 1H), 5.60 (s, 1H), 4.66 (s, 2H), 4.17 (s, 1H), 4.05 (t, J=8.0 Hz, 1H), 3.93 (s, 1H), 3.81 (s, 1H), 3.69 (s, 4H), 3.53 (t, J=6.0 Hz, 4H), 3.38˜3.34 (m, 2H), 3.07 (t, J=4.8 Hz, 1H), 3.00 (t, J=7.2 Hz, 2H), 2.05 (s, 6H). LCMS: 99.70% (220 nm), 100.00% (254 nm). MS (ESI): mass calcd. For C₂₈H₃₀F₂N₄O₄ 524.22 m/z found 525.2 [M+H]⁺.

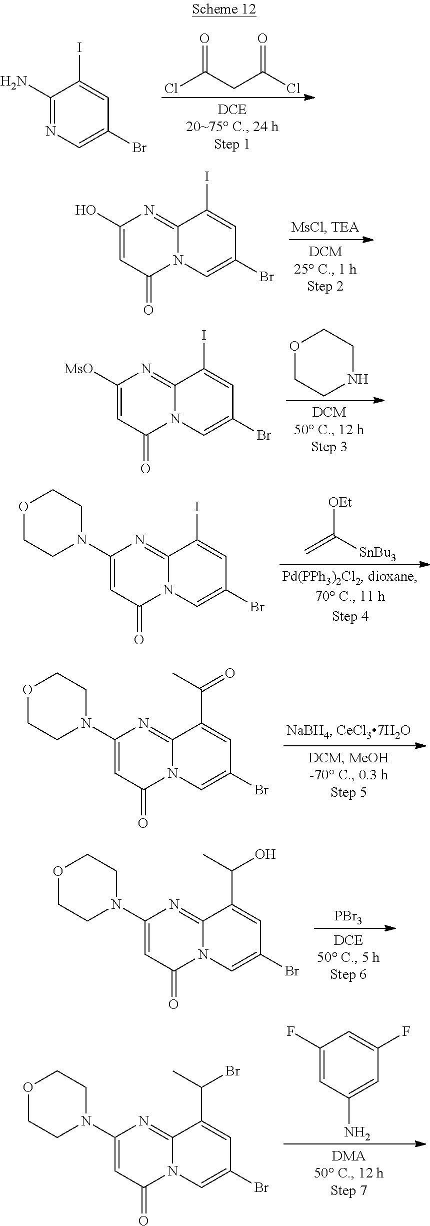

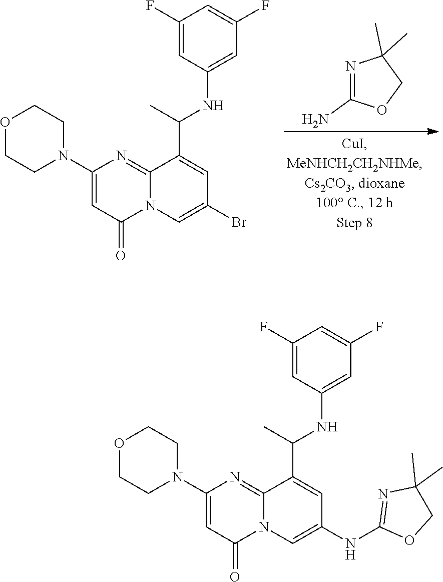

### Compound 47

### Preparation of 9-(1-((3,5-difluorophenyl)amino)ethyl)-7-((4,4-dimethyl-4,5-dihydrooxazol-2-yl)amino)-2-morpholino-4H-pyrido[1,2-a]pyrimidin-4-one (Steps 1-8 in Scheme 12)

### Preparation of 7-bromo-2-hydroxy-9-iodo-4H-pyrido[1,2-a]pyrimidin-4-one (Step 1 in Scheme 12)

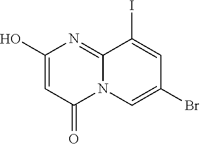 [SMILES: O=c1cc(O)nc2c(I)cc(Br)cn12]

To a mixture of 5-bromo-3-iodo-pyridin-2-amine (40 g, 133.82 mmol, 1 eq) in DCE (500 mL) was added propanedioyl dichloride (20.75 g, 147.20 mmol, 14.31 mL, 1.1 eq) dropwise at 0° C. under N₂. Then the mixture was stirred at 20° C. for an hour and then at 50° C. for 9 hours. Then the mixture was stirred at 70° C. for 10 hours. Then the mixture was stirred at 75° C. for 4 hours. TLC (Petroleum ether:Ethyl acetate=2:1), HPLC and LC-MS showed-15% 5-bromo-3-iodo-pyridin-2-amine was remained and ˜55% of desired on LCMS. There was some yellow solid formed and the yellow precipitate was collected by filtration. Compound 7-bromo-2-hydroxy-9-iodo-pyrido[1,2-a]pyrimidin-4-one (49 g, 133.54 mmol, 99.79% yield) was obtained as yellow solid. ¹H NMR (DMSO-d₆, 400 MHz) δ 9.11 (d, J=2.0 Hz, 1H), 8.67 (d, J=2.0 Hz, 1H), 5.53 (s, 1H).

### Preparation of 7-bromo-9-iodo-4-oxo-4H-pyrido[1,2-a]pyrimidin-2-yl methanesulfonate (Step 2 in Scheme 12)

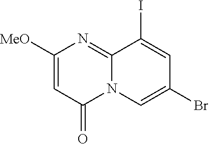 [SMILES: COc1cc(=O)n2cc(Br)cc(I)c2n1]

To a mixture of 7-bromo-2-hydroxy-9-iodo-pyrido[1,2-a]pyrimidin-4-one (10 g, 27.25 mmol, 1 eq) and Et₃N (5.52 g, 54.51 mmol, 7.59 mL, 2 eq) in DCM (80 mL) was added MsCl (6.5 g, 56.74 mmol, 4.39 mL, 2.08 eq) dropwise at 0° C. under N₂. The mixture was stirred at 25° C. for an hour. LC-MS showed 7-bromo-2-hydroxy-9-iodo-pyrido[1,2-a]pyrimidin-4-one was consumed completely. The mixture was used for next step without further purification. MS (ESI): mass calcd. For C₉H₆BrISO₄N₂ 443.83 m/z found 444.8 [M+H]⁺.

### Preparation of 7-bromo-9-iodo-2-morpholino-4H-pyrido[1,2-a]pyrimidin-4-one (Step 3 in Scheme 12)

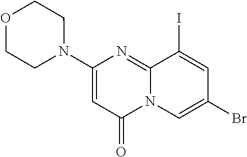 [SMILES: O=c1cc(N2CCOCC2)nc2c(I)cc(Br)cn12]

A mixture of (7-bromo-9-iodo-4-oxo-pyrido[1,2-a]pyrimidin-2-yl) methanesulfonate (12 g, 26.96 mmol, 1 eq) and morpholine (7.05 g, 80.89 mmol, 7.12 mL, 3 eq) in DCM (80 mL) was stirred at 50° C. for 12 hours. LC-MS showed (7-bromo-9-iodo-4-oxo-pyrido[1,2-a] pyrimidin-2-yl)methanesulfonate was consumed completely. The reaction mixture was quenched by addition H₂O (50 mL) at 0° C. and then extracted with DCM (100 mL×3). The combined organic layers were washed with brine (50 mL), dried over Na₂SO₄, filtered and concentrated under reduced pressure. The crude product was triturated with DCM (50 mL) at 25° C. for half an hour. Compound 7-bromo-9-iodo-2-morpholino-pyrido[1,2-a] pyrimidin-4-one (10.2 g, 23.39 mmol, 86.75% yield) was obtained as yellow solid. ¹H NMR (DMSO-d₆, 400 MHz) δ 8.80 (d, J=2.0 Hz, 1H), 8.56 (d, J=2.0 Hz, 1H), 5.62 (s, 1H), 3.72˜3.68 (m, 8H).

### Preparation of 9-acetyl-7-bromo-2-morpholino-4H-pyrido[1,2-a]pyrimidin-4-one (Step 4 in Scheme 12)

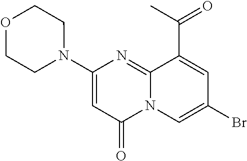 [SMILES: CC(=O)c1cc(Br)cn2c(=O)cc(N3CCOCC3)nc12]

A mixture of 7-bromo-9-iodo-2-morpholino-pyrido[1,2-a]pyrimidin-4-one (5.5 g, 12.61 mmol, 1 eq), tributyl(1-ethoxyvinyl)stannane (6.65 g, 18.42 mmol, 6.22 mL, 1.46 eq) and Pd(PPh₃)₂Cl₂ (442.67 mg, 630.67 umol, 0.05 eq) in dioxane (40 mL) was stirred at 70° C. for 11 hours under N₂. The mixture was cooled to 25° C. and HCl (2 M, 7.57 mL, 1.2 eq) was added and the mixture was stirred at 25° C. for an hour. LC-MS showed 7-bromo-9-iodo-2-morpholino-pyrido[1,2-a]pyrimidin-4-one was consumed completely. The mixture was concentrated under reduced pressure. The aqueous was made pH=7 with sat. NaHCO₃ and extracted with DCM (80 mL×3). The combined organic layer was washed with brine (80 mL×2), dried over Na₂SO₄, filtered and concentrated under reduced pressure. The crude product was triturated with petroleum ether and EtOAc (v:v=20:1, 30 mL) at 25° C. for half an hour. Compound 9-acetyl-7-bromo-2-morpholino-pyrido[1,2-a]pyrimidin-4-one (3.1 g, 8.80 mmol, 69.78% yield) was obtained as yellow solid. ¹H NMR (DMSO-d₆, 400 MHZ) δ 8392 (d, J=2.4 Hz, 1H), 8.02 (d, J=2.4 Hz, 1H), 5.69 (s, 1H), 3.68˜3.66 (m, 4H), 3.60˜3.58 (m, 4H), 2.65 (s, 3H).

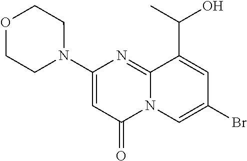 [SMILES: CC(O)c1cc(Br)cn2c(=O)cc(N3CCOCC3)nc12]

To a mixture of 9-acetyl-7-bromo-2-morpholino-pyrido[1,2-a]pyrimidin-4-one (1 g, 2.84 mmol, 1 eq) and CeCl₃·7H₂O (1.06 g, 2.84 mmol, 269.88 uL, 1 eq) in MeOH (25 mL) and DCM (6 mL) was added NaBH₄ (64.45 mg, 1.70 mmol, 0.6 eq) at −70° C. under N₂. The mixture was stirred at −70° C. for 0.3 hour. TLC (EtOAc, R_{f}=0.25) indicated 9-acetyl-7-bromo-2-morpholino-pyrido[1,2-a]pyrimidin-4-one was consumed completely. The mixture was quenched with ice water (30 mL) slowly at 0° C. and the organic solvent was removed under reduced pressure. The aqueous was extracted with EtOAc (40 mL×3). The combined organic layer was washed with brine (30 mL), dried over Na₂SO₄, filtered and concentrated under reduced pressure. Compound 7-bromo-9-(1-hydroxyethyl)-2-morpholino-pyrido[1,2-a]pyrimidin-4-one (0.67 g, 1.89 mmol, 66.62% yield) was obtained as yellow solid. ¹H NMR (DMSO-d₆, 400 MHz) δ 8.76 (d, J=2.0 Hz, 1H), 7.85 (d, J=2.0 Hz, 1H), 5.66 (s, 1H), 5.51 (d, J=4.4 Hz, 1H), 5.52˜5.15 (m, 1H), 3.69˜3.67 (m, 4H), 3.62˜3.59 (m, 4H), 1.39 (d, J=6.4 Hz, 3H).

### Preparation of 7-bromo-9-(1-bromoethyl)-2-morpholino-4H-pyrido[1,2-a]pyrimidin-4-one (Step 6 in Scheme 12)

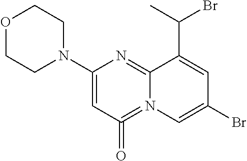

To a solution of 7-bromo-9-(1-hydroxyethyl)-2-morpholino-pyrido[1,2-a]pyrimidin-4-one (0.67 g, 1.89 mmol, 1 eq) in DCE (10 mL) was added PBr₃ (563.23 mg, 2.08 mmol, 1.1 eq) dropwise at 0° C. under N₂. The mixture was stirred at 50° C. for 5 hours. LC-MS showed 7-bromo-9-(1-hydroxyethyl)-2-morpholino-pyrido[1,2-a]pyrimidin-4-one was consumed completely. The mixture was cooled to 0° C. and adjusted to pH=6 with sat. NaHCO₃. The organic phase was separated and the aqueous phase was extracted with DCM (10 mL×2). The combined organic phase was washed with brine (10 mL), dried over anhydrous Na₂SO₄, filtered and concentrated in vacuum. Compound 7-bromo-9-(1-bromoethyl)-2-morpholino-pyrido[1,2-a]pyrimidin-4-one (0.63 g, 1.51 mmol, 79.85% yield) was obtained as yellow solid. ¹H NMR (DMSO-d₆, 400 MHz) δ 8.82 (s, 1H), 8.20 (s, 1H), 5.94˜5.90 (m, 1H), 5.68 (s, 1H), 3.72˜3.66 (m, 8H), 2.06 (d, J=6.8 Hz, 3H).

### Preparation of 7-bromo-9-(1-((3,5-difluorophenyl)amino)ethyl)-2-morpholino-4H-pyrido[1,2-a]pyrimidin-4-one (Step 7 in Scheme 12)

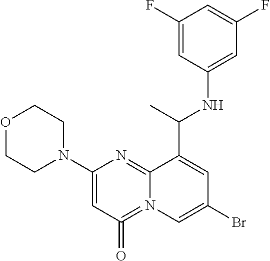

A mixture of 7-bromo-9-(1-bromoethyl)-2-morpholino-pyrido[1,2-a]pyrimidin-4-one (0.63 g, 1.51 mmol, 1 eq) and 3,5-difluoroaniline (779.81 mg, 6.04 mmol, 4 eq) in DMA (10 mL) was stirred at 50° C. for 12 hours. LC-MS showed 7-bromo-9-(1-bromoethyl)-2-morpholino-pyrido[1,2-a]pyrimidin-4-one was consumed completely. The mixture was cooled to 0° C. and quenched with water (50 mL). The aqueous phase was extracted with ethyl acetate (50 mL×3). The combined organic phase was washed with brine (30 mL×2), dried with anhydrous Na₂SO₄, filtered and concentrated in vacuum. The residue was purified by flash silica gel chromatography (Biotage; 12 g SepaFlash® Silica Flash Column, Eluent of 0˜100% Ethyl acetate/Petroleum ether gradient @60 mL/min). The solvent was concentrated. Compound 7-bromo-9-[1-(3,5-difluoroanilino)ethyl]-2-morpholino-pyrido[1,2-a] pyrimidin-4-one (0.48 g, 1.03 mmol, 68.32% yield) was obtained as yellow oil. ¹H NMR (DMSO-d₆, 400 MHz) δ 8.76 (d, J=2.0 Hz, 1H), 7.73 (d, J=2.0 Hz, 1H), 6.91 (d, J=7.2 Hz, 1H), 6.24 (t, J=9.6 Hz, 1H), 6.09 (d, J=9.6 Hz, 2H), 5.70 (s, 1H), 5.09˜5.06 (m, 1H), 3.69˜3.64 (m, 8H), 1.47 (d, J=6.8 Hz, 3H).

### Preparation of 9-(1-((3,5-difluorophenyl)amino)ethyl)-7-((4,4-dimethyl-4,5-dihydrooxazol-2-yl)amino)-2-morpholino-4H-pyrido[1,2-a]pyrimidin-4-one (Step 8 in Scheme 12)

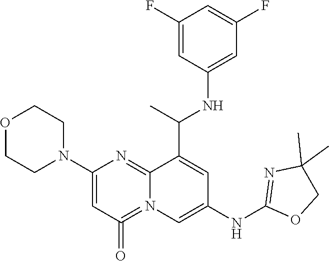

To a mixture of 7-bromo-9-[1-(3,5-difluoroanilino)ethyl]-2-morpholino-pyrido[1,2-a]pyrimidin-4-one (0.1 g, 214.92 umol, 1 eq) and 4,4-dimethyl-5H-oxazol-2-amine (167.69 mg, 859.68 umol, 4 eq, HBr) in dioxane (2 mL) was added CuI (81.86 mg, 429.84 umol, 2 eq), N,N′-dimethylethane-1,2-diamine (9.47 mg, 107.46 umol, 11.57 uL, 0.5 eq) and Cs₂CO₃ (210.07 mg, 644.76 umol, 3 eq) at 25° C. under N₂. The mixture was stirred at 100° C. for 12 hours. LC-MS and HPLC showed the reaction was completely. The mixture was filtered and the filtrate was concentrated under reduced pressure at 45° C. The residue was purified by prep-HPLC (column: Waters Xbridge BEH C18 100*30 mm*10 um; mobile phase: [water (10 mM NH₄HCO₃)-MeCN]; B %: 30%-60%, 8 mins). The solvent was removed under freeze drying. Compound 9-[1-(3,5-difluoroanilino)ethyl]-7-[(4,4-dimethyl-5H-oxazol-2-yl)amino]-2-morpholino-pyrido[1,2-a]pyrimidin-4-one (3.1 mg, 6.22 umol, 2.89% yield, 100% purity) was obtained as white solid. ¹H NMR (DMSO-d₆, 400 MHz) δ 9.33 (s, 1H), 8.39 (s, 1H), 7.70 (s, 1H), 7.45 (s, 1H), 6.96 (s, 1H), 6.20 (t, J=9.6 Hz, 1H), 6.05 (d, J=9.6 Hz, 2H), 5.63 (s, 1H), 5.07 (s, 1H), 4.13-4.10 (m, 2H), 3.70˜3.67 (m, 4H), 3.62˜3.57 (m, 4H), 1.47 (d, J=6.8 Hz, 3H), 1.23 (s, 6H) HPLC: 100% (220 nm), 96.01% (254 nm). MS (ESI): mass calcd. For C₂₅H₂₈F₂O₃N₆ 498.22 m/z found 499.3 [M+H]⁺.

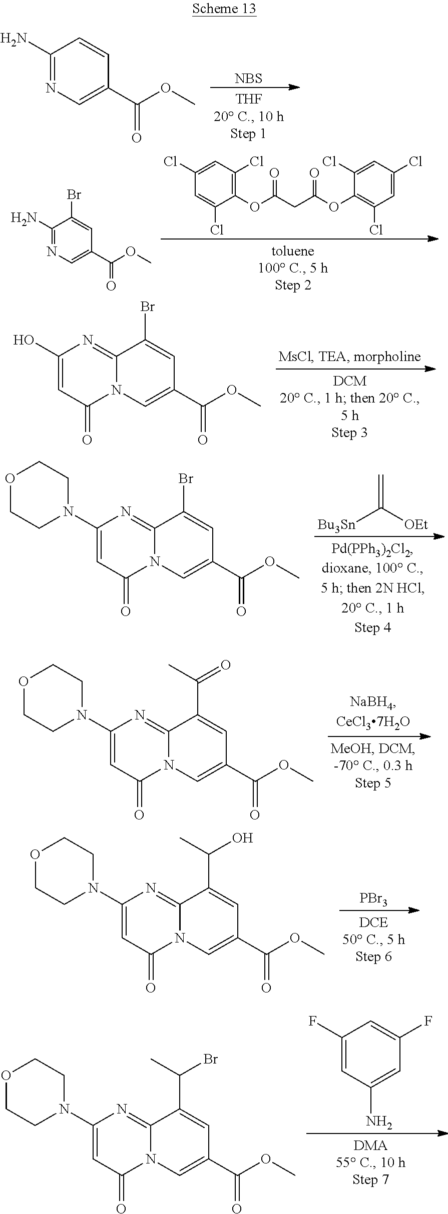

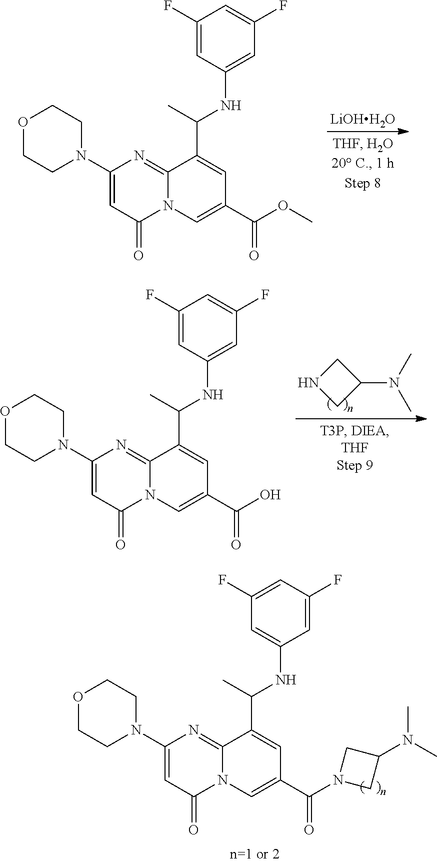

### General Procedures for Preparing Compounds in Scheme 13

### Preparation of methyl 6-amino-5-bromonicotinate (Step 1 in Scheme 13)

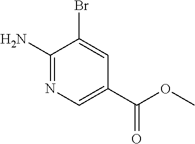

To a mixture of methyl 6-aminopyridine-3-carboxylate (50 g, 328.62 mmol, 1 eq) in THF (800 mL) was added NBS (64.34 g, 361.48 mmol, 1.1 eq) in portions at 0° C. Then the mixture was stirred at 20° C. for 10 hours. TLC (Petroleum ether:Ethyl acetate=2:1, R_{f}=0.45) showed the reaction was complete. The mixture was quenched with ice water (200 mL) and the organic solvent was removed under reduced pressure. To the residue was added water (500 mL). There was some brown solid formed. The filtrate was extracted with EtOAc (200 mL×6). The combined organic layer was washed with sat.NaHCO₃ (200 mL×4) and brine (100 mL×1), dried over Na₂SO₄, filtered and concentrated under reduced pressure. The solid (47 g) was collected without further purification. The residue was triturated with EtOAc (200 mL) and dried under reduced pressure (11 g). Compound methyl 6-amino-5-bromo-pyridine-3-carboxylate (58 g, 251.03 mmol, 76.39% yield) was obtained as brown solid. ¹H NMR (DMSO-d₆, 400 MHz) δ 8.49 (s, 1H), 8.08 (s, 1H), 7.15˜7.14 (m, 2H), 3.78 (s, 3H).

### Preparation of methyl 9-bromo-2-hydroxy-4-oxo-4H-pyrido[1,2-a]pyrimidine-7-carboxylate (Step 2 in Scheme 13)

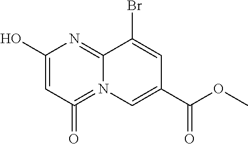

To a mixture of methyl 6-amino-5-bromo-pyridine-3-carboxylate (21 g, 90.89 mmol, 1 eq) in toluene (300 mL) at 50° C. was added bis(2,4,6-trichlorophenyl) propanedioate (50 g, 108.01 mmol, 1.19 eq) in portions at 50° C. under N₂. Then the mixture was stirred at 100° C. for 5 hours. TLC (Petroleum ether:Ethyl acetate=2:1, R_{f}=0.10) and LC-MS showed the reaction was complete. The brown precipitate was collected by filtration. The precipitate was washed with small portions of ice cold petroleum ether (200 mL) until the filtrate was colourless. Compound methyl 9-bromo-2-hydroxy-4-oxo-pyrido[1,2-a]pyrimidine-7-carboxylate (26.5 g, 88.61 mmol, 97.49% yield) was obtained as brown solid. ¹H NMR (DMSO-d₆, 400 MHz) δ 9.31 (s, 1H), 8.45 (s, 1H), 5.59 (s, 1H), 3.92 (s, 3H).

### Preparation of methyl 9-bromo-2-morpholino-4-oxo-4H-pyrido[1,2-a]pyrimidine-7-carboxylate (Step 3 in Scheme 13)

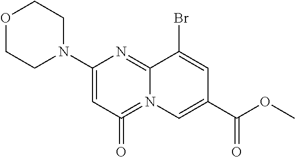

To a mixture of methyl 9-bromo-2-hydroxy-4-oxo-pyrido[1,2-a]pyrimidine-7-carboxylate (26.5 g, 88.61 mmol, 1 eq) and Et₃N (17.93 g, 177.21 mmol, 24.67 mL, 2 eq) in DCM (300 mL) was added MsCl (14.21 g, 124.05 mmol, 9.60 mL, 1.4 eq) dropwise at 0° C. under N₂. Then the mixture was stirred at 20° C. for an hour. Then morpholine (23.16 g, 265.82 mmol, 23.39 mL, 3 eq) was added and the mixture was stirred at 20° C. for 5 hours. TLC (Petroleum ether:Ethyl acetate=1:1, R_{f}=0.2) and LC-MS showed the reaction was complete. The mixture was poured into ice water (200 mL) at 0° C. and there was some solid formed. The solid (9 g, off-white solid) was collected after filtration. The organic solvent was separated. The aqueous was extracted with DCM (150 mL×4). The combined organic layer was washed with brine (100 mL×1), dried over Na₂SO₄, filtered and concentrated under reduced pressure. The residue was triturated with DCM (50 mL) and EtOAc (100 mL), then the solid (15 g, off-white solid) was dried under reduced pressure. Compound methyl 9-bromo-2-morpholino-4-oxo-pyrido[1,2-a]pyrimidine-7-carboxylate (24 g, 65.19 mmol, 73.57% yield) was obtained as off white solid. ¹H NMR (DMSO-d₆, 400 MHz) δ 9.24 (d, J=2.0 Hz, 1H), 8.37 (d, J=2.0 Hz, 1H), 5.69 (s, 1H), 3.90 (s, 3H), 3.69 (s, 8H).

### Preparation of methyl 9-acetyl-2-morpholino-4-oxo-4H-pyrido[1,2-a]pyrimidine-7-carboxylate (Step 4 in Scheme 13)

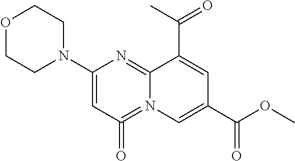

A mixture of methyl 9-bromo-2-morpholino-4-oxo-pyrido[1,2-a]pyrimidine-7-carboxylate (9 g, 24.44 mmol, 1 eq), tributyl(1-ethoxyvinyl)stannane (12.87 g, 35.64 mmol, 12.03 mL, 1.46 eq) and Pd(PPh₃)₂Cl₂ (857.88 mg, 1.22 mmol, 0.05 eq) in dioxane (100 mL) was stirred at 100° C. for 5 hours under N₂. TLC (petroleum ether:EtOAc=0:1, R_{f}=0.2) and LC-MS showed the reaction was complete. The mixture was cooled to r.t. and 14.67 mL of 2N HCl was added and the mixture was stirred at r.t. for an hour. The organic solvent was removed under reduced pressure. The aqueous was made pH=7 with sat. NaHCO₃ and extracted with DCM (100 mL×3). The combined organic layer was washed with brine (50 mL×3), dried over Na₂SO₄, filtered and concentrated under reduced pressure. The residue was triturated with petroleum ether and EtOAc (v:v=10:1, 440 mL×1). The solid was collected after filtration. Compound methyl 9-acetyl-2-morpholino-4-oxo-pyrido[1,2-a]pyrimidine-7-carboxylate (7 g, 21.13 mmol, 86.43% yield) was obtained as yellow solid. ¹H NMR (DMSO-d₆, 400 MHz) δ 9.36 (d, J=2.4 Hz, 1H), 8.18 (d, J=2.0 Hz, 1H), 5.72 (s, 1H), 3.91 (s, 3H), 3.68˜3.65 (m, 8H), 2.68 (s, 3H).

### Preparation of methyl 9-(1-hydroxyethyl)-2-morpholino-4-oxo-4H-pyrido[1,2-a]pyrimidine-7-carboxylate (Step 5 in Scheme 13)

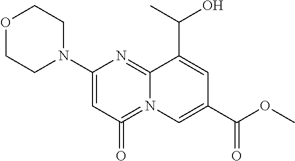

To a mixture of methyl 9-acetyl-2-morpholino-4-oxo-pyrido[1,2-a]pyrimidine-7-carboxylate (7 g, 21.13 mmol, 1 eq) and CeCl₃·7H₂O (7.87 g, 21.13 mmol, 28.69 uL, 1 eq) in DCM (15 mL) and MeOH (60 mL) was added NaBH₄ (479.55 mg, 12.68 mmol, 0.6 eq) in portions at −70° C. and then the mixture was stirred at −70° C. for 0.3 hour. TLC (petroleum ether:EtOAc=0:1, R_{f}=0.2) and LCMS showed the reaction was complete. The mixture was quenched with ice water (100 mL) slowly at −70° C. and filtered through celite. The filtrate was extracted with EtOAc (100 mL×3). The combined organic layer was washed with brine (50 mL×1), dried over Na₂SO₄, filtered and concentrated under reduced pressure. Compound methyl 9-(1-hydroxyethyl)-2-morpholino-4-oxo-pyrido[1,2-a]pyrimidine-7-carboxylate (5.6 g, 16.80 mmol, 79.52% yield) was obtained as yellow solid. ¹H NMR (DMSO-d₆, 400 MHz) δ 9.24 (d, J=2.0 Hz, 1H), 8.17 (d, J=2.0 Hz, 1H), 5.67 (s, 1H), 5.52 (d, J=4.4 Hz, 1H), 5.19˜5.15 (m, 1H), 3.90 (s, 3H), 3.68˜3.60 (m, 8H), 1.18 (t, J=7.6 Hz, 3H).

### Preparation of methyl 9-(1-bromoethyl)-2-morpholino-4-oxo-4H-pyrido[1,2-a]pyrimidine-7-carboxylate (Step 6 in Scheme 13)

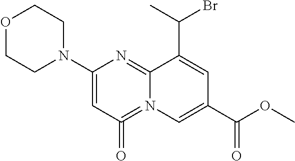

To a solution of methyl 9-(1-hydroxyethyl)-2-morpholino-4-oxo-pyrido[1,2-a]pyrimidine-7-carboxylate (3 g, 9.00 mmol, 1 eq) in DCE (50 mL) was added PBr₃ (3.17 g, 11.70 mmol, 1.11 mL, 1.3 eq) dropwise at 0° C. and then the mixture was stirred at 50° C. for 5 hours. TLC (petroleum ether:EtOAc=1:1, R_{f}=0.35) and LC-MS showed the reaction was complete. The mixture was quenched with ice water (50 mL) at 0° C. and made pH=6˜7 with sat.Na₂CO₃. The aqueous was extracted with DCM (50 mL×3). The combined organic layer was washed with brine (20 mL×1), dried over Na₂SO₄, filtered and concentrated under reduced pressure. The residue was triturated by EtOAc (30 mL). Compound methyl 9-(1-bromoethyl)-2-morpholino-4-oxo-pyrido[1,2-a]pyrimidine-7-carboxylate (1.9 g, 4.80 mmol, 53.28% yield) was obtained was white solid. ¹H NMR (DMSO-d₆, 400 MHz) δ 9.27 (d, J=2.0 Hz, 1H), 8.23 (d, J=2.0 Hz, 1H), 5.97˜5.86 (m, 1H), 5.70 (s, 1H), 3.91 (s, 3H), 3.69 (s, 8H), 2.08 (d, J=6.8 Hz, 3H).

### Preparation of methyl 9-(1-((3,5-difluorophenyl)amino)ethyl)-2-morpholino-4-oxo-4H-pyrido[1,2-a]pyrimidine-7-carboxylate (Step 7 in Scheme 13)

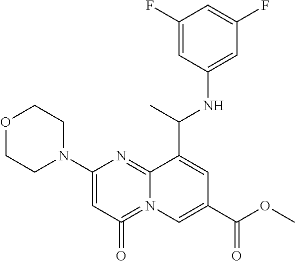

A solution of methyl 9-(1-bromoethyl)-2-morpholino-4-oxo-pyrido[1,2-a]pyrimidine-7-carboxylate (1.9 g, 4.80 mmol, 1 eq) and 3,5-difluoroaniline (1.86 g, 14.39 mmol, 3 eq) in DMA (20 mL) was stirred at 55° C. for 10 hours. TLC (Petroleum ether:Ethyl acetate=1:1, R_{f}=0.26) and LC-MS showed the reaction was complete. The mixture was quenched with ice water (50 mL) and there was some white solid formed. After filtration, the solid (1 g) was collected and concentrated under reduced pressure. The filtrate was extracted with EtOAc (30 mL×3). The combined organic layer was washed with brine (10 mL×4), dried over Na₂SO₄, filtered and concentrated under reduced pressure. The residue was triturated by MeOH (30 mL) and 1 g of solid was obtained which was used to the next step without further purification. Compound methyl 9-[1-(3,5-difluoroanilino)ethyl]-2-morpholino-4-oxo-pyrido[1,2-a] pyrimidine-7-carboxylate (2 g, 4.50 mmol, 93.85% yield) was obtained as white solid. ¹H NMR (DMSO-d₆, 400 MHz) δ 9.24 (d, J=2.0 Hz, 1H), 8.00 (d, J=2.0 Hz, 1H), 7.08 (d, J=6.8 Hz, 1H), 6.23 (t, J=7.2 Hz, 1H), 6.08 (dd, J=2.0 Hz, 10.4 Hz, 2H), 5.73 (s, 1H), 5.08˜5.05 (m, 1H), 3.86 (s, 3H), 3.70 (s, 8H), 1.48 (d, J=6.4 Hz, 3H).

### Preparation of 9-(1-((3,5-difluorophenyl)amino)ethyl)-2-morpholino-4-oxo-4H-pyrido[1,2-a]pyrimidine-7-carboxylic acid (Step 8 in Scheme 13)

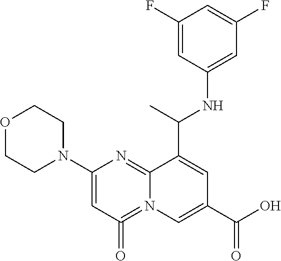

A solution of methyl 9-[1-(3,5-difluoroanilino)ethyl]-2-morpholino-4-oxo-pyrido[1,2-a]pyrimidine-7-carboxylate (2 g, 4.50 mmol, 1 eq) and LiOH·H₂O (566.48 mg, 13.50 mmol, 3 eq) in H₂O (10 mL) and THF (10 mL) was stirred at 20° C. for an hour. TLC (petroleum ether:EtOAc=1:1, R_{f}=0.05) and LC-MS showed the reaction was complete. The organic solvent was removed under reduced pressure. 5 mL of water was added and the aqueous was made pH=4 with 1N HCl at 0° C. and there was some solid formed. After filtration, the solid was collected and concentrated under reduced pressure. Compound 9-[1-(3,5-difluoroanilino)ethyl]-2-morpholino-4-oxo-pyrido[1,2-a]pyrimidine-7-carboxylic acid (1.8 g, 4.18 mmol, 92.93% yield) was obtained as white solid. ¹H NMR (DMSO-d₆, 400 MHz) δ 13.53-13.50 (m, 1H), 9.21 (d, J=2.0 Hz, 1H), 8.00 (d, J=2.0 Hz, 1H), 7.06 (d, J=6.8 Hz, 1H), 6.22 (t, J=7.2 Hz, 1H), 6.09 (dd, J=2.0 Hz, 10.4 Hz, 2H), 5.71 (s, 1H), 5.07˜5.02 (m, 1H), 3.69 (s, 8H), 1.47 (d, J=6.8 Hz, 3H).

### Preparation of Compounds in Scheme 13 (Step 9 in Scheme 13)

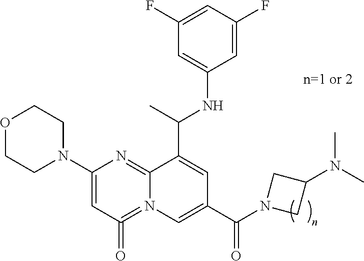

To a solution of 9-[1-(3,5-difluoroanilino)ethyl]-2-morpholino-4-oxo-pyrido[1,2-a]pyrimidine-7-carboxylic acid (697.02 umol, 1 eq), amine (1.05 mmol, 1.5 eq) and DIEA (3.49 mmol˜4.18 mmol, 5 eq˜6 eq) in THF (7 mL/mmol˜9 mL/mmol) was added T3P (906.13 umol, 50% purity, 1.3 eq) dropwise at 0° C. Then the mixture was stirred at 20° C. for 5 hours. HPLC and LCMS showed the reaction was nearly complete. The mixture was quenched with ice water and then extracted with EtOAc. The combined organic layer was washed with brine, dried over Na₂SO₄, filtered and concentrated under reduced pressure. The residue was purified by prep-HPLC (column: Phenomenex Gemini-NX 80*40 mm*3 um or Phenomenex Gemini-NX C18 75*30 mm*3 um; mobile phase: [water (10 mM NH₄HCO₃)-MeCN]; B %: 20%-50%, 8 mins or [water (10 mM NH₄HCO₃)-MeCN]; B %: 30%-50%, 8 mins). The eluent was removed under freeze drying. The aqueous solution was lyophilized to give desired product.

### Compound 48

### Preparation of 9-(1-((3,5-difluorophenyl)amino)ethyl)-7-(3-(dimethylamino)pyrrolidine-1-carbonyl)-2-morpholino-4H-pyrido[1,2-a]pyrimidin-4-one (Step 9 in Scheme 13)

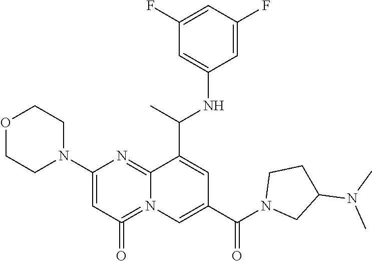

To a solution of 9-[1-(3,5-difluoroanilino)ethyl]-2-morpholino-4-oxo-pyrido[1,2-a]pyrimidine-7-carboxylic acid (0.3 g, 697.02 umol, 1 eq), N,N-dimethylpyrrolidin-3-amine (119.39 mg, 1.05 mmol, 1.5 eq) and DIEA (450.41 mg, 3.49 mmol, 607.03 uL, 5 eq) in THF (5 mL) was added T3P (576.62 mg, 906.13 umol, 538.90 uL, 50% purity, 1.3 eq) dropwise at 0° C. Then the mixture was stirred at 20° C. for 5 hours. HPLC and LCMS showed the reaction was nearly complete. The mixture was quenched with ice water (10 mL) and then extracted with EtOAc (10 mL×2). The combined organic layer was washed with brine (5 mL), dried over Na₂SO₄, filtered and concentrated under reduced pressure. The residue was purified by prep-HPLC (column: Phenomenex Gemini-NX 80*40 mm*3 um; mobile phase: [water (10 mM NH₄HCO₃)-MeCN]; B %: 20%-50%, 8 mins). The eluent was removed under freeze drying. Compound 9-[1-(3,5-difluoroanilino)ethyl]-7-[3-(dimethylamino)pyrrolidine-1-carbonyl]-2-morpholino-pyrido[1,2-a]pyrimidin-4-one (205.8 mg, 389.77 umol, 55.92% yield, 99.73% purity) was obtained as white solid. ¹H NMR (DMSO-d₆, 400 MHz) δ 8.88 (s, 1H), 7.82 (s, 1H), 7.03-6.96 (m, 1H), 6.22˜6.20 (m, 1H), 6.08 (d, J=9.6 Hz, 2H), 5.71 (s, 1H), 5.10-5.07 (m, 1H), 3.69 (s, 8H), 3.62-3.53 (m, 1H), 3.44-3.40 (m, 2H), 3.24-3.21 (m, 1H), 2.55-2.52 (m, 1H), 2.16-1.97 (m, 7H), 1.69-1.68 (m, 1H), 1.48 (d, J=6.4 Hz, 3H). HPLC: 99.73% (220 nm), 100% (254 nm). MS (ESI): mass calcd. For C₂₇H₃₂F₂N₆O₃ 526.25 m/z found 527.2 [M+H]⁺.

Chiral separation of Compound 48 was performed. Chiral Preparative Instrument: Thar 80 preparative SFC; Column: (s,s) Whelk O-1, 250×30 mm i.d., 10 m and Daicel Chiralpak IC, 250×30 mm i.d., 10 μm. Chiral Analytical Instrument: Waters Acquity Arc; Column: S,S_Whelk_O1, 3.5 μm, 0.46 cm id×10 cm L. Four isomers were obtained. Compound 48—P1 (Rt=6.501 minutes, E.E. by chiral HPLC (%)=95.8%); Compound 48—P2 (Rt=7.525 minutes, E.E. by chiral HPLC (%)=95.8=100%); Compound 48—P3 (Rt=8.298 minutes; E.E. by chiral HPLC (%)=98.8%) and Compound 48—P4 (Rt=14.409 minutes, E.E. by chiral HPLC (%)=99.46%).

### Compound 49

### 9-(1-((3,5-difluorophenyl)amino)ethyl)-7-(3-(dimethylamino)azetidine-1-carbonyl)-2-morpholino-4H-pyrido[1,2-a]pyrimidin-4-one was prepared according to the procedure described herein for Step 9 in Scheme 13

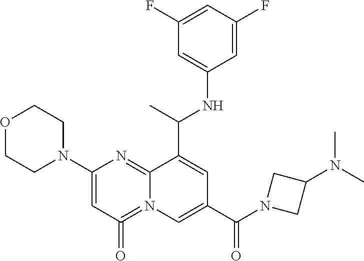

The desired compound (26 mg, 50.26 umol, 21.63% yield, 99.08% purity) was obtained as white solid. ¹H NMR (DMSO-d₆, 400 MHz) δ 8.95 (d, J=2.4 Hz, 1H), 7.84 (s, 1H), 7.04 (d, d, J=7.2 Hz, 1H), 6.25˜6.20 (m, 1H), 6.11˜6.08 (m, 2H), 5.72 (s, 1H), 5.08 (t, J=6.8 Hz, 1H), 4.22˜3.80 (m, 4H), 3.66 (s, 8H), 3.06˜3.03 (m, 1H), 2.05 (s, 6H), 1.49 (d, J=6.8 Hz, 3H). HPLC: 99.08% (220 nm), 100% (254 nm). MS (ESI): mass calcd. For C₂₆H₃₀F₂N₆O₃ 512.23 m/z found 513.2 [M+H]⁺.

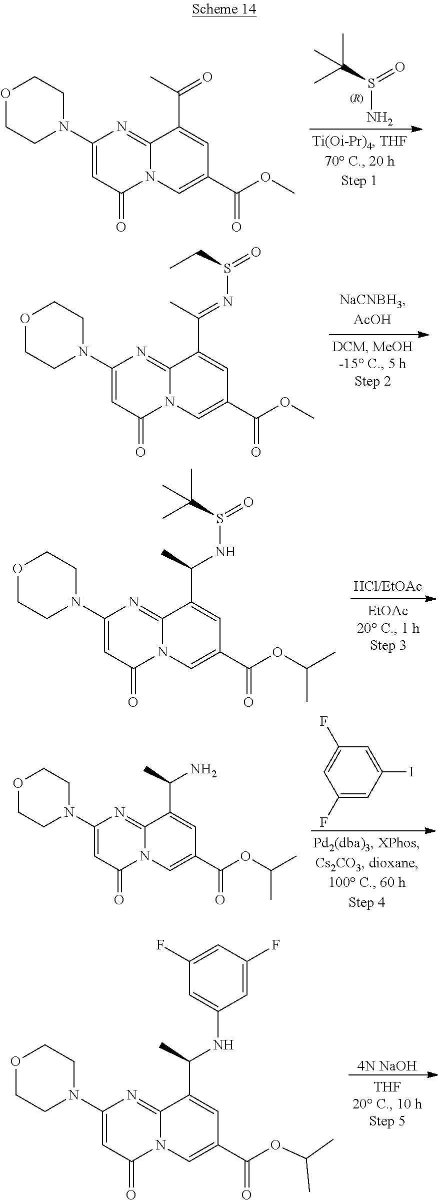

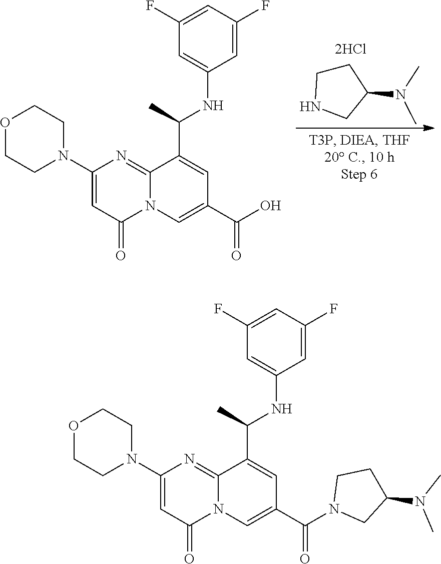

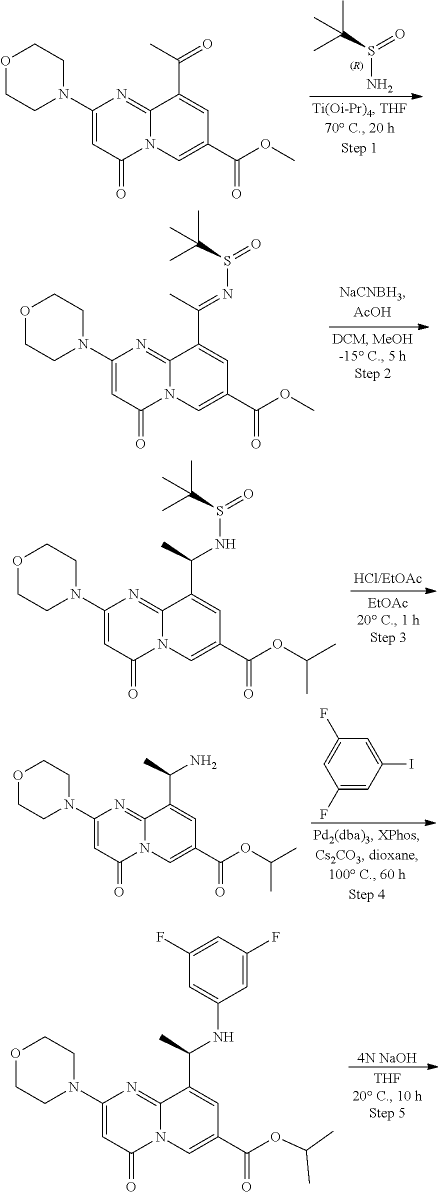

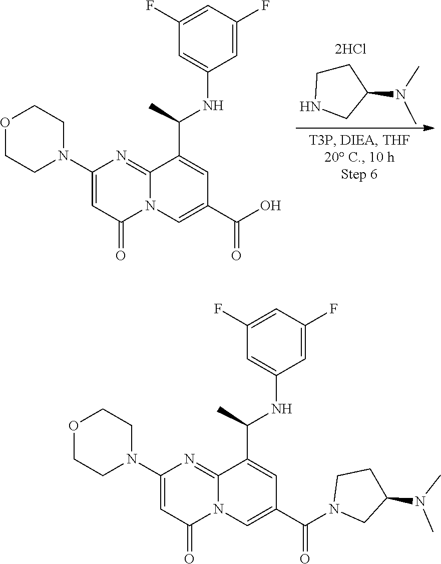

### Compound 50

### Preparation of 9-((R)-1-((3,5-difluorophenyl)amino)ethyl)-7-((R)-3-(dimethylamino)pyrrolidine-1-carbonyl)-2-morpholino-4H-pyrido[1,2-a]pyrimidin-4-one (Steps 1-6 in Scheme 14)

### Preparation of (R,E)-isopropyl 9-(1-((tert-butylsulfinyl)imino)ethyl)-2-morpholino-4-oxo-4H-pyrido[1,2-a]pyrimidine-7-carboxylate (Step 1 in Scheme 14)

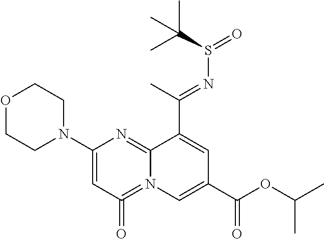

A mixture of methyl 9-acetyl-2-morpholino-4-oxo-pyrido[1,2-a]pyrimidine-7-carboxylate (4.1 g, 12.37 mmol, 1 eq), (R)-2-methylpropane-2-sulfinamide (2.70 g, 22.27 mmol, 1.8 eq) and Ti(i-PrO)₄ (14.07 g, 49.50 mmol, 14.61 mL, 4 eq) in THF (80 mL) was stirred at 70° C. for 20 hours under N₂. LC-MS and TLC (petroleum ether:EtOAc=2:1, R_{f}=0.3) showed the reaction was complete and most (R,E)-isopropyl 9-(1-((tert-butylsulfinyl)imino)ethyl)-2-morpholino-4-oxo-4H-pyrido[1,2-a]pyrimidine-7-carboxylate formed. The mixture was quenched with ice water (100 mL) at 0° C. and EtOAc (300 mL) was added. After filtration through celite, the filter cake was washed with EtOAc (50 mL×3). The organic layer was separated. The aqueous phase was extracted with EtOAc (100 mL×1). The combined organic layer was washed with brine (50 mL×1), dried over Na₂SO₄, filtered and concentrated under reduced pressure. Isopropyl 9-[(Z)—N—[(S)-tert-butylsulfinyl]-C-methyl-carbonimidoyl]-2-morpholino-4-oxo-pyrido[1,2-a]pyrimidine-7-carboxylate (5.5 g, 11.89 mmol, 96.09% yield) was obtained as brown solid. ¹H NMR (DMSO-d₆, 400 MHz) δ 9.30˜9.22 (m, 1H), 8.03˜7.96 (m, 1H), 5.71˜5.69 (m, 1H), 5.20˜5.17 (m, 1H), 3.65 (s, 8H), 2.53 (s, 3H), 1.35 (d, J=6.0 Hz, 6H), 1.21˜1.15 (m, 9H).

### Preparation of isopropyl 9-((R)-1-((R)-1,1-dimethylethylsulfinamido)ethyl)-2-morpholino-4-oxo-4H-pyrido[1,2-a]pyrimidine-7-carboxylate (Step 2 in Scheme 14)

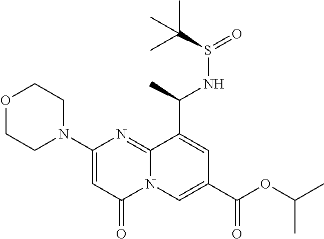 [SMILES: CC(C)OC(=O)c1cc([C@@H](C)N[S@](=O)C(C)(C)C)c2nc(N3CCOCC3)cc(=O)n2c1]

To a solution of isopropyl 9-[(E)-N—[(S)-tert-butylsulfinyl]-C-methyl-carbonimidoyl]-2-morpholino-4-oxo-pyrido[1,2-a]pyrimidine-7-carboxylate (5.5 g, 11.89 mmol, 1 eq) and AcOH (5.71 g, 95.12 mmol, 5.44 mL, 8 eq) in MeOH (50 mL) and DCM (50 mL) was added NaBH₃CN (2.24 g, 35.67 mmol, 3 eq) in portions at −15° C. and then the mixture was stirred at −15° C. for 5 hours. TLC (petroleum ether:EtOAc=0:1, R_{f}=0.10) and LC-MS showed the reaction was complete. The mixture was quenched with ice water (30 mL) at 0° C. and then sat. Na₂CO₃ solution was added until pH=7. The aqueous solution was extracted with EtOAc (50 mL×3). The combined organic layer was washed with brine (50 mL×2), dried over Na₂SO₄, filtered and concentrated under reduced pressure. The residue was purified by flash silica gel chromatography (Biotage®; 40 g SepaFlash® Silica Flash Column, Eluent of 0˜100% Ethylacetate/petroleum ether gradient @30 mL/min). The eluent was removed under reduced pressure. Compound isopropyl 9-[(1S)-1-[[(R)-tert-butylsulfinyl]amino]ethyl]-2-morpholino-4-oxo-pyrido[1,2-a]pyrimidine-7-carboxylate (2.8 g, 6.03 mmol, 50.69% yield) was obtained as pale yellow solid. ¹H NMR (DMSO-d₆, 400 MHz) δ 9.24 (s, 1H), 8.24 (s, 1H), 5.95 (d, J=8.4 Hz, 1H), 5.70 (s, 1H), 5.21˜5.17 (m, 1H), 5.03˜4.99 (m, 1H), 3.65 (s, 8H), 1.45 (d, J=6.8 Hz, 3H), 1.35 (d, J=4.4 Hz, 6H), 1.14 (s, 9H).

### Preparation of (R)-isopropyl 9-(1-aminoethyl)-2-morpholino-4-oxo-4H-pyrido[1,2-a]pyrimidine-7-carboxylate (Step 3 in Scheme 14)

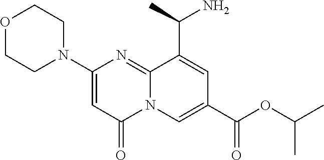 [SMILES: CC(C)OC(=O)c1cc([C@@H](C)N)c2nc(N3CCOCC3)cc(=O)n2c1]

To a solution of isopropyl 9-[(1R)-1-[[(R)-tert-butylsulfinyl]amino]ethyl]-2-morpholino-4-oxo-pyrido[1,2-a]pyrimidine-7-carboxylate (13.9 g, 29.92 mmol, 1 eq) in EtOAc (100 mL) was added HCl/EtOAc (4 M, 74.80 mL, 10 eq) and then the mixture was stirred at 20° C. for an hour. TLC (EtOAc:MeOH=20:1, R_{f}=0.1) and LC-MS showed the reaction was complete. The mixture was concentrated under reduced pressure. The residue was triturated with EtOAc (100 mL). After filtration, the solid was collected. Compound isopropyl 9-[(1R)-1-aminoethyl]-2-morpholino-4-oxo-pyrido[1,2-a]pyrimidine-7-carboxylate (11.5 g, 26.54 mmol, 88.70% yield, 2HCl, 100% ee.) was obtained as pale yellow solid. ¹H NMR (DMSO-d₆, 400 MHz) δ 9.28 (s, 1H), 8.57 (s, 2H), 8.27 (s, 1H), 5.74 (s, 1H), 5.24˜5.17 (m, 1H), 4.97 (t, J=6.0 Hz, 1H), 3.69 (s, 8H), 1.58 (d, J=6.8 Hz, 3H), 1.37 (t, J=6.0 Hz, 6H).

Chiral analytical Instrument: Waters Acquity UPC2; Column: Chiralcel OD-3 3 μm, 0.46 cm id×5 cm L. Mobile phase: A for SFC CO2 and B for MeOH (0.05% IPAm); Gradient: B in A from 5% to 50% in 3 minutes; Flow rate: 3.4 mL/min; Column temperature: 35° C.; Wavelength: 220 nm; System Back Pressure: 1800 psi. Rt=1.158 minutes; E.E. by Chiral HPLC (%)=100%.

### Preparation of (R)-isopropyl 9-(1-((3,5-difluorophenyl)amino)ethyl)-2-morpholino-4-oxo-4H-pyrido[1,2-a]pyrimidine-7-carboxylate (Step 4 in Scheme 14)

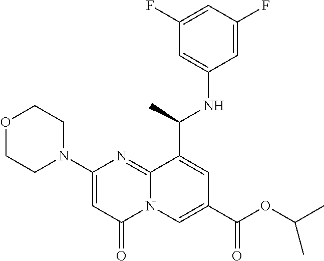 [SMILES: CC(C)OC(=O)c1cc([C@@H](C)Nc2cc(F)cc(F)c2)c2nc(N3CCOCC3)cc(=O)n2c1]

A mixture of isopropyl 9-[(1R)-1-aminoethyl]-2-morpholino-4-oxo-pyrido[1,2-a]pyrimidine-7-carboxylate (11 g, 25.38 mmol, 1 eq, 2HCl), 1,3-difluoro-5-iodo-benzene (12.18 g, 50.77 mmol, 6.09 mL, 2 eq), Pd₂(dba)₃ (2.32 g, 2.54 mmol, 0.1 eq), XPhos (1.82 g, 3.81 mmol, 0.15 eq) and Cs₂CO₃ (28.95 g, 88.85 mmol, 3.5 eq) in dioxane (110 mL) was stirred at 100° C. for 20 hours under N₂. Then 1,3-difluoro-5-iodo-benzene (6.09 g, 25.38 mmol, 3.05 mL, 1 eq), Pd₂(dba)₃ (1.16 g, 1.27 mmol, 0.05 eq), XPhos (907.60 mg, 1.90 mmol, 0.075 eq) and Cs₂CO₃ (8.27 g, 25.38 mmol, 1 eq) was added and the mixture was stirred at 100° C. for another 20 hours under N₂. TLC checking showed the starting material was not consumed, then another batch 1,3-difluoro-5-iodo-benzene (6.09 g, 25.38 mmol, 3.05 mL, 1 eq) was added and the mixture was stirred at 100° C. for 20 hours under N₂ until TLC (petroleum ether:EtOAc=1:1, R_{f}=0.2) and LC-MS showed the reaction was nearly complete. After filtration through celite, the filtrate was concentrated under reduced pressure. The residue was dissolved in EtOAc (300 mL) and washed with water (100 mL×2). The combined organic layer was washed with brine (100 mL×1), dried over Na₂SO₄, filtered and concentrated under reduced pressure. The residue was purified by flash silica gel chromatography (Biotage®; 120 g SepaFlash® Silica Flash Column, Eluent of 0˜69% Ethylacetate/petroleum ether gradient @100 mL/min. The eluent was removed under reduced pressure. Compound isopropyl 9-[(1R)-1-(3,5-difluoroanilino)ethyl]-2-morpholino-4-oxo-pyrido[1,2-a]pyrimidine-7-carboxylate (5.7 g, 12.06 mmol, 47.52% yield) was obtained as yellow solid. Also isopropyl 9-[(1R)-1-aminoethyl]-2-morpholino-4-oxo-pyrido[1,2-a]pyrimidine-7-carboxylate (1.2 g) was recovered as brown solid. ¹H NMR (DMSO-d₆, 400 MHz) δ 9.21 (s, 1H), 7.99 (s, 1H), 7.07 (d, J=7.2 Hz, 1H), 6.24˜6.19 (m, 1H), 6.10˜6.07 (m, 2H), 5.73 (s, 1H), 5.15˜5.10 (m, 1H), 5.08˜5.04 (m, 1H), 3.69 (s, 8H), 1.47 (d, J=6.8 Hz, 3H), 1.32 (d, J=7.2 Hz, 6H).

### Preparation of (R)-9-(1-((3,5-difluorophenyl)amino)ethyl)-2-morpholino-4-oxo-4H-pyrido[1,2-a]pyrimidine-7-carboxylic acid (Step 5 in Scheme 14)

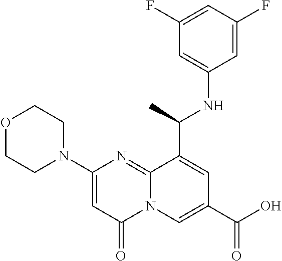 [SMILES: C[C@@H](Nc1cc(F)cc(F)c1)c1cc(C(=O)O)cn2c(=O)cc(N3CCOCC3)nc12]

A solution of isopropyl 9-[(1S)-1-(3,5-difluoroanilino)ethyl]-2-morpholino-4-oxo-pyrido[1,2-a] pyrimidine-7-carboxylate (5.7 g, 12.06 mmol, 1 eq) and aqueous NaOH solution (4 M, 60.32 mL, 20 eq) in THF (12 mL) was stirred at 20° C. for 6 hours. Then THF (10 mL) and aqueous NaOH solution (4 M, 15.08 mL, 5 eq) were added and the mixture was stirred at 20° C. for another 4 hours. TLC (petroleum ether:EtOAc=0:1, R_{f}=0.05) showed the reaction was complete. The organic solvent was removed under reduced pressure. The aqueous was extracted with MTBE (30 mL×2) to remove the impurity. The aqueous was adjusted to pH=4 with con. HCl at 0° C. and yellow solid precipitated. After filtration, the solid was collected and concentrated under reduced pressure. The filtrate was extracted with DCM and i-PrOH (v:v=3:1, 30 mL×3). The combined organic layer was washed with brine (20 mL×1), dried over Na₂SO₄, filtered and concentrated under reduced pressure. The residue was triturated with EtOAc (5 mL). Compound 9-[(1S)-1-(3,5-difluoroanilino)ethyl]-2-morpholino-4-oxo-pyrido [1,2-a]pyrimidine-7-carboxylic acid (4.3 g, 9.99 mmol, 82.81% yield, 96.84% ee.) was obtained as pale yellow solid. ¹H NMR (DMSO-d₆, 400 MHz) δ 13.53˜13.50 (m, 1H), 9.20 (s, 1H), 8.04 (s, 1H), 7.07 (d, J=7.2 Hz, 1H), 6.21 (t, J=7.6 Hz, 1H), 6.09˜6.06 (m, 2H), 5.70 (s, 1H), 5.08˜5.04 (m, 1H), 3.69 (s, 8H), 1.47 (d, J=6.4 Hz, 3H).

Chiral analytical Instrument: Waters Acquity UPC2; Column: Chiralpak IC-3 3 μm, 0.46 cm id×5 cm L; Mobile phase: A for SFC CO2 and B for EtOH (0.05% IPAm); Gradient: B in A from 5% to 50% in 3 minutes; Flow rate: 3.4 mL/min; Column temperature: 35° C.; Wavelength: 220 nm; System Back Pressure: 1800 psi Rt=1.767 minutes; E.E. by Chiral HPLC (%)=96.84%.

### Preparation of 9-((R)-1-((3,5-difluorophenyl)amino)ethyl)-7-((R)-3-(dimethylamino)pyrrolidine-1-carbonyl)-2-morpholino-4H-pyrido[1,2-a]pyrimidin-4-one (Step 6 in Scheme 14)

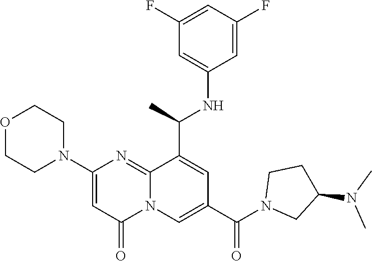 [SMILES: C[C@@H](Nc1cc(F)cc(F)c1)c1cc(C(=O)N2CC[C@@H](N(C)C)C2)cn2c(=O)cc(N3CCOCC3)nc12]

To a solution of 9-[(1R)-1-(3,5-difluoroanilino)ethyl]-2-morpholino-4-oxo-pyrido[1,2-a]pyrimidine-7-carboxylic acid (3 g, 6.97 mmol, 1 eq), (3R)—N,N-dimethylpyrrolidin-3-amine (1.96 g, 10.46 mmol, 2.17 mL, 1.5 eq, 2HCl) and DIEA (4.50 g, 34.85 mmol, 6.07 mL, 5 eq) in THF (25 mL) was added T3P (6.65 g, 10.46 mmol, 6.22 mL, 50% purity, 1.5 eq) dropwise at 0° C. Then the mixture was stirred at 20° C. for 10 hours. HPLC and LC-MS showed the reaction was complete. The mixture was quenched with ice water (30 mL) and the organic solvent was removed under reduced pressure. The aqueous was extracted with DCM and i-PrOH (v:v=3:1, 20 mL×3). The combined organic layer was washed with brine (20 mL), dried over Na₂SO₄, filtered and concentrated under reduced pressure. The residue was purified by prep-HPLC (column: Xtimate C18 10u 250 mm*80 mm; mobile phase: [water (10 mM NH₄HCO₃)-MeCN]; B %: 30%-55%, 24 mins). The eluent was removed under freeze drying. Compound 9-[(1R)-1-(3,5-difluoroanilino)ethyl]-7-[(3R)-3-(dimethylamino)pyrrolidine-1-carbonyl]-2-morpholino-pyrido[1,2-a]pyrimidin-4-one (2.29 g, 4.34 mmol, 62.25% yield, 99.75% purity, 96.34% ee.) was obtained as pale yellow solid. ¹H NMR (DMSO-d₆, 400 MHz) δ 8.88 (s, 1H), 7.83 (s, 1H), 7.02˜6.98 (m, 1H), 6.25˜6.21 (m, 1H), 6.11˜6.08 (m, 2H), 5.71 (s, 1H), 5.12˜5.08 (m, 1H), 3.73 (s, 8H), 3.66˜3.51 (m, 1H), 3.47˜3.42 (m, 2H), 3.26˜3.22 (m, 1H), 2.68˜2.62 (m, 1H), 2.17 and 2.06 (m, 6H), 2.01˜1.96 (m, 1H), 1.74˜1.71 (m, 1H), 1.49 (d, J=6.8 Hz, 3H). HPLC: 99.75% (220 nm), 100% (254 nm). MS (ESI): mass calcd. For C₂₇H₃₂F₂N₆O₃ 526.25 m/z found 527.3 [M+H]⁺.

Chiral Analytical Instrument: Waters Acquity Arc; Column: S,S_Whelk_O1, 3.5 μm, 0.46 cm id×10 cm L. Compound 50 (Rt=8.212 minutes; E.E. by chiral HPLC (%)=96.34%) was obtained and identified as Compound 48—P3 (Rt=8.298 minutes; E.E. by chiral HPLC (%)=98.8%).

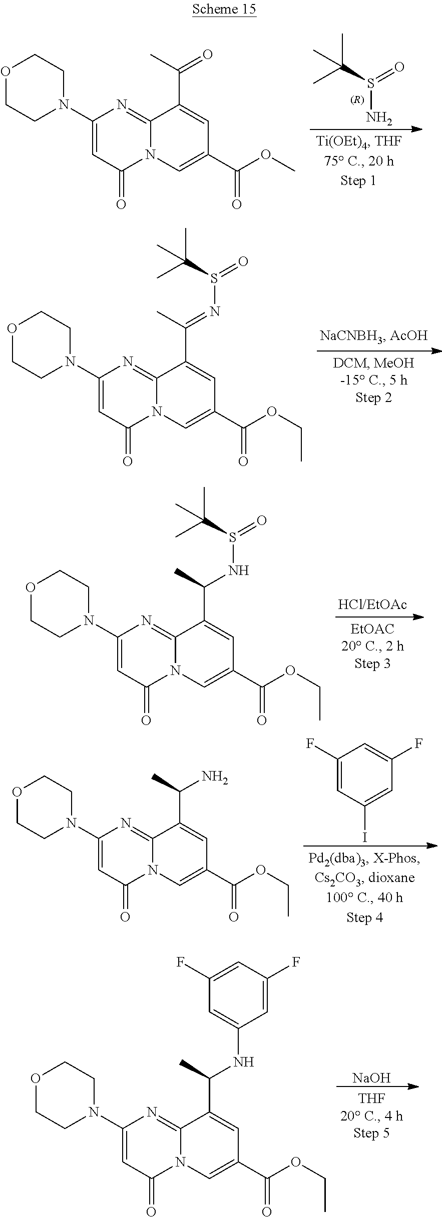

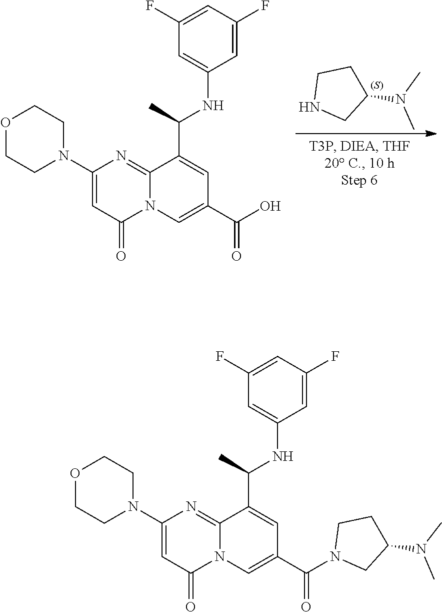

### Compound 51

### Preparation of 9-((R)-1-((3,5-difluorophenyl)amino)ethyl)-7-((S)-3-(dimethylamino)pyrrolidine-1-carbonyl)-2-morpholino-4H-pyrido[1,2-a]pyrimidin-4-one (Steps 1-6 in Scheme 15)

### Preparation of (R,E)-ethyl 9-(1-((tert-butylsulfinyl)imino)ethyl)-2-morpholino-4-oxo-4H-pyrido[1,2-a]pyrimidine-7-carboxylate (Step 1 in Scheme 15)

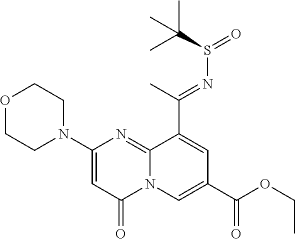 [SMILES: CCOC(=O)c1cc(/C(C)=N/[S@](=O)C(C)(C)C)c2nc(N3CCOCC3)cc(=O)n2c1]

A mixture of methyl 9-acetyl-2-morpholino-4-oxo-pyrido[1,2-a]pyrimidine-7-carboxylate (16 g, 48.40 mmol, 1 eq), (R)-2-methylpropane-2-sulfinamide (10.56 g, 87.13 mmol, 1.8 eq) and tetraethoxytitanium (44.16 g, 193.61 mmol, 40.15 mL, 4 eq) in THF (160 mL) was stirred at 75° C. for 20 hours under N₂ atmosphere. LC-MS showed the reaction was complete. The mixture was cooled to 25° C., then diluted with water (400 mL) and ethyl acetate (250 mL). The mixture was filtered through celite and the filter cake was washed with ethyl acetate (40 mL). The organic phase was separated. The aqueous phase was extracted with ethyl acetate (200 mL×4). The combined organic phases were washed with brine (300 mL), dried with anhydrous Na₂SO₄, filtered and concentrated in vacuum. The crude product was triturated with MTBE (130 mL) at 20° C. for an hour. After filtration, 13.13 g of compound ethyl 9-[(E)-N—[(R)-tert-butylsulfinyl]-C-methyl-carbonimidoyl]-2-morpholino-4-oxo-pyrido[1,2-a]pyrimidine-7-carboxylate (13.13 g, 29.27 mmol, 60.48% yield) was obtained as yellow solid. ¹H NMR (DMSO-d₆, 400 MHz) δ 9.31-9.22 (m, 1H), 8.05˜8.00 (m, 1H), 5.71-5.69 (m 1H), 4.40 (q, J=6.8 Hz, 2H), 3.65 (s, 8H), 2.58 (s, 3H), 1.36˜1.33 (m, 3H), 1.12˜1.08 (m, 9H).

### Preparation of ethyl 9-((R)-1-((R)-1,1-dimethylethylsulfinamido)ethyl)-2-morpholino-4-oxo-4H-pyrido[1,2-a]pyrimidine-7-carboxylate (Step 2 in Scheme 15)

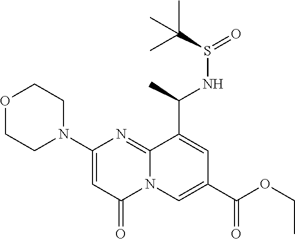 [SMILES: CCOC(=O)c1cc([C@@H](C)N[S@](=O)C(C)(C)C)c2nc(N3CCOCC3)cc(=O)n2c1]

To a solution of ethyl 9-[(E)-N—[(R)-tert-butylsulfinyl]-C-methyl-carbonimidoyl]-2-morpholino-4-oxo-pyrido[1,2-a]pyrimidine-7-carboxylate (13.13 g, 29.27 mmol, 1 eq) and AcOH (14.06 g, 234.18 mmol, 13.39 mL, 8 eq) in DCM (80 mL) and MeOH (80 mL) was added NaBH₃CN (5.52 g, 87.82 mmol, 3 eq) in portions at −15° C. The mixture was stirred at −15° C. for 5 hours. LC-MS showed the reaction was complete. The mixture was quenched with ice water (1.5 mL) and adjusted pH=7 with sat. Na₂CO₃ at 0° C. The organic phase was separated. The aqueous phase was extracted with EtOAc (150 mL×3). The combined organic phase was washed with brine (300 mL), dried with anhydrous Na₂SO₄, filtered and concentrated in vacuum. The residue was purified by flash silica gel chromatography (Biotage; 220 g SepaFlash @ Silica Flash Column, Eluent of 0˜100% Ethylacetate/petroleum ether gradient; Eluent of 0˜13% MeOH/Ethylacetate gradient @ 200 mL/min). The eluent was removed under reduced pressure. Compound ethyl 9-[(1R)-1-[[(R)-tert-butylsulfinyl]amino]ethyl]-2-morpholino-4-oxo-pyrido[1,2-a]pyrimidine-7-carboxylate (6.83 g, 15.16 mmol, 51.79% yield) was obtained as yellow solid. ¹H NMR (DMSO-d₆, 400 MHz) δ 9.25 (s, 1H), 8.25 (s, 1H), 5.96 (d, J=8.4 Hz, 1H), 5.69 (s, 1H), 5.04˜4.97 (m, 1H), 4.38˜4.34 (q, J=3.2 Hz, 2H), 3.69 (s, 8H), 1.46 (d, J=6.8 Hz, 3H), 1.35 (t, J=7.2 Hz, 3H), 1.12 (s, 9H).

### Preparation of (R)-ethyl 9-(1-aminoethyl)-2-morpholino-4-oxo-4H-pyrido[1,2-a]pyrimidine-7-carboxylate (Step 3 in Scheme 15)

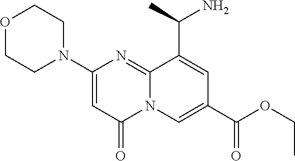 [SMILES: CCOC(=O)c1cc([C@@H](C)N)c2nc(N3CCOCC3)cc(=O)n2c1]

To a solution of ethyl 9-[(1R)-1-[[(R)-tert-butylsulfinyl]amino]ethyl]-2-morpholino-4-oxo-pyrido[1,2-a]pyrimidine-7-carboxylate (6.8 g, 15.09 mmol, 1 eq) in EtOAc (70 mL) was added HCl/EtOAc (4 M, 37.73 mL, 10 eq). The mixture was stirred at 20° C. for 2 hours. LC-MS showed the reaction was complete. The mixture was concentrated under reduced pressure. The residue was triturated with EtOAc (20 mL). After filtration, the solid was collected. Compound ethyl 9-[(1R)-1-aminoethyl]-2-morpholino-4-oxo-pyrido[1,2-a]pyrimidine-7-carboxylate (6.63 g, crude, 2HCl, 100% ee) was obtained as yellow solid. ¹H NMR (DMSO-d₆, 400 MHz) δ 9.30 (s, 1H), 8.64 (s, 2H), 8.29 (s, 1H), 5.74 (s, 1H), 4.98˜4.95 (m, 1H), 4.42 (q, J=8.0 Hz, 2H), 3.69 (s, 8H), 1.58 (d, J=6.4 Hz, 3H), 1.38 (t, J=7.2 Hz, 3H).

Chiral analytical Instrument: Waters Acquity UPC2; Column: Chiralcel OD-3 3 μm, 0.46 cm id×5 cm L; Mobile phase: A for SFC CO₂ and B for MeOH (0.05% IPAm); Gradient: B in A from 5% to 50% in 3 minutes; Flow rate: 3.4 mL/min; Column temperature: 35° C.; Wavelength: 220 nm; System Back Pressure: 1800 psi. Rt=1.217 minutes; E.E. by Chiral HPLC (%)=100% ee.

### Preparation of (R)-ethyl 9-(1-((3,5-difluorophenyl)amino)ethyl)-2-morpholino-4-oxo-4H-pyrido[1,2-a]pyrimidine-7-carboxylate (Step 4 in Scheme 15)

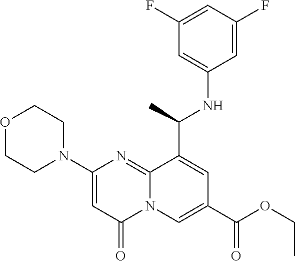 [SMILES: CCOC(=O)c1cc([C@@H](C)Nc2cc(F)cc(F)c2)c2nc(N3CCOCC3)cc(=O)n2c1]

A mixture of ethyl 9-[(1R)-1-aminoethyl]-2-morpholino-4-oxo-pyrido[1,2-a]pyrimidine-7-carboxylate (6.62 g, 15.79 mmol, 1 eq, 2 HCl), 1,3-difluoro-5-iodo-benzene (5.68 g, 23.68 mmol, 2.84 mL, 1.5 eq), Pd₂(dba)₃ (1.45 g, 1.58 mmol, 0.1 eq), Cs₂CO₃ (18.00 g, 55.26 mmol, 3.5 eq) and dicyclohexyl-[2-(2,4,6-triisopropylphenyl)phenyl]phosphane (1.13 g, 2.37 mmol, 0.15 eq) in dioxane (70 mL) was degassed and purged with N₂ for 3 times and then the mixture was stirred at 100° C. for 20 hours under N₂ atmosphere. Then another batch 1,3-difluoro-5-iodo-benzene (3.79 g, 15.79 mmol, 1.89 mL, 1 eq), Pd₂(dba)₃ (722.87 mg, 789.41 umol, 0.05 eq), Cs₂CO₃ (5.14 g, 15.79 mmol, 1 eq) and dicyclohexyl-[2-(2,4,6-triisopropylphenyl)phenyl]phosphane (564.48 mg, 1.18 mmol, 0.075 eq) were added and then the mixture was stirred at 100° C. for 20 hours under N₂ atmosphere. LC-MS showed the reaction was nearly complete. The mixture was filtered and the filter cake was washed with EtOAc (20 mL). The filtrate was concentrated in vacuum. The residue was purified by flash silica gel chromatography (Biotage; 40 g SepaFlash® Silica Flash Column, Eluent of 0˜60% Ethylacetate/petroleum ether gradient). The eluent was removed under reduced pressure. Compound ethyl 9-[(1R)-1-(3,5-difluoroanilino)ethyl]-2-morpholino-4-oxo-pyrido[1,2-a]pyrimidine-7-carboxylate (3.35 g, 7.31 mmol, 46.28% yield) was obtained as yellow solid. And ethyl 9-[(1R)-1-aminoethyl]-2-morpholino-4-oxo-pyrido[1,2-a] pyrimidine-7-carboxylate (1.11 g) was recovered as brown solid. ¹H NMR (DMSO-d₆, 400 MHz) δ 9.23 (s, 1H), 7.99 (s, 1H), 7.08 (d, J=7.2 Hz, 1H), 6.25 (t, J=9.2 Hz, 1H), 6.09 (d, J=10.0 Hz, 2H), 5.73 (s, 1H), 5.06 (t, J=6.8 Hz, 1H), 4.35˜4.30 (m, 2H), 3.69 (s, 8H), 1.47 (d, J=6.8 Hz, 3H), 1.30 (t, J=7.2 Hz, 3H).

### Preparation of (R)-9-(1-((3,5-difluorophenyl)amino)ethyl)-2-morpholino-4-oxo-4H-pyrido[1,2-a]pyrimidine-7-carboxylic acid (Step 5 in Scheme 15)

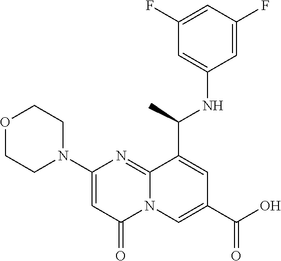 [SMILES: C[C@@H](Nc1cc(F)cc(F)c1)c1cc(C(=O)O)cn2c(=O)cc(N3CCOCC3)nc12]

To a solution of ethyl 9-[(1R)-1-(3,5-difluoroanilino)ethyl]-2-morpholino-4-oxo-pyrido[1,2-a] pyrimidine-7-carboxylate (3.35 g, 7.31 mmol, 1 eq) in THF (6 mL) was added NaOH (4 M, 7.31 mL, 4 eq). The mixture was stirred at 20° C. for 2 hours. NaOH (4 M, 7.31 mL, 4 eq) was added and the mixture was stirred at 20° C. for 2 hours. LC-MS and TLC (Petroleum ether:Ethyl acetate=1:1, R_{f}═O) showed the reaction was complete. HCl (4N) was added dropwise at 0° C. until pH=3. The precipitation was collected by filtration and concentration in vacuum. Compound 9-[(1R)-1-(3,5-difluoroanilino)ethyl]-2-morpholino-4-oxo-pyrido[1,2-a]pyrimidine-7-carboxylic acid (3.03 g, 7.04 mmol, 96.37% yield, 100% ee) was obtained as yellow solid. ¹H NMR (DMSO-d₆, 400 MHz) δ 9.22 (s, 1H), 8.00 (s, 1H), 7.09 (d, J=7.2 Hz, 1H), 6.24 (t, J=9.2 Hz, 1H), 6.09 (d, J=9.6 Hz, 2H), 5.71 (s, 1H), 5.08˜5.02 (m, 1H), 3.69 (s, 8H), 1.48 (d, J=6.8 Hz, 3H).

Chiral analytical Instrument: Waters Acquity UPC2; Column: Chiralpak IC-3 3 μm, 0.46 cm id×5 cm L; Mobile phase: A for SFC CO2 and B for EtOH (0.05% IPAm); Gradient: B in A from 5% to 50% in 3 minutes; Flow rate: 3.4 mL/min; Column temperature: 35° C.; Wavelength: 220 nm; System Back Pressure: 1800 psi. Rt=1.771 minutes; E.E. by Chiral HPLC (%)=100% ee.

### Preparation of 9-((R)-1-((3,5-difluorophenyl)amino)ethyl)-7-((S)-3-(dimethylamino)pyrrolidine-1-carbonyl)-2-morpholino-4H-pyrido[1,2-a]pyrimidin-4-one (Step 6 in Scheme 15)

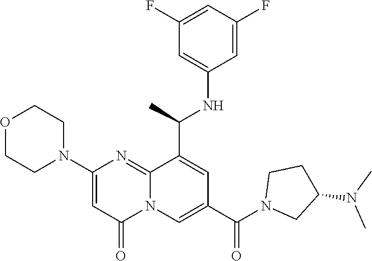 [SMILES: C[C@@H](Nc1cc(F)cc(F)c1)c1cc(C(=O)N2CC[C@H](N(C)C)C2)cn2c(=O)cc(N3CCOCC3)nc12]

To a solution of 9-[(1R)-1-(3,5-difluoroanilino)ethyl]-2-morpholino-4-oxo-pyrido[1,2-a]pyrimidine-7-carboxylic acid (2.8 g, 6.51 mmol, 1 eq), (3S)—N,N-dimethylpyrrolidin-3-amine (1.11 g, 9.76 mmol, 1.5 eq) and DIEA (4.20 g, 32.53 mmol, 5.67 mL, 5 eq) in THF (28 mL) was added T3P (6.21 g, 9.76 mmol, 5.80 mL, 50% purity, 1.5 eq) dropwise at 0° C. The resulting mixture was stirred at 20° C. for 10 hours. LC-MS and HPLC showed the reaction was complete. The mixture was quenched with ice water (1.5 mL) and concentrated in vacuum. Water (40 mL) was added to the residue and the mixture was extracted with DCM and i-PrOH (v:v=3:1, 40 mL×3). The combined organic phase was washed with brine (70 mL), dried with anhydrous Na₂SO₄, filtered and concentrated in vacuum. The residue was purified by prep-HPLC (column: Xtimate C18 10 u 250 mm*80 mm; mobile phase: [water (10 mM NH₄HCO₃)-MeCN]; B %: 25%-55%, 26 mins). The eluent was removed under freeze drying. Compound 9-[(1R)-1-(3,5-difluoroanilino)ethyl]-7-[(3S)-3-(dimethylamino)pyrrolidine-1-carbonyl]-2-morpholino-pyrido[1,2-a]pyrimidin-4-one (2.16 g, 4.06 mmol, 62.37% yield, 98.91% purity, 94.16% ee) was obtained as pale yellow solid. ¹H NMR (DMSO-d₆, 400 MHz) δ 8.87 (s, 1H), 7.82 (s, 1H), 6.97˜6.96 (m, 1H), 6.22˜6.20 (m, 1H), 6.10 (d, J=10.8 Hz, 2H), 5.70 (s, 1H), 5.10˜5.08 (m, 1H), 3.69 (s, 8H), 3.60˜3.53 (m, 1H), 3.42˜3.40 (m, 2H), 3.24˜3.21 (m, 1H), 2.45˜2.42 (m, 1H), 2.16 (s, 3H), 2.05 (s, 1H), 1.97 (s, 3H), 1.68˜1.67 (m, 1H), 1.48 (d, J=6.4 Hz, 3H). HPLC: 98.91% (220 nm), 99.01% (254 nm). MS (ESI): mass calcd. For C₂₇H₃₂F₂N₆O₃ 526.25 m/z found 527.3 [M+H]⁺.

Chiral Analytical Instrument: Waters Acquity Arc; Column: S,S_Whelk_O1, 3.5 μm, 0.46 cm id×10 cm L. Compound 51 (Rt=RT=14.281 minutes; E.E. by chiral HPLC (%)=94.16%) was obtained and identified as Compound 48—P4 (Rt=14.409 minutes, E.E. by chiral HPLC (%)=99.46%).

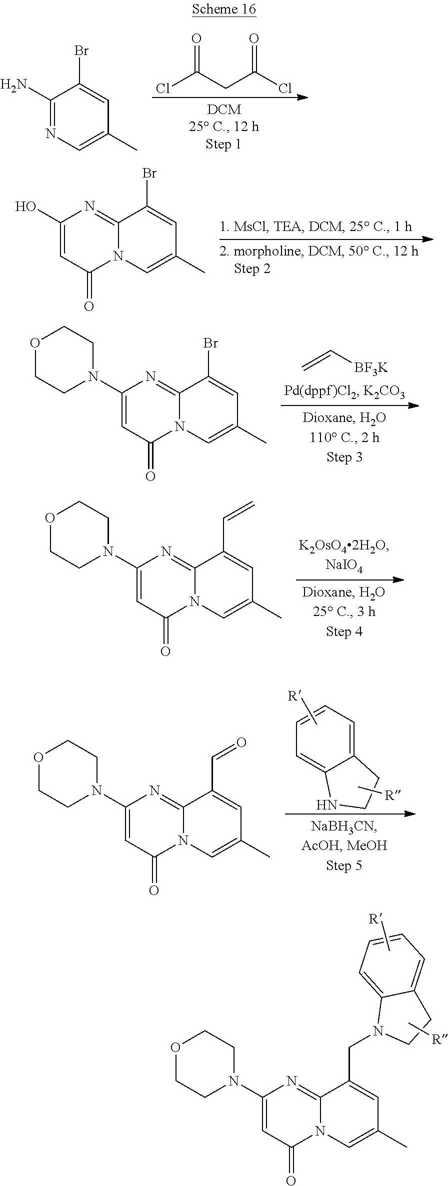

### General Procedures for Preparing Compounds in Scheme 16

### Preparation of 9-bromo-2-hydroxy-7-methyl-pyrido[1,2-a] pyrimidin-4-one (Step 1 in Scheme 16)

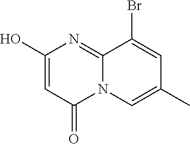

To a solution of 3-bromo-5-methyl-pyridin-2-amine (22 g, 117.62 mmol, 1 eq) in DCM (300 mL) was added propanedioyl dichloride (18.24 g, 129.39 mmol, 12.58 mL, 1.1 eq) dropwise at 0° C. The mixture was stirred at 25° C. for 12 hours. LC-MS showed the reaction was complete. The solid was formed and collected by filtration, washed with DCM (10 mL×2), and dried under reduced pressure. Compound 9-bromo-2-hydroxy-7-methyl-pyrido[1,2-a] pyrimidin-4-one (46 g, 180.34 mmol, 76.66% yield) was obtained as light yellow solid. ¹H NMR (DMSO-d₆, 400 MHz) δ 8.72 (s, 1H), 8.28 (s, 1H), 5.54 (s, 1H), 2.34 (s, 3H).

### Preparation of 9-bromo-7-methyl-2-morpholinopyrido[1,2-a]pyrimidin-4-one (Step 2 in Scheme 16)

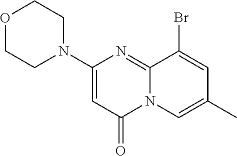 [SMILES: Cc1cc(Br)c2nc(N3CCOCC3)cc(=O)n2c1]

To a solution of 9-bromo-2-hydroxy-7-methyl-pyrido[1,2-a]pyrimidin-4-one (10 g, 39.21 mmol, 1 eq) in DCM (100 mL) was added TEA (7.93 g, 78.41 mmol, 10.91 mL, 2 eq) and MsCl (6.29 g, 54.89 mmol, 4.25 mL, 1.4 eq) dropwise at 0° C. The mixture was stirred at 25° C. for 1 hour. Then morpholine (10.25 g, 117.62 mmol, 10.35 mL, 3 eq) was added. The mixture was stirred at 50° C. for 12 hours. LC-MS showed the reaction was complete. The reaction mixture was quenched by addition H₂O (50 mL) at 0° C., and then extracted with DCM (100 mL×3). The combined organic layers were washed with brine (50 mL), dried over Na₂SO₄, filtered and concentrated under reduced pressure to give a residue. The residue was purified by re-crystallization from DCM (30 mL) and washed with EtOAc (20 mL×2) to give the pure product 9-bromo-7-methyl-2-morpholino-pyrido[1,2-a]pyrimidin-4-one (5 g) as yellow solid. And the residue was purified by flash silica gel chromatography (Biotage®; 40 g SepaFlash® Silica Flash Column, Eluent of 0˜60% Ethyl acetate/Petroleum ether gradient @ 100 mL/min) to give the pure product 9-bromo-7-methyl-2-morpholinopyrido[1,2-a]pyrimidin-4-one (2 g) as yellow solid. The yellow solid compound 9-bromo-7-methyl-2-morpholinepyrido[1,2-a]pyrimidin-4-one (7 g, yield 55.1%) was obtained after mixing the two batches of products. ¹H NMR (DMSO-d₆, 400 MHz) δ 8.60 (s, 1H), 8.17 (d, J=1.6 Hz, 1H), 5.61 (s, 1H), 3.68˜3.64 (m, 8H), 2.29 (s, 3H).

### Preparation of 7-methyl-2-morpholino-9-vinylpyrido[1,2-a]pyrimidin-4-one (Step 3 in Scheme 16)

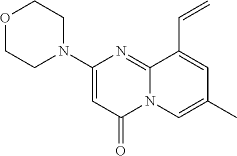 [SMILES: C=Cc1cc(C)cn2c(=O)cc(N3CCOCC3)nc12]

A mixture of 9-bromo-7-methyl-2-morpholino-pyrido[1,2-a]pyrimidin-4-one (3 g, 9.25 mmol, 1 eq), potassium hydride;trifluoro(vinyl)boron (1.86 g, 13.88 mmol, 1.5 eq), Pd(dppf)Cl₂ (338.57 mg, 462.72 umol, 0.05 eq) and K₂CO₃ (3.84 g, 27.76 mmol, 3 eq) in dioxane (40 mL) and H₂O (10 mL) was degassed and purged with N₂ for 3 times, and then the mixture was stirred at 110° C. for 2 hours under N₂ atmosphere. LC-MS showed the reaction was complete. The reaction mixture was filtered to removed insoluble and concentrated under reduced pressure to removed dioxane. And then the aqueous was extracted with DCM (20 mL×3). The combined organic layers were washed with brine (10 mL), dried over Na₂SO₄, filtered and concentrated under reduced pressure to give a residue. The residue was purified by re-crystallization from DCM (10 mL) and washed with EtOAc (10 mL×2) to give the pure product 7-methyl-2-morpholino-9-vinyl-pyrido[1,2-a]pyrimidin-4-one (1 g) as yellow solid. And the residue was purified by flash silica gel chromatography (Biotage®; 12 g SepaFlash® Silica Flash Column, Eluent of 0˜10% Methanol. Dichloromethane gradient @ 60 mL/min) to give the crude product 7-methyl-2-morpholino-9-vinylpyrido[1,2-a]pyrimidin-4-one (1.2 g) as yellow solid. The yellow solid compound 7-methyl-2-morpholine-9-vinylpyrido[1,2-a]pyrimidin-4-one (2.2 g, yield 87.9%) was obtained after mixing the two batches of products. ¹H NMR (DMSO-d₆, 400 MHz) δ 8.58 (s, 1H), 7.97 (d, J=1.6 Hz, 1H), 7.33 (dd, J=17.6 Hz, 11.2 Hz, 1H), 6.06 (d, J=17.6 Hz, 1H), 5.62 (s, 1H), 5.48 (d, J=11.6 Hz, 1H), 3.69˜3.67 (m, 4H), 3.61˜3.59 (m, 4H), 2.33 (s, 3H).

### Preparation of 7-methyl-2-morpholino-4-oxo-pyrido[1,2-a]pyrimidine-9-carbaldehyde (Step 4 in Scheme 16)

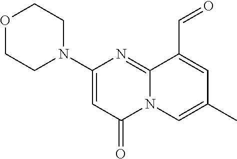 [SMILES: Cc1cc(C=O)c2nc(N3CCOCC3)cc(=O)n2c1]

To a solution of 7-methyl-2-morpholino-9-vinyl-pyrido[1,2-a]pyrimidin-4-one (750 mg, 2.76 mmol, 1 eq) in dioxane (100 mL) and H₂O (20 mL) was added dipotassium;dioxide (dioxo)osmium;dihydrate (101.85 mg, 276.43 umol, 0.1 eq) at 25° C. After addition, the mixture was stirred at 25° C. for 1 hour, and then NaIO4 (1.77 g, 8.29 mmol, 459.53 uL, 3 eq) was added at 0° C. The resulting mixture was stirred at 25° C. for 2 hours. LC-MS showed the reaction was complete. The reaction mixture was filtered to removed insoluble and concentrated under reduced pressure to removed dioxane. And then the aqueous was extracted with DCM (20 mL×3). The combined organic layers were washed with brine (10 mL), dried over Na₂SO₄, filtered and concentrated under reduced pressure to give a residue. The residue was purified by flash silica gel chromatography (Biotage®; 12 g SepaFlash® Silica Flash Column, Eluent of 0˜60% Dichloromethane:Ethyl acetate gradient @ 100 mL/min). The eluent was removed under reduced pressure. Compound 7-methyl-2-morpholino-4-oxo-pyrido[1,2-a]pyrimidine-9-carbaldehyde (300 mg, 1.10 mmol, 39.71% yield) was obtained as yellow solid. ¹H NMR (DMSO-d₆, 400 MHz) δ 10.82 (s, 1H), 8.95 (s, 1H), 8.11 (d, J=2.4 Hz, 1H), 5.65 (s, 1H), 3.83˜3.80 (m, 4H), 3.71-3.68 (m, 4H), 2.41 (s, 3H).

### Preparation of Compounds in Scheme 16 (Step 5 in Scheme 16)

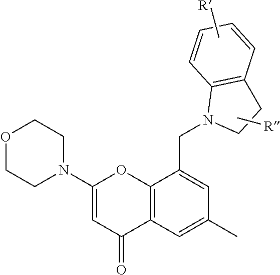

A solution of 7-methyl-2-morpholino-4-oxo-pyrido[1,2-a]pyrimidine-9-carbaldehyde (365.92 umol, 1 eq), AcOH (10.98 mmol, 30 eq) and indoline (474.50 umol˜731.84 umol, 1.1 eq 2.0 eq) in MeOH (5 mL/mmol) was stirred at 20° C. for 1 hour. Then NaCNBH₃ (365.92 umol 731.84 umol, 1 eq˜2 eq) was added in portions at 0° C. and the mixture was stirred at 20° C. for 1 hour 2 hours. TLC (petroleum ether:EtOAc=0:1, R_{f}=0.15) and LCMS showed the reaction was complete. The mixture was quenched with ice water at 0° C. and the organic solvent was removed under reduced pressure. The aqueous was made pH=8 with sat. NaHCO₃, then extracted with DCM. The combined organic layer was washed with brine, dried over Na₂SO₄, filtered and concentrated under reduced pressure. The residue was purified by flash silica gel chromatography (Biotage®; 12 g SepaFlash® Silica Flash Column, Eluent of 0˜60% Ethyl acetate/petroleum ether gradient @ 80 mL/min) or by prep-HPLC (column: Welch Xtimate C18 100*25 mm*3 um or Waters Xbridge Prep OBD C18 150*40 mm*10 um or Phenomenex luna C18 80*40 mm*3 um; mobile phase: [water (0.05% HCl)-MeCN]; B %: 15%-35%, 8 mins or [water (0.04% HCl)-MeCN]; B %: 45%-55%, 7 mins or [water (0.04% HCl)-MeCN]; B %: 18%-55%, 7 mins or [water (10 mM NH₄HCO₃)-MeCN]; B %: 25%-55%, 8 mins). The eluent was removed under reduced pressure or the aqueous solution was lyophilized to give desired product.

### Compound 52

### Preparation of 9-[(4-fluoroindolin-1-yl)methyl]-7-methyl-2-morpholino-pyrido[1,2-a]pyrimidin-4-one (Step 5 in Scheme 16)

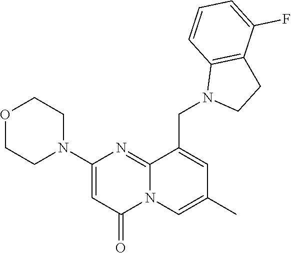 [SMILES: Cc1cc(CN2CCc3c(F)cccc32)c2nc(N3CCOCC3)cc(=O)n2c1]

A solution of 7-methyl-2-morpholino-4-oxo-pyrido[1,2-a]pyrimidine-9-carbaldehyde (0.1 g, 365.92 umol, 1 eq), AcOH (659.22 mg, 10.98 mmol, 627.83 uL, 30 eq) and 4-fluoroindoline (70.26 mg, 512.29 umol, 1.4 eq) in MeOH (3 mL) was stirred at 20° C. for 1 hour. Then NaCNBH₃ (83.45 mg, 731.84 umol, 54.19 uL, 2 eq) at 0° C. was added in portions and the mixture was stirred at 20° C. for 1 hour. TLC (petroleum ether:EtOAc=0:1, R_{f}=0.15) showed the reaction was complete. The mixture was quenched with ice water (10 mL) at 0° C. and the organic solvent was removed under reduced pressure. The aqueous was made pH=8 with sat. NaHCO₃, then extracted with DCM (10 mL×4). The combined organic layer was washed with brine (10 mL×1), dried over Na₂SO₄, filtered and concentrated under reduced pressure. The residue was purified by flash silica gel chromatography (Biotage®; 12 g SepaFlash® Silica Flash Column, Eluent of 0˜60% Ethyl acetate/petroleum ether gradient @ 80 mL/min). The eluent was removed under reduced pressure. The residue was diluted with H₂O (20 mL) and the solvent was removed under freeze drying. Compound 9-[(4-fluoroindolin-1-yl)methyl]-7-methyl-2-morpholino-pyrido[1,2-a]pyrimidin-4-one (66.4 mg, 167.01 umol, 45.64% yield, 99.21% purity) was obtained as white solid. ¹H NMR (DMSO-d₆, 400 MHz) δ 8.56 (s, 1H), 7.61 (s, 1H), 7.00 (dd, J=14.0 Hz, 8.8 Hz, 1H), 6.39 (t, J=8.8 Hz, 1H), 6.32 (d, J=8.0 Hz, 1H), 5.63 (s, 1H), 4.50 (s, 2H), 3.66˜3.64 (m, 4H), 3.61˜3.59 (m, 4H), 3.53 (t, J=8.4 Hz, 2H), 3.02 (t, J=8.4 Hz, 2H), 2.29 (s, 3H). LCMS: 99.21% (220 nm), 100.00% (254 nm). MS (ESI): mass calcd. For C₂₂H₂₃FN₄O₂ 394.18, m/z found 395.1 [M+H]⁺.

### Compound 53

### 9-((4-fluoro-2-methylindolin-1-yl)methyl)-7-methyl-2-morpholino-4H-pyrido[1,2-a]pyrimidin-4-one was prepared according to the procedure described herein for Step 5 in Scheme 16

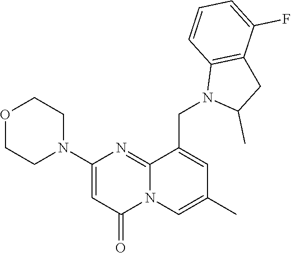 [SMILES: Cc1cc(CN2c3cccc(F)c3CC2C)c2nc(N3CCOCC3)cc(=O)n2c1]

The desired compound (51 mg, 118.63 umol, 46.31% yield, 95.01% purity) was obtained as white solid. ¹H NMR (DMSO-d₆, 400 MHz) δ 8.55 (s, 1H), 7.53 (s, 1H), 6.94 (dd, J=14.0 Hz, 8.8 Hz, 1H), 6.37 (t, J=8.8 Hz, 1H), 6.03 (d, J=8.0 Hz, 1H), 5.64 (s, 1H), 4.46 (dd, J=18.4 Hz, 10.8 Hz, 2H), 3.95˜3.89 (m, 1H), 3.68˜3.66 (m, 4H), 3.61˜3.59 (m, 4H), 3.29˜3.25 (m, 1H), 2.69˜2.62 (m, 1H), 2.27 (s, 3H), 1.27 (d, J=6.0 Hz, 3H). LCMS: 95.01% (220 nm), 96.20% (254 nm). MS (ESI): mass calcd. For C₂₃H₂₅FN₄O₂ 408.20, m/z found 409.2 [M+H]⁺.

### Compound 54

### 9-((4,6-difluoroindolin-1-yl)methyl)-7-methyl-2-morpholino-4H-pyrido[1,2-a]pyrimidin-4-one was prepared according to the procedure described herein for Step 5 in Scheme 16

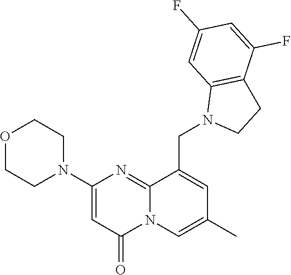 [SMILES: Cc1cc(CN2CCc3c(F)cc(F)cc32)c2nc(N3CCOCC3)cc(=O)n2c1]

The desired compound (12.5 mg, 29.49 umol, 20.15% yield, 97.30% purity) was obtained as white solid. ¹H NMR (DMSO-d₆, 400 MHz) δ 8.56 (s, 1H), 7.60 (s, 1H), 6.30˜6.24 (m, 2H), 5.62 (s, 1H), 4.53 (s, 2H), 3.65 (t, J=4.8 Hz, 4H), 3.60 (t, J=7.2 Hz, 4H), 3.57˜3.55 (m, 2H), 2.98 (t, J=8.0 Hz, 2H), 2.30 (s, 3H). MS: 97.30% (220 nm), 99.80% (254 nm). MS (ESI): mass calcd. For C₂₂H₂₂F₂N₄O₂ 412.17 m/z found 413.2 [M+H]⁺.

### Compound 55

### 9-((4,6-difluoro-2-methylindolin-1-yl)methyl)-7-methyl-2-morpholino-4H-pyrido[1,2-a]pyrimidin-4-one was prepared according to the procedure described herein for Step 5 in Scheme 16

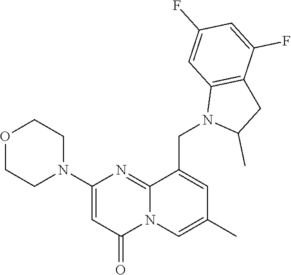 [SMILES: Cc1cc(CN2c3cc(F)cc(F)c3CC2C)c2nc(N3CCOCC3)cc(=O)n2c1]

The desired compound (3.9 mg, 8.75 umol, 5.98% yield, 95.66% purity) was obtained as white solid. ¹H NMR (MeOD, 400 MHz) δ 8.62 (s, 1H), 7.59 (s, 1H), 6.11 (t, J=8.0 Hz, 1H), 5.82 (d, J=8.0 Hz, 1H), 5.74 (s, 1H), 4.54 (d, J=4.4 Hz, 2H), 4.06˜3.93 (m, 1H), 3.80˜3.76 (m, 4H), 3.73˜3.69 (m, 4H), 3.28˜3.25 (m, 1H), 2.70˜2.64 (m, 1H), 2.34 (s, 3H), 1.33 (d, J=6.4 Hz, 3H) LCMS: 95.66% (220 nm), 98.49% (254 nm). MS (ESI): mass calcd. For C₂₃H₂₄F₂N₄O₂ 426.19, m/z found 427.2 [M+H]⁺.

### Compound 56

### 9-((5-fluoroindolin-1-yl)methyl)-7-methyl-2-morpholino-4H-pyrido[1,2-a]pyrimidin-4-one was prepared according to the procedure described herein for Step 5 in Scheme 16

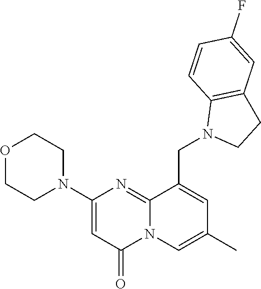 [SMILES: Cc1cc(CN2CCc3cc(F)ccc32)c2nc(N3CCOCC3)cc(=O)n2c1]

The desired compound (5.2 mg, 12.46 umol, 8.52% yield, 94.55% purity) was obtained as white solid. ¹H NMR (DMSO-d6, 400 MHz) δ 8.56 (s, 1H), 7.65 (s, 1H), 6.96 (d, J=8.0 Hz, 1H), 6.80˜6.70 (m, 1H), 6.45˜6.42 (m, 1H), 5.63 (s, 1H), 4.42 (s, 2H), 3.66 (t, J=5.6 Hz, 4H), 3.61 (t, J=4.4 Hz, 4H), 3.45˜3.43 (m, 2H), 2.97 (t, J=8.3 Hz, 2H), 2.30 (s, 3H). MS: 94.55% (220 nm), 97.88% (254 nm). MS (ESI): mass calcd. For C₂₂H₂₃FN₄O₂ 394.18 m/z found 395.2 [M+H]⁺.

### Compound 57

### 7-methyl-9-((2-methylindolin-1-yl)methyl)-2-morpholino-4H-pyrido[1,2-a]pyrimidin-4-one was prepared according to the procedure described herein for Step 5 in Scheme 16

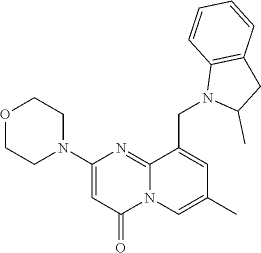 [SMILES: Cc1cc(CN2c3ccccc3CC2C)c2nc(N3CCOCC3)cc(=O)n2c1]

The desired compound (11.2 mg, 27.37 umol, 18.70% yield, 95.42% purity) was obtained as white solid. ¹H NMR (DMSO-d6, 400 MHz) δ 8.55 (s, 1H), 7.55 (s, 1H), 7.05 (d, J=6.4 Hz, 1H), 6.92 (t, J=8.0 Hz, 1H), 6.60 (t, J=7.2 Hz, 1H), 6.16 (d, J=7.6 Hz, 1H), 5.64 (s, 1H), 4.48˜4.34 (m, 2H), 3.81˜3.80 (m, 1H), 3.67 (t, J=4.8 Hz, 4H), 3.62 (t, J=4.8 Hz, 4H), 3.18˜3.10 (m, 1H), 2.63 (s, 1H), 2.27 (s, 3H), 1.27 (d, J=6.0 Hz, 3H). MS: 95.42% (220 nm), 98.90% (254 nm). MS (ESI): mass calcd. For C₂₃H₂₆N₄O₂ 390.21 m/z found 391.2 [M+H]⁺.

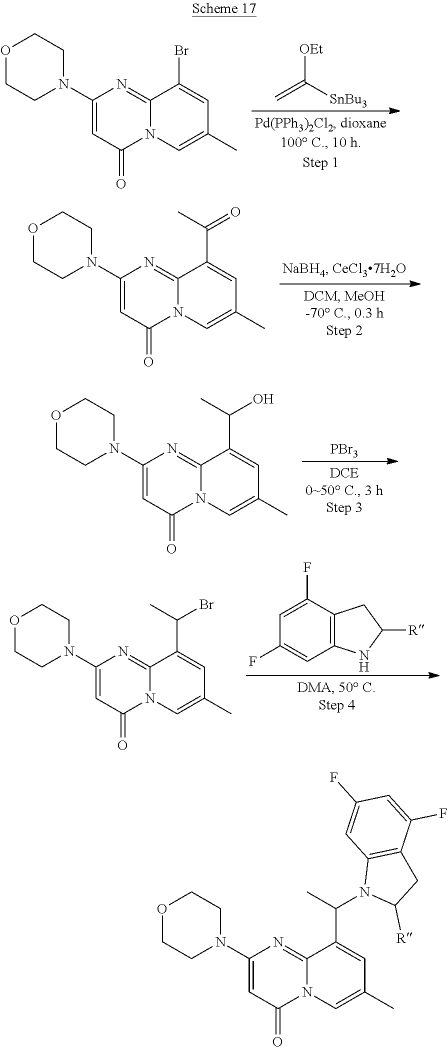

### General Procedures for Preparing Compounds in Scheme 17

### Preparation of 9-acetyl-7-methyl-2-morpholino-4H-pyrido[1,2-a]pyrimidin-4-one (Step 1 in Scheme 17)

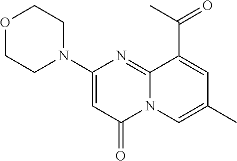 [SMILES: CC(=O)c1cc(C)cn2c(=O)cc(N3CCOCC3)nc12]

A mixture of 9-bromo-7-methyl-2-morpholino-pyrido[1,2-a]pyrimidin-4-one (4.2 g, 12.96 mmol, 1 eq), tributyl(1-ethoxyvinyl)stannane (6.820 g, 18.88 mmol, 6.37 mL, 1.46 eq) and Pd(PPh₃)₂Cl₂ (454.69 mg, 647.80 umol, 0.05 eq) in dioxane (50 mL) was stirred at 100° C. for 10 hours under N₂. LC-MS showed 9-bromo-7-methyl-2-morpholino-pyrido[1,2-a] pyrimidin-4-one (4.2 g, 12.96 mmol, 1 eq) was consumed completely. The mixture was cooled to 25° C., HCl (2 M, 7.77 mL, 1.2 eq) was added and the mixture was stirred at 25° C. for an hour. LC-MS showed the reaction was completely. The organic solvent was removed under reduced pressure. The aqueous was made pH=7 with sat. NaHCO₃ and extracted with DCM (60 mL×3). The combined organic layer was washed with brine (50 mL×2), dried over Na₂SO₄, filtered and concentrated under reduced pressure. The residue was triturated with petroleum ether and EtOAc (v:v=10:1, 20 mL×2). The solid was collected after filtration. Compound 9-acetyl-7-methyl-2-morpholino-pyrido[1,2-a]pyrimidin-4-one (2.8 g, 9.75 mmol, 75.22% yield) was obtained as yellow solid. ¹H NMR (DMSO-d₆, 400 MHz) δ 8.74 (d, J=2.0 Hz, 1H), 7.87 (d, J=2.0 Hz, 1H), 5.65 (s, 1H), 3.69˜3.65 (m, 4H), 3.60˜3.55 (m, 4H), 2.67 (s, 3H), 2.33 (s, 3H).

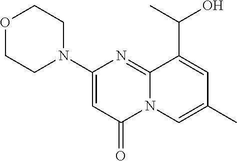 [SMILES: Cc1cc(C(C)O)c2nc(N3CCOCC3)cc(=O)n2c1]

To a mixture of 9-acetyl-7-methyl-2-morpholino-pyrido[1,2-a]pyrimidin-4-one (2.6 g, 9.05 mmol, 1 eq) and CeCl₃·7H₂O (3.37 g, 9.05 mmol, 860.11 uL, 1 eq) in MeOH (15 mL) and DCM (4 mL) was added NaBH₄ (205.42 mg, 5.43 mmol, 0.6 eq) in portions at −70° C. under N₂. The mixture was stirred at −70° C. for 0.3 hour. LC-MS showed the reaction was complete. The mixture was quenched with ice water (30 mL) slowly at 0° C. and the organic solvent was removed under reduced pressure. The aqueous was extracted with EtOAc (40 mL×3). The combined organic layer was washed with brine (30 mL×1), dried over Na₂SO₄, filtered and concentrated under reduced pressure. Compound 9-(1-hydroxyethyl)-7-methyl-2-morpholino-pyrido[1,2-a]pyrimidin-4-one (1.4 g, 4.84 mmol, 53.47% yield) was obtained as yellow solid. ¹H NMR (DMSO-d₆, 400 MHz) δ 8.53 (s, 1H), 7.76 (s, 1H), 5.60 (s, 1H), 5.36 (d, J=4.4 Hz, 1H), 5.26˜5.18 (m, 1H), 3.70˜3.65 (m, 4H), 3.61-3.54 (m, 4H), 2.32 (s, 3H), 1.38 (d, J=6.4 Hz, 3H).

### Preparation of 9-(1-bromoethyl)-7-methyl-2-morpholino-4H-pyrido[1,2-a]pyrimidin-4-one (Step 3 in Scheme 17)

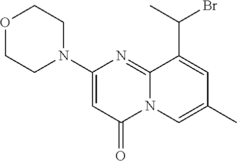 [SMILES: Cc1cc(C(C)Br)c2nc(N3CCOCC3)cc(=O)n2c1]

To a mixture of 9-(1-hydroxyethyl)-7-methyl-2-morpholino-pyrido[1,2-a]pyrimidin-4-one (0.6 g, 2.07 mmol, 1 eq) in DCE (10 mL) was added PBr₃ (617.47 mg, 2.28 mmol, 1.1 eq) at 0° C. under N₂. The mixture was stirred at 50° C. for 3 hours. LC-MS showed the reaction was complete. The mixture was cooled to 0° C. and adjusted to pH=7 with sat. NaHCO₃. The aqueous phase was extracted with DCM (30 mL×2). The combined organic phase was washed with brine (20 mL), dried with anhydrous Na₂SO₄, filtered and concentrated in vacuum. Compound 9-(1-bromoethyl)-7-methyl-2-morpholino-pyrido[1,2-a] pyrimidin-4-one (0.52 g, 1.48 mmol, 71.19% yield) was obtained as yellow solid. ¹H NMR (DMSO-d₆, 400 MHz) δ 8.61 (d, J=1.6 Hz, 1H), 8.02 (d, J=1.6 Hz, 1H), 6.05˜6.01 (m, 1H), 5.64 (s, 1H), 3.73˜3.68 (m, 4H), 3.66˜3.63 (m, 4H), 2.34 (s, 3H), 2.07 (d, J=7.2 Hz, 3H).

### Preparation of Compounds in Scheme 17 (Step 4 in Scheme 17)

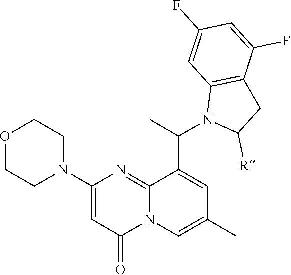

A mixture of 9-(1-bromoethyl)-7-methyl-2-morpholino-pyrido[1,2-a]pyrimidin-4-one (1.70 mmol, 1 eq) and 4,6-difluoro-2-methyl-indoline (3.41 mmol, 2 eq) or 4,6-difluoroindoline (1.70 mmol, 1 eq) under no base or DIEA (8.5 mmol, 5 eq) condition in DMA (5 mL/mmol) was stirred at 50° C. for 10 hours˜12 hours. LCMS and HPLC showed the reaction was complete. The mixture was quenched with water and the mixture was extracted with ethyl acetate. The combined organic phase was washed with brine, dried with anhydrous Na₂SO₄, filtered and concentrated in vacuum. The residue was purified by prep-HPLC (column: Phenomenex luna C18 250*50 mm*10 um or Phenomenex Gemini-NX 80*40 mm*3 um; mobile phase: [water (0.04% HCl)-MeCN]; B %: 55%-85%, 10 mins or [water (10 mM NH₄HCO₃)-MeCN]; B %: 30%-60%, 8 mins)). The solvent was removed under freeze drying to obtain the desired product.

### Compound 58

### Preparation of 9-(1-(4,6-difluoro-2-methylindolin-1-yl)ethyl)-7-methyl-2-morpholino-4H-pyrido[1,2-a]pyrimidin-4-one (Step 4 in Scheme 17)

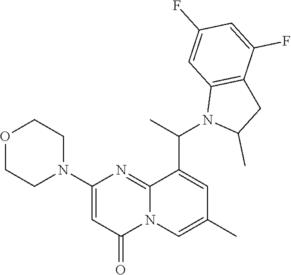 [SMILES: Cc1cc(C(C)N2c3cc(F)cc(F)c3CC2C)c2nc(N3CCOCC3)cc(=O)n2c1]

A mixture of 9-(1-bromoethyl)-7-methyl-2-morpholino-pyrido[1,2-a]pyrimidin-4-one (0.6 g, 1.70 mmol, 1 eq) and 4,6-difluoro-2-methyl-indoline (576.35 mg, 3.41 mmol, 2 eq) in DMA (10 mL) was stirred at 50° C. for 12 hours. LC-MS and HPLC showed the reaction was complete. The mixture was quenched with water (50 mL) and the mixture was extracted with ethyl acetate (50 mL×3). The combined organic phase was washed with brine (40 mL), dried with anhydrous Na₂SO₄, filtered and concentrated in vacuum. The residue was purified by prep-HPLC (column: Phenomenex luna C18 250*50 mm*10 um; mobile phase: [water (0.04% HCl)-MeCN]; B %: 55%-85%, 10 mins). The solvent was removed under freeze drying. Compound 9-[1-(4,6-difluoro-2-methyl-indolin-1-yl)ethyl]-7-methyl-2-morpholino-pyrido[1,2-a]pyrimidin-4-one (0.1716 g, 359.79 umol, 21.12% yield, HCl) was obtained as white solid. ¹H NMR (DMSO-d₆, 400 MHz) δ 8.61 (d, J=11.6 Hz, 1H), 7.85 (d, J=26.0 Hz, 1H), 6.24˜5.94 (m, 2H), 5.64 (d, J=5.6 Hz, 1H), 5.37˜5.02 (m, 1H), 4.37˜4.13 (m, 1H), 3.63˜3.58 (m, 4H), 3.51˜3.36 (m, 4H), 3.28˜3.19 (m, 1H), 2.43˜2.39 (m, 1H), 2.33 (d, J=13.0 Hz, 3H), 1.60 (dd, J=4.0 Hz, 7.2 Hz, 3H), 1.11 (dd, J=2.8, 6.2 Hz, 3H). A solution of 9-[1-(4,6-difluoro-2-methyl-indolin-1-yl)ethyl]-7-methyl-2-morpholino-pyrido[1,2-a]pyrimidin-4-one (0.17 g, 356.43 umol, 1 eq, HCl) in EtOAc (5 mL) was adjusted to pH=7 with sat. NaHCO₃. The organic layer was separated and the aqueous phase was extracted with ethyl acetate (10 mL×3). The combined organic phase was washed with brine (5 mL×2), dried with anhydrous Na₂SO₄, filtered and concentrated in vacuum. The crude product was triturated with MTBE and CH₃CN (v:v=20:1, 30 mL). Compound 9-[1-(4,6-difluoro-2-methyl-indolin-1-yl)ethyl]-7-methyl-2-morpholino-pyrido [1,2-a]pyrimidin-4-one (140.2 mg, 312.56 umol, 87.69% yield, 98.2% purity) was obtained as off-white solid. ¹H NMR (DMSO-d₆, 400 MHz) δ 8.61 (d, J=12.0 Hz, 1H), 7.84 (d, J=24.0 Hz, 1H), 6.27˜5.92 (m, 2H), 5.67˜5.52 (m, 1H), 5.37˜5.05 (m, 1H), 4.33˜4.17 (m, 1H), 3.63˜3.59 (m, 4H), 3.51˜3.37 (m, 4H), 3.25˜3.17 (m, 1H), 2.46˜2.38 (m, 1H), 2.33 (d, J=13.0 Hz, 3H), 1.60 (dd, J=4.0 Hz, 7.2 Hz, 3H), 1.11 (dd, J=3.2 Hz, 6.2 Hz, 3H). HPLC: 98.20% (220 nm), 99.52% (254 nm). MS (ESI): mass calcd. For C₂₄H₂₆F₂O₂N₄ 440.20 m/z found 441.2 [M+H]⁺.

### Compound 59

### 9-(1-(4,6-difluoroindolin-1-yl)ethyl)-7-methyl-2-morpholino-4H-pyrido[1,2-a]pyrimidin-4-one was prepared according to the procedure described herein for Step 4 in Scheme 17

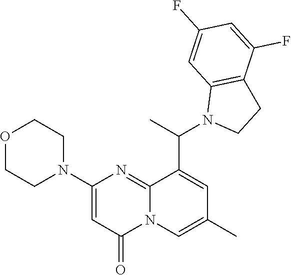 [SMILES: Cc1cc(C(C)N2CCc3c(F)cc(F)cc32)c2nc(N3CCOCC3)cc(=O)n2c1]

The desired compound (43.3 mg, 97.00 umol, 22.78% yield, 95.53% purity) was obtained as pale yellow solid. ¹H NMR (DMSO-d₆, 400 MHz) δ 8.59 (s, 1H), 8.76 (s, 1H), 6.19 (t, J=9.6 Hz, 1H), 6.08 (dd, J=2.0 Hz, J=10.8 Hz, 1H), 5.62 (s, 1H), 5.42 (q, J=7.2 Hz, 1H), 3.66˜3.56 (m, 9H), 3.43˜3.34 (m, 1H), 2.91-2.88 (m, 2H), 2.32 (s, 3H), 1.50 (d, J=7.2 Hz, 3H). HPLC: 95.53% (220 nm), 98.98% (254 nm). MS (ESI): mass calcd. For C₂₃H₂₄F₂N₄O₂ 426.19 m/z found 427.2 [M+H]⁺.

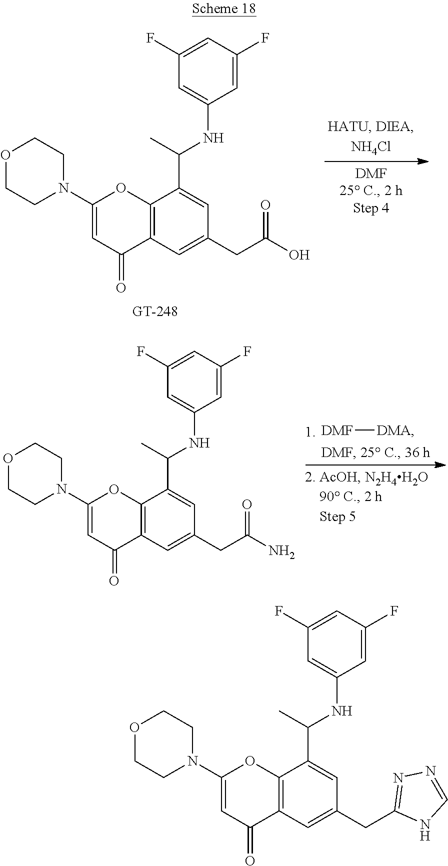

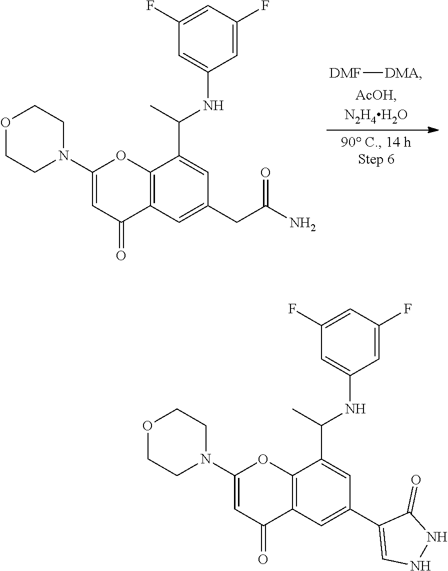

### Compound 60

### Preparation of 4-(8-(1-((3,5-difluorophenyl)amino)ethyl)-2-morpholino-4-oxo-4H-chromen-6-yl)-1H-pyrazol-3(2H)-one (Step 1-3 in Scheme 18)

### Preparation of 2-(8-(1-((3,5-difluorophenyl)amino)ethyl)-2-morpholino-4-oxo-4H-chromen-6-yl)acetamide (Step 1 in Scheme 18)

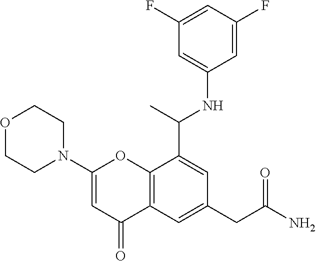 [SMILES: CC(Nc1cc(F)cc(F)c1)c1cc(CC(N)=O)cc2c(=O)cc(N3CCOCC3)oc12]

To a mixture of 2-[8-[1-(3,5-difluoroanilino)ethyl]-2-morpholino-4-oxo-chromen-6-yl]acetic acid (0.25 g, 562.52 umol, 1 eq) and NH₄Cl (90.27 mg, 1.69 mmol, 3 eq) in DMF (5 mL) was added DIEA (218.11 mg, 1.69 mmol, 293.94 uL, 3 eq) and HATU (320.83 mg, 843.78 umol, 1.5 eq) at 0° C. under N₂. The reaction mixture was stirred at 25° C. for 2 hours. LC-MS showed 2-[8-[1-(3,5-difluoroanilino)ethyl]-2-morpholino-4-oxo-chromen-6-yl]acetic acid was consumed completely. The reaction mixture was quenched with ice water (20 mL) and the aqueous phase was extracted with ethyl acetate (15 mL×3). The combined organic phase was washed with brine (15 mL), dried with anhydrous Na₂SO₄, filtered and concentrated in vacuum. The residue was purified by flash silica gel chromatography (Biotage®; 4 g SepaFlash® Silica Flash Column, Eluent of 0˜100% Ethyl acetate/Petroleum ether gradient @ 45 mL/min, Eluent of 0˜20% MeOH/Ethyl acetate@ 45 mL/min). Compound 2-[8-[1-(3,5-difluoroanilino)ethyl]-2-morpholino-4-oxo-chromen-6-yl]acetamide (0.173 g, 390.13 umol, 69.35% yield) was obtained as yellow solid. ¹H NMR (DMSO-d₆, 400 MHz) δ 7.73 (s, 1H), 7.51 (s, 1H), 7.46 (s, 1H), 6.93 (d, J=6.4 Hz, 1H), 6.88 (s, 1H), 6.20 (t, J=9.6 Hz, 1H), 6.13 (d, J=9.6 Hz, 2H), 5.53 (s, 1H), 4.98˜4.91 (m, 1H), 3.76˜3.70 (m, 4H), 3.55˜3.50 (m, 4H), 3.43˜3.79 (m, 2H), 1.48 (d, J=6.8 Hz, 3H).

### Preparation of 6-((4H-1,2,4-triazol-3-yl)methyl)-8-(1-((3,5-difluorophenyl)amino)ethyl)-2-morpholino-4H-chromen-4-one (Step 2 in Scheme 18)

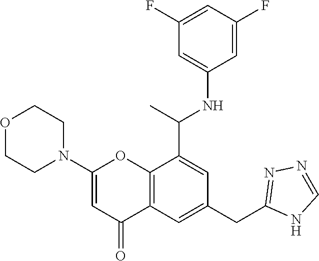 [SMILES: CC(Nc1cc(F)cc(F)c1)c1cc(Cc2nnc[nH]2)cc2c(=O)cc(N3CCOCC3)oc12]

A mixture of 2-[8-[1-(3,5-difluoroanilino)ethyl]-2-morpholino-4-oxo-chromen-6-yl]acetamide (60 mg, 135.31 umol, 1 eq) and DMF-DMA (96.74 mg, 811.83 umol, 107.85 uL, 6 eq) in DMF (1 mL) was stirred at 25° C. for 36 hours. The mixture was concentrated under reduced pressure at 45° C. To the residue was added AcOH (8.13 mg, 135.31 umol, 7.74 uL, 1 eq) and NH₂NH₂—H₂O (13.82 mg, 270.61 umol, 13.42 uL, 98% purity, 2 eq) at 25° C. The mixture was stirred at 90° C. for 2 hours. LC-MS showed 2-[8-[1-(3,5-difluoroanilino)ethyl]-2-morpholino-4-oxo-chromen-6-yl]acetamide was consumed completely. The mixture was concentrated under reduced pressure at 45° C. The residue was purified by prep-HPLC (column: Waters Xbridge BEH C18 100*25 mm*5 um; mobile phase: [water (10 mM NH₄HCO₃)-MeCN]; B %: 20%-50%, 9 mins). The solvent was removed under freeze drying. Compound 8-[1-(3,5-difluoroanilino)ethyl]-2-morpholino-6-(4H-1,2,4-triazol-3-ylmethyl)chromen-4-one (18.8 mg, 38.35 umol, 28.35% yield, 95.37% purity) was obtained as yellow solid. ¹H NMR (DMSO-d₆, 400 MHz) δ 13.75 (s, 1H), 8.40 and 7.81 (s, 1H), 7.66 (s, 1H), 7.49 (s, 1H), 6.92 (d, J=6.4 Hz, 1H), 6.20 (t, J=9.6 Hz, 1H), 6.11 (d, J=9.6 Hz, 1H), 5.53 (s, 1H), 4.97˜4.91 (m, 1H), 4.04˜4.01 (m, 2H), 3.71 (t, J=4.8 Hz, 4H), 3.52 (t, J=4.8 Hz, 4H), 1.48 (d, J=5.6 Hz, 3H). HPLC: 95.37% (220 nm), 100% (254 nm). MS (ESI): mass calcd. For C₂₄H₂₃F₂O₃N₅ 467.18 m/z found 468.2 [M+H]⁺.

### Compound 61

### Preparation of 4-(8-(1-((3,5-difluorophenyl)amino)ethyl)-2-morpholino-4-oxo-4H-chromen-6-yl)-1H-pyrazol-3(2H)-one (Step 3 in Scheme 18)

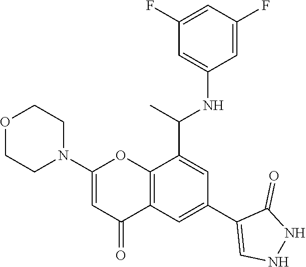 [SMILES: CC(Nc1cc(F)cc(F)c1)c1cc(-c2c[nH][nH]c2=O)cc2c(=O)cc(N3CCOCC3)oc12]

A solution of 2-[8-[1-(3,5-difluoroanilino)ethyl]-2-morpholino-4-oxo-chromen-6-yl]acetamide (50 mg, 112.75 umol, 1 eq) in DMF-DMA (897.00 mg, 7.53 mmol, 1 mL, 66.76 eq) was stirred at 90° C. for 12 hours. The mixture was concentrated under reduced pressure. Then AcOH (437.50 mg, 7.29 mmol, 416.67 uL, 64.61 eq) and NH₂NH₂—H₂O (83.31 mg, 1.66 mmol, 80.89 uL, 14.76 eq) were added at 25° C. The reaction mixture was stirred at 90° C. for 2 hours. LC-MS and HPLC showed 2-[8-[1-(3,5-difluoroanilino)ethyl]-2-morpholino-4-oxo-chromen-6-yl]acetamide was consumed completely. The mixture was concentrated under reduced pressure at 45° C. The residue was purified by prep-HPLC (column: Waters Xbridge BEH C18 100*30 mm*10 um; mobile phase: [water (10 mM NH₄HCO₃)-MeCN]; B %: 20%-45%, 10 mins). The solvent was removed under freeze drying. Compound 4-[8-[1-(3,5-difluoroanilino)ethyl]-2-morpholino-4-oxo-chromen-6-yl]-1,2-dihydropyrazol-3-one (12.3 mg, 24.12 umol, 21.39% yield, 91.87% purity) was obtained as yellow solid. ¹H NMR (DMSO-d₆, 400 MHz) δ 8.11 (s, 1H), 7.91 (s, 1H), 7.86 (s, 1H), 6.90 (d, J=6.4 Hz, 1H), 6.21˜6.14 (m, 3H), 5.53 (s, 1H), 4.98˜4.93 (m, 1H), 3.73˜3.68 (m, 4H), 3.54˜3.50 (m, 4H), 1.52 (d, J=6.0 Hz, 3H). HPLC: 91.87% (220 nm), 93.09% (254 nm). MS (ESI): mass calcd. For C₂₄H₂₂F₂O₄N₄ 468.16 m/z found 469.3 [M+H].

### Compound 62

### Preparation of 8-((7-chloro-4-fluoroindolin-1-yl)methyl)-N,N-dimethyl-2-morpholino-4-oxo-4H-chromene-6-carboxamide (Step 6 in Scheme 9)

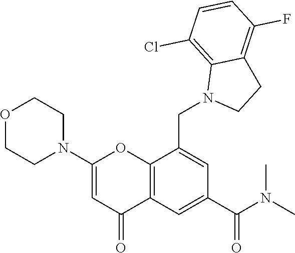 [SMILES: CN(C)C(=O)c1cc(CN2CCc3c(F)ccc(Cl)c32)c2oc(N3CCOCC3)cc(=O)c2c1]

Compound 62 (18 mg, 37.51 umol, 15.49% yield, 97.51% purity) was obtained as off white solid. ¹H NMR (400 MHz, DMSO-d₆) δ 7.84 (s, 1H), 7.62 (s, 1H), 7.07˜7.02 (m, 1H), 6.52 (t, J=8.4 Hz, 1H), 5.59 (s, 1H), 5.02 (s, 2H), 3.72˜3.70 (m, 4H), 3.62 (t, J=8.8 Hz, 2H), 3.59˜3.54 (m, 4H), 3.08 (t, J=8.4 Hz, 2H), 2.97 (s, 3H), 2.86 (s, 3H). LCMS: 97.51% (220 nm), 99.52% (254 nm). MS (ESI): mass calcd. For C₂₅H₂₅ClFN₃O₄ 485.15 m/z found 486.1 [M+H]⁺.

### Compound 63

### Preparation of 8-((4,6-difluoro-2-methylindolin-1-yl)methyl)-N,N-dimethyl-2-morpholino-4-oxo-4H-chromene-6-carboxamide (Step 6 in Scheme 9)

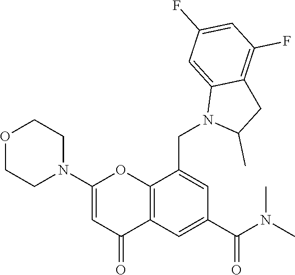 [SMILES: CC1Cc2c(F)cc(F)cc2N1Cc1cc(C(=O)N(C)C)cc2c(=O)cc(N3CCOCC3)oc12]

Compound 63 (153.4 mg, 316.06 umol, 20.88% yield, 99.62% purity) was obtained as white solid. ¹H NMR (DMSO-d₆, 400 MHz) δ 7.82 (s, 1H), 7.47 (s, 1H), 6.28 (d, J=9.2 Hz, 1H), 6.10 (d, J=9.6 Hz, 1H), 5.60 (s, 1H), 4.69˜4.58 (m, 2H), 3.99˜3.91 (m, 1H), 3.71 (t, J=4.8 Hz, 4H), 3.53 (t, J=4.8 Hz, 4H), 3.27˜3.19 (m, 1H), 2.95 (s, 3H), 2.85 (s, 3H), 2.68˜2.63 (m, 1H), 1.23 (d, J=6.0 Hz, 3H). HPLC: 99.62% (220 nm), 100% (254 nm). MS (ESI): mass calcd. For C₂₆H₂₇F₂N₃O₄ 483.20 m/z found 484.2 [M+H]⁺.

### Compound 64 and Compound 65

### Preparation of (S)-8-((4,6-difluoro-2-methylindolin-1-yl)methyl)-N,N-dimethyl-2-morpholino-4-oxo-4H-chromene-6-carboxamide and (R)-8-((4,6-difluoro-2-methylindolin-1-yl)methyl)-N,N-dimethyl-2-morpholino-4-oxo-4H-chromene-6-carboxamide (Step 6 in Scheme 9)

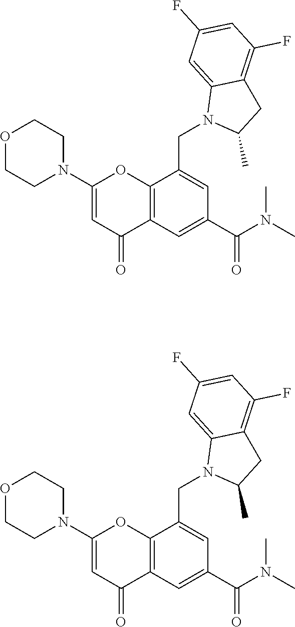 [SMILES: C[C@H]1Cc2c(F)cc(F)cc2N1Cc1cc(C(=O)N(C)C)cc2c(=O)cc(N3CCOCC3)oc12 ; C[C@@H]1Cc2c(F)cc(F)cc2N1Cc1cc(C(=O)N(C)C)cc2c(=O)cc(N3CCOCC3)oc12]

Chiral separation of Compound 63 was performed. Chiral Preparative Instrument: CAS-TJ-ANA-SFC-L (Waters UPCC with PDA); Column: Chiralpak AD-3, 50×4.6 mm I.D., 3 um. Two isomers were obtained. The structures were assigned randomly. Compound 64 (Rt=1.318 minutes, E.E. by chiral HPLC (%)=100%); ¹H NMR (DMSO-d₆, 400 MHz) δ 7.81 (s, 1H), 7.48 (s, 1H), 6.28 (t, J=8.8 Hz, 1H), 6.09 (d, J=10.2 Hz, 1H), 5.60 (s, 1H), 4.70˜4.51 (m, 2H), 3.97˜3.94 (m, 1H), 3.70 (t, J=4.8 Hz, 4H), 3.54 (t, J=4.8 Hz, 4H), 3.27˜3.24 (m, 1H), 2.95 (s, 3H), 2.85 (s, 3H), 1.23 (d, J=6.0 Hz, 3H). HPLC: 100% (220 nm), 100% (254 nm). MS (ESI): mass calcd. For C₂₆H₂₇F₂N₃O₄ 484.2 m/z found 483.2 [M+H]⁺. Compound 65 (Rt=1.578 minutes, E.E. by chiral HPLC (%)=100%). ¹H NMR (DMSO-d₆, 400 MHz) δ 7.82 (s, 1H), 7.48 (s, 1H), 6.28 (t, J=9.2 Hz, 1H), 6.09 (d, J=10.4 Hz, 1H), 5.60 (s, 1H), 4.72˜4.53 (m, 2H), 4.02˜3.88 (m, 1H), 3.71 (t, J=4.8 Hz, 4H), 3.53 (t, J=4.8 Hz, 4H), 3.22˜3.26 (m, 1H), 2.95 (s, 3H), 2.85 (s, 3H), 2.69˜2.64 (m, 1H), 1.23 (d, J=6.0 Hz, 3H). HPLC: 100% (220 nm), 100% (254 nm). MS (ESI): mass calcd. For C₂₆H₂₇F₂N₃O₄ 484.2 m/z found 483.2 [M+H]⁺.

### Compound 66

### Preparation of 8-((4,6-difluoro-2,2-dimethylindolin-1-yl)methyl)-N,N-dimethyl-2-morpholino-4-oxo-4H-chromene-6-carboxamide (Step 6 in Scheme 9)

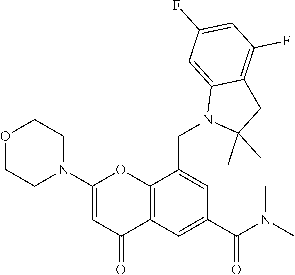 [SMILES: CN(C)C(=O)c1cc(CN2c3cc(F)cc(F)c3CC2(C)C)c2oc(N3CCOCC3)cc(=O)c2c1]

Compound 66 (15.5 mg, 30.85 umol, 11.32% yield, 99.02% purity) was obtained as off white solid. ¹H NMR (400 MHz, DMSO-d₆) δ 7.80 (s, 1H), 7.41 (s, 1H), 6.25 (t, J=8.8 Hz, 1H), 5.82 (d, J=10.4 Hz, 1H), 5.61 (s, 1H), 4.56 (s, 2H), 3.73 (t, J=4.4 Hz, 4H), 3.56 (t, J=4.4 Hz, 4H), 2.95˜2.94 (m, 6H), 2.82 (s, 2H), 1.28 (s, 6H). LCMS: 99.02% (220 nm), 100.00% (254 nm). MS (ESI): mass calcd. For C₂₇H₂₉F₂N₃O₄ 497.21 m/z found 498.2 [M+H]⁺.

### Compound 67

### Preparation of 8-(indolin-1-ylmethyl)-N,N-dimethyl-2-morpholino-4-oxo-4H-chromene-6-carboxamide (Step 6 in Scheme 9)

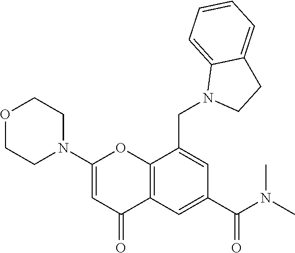 [SMILES: CN(C)C(=O)c1cc(CN2CCc3ccccc32)c2oc(N3CCOCC3)cc(=O)c2c1]

Compound 67 (15.5 mg, 30.85 umol, 11.32% yield, 99.02% purity) was obtained as off white solid. ¹H NMR (400 MHz, DMSO-d₆) δ 7.84 (s, 1H), 7.64 (s, 1H), 7.07 (d, J=7.2 Hz, 1H), 6.99 (t, J=7.6 Hz, 1H), 6.65˜6.56 (m, 2H), 5.59 (s, 1H), 4.54 (s, 2H), 3.67 (t, J=4.4 Hz, 4H), 3.52 (t, J=4.8 Hz, 4H), 3.31-3.28 (m, 2H), 2.98˜2.86 (m, 8H). LCMS: 99.13% (220 nm), 100.00% (254 nm). MS (ESI): mass calcd. For C₂₅H₂₇N₃O₄ 433.20 m/z found 434.2 [M+H]⁺.

### Compound 68

### Preparation of 8-((4-fluoroindolin-1-yl)methyl)-N,N-dimethyl-2-morpholino-4-oxo-4H-chromene-6-carboxamide (Step 6 in Scheme 9)

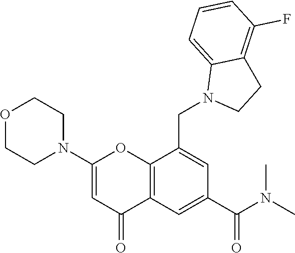 [SMILES: CN(C)C(=O)c1cc(CN2CCc3c(F)cccc32)c2oc(N3CCOCC3)cc(=O)c2c1]

Compound 68 (50.4 mg, 111.63 umol, 46.09% yield, 100% purity) was obtained as off white solid. ¹H NMR (DMSO-d6, 400 MHz) δ 7.84 (s, 1H), 7.62 (s, 1H), 7.05˜7.00 (m, 1H), 6.47˜6.39 (m, 2H), 5.60 (s, 1H), 4.59 (s, 2H), 3.67 (t, J=4.4 Hz, 4H), 3.52 (t, J=4.4 Hz, 4H), 3.43 (t, J=8.4 Hz, 2H), 3.01 (t, J=8.4 Hz, 2H), 2.97 (s, 3H), 2.88 (s, 3H). MS: 100.00% (220 nm), 100.00% (254 nm). MS (ESI): mass calcd. For C₂₅H₂₆FN₃O₄ 451.19 m/z found 452.2 [M+H]⁺.

### Compound 69

### Preparation of 8-((5-chloroindolin-1-yl)methyl)-N,N-dimethyl-2-morpholino-4-oxo-4H-chromene-6-carboxamide (Step 6 in Scheme 9)

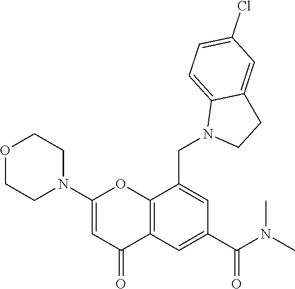 [SMILES: CN(C)C(=O)c1cc(CN2CCc3cc(Cl)ccc32)c2oc(N3CCOCC3)cc(=O)c2c1]

Compound 69 (51.1 mg, 108.54 umol, 44.82% yield, 99.39% purity) was obtained as off white solid. ¹H NMR (DMSO-d6, 400 MHz) δ 7.84 (s, 1H), 7.62 (s, 1H), 7.12˜7.08 (m, 1H), 7.05˜7.00 (m, 1H), 6.64˜6.61 (m, 1H), 5.60 (s, 1H), 4.55 (s, 2H), 3.68 (t, J=4.4 Hz, 4H), 3.52 (t, J=4.4 Hz, 4H), 3.38-3.37 (m, 2H), 2.97 (s, 6H), 2.91-2.76 (m, 2H). MS: 99.39% (220 nm), 100.00% (254 nm). MS (ESI): mass calcd. For C₂₅H₂₆ClN₃O₄ 467.16 m/z found 468.2 [M+H]⁺.

### Compound 70

### Preparation of 8-((6-fluoroindolin-1-yl)methyl)-N,N-dimethyl-2-morpholino-4-oxo-4H-chromene-6-carboxamide (Step 6 in Scheme 9)

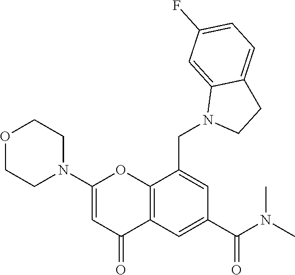 [SMILES: CN(C)C(=O)c1cc(CN2CCc3ccc(F)cc32)c2oc(N3CCOCC3)cc(=O)c2c1]

Compound 70 (29.8 mg, 65.26 umol, 26.95% yield, 98.87% purity) was obtained as off white solid. ¹H NMR (400 MHz, DMSO-d₆) δ 7.84 (s, 1H), 7.61 (s, 1H), 7.01 (t, J=8.0 Hz, 1H), 6.51˜6.48 (m, 1H), 6.35˜6.31 (m, 1H), 5.59 (s, 1H), 4.58 (s, 2H), 3.68 (t, J=4.8 Hz, 4H), 3.52 (t, J=5.2 Hz, 4H), 3.39 (t, J=8.4 Hz, 2H), 2.97˜2.50 (m, 8H). LCMS: 98.87% (220 nm), 100.00% (254 nm). MS (ESI): mass calcd. For C₂₅H₂₆FN₃O₄ 451.19 m/z found 452.2 [M+H]⁺.

### Compound 71

### Preparation of 8-((4-chloroindolin-1-yl)methyl)-N,N-dimethyl-2-morpholino-4-oxo-4H-chromene-6-carboxamide (Step 6 in Scheme 9)

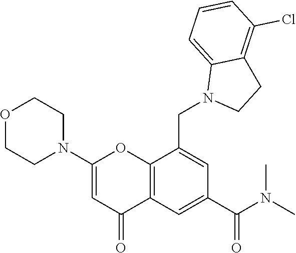 [SMILES: CN(C)C(=O)c1cc(CN2CCc3c(Cl)cccc32)c2oc(N3CCOCC3)cc(=O)c2c1]

Compound 71 (22.1 mg, 45.74 umol, 21.59% yield, 96.86% purity) was obtained as off white solid. ¹H NMR (400 MHz, DMSO-d₆) δ 7.84 (s, 1H), 7.61 (s, 1H), 7.01 (t, J=8.0 Hz, 1H), 6.62 (d, J=8.0 Hz, 1H), 6.56 (d, J=8.0 Hz, 1H), 5.60 (s, 1H), 4.59 (s, 2H), 3.68 (t, J=4.4 Hz, 4H), 3.52 (t, J=4.4 Hz, 4H), 3.43 (t, J=8.8 Hz, 2H), 3.02˜2.87 (m, 8H). LCMS: 96.86% (220 nm), 98.21% (254 nm). MS (ESI): mass calcd. For C₂₅H₂₆ClN₃O₄ 467.16 m/z found 468.2 [M+H]⁺.

### Compound 72

### Preparation of 8-((7-methoxyindolin-1-yl)methyl)-N,N-dimethyl-2-morpholino-4-oxo-4H-chromene-6-carboxamide (Step 6 in Scheme 9)

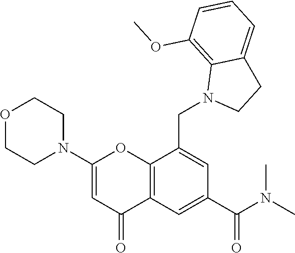 [SMILES: COc1cccc2c1N(Cc1cc(C(=O)N(C)C)cc3c(=O)cc(N4CCOCC4)oc13)CC2]

Compound 72 (30.9 mg, 65.12 umol, 26.89% yield, 97.68% purity) was obtained as off white solid. ¹H NMR (400 MHz, DMSO-d₆) δ 7.83 (s, 1H), 7.67 (s, 1H), 6.82˜6.62 (m, 3H), 5.58 (s, 1H), 4.82 (s, 2H), 3.71 (s, 3H), 3.68 (t, J=4.4 Hz, 4H), 3.54 (t, J=4.0 Hz, 4H), 3.20 (t, J=9.2 Hz, 2H), 2.97 (s, 3H), 2.92˜2.84 (m, 5H). LCMS: 97.68% (220 nm), 100.00% (254 nm). MS (ESI): mass calcd. For C₂₆H₂₉N₃O₅ 463.21 m/z found 464.2 [M+H]⁺.

### Compound 73

### Preparation of 8-((2,3-dihydro-1H-pyrrolo[2,3-b]pyridin-1-yl)methyl)-N,N-dimethyl-2-morpholino-4-oxo-4H-chromene-6-carboxamide (Step 6 in Scheme 9)

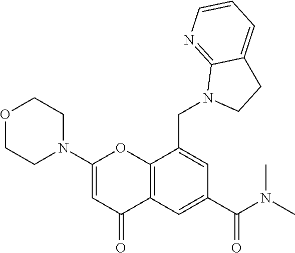 [SMILES: CN(C)C(=O)c1cc(CN2CCc3cccnc32)c2oc(N3CCOCC3)cc(=O)c2c1]

Compound 73 (12 mg, 27.13 umol, 8.96% yield, 98.23% purity) was obtained as yellow solid. ¹H NMR (DMSO-d₆, 400 MHz) δ 7.84 (s, 1H), 7.79 (d, J=5.2 Hz, 1H), 7.66 (s, 1H), 7.30 (d, J=7.2 Hz, 1H), 6.50 (t, J=5.2 Hz, 1H), 5.57 (s, 1H), 4.75 (s, 2H), 3.66 (t, J=4.8 Hz, 4H), 3.55 (t, J=4.8 Hz, 4H), 3.41 (t, J=8.2 Hz, 2H), 2.97˜2.90 (m, 8H). HPLC: 98.23% (220 nm), 99.69% (254 nm). MS (ESI): mass calcd. For C₂₄H₂₆O₄N₄ 434.20 m/z found 435.2 [M+H]⁺.

### Compound 74

### Preparation of 8-((2,3-dihydro-1H-pyrrolo[2,3-c]pyridin-1-yl)methyl)-N,N-dimethyl-2-morpholino-4-oxo-4H-chromene-6-carboxamide (Step 6 in Scheme 9)

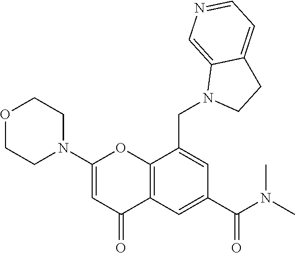 [SMILES: CN(C)C(=O)c1cc(CN2CCc3ccncc32)c2oc(N3CCOCC3)cc(=O)c2c1]

Compound 74 (27.1 mg, 62.26 umol, 25.71% yield, 99.82% purity) was obtained as white solid. ¹H NMR (DMSO-d6, 400 MHz) δ 7.93 (s, 1H), 7.88 (d, J=4.4 Hz, 1H), 7.85 (s, 1H), 7.65 (s, 1H), 7.12 (d, J=4.4 Hz, 1H), 5.60 (s, 1H), 4.62 (s, 2H), 3.67 (t, J=4.4 Hz, 4H), 3.53 (t, J=4.4 Hz, 4H), 3.39˜3.35 (m, 2H), 3.02˜2.99 (m, 2H), 2.97 (s, 3H), 2.88 (s, 3H). MS: 99.82% (220 nm), 100.00% (254 nm). MS (ESI): mass calcd. For C₂₄H₂₆N₄O₄ 434.20 m/z found 435.2 [M+H]⁺.

### Compound 75

### Preparation of 8-((6-fluoro-2,3-dihydro-1H-pyrrolo[3,2-b]pyridin-1-yl)methyl)-N,N-dimethyl-2-morpholino-4-oxo-4H-chromene-6-carboxamide (Step 6 in Scheme 9)

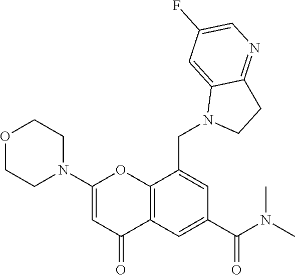 [SMILES: CN(C)C(=O)c1cc(CN2CCc3ncc(F)cc32)c2oc(N3CCOCC3)cc(=O)c2c1]

Compound 75 (42.9 mg, 94.81 umol, 39.15% yield, 100% purity) was obtained as white solid. ¹H NMR (DMSO-d₆, 400 MHz) δ 7.85 (s, 1H), 7.61 (s, 1H), 7.57 (s, 1H), 6.88 (d, J=10.4 Hz, 1H), 5.60 (s, 1H), 4.64 (s, 2H), 3.68 (t, J=4.4 Hz, 4H), 3.53 (t, J=4.8 Hz, 4H), 3.52˜3.50 (m, 2H), 3.03˜3.00 (m, 2H), 2.98 (s, 3H), 2.89 (s, 3H). MS: 100.00% (220 nm), 100.00% (254 nm). MS (ESI): mass calcd. For C₂₄H₂₅FN₄O₄ 452.19 m/z found 452.3 [M+H]⁺.

### Compound 76

### Preparation of 8-((2,3-dihydro-1H-pyrrolo[3,2-b]pyridin-1-yl)methyl)-N,N-dimethyl-2-morpholino-4-oxo-4H-chromene-6-carboxamide (Step 6 in Scheme 9)

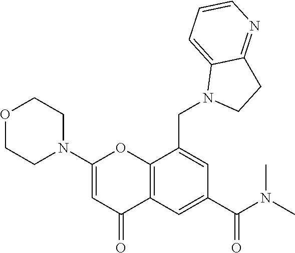 [SMILES: CN(C)C(=O)c1cc(CN2CCc3ncccc32)c2oc(N3CCOCC3)cc(=O)c2c1]

Compound 76 (25.9 mg, 59.61 umol, 24.61% yield, 100.00% purity) was obtained as off white solid. ¹H NMR (400 MHz, DMSO-d₆) δ 7.86 (s, 1H), 7.70 (d, J=4.4 Hz, 1H), 7.64 (s, 1H), 6.94 (t, J=7.6 Hz, 1H)), 6.88 (d, J=8.0 Hz, 1H), 5.60 (s, 1H), 4.59 (s, 2H), 3.68˜3.64 (m, 4H), 3.53˜3.50 (m, 4H), 3.42 (t, J=8.4 Hz, 2H), 3.03 (t, J=8.4 Hz, 2H), 2.98 (s, 3H), 2.90 (s, 3H). LCMS: 100.00% (220 nm), 100.00% (254 nm). MS (ESI): mass calcd. For C₂₄H₂₆N₄O₄ 434.20 m/z found 435.2 [M+H]⁺.

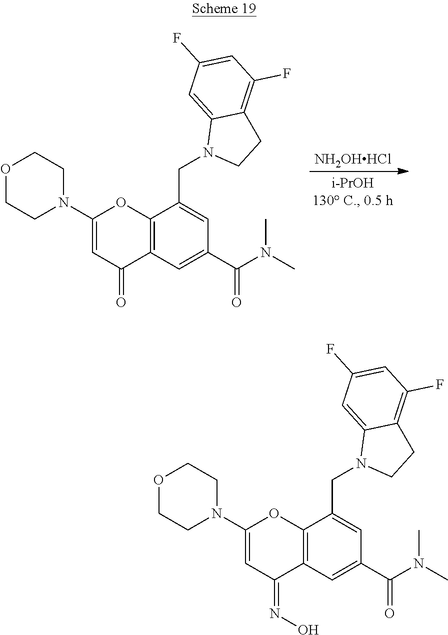

### Compound 77

### Preparation of (Z)-8-((4,6-difluoroindolin-1-yl)methyl)-4-(hydroxyimino)-N,N-dimethyl-2-morpholino-4H-chromene-6-carboxamide (Scheme 19)

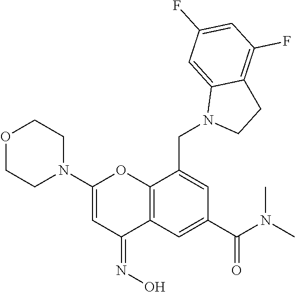 [SMILES: CN(C)C(=O)c1cc(CN2CCc3c(F)cc(F)cc32)c2oc(N3CCOCC3)c/c(=N/O)c2c1]

A mixture of 8-[(4,6-difluoroindolin-1-yl)methyl]-N,N-dimethyl-2-morpholino-4-oxo-chromene-6-carboxamide (0.1 g, 213.00 umol, 1 eq) and NH₂OH—HCl (29.60 mg, 426.00 umol, 2 eq) in i-PrOH (3 mL) was heated at 130° C. for half an hour under microwave. LCMS showed 8-[(4,6-difluoroindolin-1-yl)methyl]-N,N-dimethyl-2-morpholino-4-oxo-chromene-6-carboxamide was consumed completely. The mixture was concentrated under reduced pressure at 45° C. The residue was purified by prep-HPLC (column: Waters Xbridge BEH C18 100*30 mm*10 um; mobile phase: [water (10 mM NH₄HCO₃)-MeCN]; B %: 30%-60%, 10 mins). The solvent was removed under freeze drying. Compound (4Z)-8-[(4,6-difluoroindolin-1-yl)methyl]-4-hydroxyimino-N,N-dimethyl-2-morpholino-chromene-6-carboxamide (15.9 mg, 32.82 umol, 15.41% yield) was obtained as pale yellow solid. ¹H NMR (DMSO-d₆, 400 MHz) δ 7.63 (s, 1H), 7.26 (s, 1H), 6.81 (s, 1H), 6.31˜6.11 (m, 2H), 4.35 (s, 2H), 3.71 (t, J=4.4 Hz, 4H), 3.49 (t, J=8.8 Hz, 2H), 3.21 (t, J=4.8 Hz, 4H), 2.98˜2.88 (m, 8H). HPLC: 100% (220 nm), 100% (254 nm). MS (ESI): mass calcd. For C₂₅H₂₆O₄N₄F₂ 484.19 m/z found 485.3 [M+H]⁺.

### Compound 78

### Preparation of 8-((4,6-difluoroindolin-1-yl)methyl)-N,N-diethyl-2-morpholino-4-oxo-4H-chromene-6-carboxamide (Step 4 in Scheme 11)

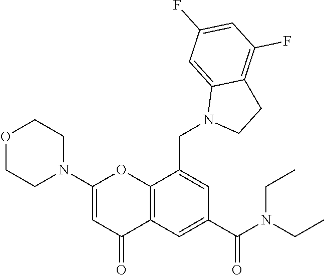 [SMILES: CCN(CC)C(=O)c1cc(CN2CCc3c(F)cc(F)cc32)c2oc(N3CCOCC3)cc(=O)c2c1]

Compound 78 (43.5 mg, 85.62 umol, 37.88% yield, 97.93% purity) was obtained as white solid. ¹H NMR (DMSO-d6, 400 MHz) δ 7.78 (s, 1H), 7.52 (s, 1H), 6.41 (d, J=8.4 Hz, 1H), 6.33˜6.28 (m, 1H), 5.59 (s, 1H), 4.64 (s, 2H), 3.70 (t, J=4.8 Hz, 4H), 3.54˜3.52 (m, 4H), 3.49˜3.47 (m, 2H), 3.29˜3.27 (m, 4H), 2.99 (t, J=8.0 Hz, 2H), 1.12 (s, 3H), 1.00 (s, 3H). MS: 97.93% (220 nm), 99.19% (254 nm). MS (ESI): mass calcd. For C₂₇H₂₉F₂N₃O₄ 497.21 m/z found 498.3 [M+H]⁺.

### Compound 79

### Preparation of 8-((4,6-difluoroindolin-1-yl)methyl)-N-ethyl-N-methyl-2-morpholino-4-oxo-4H-chromene-6-carboxamide (Step 4 in Scheme 11)

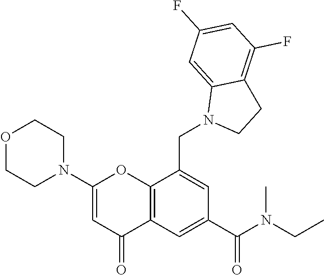 [SMILES: CCN(C)C(=O)c1cc(CN2CCc3c(F)cc(F)cc32)c2oc(N3CCOCC3)cc(=O)c2c1]

Compound 79 (18 mg, 36.39 umol, 16.10% yield, 97.74% purity) was obtained as white solid. ¹H NMR (400 MHz, DMSO-d₆) δ 7.81 (s, 1H), 7.53 (s, 1H), 6.41-6.30 (m, 2H), 5.59 (s, 1H), 4.64 (s, 2H), 3.68 (s, 4H), 3.53˜3.49 (m, 6H), 3.19˜3.12 (m, 2H), 3.01-2.79 (m, 5H), 1.12˜0.94 (m, 3H). LCMS: 97.74% (220 nm), 100.00% (254 nm). MS (ESI): mass calcd. For C₂₆H₂₇F₂N₃O₄ 483.20 m/z found 484.2 [M+H]⁺.

### Compound 80

### Preparation of 8-((4,6-difluoroindolin-1-yl)methyl)-N-ethyl-2-morpholino-4-oxo-4H-chromene-6-carboxamide (Step 4 in Scheme 11)

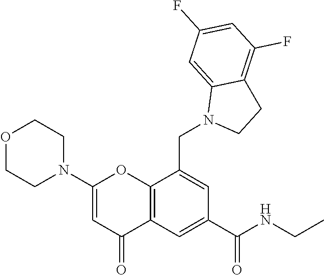 [SMILES: CCNC(=O)c1cc(CN2CCc3c(F)cc(F)cc32)c2oc(N3CCOCC3)cc(=O)c2c1]

Compound 80 (15.7 mg, 32.00 umol, 28.31% yield, 95.68% purity) was obtained as white solid. ¹H NMR (400 MHz, DMSO-d₆) δ 8.75˜8.73 (m, 1H), 8.40 (s, 1H), 8.04 (s, 1H), 6.44 (d, J=8.4 Hz, 1H), 6.32 (t, J=7.6 Hz, 1H), 5.59 (s, 1H), 4.62 (s, 2H), 3.69˜3.66 (m, 4H), 3.54˜3.50 (m, 4H), 3.46 (t, J=8.0 Hz, 2H), 3.24˜3.20 (m, 2H), 2.96 (t, J=8.0 Hz, 2H), 1.12 (t, J=7.2 Hz, 3H). LCMS: 95.68% (220 nm), 99.33% (254 nm). MS (ESI): mass calcd. For C₂₅H₂₅F₂N₃O₄ 469.18 m/z found 470.2 [M+H]⁺.

### Compound 81

### Preparation of 8-((4,6-difluoroindolin-1-yl)methyl)-N-isopropyl-2-morpholino-4-oxo-4H-chromene-6-carboxamide (Step 4 in Scheme 11)

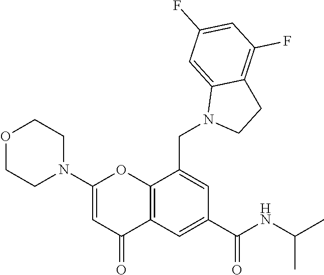 [SMILES: CC(C)NC(=O)c1cc(CN2CCc3c(F)cc(F)cc32)c2oc(N3CCOCC3)cc(=O)c2c1]

Compound 81 (9.5 mg, 19.50 umol, 17.25% yield, 99.24% purity) was obtained as white solid. ¹H NMR (400 MHz, DMSO-d₆) δ 8.55 (d, J=7.6 Hz, 1H), 8.41 (s, 1H), 8.04 (s, 1H), 6.43 (d, J=8.8 Hz, 1H), 6.33 (t, J=8.0 Hz, 1H), 5.59 (s, 1H), 4.62 (s, 2H), 4.12˜4.05 (m, 1H), 3.73˜3.63 (m, 4H), 3.55˜3.50 (m, 4H), 3.46 (t, J=8.4 Hz, 2H), 2.95 (t, J=8.4 Hz, 2H), 1.16 (d, J=6.8 Hz, 6H). LCMS: 99.24% (220 nm), 99.84% (254 nm). MS (ESI): mass calcd. For C₂₆H₂₇F₂N₃O₄ 483.20 m/z found 484.2 [M+H]⁺.

### Compound 82

### Preparation of 8-((4,6-difluoroindolin-1-yl)methyl)-N-(1-methylpiperidin-4-yl)-2-morpholino-4-oxo-4H-chromene-6-carboxamide (Step 4 in Scheme 11)

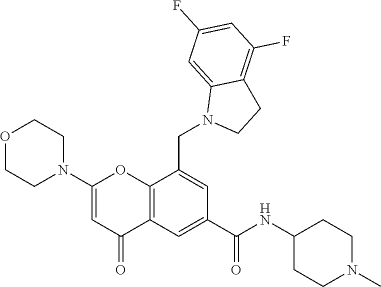 [SMILES: CN1CCC(NC(=O)c2cc(CN3CCc4c(F)cc(F)cc43)c3oc(N4CCOCC4)cc(=O)c3c2)CC1]

Compound 82 (14.1 mg, 25.58 umol, 22.63% yield, 97.71% purity) was obtained as white solid. ¹H NMR (DMSO-d₆, 400 MHz) δ 8.56 (d, J=8.0 Hz, 1H), 8.41 (s, 1H), 8.03 (s, 1H), 6.47˜6.44 (m, 1H), 6.34˜6.29 (m, 1H), 5.60 (s, 1H), 4.63 (s, 2H), 3.78˜3.71 (m, 1H), 3.69˜3.67 (m, 4H), 3.53˜3.51 (m, 4H), 3.44 (t, J=8.8 Hz, 2H), 2.95 (t, J=8.4 Hz, 2H), 2.78 (d, J=10.4 Hz, 2H), 2.16 (s, 3H), 1.97˜1.91 (m, 2H), 1.75˜1.72 (m, 2H), 1.64˜1.56 (m, 2H). MS: 97.71% (220 nm), 98.11% (254 nm). MS (ESI): mass calcd. For C₂₉H₃₂F₂N₄O₄ 538.24 m/z found 539.3 [M+H]⁺.

### Compound 83

### Preparation of 8-((4,6-difluoroindolin-1-yl)methyl)-2-morpholino-4-oxo-N-(tetrahydro-2H-pyran-4-yl)-4H-chromene-6-carboxamide (Step 4 in Scheme 11)

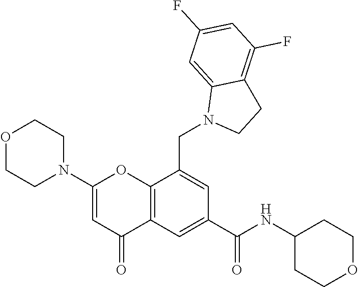 [SMILES: O=C(NC1CCOCC1)c1cc(CN2CCc3c(F)cc(F)cc32)c2oc(N3CCOCC3)cc(=O)c2c1]

Compound 83 (18.8 mg, 35.45 umol, 31.37% yield, 99.11% purity) was obtained as white solid. ¹H NMR (DMSO-d₆, 400 MHz) δ 8.64 (d, J=7.6 Hz, 1H), 8.43 (s, 1H), 8.05 (s, 1H), 6.47˜6.44 (m, 1H), 6.34˜6.30 (m, 1H), 5.60 (s, 1H), 4.63 (s, 2H), 4.01˜3.99 (m, 1H), 3.89 (d, J=4.8 Hz, 2H), 3.69˜3.67 (m, 4H), 3.53˜3.51 (m, 4H), 3.45 (t, J=8.8 Hz, 2H), 3.40 (s, 1H), 3.37 (d, J=1.6 Hz, 1H), 2.95 (t, J=9.2 Hz, 2H), 1.75 (d, J=9.6 Hz, 2H), 1.66˜1.59 (m, 2H). MS: 99.11% (220 nm), 100.00% (254 nm). MS (ESI): mass calcd. For C₂₈H₂₉F₂N₃O₅ 525.21 m/z found 526.2 [M+H]⁺.

### Compound 84

### Preparation of 8-((4,6-difluoroindolin-1-yl)methyl)-N-(2-hydroxy-2-methylpropyl)-2-morpholino-4-oxo-4H-chromene-6-carboxamide (Step 4 in Scheme 11)

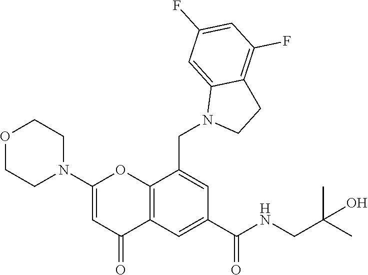 [SMILES: CC(C)(O)CNC(=O)c1cc(CN2CCc3c(F)cc(F)cc32)c2oc(N3CCOCC3)cc(=O)c2c1]

Compound 84 (21 mg, 40.89 umol, 36.18% yield, 100% purity) was obtained as white solid. ¹H NMR (DMSO-d₆, 400 MHz) δ 8.55 (t, J=6.0 Hz, 1H), 8.42 (s, 1H), 8.05 (s, 1H), 6.46 (d, J=10.4 Hz, 1H), 6.31 (t, J=8.0 Hz, 1H), 5.60 (s, 1H), 4.63 (s, 2H), 4.56 (s, 1H), 3.69˜3.67 (m, 4H), 3.54˜3.51 (m, 4H), 3.46 (t, J=8.8 Hz, 2H), 3.24 (d, J=6.0 Hz, 2H), 2.96 (t, J=8.4 Hz, 2H). HPLC: 100% (220 nm), 99.72% (254 nm). MS (ESI): mass calcd. For C₂₇H₂₉F₂O₅N₃ 513.21 m/z found 514.2 [M+H]⁺.

### Compound 85

### Preparation of 8-((4,6-difluoroindolin-1-yl)methyl)-N-isobutyl-2-morpholino-4-oxo-4H-chromene-6-carboxamide (Step 4 in Scheme 11)

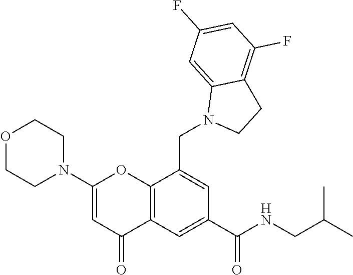 [SMILES: CC(C)CNC(=O)c1cc(CN2CCc3c(F)cc(F)cc32)c2oc(N3CCOCC3)cc(=O)c2c1]

Compound 85 (24.1 mg, 47.57 umol, 42.09% yield, 98.20% purity) was obtained as white solid. ¹H NMR (DMSO-d6, 400 MHz) δ 8.75 (t, J=5.6 Hz, 1H), 8.41 (s, 1H), 8.04 (s, 1H), 6.45 (d, J=10.4 Hz, 1H), 6.31 (t, J=8.0 Hz, 1H), 5.60 (s, 1H), 4.63 (s, 2H), 3.69˜3.67 (m, 4H), 3.53˜3.51 (m, 4H), 3.46 (t, J=8.8 Hz, 2H), 3.07 (t, J=6.0 Hz 2H), 2.98 (t, J=8.4 Hz, 2H), 1.90˜1.80 (m, 1H), 0.87 (d, J=6.8 Hz, 6H). HPLC: 98.20% (220 nm), 98.72% (254 nm). MS (ESI): mass calcd. For C₂₇H₂₉O₄N₃F₂ 497.21 m/z found 498.2 [M+H]⁺.

### Compound 86

### Preparation of 8-((4,6-difluoroindolin-1-yl)methyl)-N-methyl-2-morpholino-4-oxo-4H-chromene-6-carboxamide (Step 4 in Scheme 11)

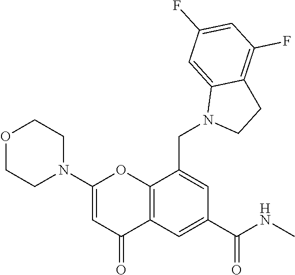 [SMILES: CNC(=O)c1cc(CN2CCc3c(F)cc(F)cc32)c2oc(N3CCOCC3)cc(=O)c2c1]

Compound 86 (8.1 mg, 16.45 umol, 14.55% yield, 92.48% purity) was obtained as pale yellow solid. ¹H NMR (400 MHz, DMSO-d₆) δ 8.68˜8.67 (m, 1H), 8.38 (s, 1H), 8.05s (s, 1H), 6.43 (d, J=10.8 Hz, 1H), 6.32 (t, J=7.6 Hz, 1H), 5.59 (s, 1H), 4.63 (s, 2H), 3.69˜3.67 (m, 4H), 3.55˜3.52 (m, 4H), 3.46 (t, J=8.4 Hz, 2H), 2.95 (t, J=8.4 Hz, 2H), 2.77 (d, J=4.4 Hz, 3H). LCMS: 92.48% (220 nm), 90.06% (254 nm). MS (ESI): mass calcd. For C₂₄H₂₃F₂N₃O₄ 455.17 m/z found 456.1 [M+H]⁺.

### Compound 87

### Preparation of 8-((4,6-difluoroindolin-1-yl)methyl)-6-(3,3-dimethylazetidine-1-carbonyl)-2-morpholino-4H-chromen-4-one (Step 4 in Scheme 11)

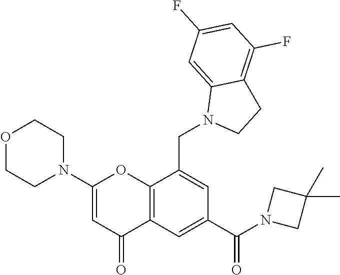 [SMILES: CC1(C)CN(C(=O)c2cc(CN3CCc4c(F)cc(F)cc43)c3oc(N4CCOCC4)cc(=O)c3c2)C1]

Compound 87 (47.1 mg, 92.03 umol, 50.89% yield, 99.56% purity) was obtained as white solid. ¹H NMR (DMSO-d₆, 400 MHz) δ 8.09 (s, 1H), 7.76 (s, 1H), 6.44 (d, J=12.0 Hz, 1H), 6.31 (t, J=8.0 Hz, 1H), 5.60 (s, 1H), 4.67 (s, 2H), 3.86 (s, 2H), 3.72 (s, 2H), 3.70˜3.68 (m, 4H), 3.54˜3.53 (m, 4H), 3.49 (d, J=8.0 Hz, 2H), 2.98 (t, J=8.0 Hz, 2H), 1.22 (s, 6H). MS: 99.56% (220 nm), 100.00% (254 nm). MS (ESI): mass calcd. For C₂₈H₂₉F₂N₃O₄ 509.21 m/z found 510.3 [M+H]⁺.

### Compound 88

### Preparation of 1-(8-((4,6-difluoroindolin-1-yl)methyl)-2-morpholino-4-oxo-4H-chromene-6-carbonyl)-3-methylazetidine-3-carbonitrile (Step 4 in Scheme 11)

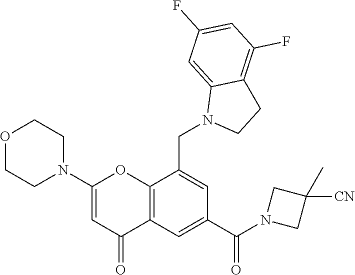 [SMILES: CC1(C#N)CN(C(=O)c2cc(CN3CCc4c(F)cc(F)cc43)c3oc(N4CCOCC4)cc(=O)c3c2)C1]

Compound 88 (21.2 mg, 40.73 umol, 22.52% yield, 100% purity) was obtained as white solid. ¹H NMR (400 MHz, DMSO-d6) δ 8.10 (s, 1H), 7.80 (s, 1H), 6.48 (d, J=8.4 Hz, 1H), 6.31 (t, J=8.4 Hz, 1H), 5.62 (s, 1H), 4.66 (s, 2H), 4.62˜4.60 (m, 1H), 4.38˜4.32 (m, 1H), 4.19˜4.15 (m, 1H), 4.06˜4.01 (m, 1H), 3.71-3.66 (m, 4H), 3.56˜3.52 (m, 4H), 3.49 (t, J=8.8 Hz, 2H), 2.98 (t, J=8.4 Hz, 2H), 1.64 (s, 3H). LCMS: 100.00% (220 nm), 100.00% (254 nm). MS (ESI): mass calcd. For C₂₈H₂₆F₂N₄O₄ 520.19 m/z found 521.3 [M+H]⁺.

### Compound 89

### Preparation of 8-((4,6-difluoroindolin-1-yl)methyl)-6-(3-hydroxyazetidine-1-carbonyl)-2-morpholino-4H-chromen-4-one (Step 4 in Scheme 11)

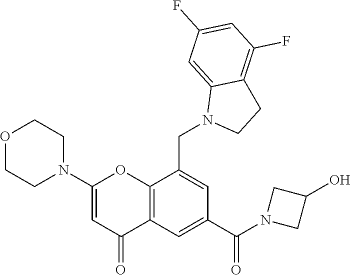 [SMILES: O=C(c1cc(CN2CCc3c(F)cc(F)cc32)c2oc(N3CCOCC3)cc(=O)c2c1)N1CC(O)C1]

Compound 89 (19.5 mg, 39.20 umol, 21.68% yield, 100.00% purity) was obtained as white solid. ¹H NMR (400 MHz, DMSO-d₆) δ 8.06 (s, 1H), 7.76 (s, 1H), 6.44 (d, J=10.4 Hz, 1H), 6.32 (t, J=8.4 Hz, 1H), 5.76 (d, J=6.4 Hz, 1H), 5.60 (s, 1H), 4.65 (s, 2H), 4.49˜4.45 (m, 1H), 4.39˜4.36 (m, 1H), 4.27˜4.23 (m, 1H), 3.99˜3.94 (m, 1H), 3.81-3.77 (m, 1H), 3.72˜3.65 (m, 4H), 3.53˜3.52 (m, 4H), 3.48 (t, J=8.0 Hz, 2H), 2.97 (t, J=8.0 Hz, 2H). LCMS: 100.00% (220 nm), 100.00% (254 nm). MS (ESI): mass calcd. For C₂₆H₂₅F₂N₃O₅ 497.18 m/z found 498.2 [M+H]⁺.

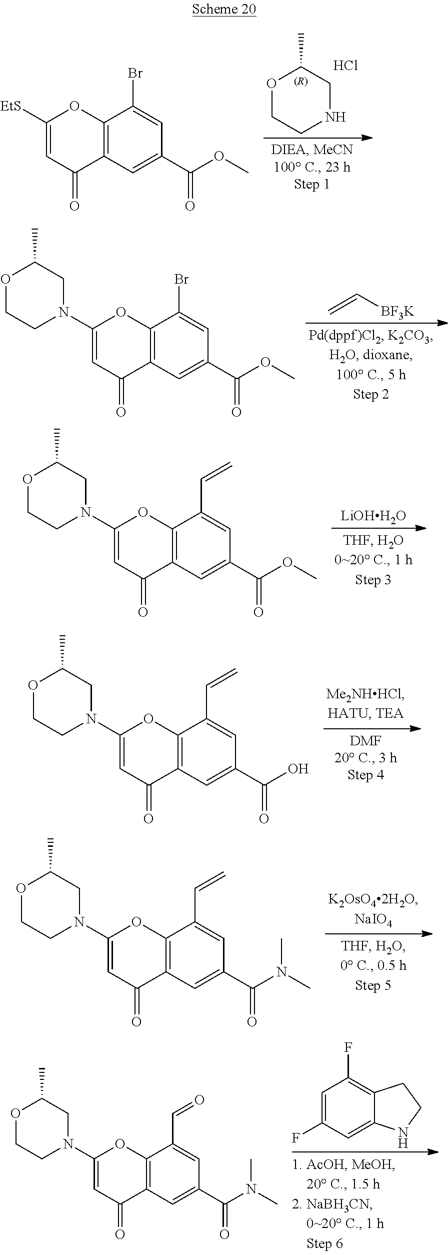

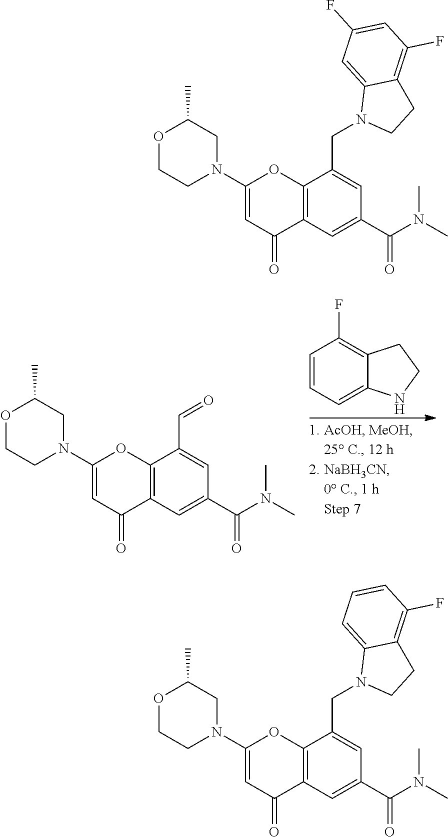 [SMILES: C[C@@H]1CN(c2cc(=O)c3cc(C(=O)N(C)C)cc(CN4CCc5c(F)cc(F)cc54)c3o2)CCO1 ; C[C@@H]1CN(c2cc(=O)c3cc(C(=O)N(C)C)cc(C=O)c3o2)CCO1 ; Fc1cccc2c1CCN2 ; C[C@@H]1CN(c2cc(=O)c3cc(C(=O)N(C)C)cc(CN4CCc5c(F)cccc54)c3o2)CCO1]

### Compound 90

### Preparation of (R)-8-((4,6-difluoroindolin-1-yl)methyl)-N,N-dimethyl-2-(2-methylmorpholino)-4-oxo-4H-chromene-6-carboxamide and (R)-8-((4-fluoroindolin-1-yl)methyl)-N,N-dimethyl-2-(2-methylmorpholino)-4-oxo-4H-chromene-6-carboxamide (Steps 1-7 in Scheme 20)

### Preparation of (R)-methyl 8-bromo-2-(2-methylmorpholino)-4-oxo-4H-chromene-6-carboxylate (Step 1 in Scheme 20)

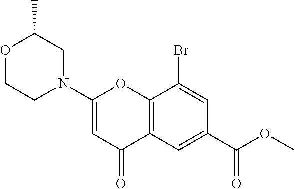 [SMILES: COC(=O)c1cc(Br)c2oc(N3CCO[C@H](C)C3)cc(=O)c2c1]

A solution of methyl 8-bromo-2-ethylsulfanyl-4-oxo-chromene-6-carboxylate (8 g, 23.31 mmol, 1 eq), (2R)-2-methylmorpholine (4.81 g, 34.97 mmol, 1.03 mL, 1.5 eq, HCl) and DIEA (9.04 g, 69.93 mmol, 12.18 mL, 3 eq) in MeCN (80 mL) was stirred at 105° C. for 15 hours. Then DIEA (3.01 g, 23.31 mmol, 4.06 mL, 1 eq) was added and the reaction mixture was stirred at 110° C. for 15 hours. TLC (petroleum ether:EtOAc=1:1, R_{f}=0.15) and LCMS showed ˜40% of methyl 8-bromo-2-[(2R)-2-methylmorpholin-4-yl]-4-oxo-chromene-6-carboxylate was formed and ˜18% of methyl 8-bromo-2-ethylsulfanyl-4-oxo-chromene-6-carboxylate was remained. The solvent was removed under reduced pressure. The residue was dissolved in EtOAc (100 mL) and water (100 mL). There was some brown solid formed. The solid was collected after filtration. For the filtrate, the organic layer was separated and the aqueous was extracted with EtOAc (50 mL×2). The combined organic layer washed with water (30 mL×2) and brine (30 mL×1), dried over Na₂SO₄, filtered and concentrated under reduced pressure. The crude product was triturated with EtOAc (15 mL) and petroleum ether (60 mL). The solid was collected after filtration. Compound methyl 8-bromo-2-[(2R)-2-methylmorpholin-4-yl]-4-oxo-chromene-6-carboxylate (6.7 g, 17.53 mmol, 75.20% yield) was obtained as brown solid. ¹H NMR (DMSO-d₆, 400 MHz) δ 8.40 (s, 1H), 8.32 (s, 1H), 5.65 (s, 1H), 4.02˜3.89 (m, 6H), 3.64˜3.61 (m, 2H), 3.17˜3.15 (m, 1H), 2.84 (t, J=2.4 Hz, 1H), 1.10 (d, J=6.4 Hz, 3H).

### Preparation of (R)-methyl 2-(2-methylmorpholino)-4-oxo-8-vinyl-4H-chromene-6-carboxylate (Step 2 in Scheme 20)

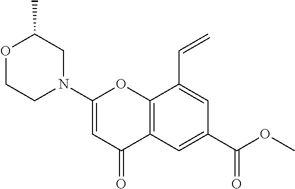 [SMILES: C=Cc1cc(C(=O)OC)cc2c(=O)cc(N3CCO[C@H](C)C3)oc12]

A mixture of methyl 8-bromo-2-[(2R)-2-methylmorpholin-4-yl]-4-oxo-chromene-6-carboxylate (6.7 g, 17.53 mmol, 1 eq), Pd(dppf)Cl₂ (1.28 g, 1.75 mmol, 0.1 eq), K₂CO₃ (4.85 g, 35.06 mmol, 2 eq) and potassium hydride trifluoro(vinyl)boron (3.52 g, 26.29 mmol, 1.5 eq) in dioxane (60 mL) and H₂O (10 mL) was stirred at 100° C. for 5 hours under N₂. TLC (petroleum ether:EtOAc=0:1, R_{f}=0.18) and LCMS showed the reaction was complete. After filtration through a pad of the Celite, the filtrate was concentrated under reduced pressure. The residue was dissolved in EtOAc (50 mL) and water (50 mL). There was some off-white solid formed. The solid was collected after filtration. The filtrate was extracted with DCM (50 mL×3). The combined organic layer was washed with brine (50 mL×4), dried over Na₂SO₄, filtered and concentrated under reduced pressure. The combined crude product was triturated with EtOAc (15 mL) and petroleum ether (60 mL). The solid was collected after filtration. Compound methyl 2-[(2R)-2-methylmorpholin-4-yl]-4-oxo-8-vinyl-chromene-6-carboxylate (5.7 g, 17.31 mmol, 98.73% yield) was obtained as brown solid. ¹H NMR (DMSO-d₆, 400 MHz) δ 8.39 (s, 1H), 8.31 (s, 1H), 7.20˜7.13 (m, 1H), 6.07 (dd, J=6.0 Hz, 18.0 Hz, 1H), 5.62˜5.58 (m, 2H), 4.02˜3.90 (m, 6H), 3.62˜3.56 (m, 2H), 3.15˜3.09 (m, 1H), 2.79 (t, J=10.8 Hz, 1H), 1.17 (d, J=6.0 Hz, 3H).

### Preparation of (R)-2-(2-methylmorpholino)-4-oxo-8-vinyl-4H-chromene-6-carboxylic acid (Step 3 in Scheme 20)

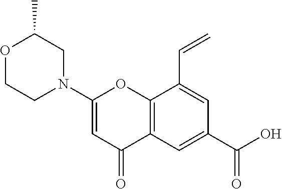 [SMILES: C=Cc1cc(C(=O)O)cc2c(=O)cc(N3CCO[C@H](C)C3)oc12]

A solution of methyl 2-[(2R)-2-methylmorpholin-4-yl]-4-oxo-8-vinyl-chromene-6-carboxylate (5.7 g, 17.31 mmol, 1 eq) and LiOH·H₂O (2.18 g, 51.92 mmol, 3 eq) in THF (30 mL) and H₂O (30 mL) was stirred at 20° C. for an hour. TLC (petroleum ether:EtOAc=0:1, R_{f}=0.20) and LCMS showed the reaction was complete. Half of the organic solvent was removed under reduced pressure. The remained aqueous and half THF phase were made pH=4 with 1N HCl at 0° C. and there was some white solid formed. The solid was collected after filtration. Compound 2-[(2R)-2-methylmorpholin-4-yl]-4-oxo-8-vinyl-chromene-6-carboxylic acid (5.1 g, 16.17 mmol, 93.45% yield) was obtained as off white solid. ¹H NMR (DMSO-d₆, 400 MHz) δ 13.22 (s, 1H), 8.39 (s, 1H), 8.33 (s, 1H), 7.56˜7.49 (m, 1H), 6.06 (d, J=17.6 Hz, 1H), 5.61˜5.57 (m, 2H), 3.95˜3.92 (m, 3H), 3.63˜3.57 (m, 2H), 3.15˜3.12 (m, 1H), 2.82˜2.79 (m, 1H), 1.17 (d, J=6.4 Hz, 3H).

### Preparation of (R)—N,N-dimethyl-2-(2-methylmorpholino)-4-oxo-8-vinyl-4H-chromene-6-carboxamide (Step 4 in Scheme 20)

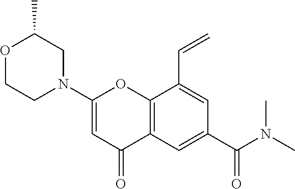 [SMILES: C=Cc1cc(C(=O)N(C)C)cc2c(=O)cc(N3CCO[C@H](C)C3)oc12]

To a mixture of 2-[(2R)-2-methylmorpholin-4-yl]-4-oxo-8-vinyl-chromene-6-carboxylic acid (5.1 g, 16.17 mmol, 1 eq), DIEA (10.45 g, 80.87 mmol, 14.09 mL, 5 eq) and N-methylmethanamine (3.96 g, 48.52 mmol, 4.45 mL, 3 eq, HCl) in THF (50 mL) was added T3P (15.44 g, 24.26 mmol, 14.43 mL, 50% purity, 1.5 eq) dropwise at 0° C. The mixture was stirred at 20° C. for 8 hours. TLC (EtOAc:MeOH=15:1, R_{f}=0.12) and LCMS showed the reaction was nearly complete. The reaction mixture was poured into ice water (200 mL) and made pH=6 with 1N HCl. The mixture was extracted with EtOAc (80 mL×4). The combined organic layer was washed with brine (50 mL×1), dried over Na₂SO₄, filtered and concentrated under reduced pressure. The aqueous was further extracted with DCM and i-PrOH (v:v=3:1, 80 mL×4). The combined organic layer was washed with brine (50 mL×1), dried over Na₂SO₄, filtered and concentrated under reduced pressure. Compound N,N-dimethyl-2-[(2R)-2-methylmorpholin-4-yl]-4-oxo-8-vinyl-chromene-6-carboxamide (3 g, 8.76 mmol, 54.17% yield) was obtained as brown solid. ¹H NMR (DMSO-d₆, 400 MHz) δ 7.93 (s, 1H), 7.81 (s, 1H), 7.18 (dd, J=7.6 Hz, 1H), 6.08 (d, J=22.0 Hz, 1H), 5.61 (s, 1H), 5.56 (d, J=22.0 Hz, 1H), 4.02˜3.92 (m, 3H), 3.63˜3.60 (m, 2H), 3.13˜3.10 (m, 1H), 3.01 and 2.94 (s, 6H), 2.79˜2.72 (m, 1H), 1.16 (d, J=6.0 Hz, 3H).

### Preparation of (R)-8-formyl-N,N-dimethyl-2-(2-methylmorpholino)-4-oxo-4H-chromene-6-carboxamide (Step 5 in Scheme 20)

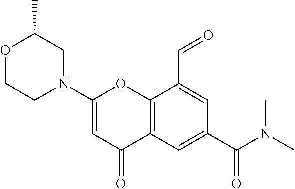 [SMILES: C[C@@H]1CN(c2cc(=O)c3cc(C(=O)N(C)C)cc(C=O)c3o2)CCO1]

A mixture of N,N-dimethyl-2-[(2R)-2-methylmorpholin-4-yl]-4-oxo-8-vinyl-chromene-6-carboxamide (0.5 g, 1.46 mmol, 1 eq) and K₂OsO4·2H₂O (53.81 mg, 146.03 umol, 0.1 eq) in THF (10 mL) and H₂O (10 mL) was stirred at 20° C. for half an hour. Then the mixture was cooled to 0° C. and NaIO₄ (937.05 mg, 4.38 mmol, 242.76 uL, 3 eq) was added in portions at 0° C. and the mixture was stirred at 0° C. for half an hour. TLC (EtOAc:MeOH=15:1, R_{f}=0.15) and LCMS showed the reaction was complete. The mixture was extracted with DCM and i-PrOH (v:v=3:1, 20 mL×3). The combined organic layer was washed with brine (10 mL×1), dried over Na₂SO₄, filtered and concentrated under reduced pressure. Compound 8-formyl-N,N-dimethyl-2-[(2R)-2-methylmorpholin-4-yl]-4-oxo-chromene-6-carboxamide (0.5 g, 1.45 mmol, 99.43% yield) was obtained as brown solid. ¹H NMR (DMSO-d₆, 400 MHz) δ 10.41 (s, 1H), 8.17 (s, 1H), 8.14 (s, 1H), 5.68 (s, 1H), 4.10˜4.02 (m, 3H), 3.62˜3.59 (m, 2H), 3.02˜2.99 (m, 1H), 2.99 and 2.92 (s, 6H), 2.82˜2.79 (m, 1H), 1.05 (d, J=6.4 Hz, 3H).

### Preparation of (R)-8-((4,6-difluoroindolin-1-yl)methyl)-N,N-dimethyl-2-(2-methylmorpholino)-4-oxo-4H-chromene-6-carboxamide (Step 6 in Scheme 20)

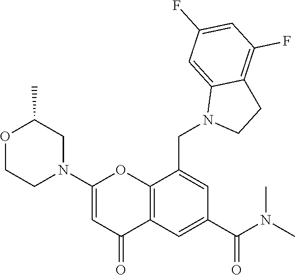 [SMILES: C[C@@H]1CN(c2cc(=O)c3cc(C(=O)N(C)C)cc(CN4CCc5c(F)cc(F)cc54)c3o2)CCO1]

A solution of 8-formyl-N,N-dimethyl-2-[(2R)-2-methylmorpholin-4-yl]-4-oxo-chromene-6-carboxamide (0.5 g, 1.16 mmol, 80% purity, 1 eq), AcOH (2.09 g, 34.85 mmol, 1.99 mL, 30 eq) and 4,6-difluoroindoline (270.32 mg, 1.74 mmol, 1.5 eq) in MeOH (3 mL) was stirred at 20° C. for 1.5 hours. Then NaBH₃CN (145.99 mg, 2.32 mmol, 2 eq) was added in portions at 0° C. and the mixture was stirred at 20° C. for an hour. HPLC and LCMS showed the reaction was complete. The mixture was quenched with ice water (10 mL) at 0° C. and the organic solvent was removed under reduced pressure. The aqueous was made pH=8 with sat. NaHCO₃, then extracted with EtOAc (10 mL×4). The combined organic layer was washed with brine (10 mL×1), dried over Na₂SO₄, filtered and concentrated under reduced pressure. The residue was purified by prep-HPLC (column: Waters Xbridge C18 150*50 mm*10 um; mobile phase: [water (10 mM NH₄HCO₃)-MeCN]; B %: 30%-50%, 10 mins). The eluent was removed under freeze drying. Compound 8-[(4,6-difluoroindolin-1-yl)methyl]-N,N-dimethyl-2-[(2R)-2-methylmorpholin-4-yl]-4-oxo-chromene-6-carboxamide (140.6 mg, 289.02 umol, 24.88% yield, 99.39% purity) was obtained as white solid. ¹H NMR (DMSO-d₆, 400 MHz) δ 7.85 (s, 1H), 7.61 (s, 1H), 6.50˜6.46 (m, 1H), 6.34˜6.29 (m, 1H), 5.61 (s, 1H), 4.66˜4.60 (m, 2H), 3.94˜3.87 (m, 3H), 3.56˜3.50 (m, 2H), 3.43 (t, J=8.8 Hz, 2H), 2.98˜2.90 (m, 9H), 2.75˜2.69 (m, 1H), 1.07 (d, J=6.4 Hz, 3H). HPLC: 99.39% (220 nm), 98.68% (254 nm). MS (ESI): mass calcd. For C₂₆H₂₇F₂N₃O₄ 483.20 m/z found 484.2 [M+H]⁺.

### Compound 91

### Preparation of (R)-8-((4-fluoroindolin-1-yl)methyl)-N,N-dimethyl-2-(2-methylmorpholino)-4-oxo-4H-chromene-6-carboxamide (Step 7 in Scheme 20)

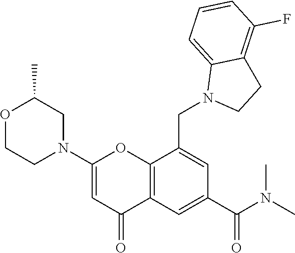 [SMILES: C[C@@H]1CN(c2cc(=O)c3cc(C(=O)N(C)C)cc(CN4CCc5c(F)cccc54)c3o2)CCO1]

A solution of 8-formyl-N,N-dimethyl-2-[(2R)-2-methylmorpholin-4-yl]-4-oxo-chromene-6-carboxamide (5.5 g, 15.97 mmol, 1 eq), AcOH (1.92 g, 31.94 mmol, 1.83 mL, 2 eq) and 4-fluoroindoline (2.19 g, 15.97 mmol, 1 eq) in MeOH (50 mL) was stirred at 20° C. for 1.5 hours. Then NaBH₃CN (1.51 g, 23.96 mmol, 1.5 eq) was added in portions at 0° C. under N₂. The mixture was stirred at 0° C. for an hour. LC-MS and HPLC showed 8-formyl-N,N-dimethyl-2-[(2R)-2-methylmorpholin-4-yl]-4-oxo-chromene-6-carboxamide was consumed completely. The mixture was quenched with ice water (50 mL) at 0° C. and the organic solvent was removed under reduced pressure. The aqueous was made pH=8 with sat. NaHCO₃, then extracted with DCM and i-PrOH (v:v=3:1, 80 mL×3). The combined organic layer was washed with brine (30 mL×1), dried over Na₂SO₄, filtered and concentrated under reduced pressure. The residue was purified by flash silica gel chromatography (Biotage®; 80 g SepaFlash® Silica Flash Column, Eluent of 0˜14% gradient methanol/Ethyl acetate @130 mL/min). The eluent was removed under reduced pressure. The crude (4.49 g) was further purified by prep-HPLC (column: Xtimate C18 10 u 250*80 mm; mobile phase: [water (10 mM NH₄HCO₃)-MeCN]; B %: 25%-55%, 30 mins). The eluent was removed under freeze drying. The solvent was removed under freeze drying. Compound 8-[(4-fluoroindolin-1-yl)methyl]-N,N-dimethyl-2-[(2R)-2-methylmorpholin-4-yl]-4-oxo-chromene-6-carboxamide (3.16 g, 6.78 mmol, 42.48% yield, 99.94% purity) was obtained as white solid. This batch was combined with ET34641-507 and ET34641-509 together and dried under freeze drying then QC checking together for delivery. Total 0.65 g+1.86 g+3.16 g=5.67 g was obtained. ¹H NMR (400 MHz, DMSO-d₆) δ 7.85 (s, 1H), 7.63 (s, 1H), 7.06˜7.00 (m, 1H), 6.50 (d, J=7.6 Hz, 1H), 6.42 (t, J=8.8 Hz, 1H), 5.61 (s, 1H), 4.57 (s, 2H), 3.97˜3.86 (m, 3H), 3.54˜3.53 (m, 2H), 3.45˜3.40 (m, 2H), 3.07˜3.06 (m, 1H), 2.98˜2.88 (m, 8H), 2.76˜2.72 (m, 1H), 1.05 (d, J=6.4 Hz, 3H). HPLC: 99.94% (220 nm), 99.70% (254 nm). MS (ESI): mass calcd. For C₂₆H₂₈FN₃O₄ 465.21 m/z found 466.2 [M+H]⁺.

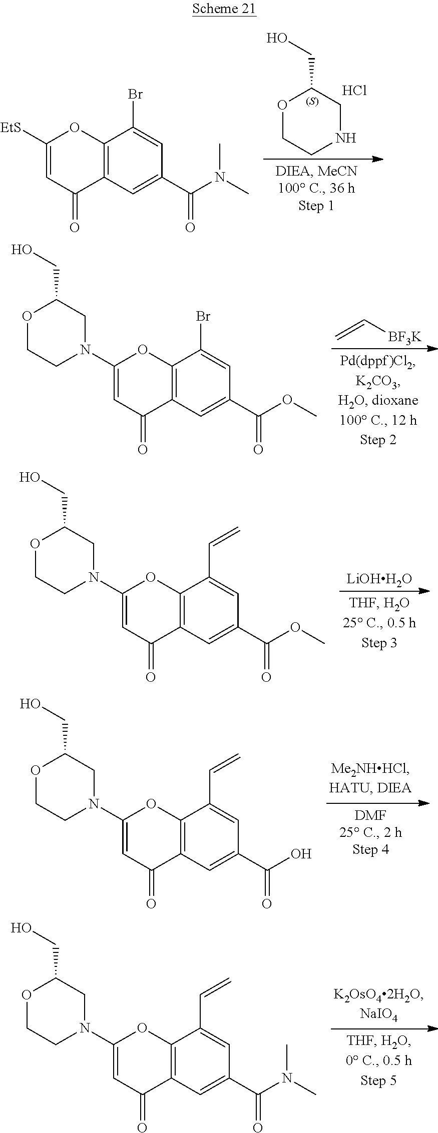

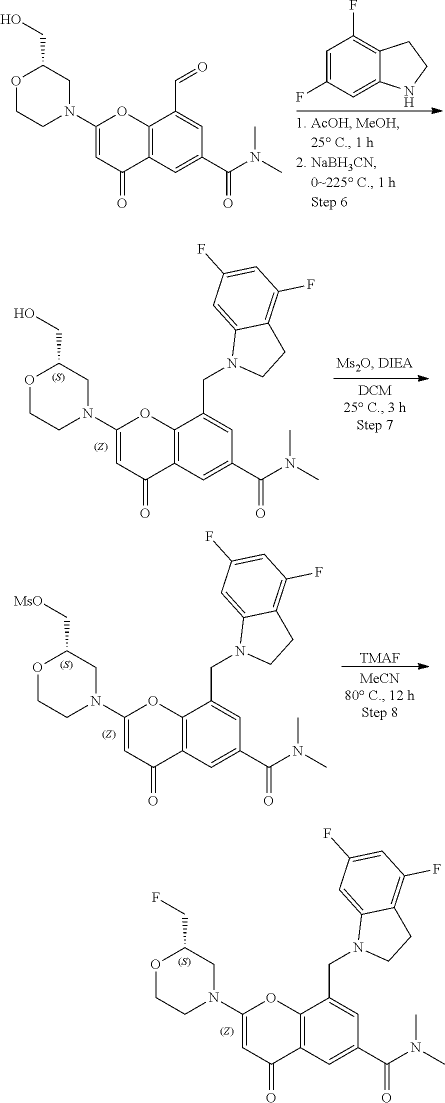

### Compound 92

### Preparation of (S)-8-((4,6-difluoroindolin-1-yl)methyl)-2-(2-(fluoromethyl)morpholino)-N,N-dimethyl-4-oxo-4H-chromene-6-carboxamide (Steps 1-8 in Scheme 21)

### Preparation of (S)-methyl 8-bromo-2-(2-(hydroxymethyl)morpholino)-4-oxo-4H-chromene-6-carboxylate (Step 1 in Scheme 21)

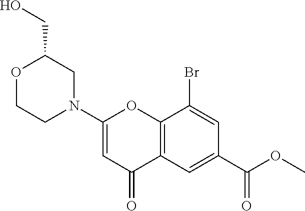 [SMILES: COC(=O)c1cc(Br)c2oc(N3CCO[C@H](CO)C3)cc(=O)c2c1]

To a mixture of methyl 8-bromo-2-ethylsulfanyl-4-oxo-chromene-6-carboxylate (10 g, 29.14 mmol, 1 eq) and [(2S)-morpholin-2-yl]methanol (6.83 g, 44.45 mmol, 1.53 eq, HCl) in CH₃CN (100 mL) was added DIEA (15.06 g, 116.56 mmol, 20.30 mL, 4 eq). The mixture was stirred at 100° C. for 36 hours under N₂. LCMS showed methyl 8-bromo-2-ethylsulfanyl-4-oxo-chromene-6-carboxylate was nearly consumed. The mixture was concentrated under reduced pressure at 45° C. The residue was dissolved in EtOAc (150 mL) and H₂O (200 mL). The organic phase was separated and the aqueous phase was extracted with ethyl acetate (80 mL×2). The combined organic phase was washed with brine (50 mL×2), dried over anhydrous Na₂SO₄, filtered and concentrated in vacuum. The residue was purified by flash silica gel chromatography (Biotage®; 80 g SepaFlash® Silica Flash Column, Eluent of 0˜80% Ethyl acetate/Petroleum ether gradient @ 60 mL/min). The solvent was concentrated in vacuum. Compound methyl 8-bromo-2-[(2S)-2-(hydroxymethyl)morpholin-4-yl]-4-oxo-chromene-6-carboxylate (6.4 g, 16.07 mmol, 55.15% yield) was obtained as yellow solid. ¹H NMR (DMSO-d₆, 400 MHz) δ 8.42 (s, 1H), 8.35 (s, 1H), 5.63 (s, 1H), 4.89 (t, J=5.6 Hz, 1H), 4.04˜3.99 (m, 1H), 3.96 (d, J=8.4 Hz, 2H), 3.89 (s, 3H), 3.63˜3.61 (m, 2H), 3.51˜3.47 (m, 2H), 3.24 (t, J=11.6 Hz, 1H), 2.99 (t, J=11.6 Hz, 1H).

### Preparation of (S)-methyl 2-(2-(hydroxymethyl)morpholino)-4-oxo-8-vinyl-4H-chromene-6-carboxylate (Step 2 in Scheme 21)

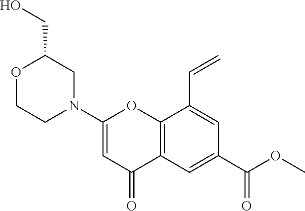 [SMILES: C=Cc1cc(C(=O)OC)cc2c(=O)cc(N3CCO[C@H](CO)C3)oc12]

To a mixture of methyl 8-bromo-2-[(2S)-2-(hydroxymethyl)morpholin-4-yl]-4-oxo-chromene-6-carboxylate (6.4 g, 16.07 mmol, 1 eq) and potassium trifluoro(vinyl)boranuide (3.23 g, 24.11 mmol, 1.5 eq) in dioxane (50 mL) and H₂O (10 mL) was added K₂CO₃ (4.44 g, 32.14 mmol, 2 eq) and Pd(dppf)Cl₂ (588.01 mg, 803.61 umol, 0.05 eq). The mixture was stirred at 100° C. for 12 hours under N₂. LC-MS showed methyl 8-bromo-2-[(2S)-2-(hydroxymethyl) morpholin-4-yl]-4-oxo-chromene-6-carboxylate was consumed completely. The mixture was cooled to room temperature and filtered through a pad of the Celite. The filtrate was concentrated under reduced pressure. The residue was dissolved in EtOAc (70 mL) and H₂O (100 mL). The organic layer was separated and the aqueous phase was extracted with ethyl acetate (50 mL×2). The combined organic phase was washed with brine (35 mL), dried over anhydrous Na₂SO₄, filtered and concentrated in vacuum. Compound methyl 2-[(2S)-2-(hydroxymethyl)morpholin-4-yl]-4-oxo-8-vinyl-chromene-6-carboxylate (4.3 g, 12.45 mmol, 77.47% yield) was obtained as yellow solid. ¹H NMR (DMSO-d₆, 400 MHz) δ 8.39 (s, 1H), 8.33 (s, 1H), 7.19˜7.12 (dd, J=10.8 Hz, 17.6 Hz, 1H), 6.08 (d, J=17.6 Hz, 1H), 5.61˜5.59 (m, 2H), 4.83 (t, J=6.4 Hz, 1H), 3.99˜3.94 (m, 2H), 3.89 (s, 3H), 3.64˜3.56 (m, 2H), 3.51-3.47 (m, 2H), 3.17 (t, J=11.6 Hz, 1H), 2.95 (t, J=11.6 Hz, 1H).

### Preparation of (S)-2-(2-(hydroxymethyl)morpholino)-4-oxo-8-vinyl-4H-chromene-6-carboxylic acid (Step 3 in Scheme 21)

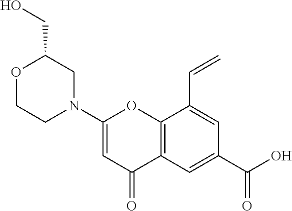 [SMILES: C=Cc1cc(C(=O)O)cc2c(=O)cc(N3CCO[C@H](CO)C3)oc12]

To a solution of methyl 2-[(2S)-2-(hydroxymethyl)morpholin-4-yl]-4-oxo-8-vinyl-chromene-6-carboxylate (4.3 g, 12.45 mmol, 1 eq) in THF (30 mL) was added a solution of LiOH·H₂O (1.57 g, 37.35 mmol, 3 eq) in H₂O (10 mL) at 0° C. The mixture was stirred at 25° C. for half an hour. LC-MS showed the reaction was complete. The mixture was cooled to 0° C. and adjusted to pH=4 with 2N HCl. The aqueous phase was extracted with DCM and i-PrOH (v:v=3:1, 45 mL×5). The combined organic phase was washed with brine (35 mL), dried over anhydrous Na₂SO₄, filtered and concentrated in vacuum. Compound 2-[(2S)-2-(hydroxymethyl)morpholin-4-yl]-4-oxo-8-vinyl-chromene-6-carboxylic acid (2.5 g, 7.55 mmol, 60.60% yield) was obtained as yellow solid. ¹H NMR (DMSO-d₆, 400 MHz) δ 13.25 (s, 1H), 8.40 (s, 1H), 8.33 (s, 1H), 7.15 (dd, J=11.2 Hz, 17.6 Hz, 1H), 6.07 (d, J=17.6 Hz, 1H), 5.59˜5.56 (m, 2H), 3.99˜3.92 (m, 3H), 3.63˜3.61 (m, 2H), 3.55˜3.50 (m, 2H), 3.17 (t, J=11.6 Hz, 1H), 2.94 (t, J=11.6 Hz, 1H).

### Preparation of (S)-2-(2-(hydroxymethyl)morpholino)-N,N-dimethyl-4-oxo-8-vinyl-4H-chromene-6-carboxamide (Step 4 in Scheme 21)

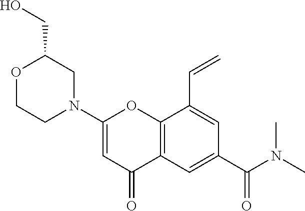 [SMILES: C=Cc1cc(C(=O)N(C)C)cc2c(=O)cc(N3CCO[C@H](CO)C3)oc12]

To a mixture of 2-[(2S)-2-(hydroxymethyl)morpholin-4-yl]-4-oxo-8-vinyl-chromene-6-carboxylic acid (2.5 g, 7.55 mmol, 1 eq) and N-methylmethanamine (1.85 g, 22.64 mmol, 2.07 mL, 3 eq, HCl) in DMF (20 mL) was added DIEA (4.88 g, 37.73 mmol, 6.57 mL, 5 eq) and HATU (4.30 g, 11.32 mmol, 1.5 eq) at 0° C. under N₂. The mixture was stirred at 25° C. for 2 hours. LC-MS showed the reaction was complete. The mixture was poured into ice water (10 mL) and then extracted with DCM and i-PrOH (v:v=3:1, 15 mL×4). The combined organic phase was washed with brine (10 mL), dried with anhydrous Na₂SO₄, filtered and concentrated in vacuum. Compound 2-[(2S)-2-(hydroxymethyl)morpholin-4-yl]-N,N-dimethyl-4-oxo-8-vinyl-chromene-6-carboxamide (5 g, crude) was obtained as yellow oil. ¹H NMR (DMSO-d₆, 400 MHz) δ 7.94 (s, 1H), 7.81 (s, 1H), 7.17 (dd, J=11.2 Hz, 17.6 Hz, 1H), 6.10 (d, J=17.2 Hz, 1H), 5.59˜5.55 (m, 2H), 3.99˜3.91 (m, 3H), 3.63˜3.54 (m, 4H), 3.16˜3.13 (m, 2H), 3.01 (s, 3H), 2.95 (s, 3H).

### Preparation of (S)-8-formyl-2-(2-(hydroxymethyl)morpholino)-N,N-dimethyl-4-oxo-4H-chromene-6-carboxamide (Step 5 in Scheme 21)

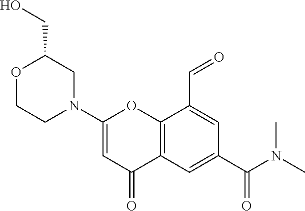 [SMILES: CN(C)C(=O)c1cc(C=O)c2oc(N3CCO[C@H](CO)C3)cc(=O)c2c1]

To a solution of 2-[(2S)-2-(hydroxymethyl)morpholin-4-yl]-N,N-dimethyl-4-oxo-8-vinyl-chromene-6-carboxamide (4.5 g, 12.56 mmol, 1 eq) in THF (30 mL) and H₂O (10 mL) was added K₂OsO₄·2H₂O (462.64 mg, 1.26 mmol, 0.1 eq) at 25° C. and the mixture was stirred at 25° C. for half an hour. Then the mixture was cooled to 0° C. and NaIO₄ (8.06 g, 37.67 mmol, 2.09 mL, 3 eq) was added in portions. The reaction mixture was stirred at 0° C. for half an hour. LCMS showed the reaction was complete. The mixture was quenched with ice water (80 mL) and then extracted with DCM and i-PrOH (v:v=3:1, 50 mL×5). The combined organic phase was washed with brine (30 mL), dried over anhydrous Na₂SO₄, filtered and concentrated in vacuum. Compound 8-formyl-2-[(2S)-2-(hydroxymethyl)morpholin-4-yl]-N,N-dimethyl-4-oxo-chromene-6-carboxamide (2.7 g, crude) was obtained as yellow oil. ¹H NMR (DMSO-d₆, 400 MHz) δ 10.40 (s, 1H), 8.17 (s, 1H), 7.88 (s, 1H), 5.64 (s, 1H), 3.81˜3.74 (m, 2H), 3.51˜3.47 (m, 3H), 3.17 (s, 6H), 2.94˜2.90 (m, 2H), 2.87˜2.83 (m, 2H).

### Preparation of (S)-8-((4,6-difluoroindolin-1-yl)methyl)-2-(2-(hydroxymethyl)morpholino)-N,N-dimethyl-4-oxo-4H-chromene-6-carboxamide (Step 6 in Scheme 21)

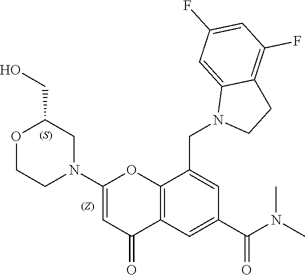

To a mixture of 8-formyl-2-[(2S)-2-(hydroxymethyl)morpholin-4-yl]-N,N-dimethyl-4-oxo-chromene-6-carboxamide (0.2 g, 277.50 umol, 50% purity, 1 eq) and 4,6-difluoroindoline (64.58 mg, 416.25 umol, 1.5 eq) in MeOH (5 mL) was added AcOH (499.93 mg, 8.32 mmol, 476.13 uL, 30 eq). The mixture was stirred at 25° C. for an hour under N₂. The mixture was cooled to 0° C. and NaBH₃CN (34.88 mg, 555.00 umol, 2 eq) was added in portions. The mixture was stirred at 25° C. for an hour. LCMS showed the reaction was complete. The mixture was quenched with ice water (0.5 mL) and then concentrated in vacuum. The residue was purified by prep-HPLC (column: Waters Xbridge BEH C18 100*25 mm*5 um; mobile phase: [water (10 mM NH₄HCO₃)-MeCN]; B %: 20%-55%, 10 mins). The solvent was concentrated in vacuum. Compound 8-[(4,6-difluoroindolin-1-yl)methyl]-2-[(2S)-2-(hydroxymethyl) morpholin-4-yl]-N,N-dimethyl-4-oxo-chromene-6-carboxamide (5.8 mg, 11.02 umol, 3.97% yield, 94.9% purity) was obtained as white solid. ¹H NMR (DMSO-d₆, 400 MHz) δ 7.84 (s, 1H), 7.59 (s, 1H), 6.43 (d, J=10.0 Hz, 1H), 6.31 (t, J=12.8 Hz, 1H), 5.58 (s, 1H), 4.82 (t, J=5.6 Hz, 1H), 4.62 (s, 2H), 4.02˜3.84 (m, 3H), 3.63˜3.42 (m, 5H), 3.37˜3.33 (m, 2H), 3.15˜3.11 (m, 1H), 2.99˜2.92 (m, 6H), 2.90˜2.87 (m, 2H). HPLC: 94.91% (220 nm), 95.24% (254 nm). MS (ESI): mass calcd. For C₂₆H₂₇O₅N₃F₂ 499.19 m/z found 500.2 [M+H]⁺.

### Preparation of (S)-(4-(8-((4,6-difluoroindolin-1-yl)methyl)-6-(dimethylcarbamoyl)-4-oxo-4H-chromen-2-yl)morpholin-2-yl)methyl methanesulfonate (Step 7 in Scheme 21)

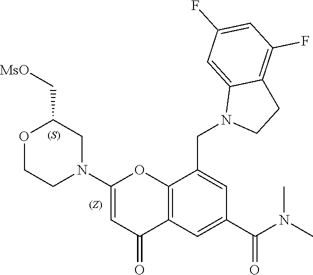

To a mixture of 8-[(4,6-difluoroindolin-1-yl)methyl]-2-[(2S)-2-(hydroxymethyl)morpholin-4-yl]-N,N-dimethyl-4-oxo-chromene-6-carboxamide (150 mg, 300.30 umol, 1 eq) and DIEA (116.43 mg, 900.89 umol, 156.92 uL, 3 eq) in DCM (10 mL) was added methylsulfonyl methanesulfonate (62.77 mg, 360.36 umol, 1.2 eq) in portions at 0° C. under N₂. The mixture was stirred at 25° C. for 3 hours. LCMS showed ˜14% of 8-[(4,6-difluoroindolin-1-yl)methyl]-2-[(2S)-2-(hydroxymethyl)morpholin-4-yl]-N,N-dimethyl-4-oxo-chromene-6-carboxamide was remained and ˜43% of [(2S)-4-[8-[(4,6-difluoroindolin-1-yl) methyl]-6-(dimethyl carbamoyl)-4-oxo-chromen-2-yl]morpholin-2-yl]methyl methanesulfonate was detected. The mixture was quenched with ice water (30 mL) and then extracted with DCM and i-PrOH (v:v=3:1, 15 mL×3). The combined organic phase was washed with brine (10 mL), dried with anhydrous Na₂SO₄, filtered and concentrated in vacuum. The residue was purified by flash silica gel chromatography (Biotage®; 4 g SepaFlash® Silica Flash Column, Eluent of 0˜100% Ethyl acetate/Petroleum ether gradient and then eluent of 0˜₄₀% Methanol/Ethyl acetate gradient @ 50 mL/min). The solvent was concentrated in vacuum. Compound [(2S)-4-[8-[(4,6-difluoroindolin-1-yl)methyl]-6-(dimethylcarbamoyl)-4-oxo-chromen-2-yl]morpholin-2-yl]methyl methanesulfonate (0.1 g, 173.13 umol, 57.65% yield) was obtained as yellow solid. MS (ESI): mass calcd. For C₂₇H₂₉O₇N₃F₂S 577.17 m/z found 578.2 [M+H]⁺.

### Compound 93

### Preparation of (S)-8-((4,6-difluoroindolin-1-yl)methyl)-2-(2-(fluoromethyl)morpholino)-N,N-dimethyl-4-oxo-4H-chromene-6-carboxamide (Step 8 in Scheme 21)

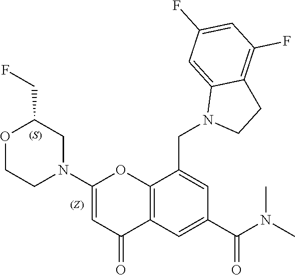

To a solution of [(2S)-4-[8-[(4,6-difluoroindolin-1-yl)methyl]-6-(dimethylcarbamoyl)-4-oxo-chromen-2-yl]morpholin-2-yl]methyl methanesulfonate (0.1 g, 173.13 umol, 1 eq) in CH₃CN (2 mL) was added tetramethylammonium fluoride tetrahydrate (143.01 mg, 865.66 umol, 5 eq) at 80° C. under N₂ and the mixture was stirred at 80° C. for 12 hours. LCMS showed the reaction was complete. The mixture was concentrated under reduced pressure at 45° C. The residue was purified by prep-HPLC (column: Phenomenex C18 80*40 mm*3 um; mobile phase: [water (NH₄HCO₃)-MeCN]; B %: 35%-65%, 8 mins). The solvent was removed freeze drying. Compound 8-[(4,6-difluoroindolin-1-yl)methyl]-2-[(2S)-2-(fluoromethyl)morpholin-4-yl]-N,N-dimethyl-4-oxo-chromene-6-carboxamide (13.5 mg, 25.71 umol, 14.85% yield, 95.5% purity) was obtained as white solid. ¹H NMR (DMSO-d₆, 400 MHz) δ 7.85 (s, 1H), 7.61 (s, 1H), 6.46 (d, J=8.8 Hz, 1H), 6.34˜6.30 (m, 1H), 5.63 (s, 1H), 4.63 (d, J=8.8 Hz, 2H), 4.54˜4.34 (m, 2H), 4.06˜3.92 (m, 3H), 3.88˜3.72 (m, 2H), 3.60 (t, J=8.4 Hz, 1H), 3.46 (t, J=8.4 Hz, 2H), 3.13 (t, J=8.4 Hz 1H), 2.98˜2.90 (m, 8H). HPLC: 95.95% (220 nm), 97.29% (254 nm). MS (ESI): mass calcd. For C₂₆H₂₆O₄N₃F₃ 501.19 m/z found 502.2 [M+H]⁺.

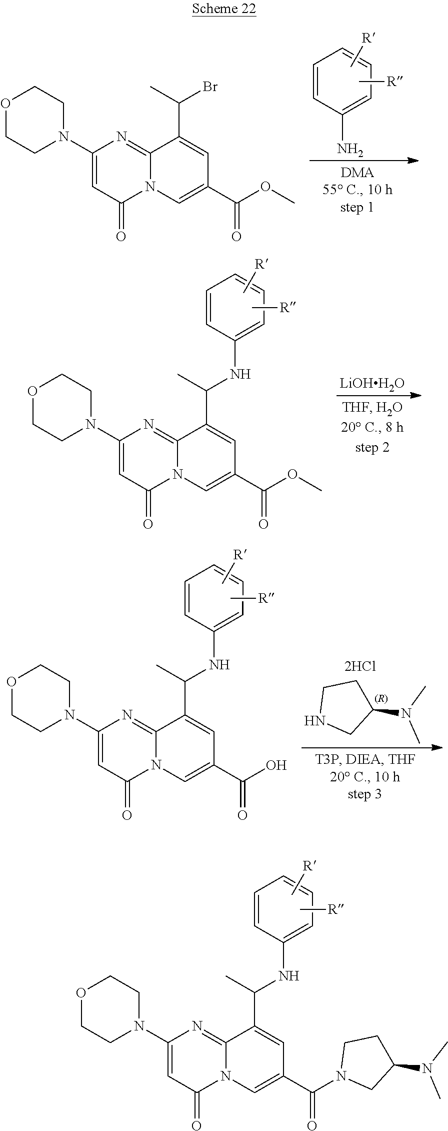

### Compound 94

### Preparation of 9-(1-((3,4-difluorophenyl)amino)ethyl)-7-((R)-3-(dimethylamino)pyrrolidine-1-carbonyl)-2-morpholino-4H-pyrido[1,2-a]pyrimidin-4-one (Step 1-3 in Scheme 13)

### Preparation of methyl 9-(1-((3,4-difluorophenyl)amino)ethyl)-2-morpholino-4-oxo-4H-pyrido[1,2-a]pyrimidine-7-carboxylate (Step 1 in Scheme 22)

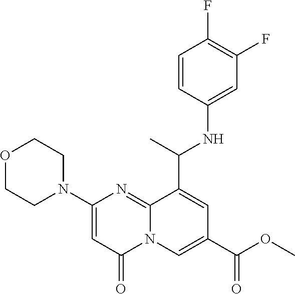 [SMILES: COC(=O)c1cc(C(C)Nc2ccc(F)c(F)c2)c2nc(N3CCOCC3)cc(=O)n2c1]

To A solution of methyl 9-(1-bromoethyl)-2-morpholino-4-oxo-pyrido[1,2-a]pyrimidine-7-carboxylate (0.5 g, 1.26 mmol, 1 eq) and 3,4-difluoroaniline (488.75 mg, 3.79 mmol, 3 eq) in DMA (5 mL) was stirred at 55° C. for 10 hours. TLC (Petroleum ether:Ethyl acetate=1:1, R_{f}=0.26) and LCMS showed the reaction was complete. The mixture was quenched with ice water (20 mL) and extracted with EtOAc (10 mL×3). The combined organic layer was washed with brine (5 mL×4), dried over Na₂SO₄, filtered and concentrated under reduced pressure. Compound methyl 9-[1-(3,4-difluoroanilino)ethyl]-2-morpholino-4-oxo-pyrido[1,2-a] pyrimidine-7-carboxylate (0.9 g, crude) was obtained as yellow solid. ¹H NMR (DMSO-d₆, 400 MHz) δ 9.23 (s, 1H), 8.00 (s, 1H), 7.05˜7.00 (m, 1H), 6.71 (d, J=6.8 Hz, 1H), 6.49˜6.46 (m, 1H), 6.31-6.29 (m, 1H), 5.72 (s, 1H), 5.00 (t, J=6.8 Hz, 1H), 3.85 (s, 3H), 3.70 (s, 8H), 1.46 (d, J=6.8 Hz, 3H).

### Preparation of 9-(1-((3,4-difluorophenyl)amino)ethyl)-2-morpholino-4-oxo-4H-pyrido[1,2-a]pyrimidine-7-carboxylic acid (Step 2 in Scheme 22)

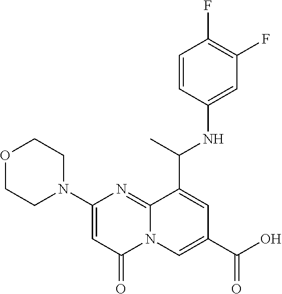 [SMILES: CC(Nc1ccc(F)c(F)c1)c1cc(C(=O)O)cn2c(=O)cc(N3CCOCC3)nc12]

A solution of methyl 9-[1-(3,4-difluoroanilino)ethyl]-2-morpholino-4-oxo-pyrido[1,2-a]pyrimidine-7-carboxylate (0.9 g, 1.01 mmol, 50% purity, 1 eq) and LiOH·H₂O (127.46 mg, 3.04 mmol, 3 eq) in H₂O (5 mL) and THF (5 mL) was stirred at 20° C. for an hour. TLC (petroleum ether:EtOAc=0:1, R_{f}=0.05) and LCMS showed the reaction was complete. The organic solvent was removed under reduced pressure. 10 mL of water was added and the aqueous was extracted with MTBE (5 mL×3) to remove the impurity. The aqueous was adjusted to pH=4 with 1N HCl at 0° C. and there was some solid formed. After filtration, the solid was collected and concentrated under reduced pressure. Compound 9-[1-(3,4-difluoroanilino) ethyl]-2-morpholino-4-oxo-pyrido[1,2-a]pyrimidine-7-carboxylic acid (0.35 g, 813.19 umol, 80.31% yield) was obtained as pale yellow solid. ¹H NMR (DMSO-d₆, 400 MHz) δ 9.20 (s, 1H), 8.02 (s, 1H), 7.08˜7.00 (m, 1H), 6.64 (d, J=6.8 Hz, 1H), 6.41˜6.36 (m, 1H), 6.21˜6.16 (m, 1H), 5.70 (s, 1H), 5.00 (t, J=6.8 Hz, 1H), 3.70 (s, 8H), 1.46 (d, J=6.8 Hz, 3H).

### Preparation of 9-(1-((3,4-difluorophenyl)amino)ethyl)-7-((R)-3-(dimethylamino)pyrrolidine-1-carbonyl)-2-morpholino-4H-pyrido[1,2-a]pyrimidin-4-one (Step 3 in Scheme 22)

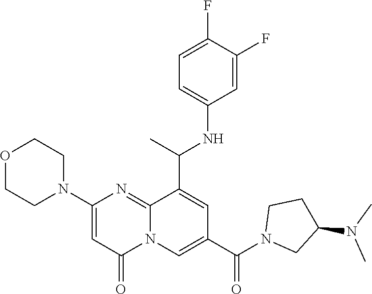 [SMILES: CC(Nc1ccc(F)c(F)c1)c1cc(C(=O)N2CC[C@@H](N(C)C)C2)cn2c(=O)cc(N3CCOCC3)nc12]

To a solution of 9-[1-(3,4-difluoroanilino)ethyl]-2-morpholino-4-oxo-pyrido[1,2-a]pyrimidine-7-carboxylic acid (0.15 g, 348.51 umol, 1 eq), (3R)—N,N-dimethylpyrrolidin-3-amine (97.81 mg, 522.76 umol, 108.68 uL, 1.5 eq, 2HCl) and DIEA (225.21 mg, 1.74 mmol, 303.51 uL, 5 eq) in THF (2 mL) was added T3P (332.67 mg, 522.76 umol, 310.90 uL, 50% purity, 1.5 eq) dropwise at 0° C. Then the mixture was stirred at 20° C. for 10 hours. HPLC and LCMS showed the reaction was complete. The mixture was quenched with ice water (3 mL) and the organic solvent was removed under reduced pressure. The aqueous was extracted with DCM and i-PrOH (v:v=3:1, 2 mL×3). The combined organic layer was washed with brine (2 mL), dried over Na₂SO₄, filtered and concentrated under reduced pressure. The residue was purified by prep-HPLC (column: Phenomenex Gemini-NX 80*40 mm*3 um; mobile phase: [water (10 mM NH₄HCO₃)-MeCN]; B %: 15%-45%, 8 mins). The eluent was removed under freeze drying. Compound 9-[1-(3,4-difluoroanilino)ethyl]-7-[(3R)-3-(dimethylamino)pyrrolidine-1-carbonyl]-2-morpholino-pyrido [1,2-a]pyrimidin-4-one (96.4 mg, 180.25 umol, 51.72% yield, 98.46% purity) was obtained as white solid. ¹H NMR (DMSO-d₆, 400 MHz) δ 8.86 (s, 1H), 7.80 (s, 1H), 7.06˜7.02 (m, 1H), 6.61˜6.54 (m, 1H), 6.42˜6.39 (m, 1H), 6.19˜6.14 (m, 1H), 5.71 (s, 1H), 5.06˜5.01 (m, 1H), 3.70 (s, 8H), 3.40˜3.33 (m, 2H), 3.20˜3.03 (m, 2H), 2.69˜2.61 (m, 1H), 2.15˜1.94 (m, 7H), 1.68˜1.63 (m, 1H), 1.47 (d, J=6.0 Hz, 3H). HPLC: 98.46% (220 nm), 100% (254 nm). MS (ESI): mass calcd. For C₂₇H₃₂F₂N₆O₃ 526.25 m/z found 527.3 [M+H]⁺.

### Compound 95

### Preparation of 9-(1-((2,3-difluorophenyl)amino)ethyl)-7-((R)-3-(dimethylamino)pyrrolidine-1-carbonyl)-2-morpholino-4H-pyrido[1,2-a]pyrimidin-4-one (Step 1 in Scheme 22)

### Preparation of methyl 9-(1-((2,3-difluorophenyl)amino)ethyl)-2-morpholino-4-oxo-4H-pyrido[1,2-a]pyrimidine-7-carboxylate (Step 2 in Scheme 22)

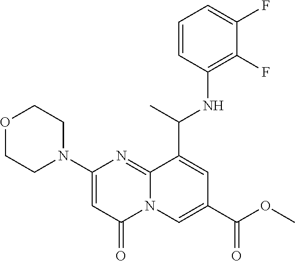 [SMILES: COC(=O)c1cc(C(C)Nc2cccc(F)c2F)c2nc(N3CCOCC3)cc(=O)n2c1]

To a solution of methyl 9-(1-bromoethyl)-2-morpholino-4-oxo-pyrido[1,2-a]pyrimidine-7-carboxylate (0.3 g, 757.13 umol, 1 eq) in DMA (3 mL) was added 2,3-difluoroaniline (293.25 mg, 2.27 mmol, 230.91 uL, 3 eq). The mixture was stirred at 55° C. for 10 hours. LCMS showed the reaction was complete. The mixture was quenched with ice water (2 mL) slowly at 0° C. and there was some precipitate formed. The precipitate was collected after filtration. Compound methyl 9-[1-(2,3-difluoroanilino)ethyl]-2-morpholino-4-oxo-pyrido[1,2-a]pyrimidine-7-carboxylate (203 mg, 456.76 umol, 60.33% yield) was obtained as white solid. ¹H NMR (DMSO-d₆, 400 MHz) δ 9.23 (s, 1H), 8.11 (s, 1H), 6.83˜6.77 (m, 1H), 6.62 (d, J=7.6 Hz, 1H), 6.54˜6.48 (m, 1H), 6.15 (t, J=8.0 Hz, 1H), 5.73 (s, 1H), 5.17 (t, J=7.2 Hz, 1H), 3.85 (s, 3H), 3.70 (s, 8H), 1.53 (d, J=6.4 Hz, 3H).

### Preparation of 9-(1-((2,3-difluorophenyl)amino)ethyl)-2-morpholino-4-oxo-4H-pyrido[1,2-a]pyrimidine-7-carboxylic acid (Step 2 in Scheme 22)

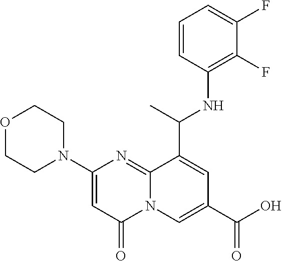 [SMILES: CC(Nc1cccc(F)c1F)c1cc(C(=O)O)cn2c(=O)cc(N3CCOCC3)nc12]

To a solution of methyl 9-[1-(2,3-difluoroanilino)ethyl]-2-morpholino-4-oxo-pyrido[1,2-a] pyrimidine-7-carboxylate (200 mg, 450.01 umol, 1 eq) in THF (1 mL) and H₂O (0.5 mL) was added LiOH·H₂O (56.65 mg, 1.35 mmol, 3 eq). The mixture was stirred at 20° C. for 8 hours and the reaction mixture became clear solution. LCMS and TLC (Petroleum ether:Ethyl acetate=0:1, R_{f}=0) showed the reaction was complete. The mixture was adjusted to pH=4 with HCl (2M) at 0° C. and there was some precipitate formed. The precipitate was collected after filtration. Compound 9-[1-(2,3-difluoroanilino)ethyl]-2-morpholino-4-oxo-pyrido[1,2-a] pyrimidine-7-carboxylic acid (173 mg, 401.95 umol, 89.32% yield) was obtained as white solid. ¹H NMR (DMSO-d₆, 400 MHz) δ 13.54 (s, 1H), 9.20 (s, 1H), 8.11 (s, 1H), 6.81-6.77 (m, 1H), 6.58 (d, J=7.6 Hz, 1H), 6.54˜6.50 (m, 1H), 6.15 (t, J=7.6 Hz, 1H), 5.71 (s, 1H), 5.17 (t, J=6.4 Hz, 1H), 3.69 (s, 8H), 1.53 (d, J=6.4 Hz, 3H).

### Preparation of 9-(1-((2,3-difluorophenyl)amino)ethyl)-7-((R)-3-(dimethylamino)pyrrolidine-1-carbonyl)-2-morpholino-4H-pyrido[1,2-a]pyrimidin-4-one (Step 3 in Scheme 13)

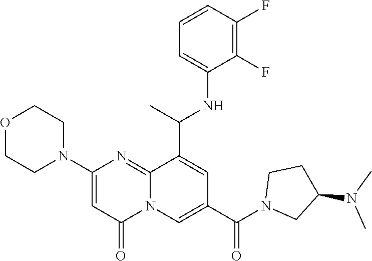 [SMILES: CC(Nc1cccc(F)c1F)c1cc(C(=O)N2CC[C@@H](N(C)C)C2)cn2c(=O)cc(N3CCOCC3)nc12]

To a solution of 9-[1-(2,3-difluoroanilino)ethyl]-2-morpholino-4-oxo-pyrido[1,2-a]pyrimidine-7-carboxylic acid (100 mg, 232.34 umol, 1 eq) and (3R)—N,N-dimethylpyrrolidin-3-amine dihydrochloride (130.42 mg, 697.02 umol, 3 eq) in THF (1 mL) was added DIEA (180.17 mg, 1.39 mmol, 242.81 uL, 6 eq). Then T3P (221.78 mg, 348.51 umol, 207.27 uL, 50% purity, 1.5 eq) was added dropwise at 0° C. under N₂. The reaction mixture was stirred at 20° C. for 10 hours. LCMS and HPLC showed the reaction was complete. The mixture was quenched with ice water (0.1 mL) and concentrated in vacuum. The residue was purified by prep-HPLC (column: Waters Xbridge BEH C18 100*30 mm*10 um; mobile phase: [water (10 mM NH₄HCO₃)-MeCN]; B %: 20%-50%, 10 mins). The solvent was removed under freeze drying. Compound 9-[1-(2,3-difluoroanilino)ethyl]-7-[(3R)-3-(dimethylamino)pyrrolidine-1-carbonyl]-2-morpholino-pyrido [1,2-a]pyrimidin-4-one (44.7 mg, 84.36 umol, 36.31% yield, 99.38% purity) was obtained as white solid. ¹H NMR (DMSO-d₆, 400 MHz) δ 8.86 (s, 1H), 7.95˜7.90 (m, 1H), 6.79 (s, 1H), 6.57˜6.49 (m, 2H), 6.18 (t, J=1.0 Hz, 1H), 5.71 (s, 1H), 5.19 (t, J=6.8 Hz, 1H), 3.69 (8 s, 8H), 3.52˜3.48 (m, 1H), 3.40 (s, 2H), 3.25˜3.12 (m, 1H), 2.69 (d, J=7.2 Hz, 1H), 2.15 (s, 3H), 2.07˜2.04 (m, 3H), 1.97˜1.93 (m, 1H), 1.70˜1.65 (m, 1H), 1.55 (d, J=6.4 Hz, 3H). MS: 99.38% (220 nm), 100.00% (254 nm). MS (ESI): mass calcd. For C₂₇H₃₂F₂N₆O₃ 526.25 m/z found 527.3 [M+H]⁺.

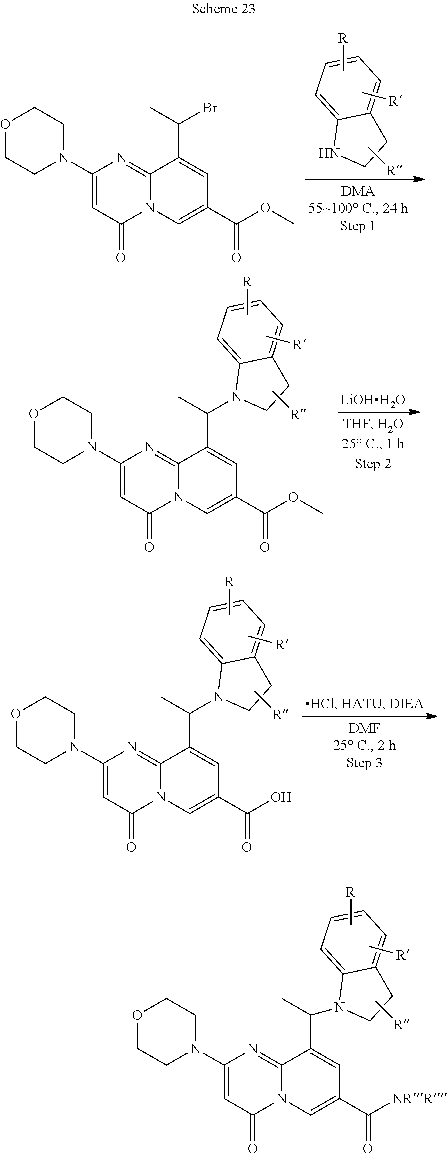

### Compound 96

### Preparation of 9-(1-(4,6-difluoro-2-methylindolin-1-yl)ethyl)-N,N-dimethyl-2-morpholino-4-oxo-4H-pyrido[1,2-a]pyrimidine-7-carboxamide (Step 1-3 in Scheme 23)

### Preparation of methyl 9-(1-(4,6-difluoro-2-methylindolin-1-yl)ethyl)-2-morpholino-4-oxo-4H-pyrido[1,2-a]pyrimidine-7-carboxylate (Step 1 in Scheme 23)

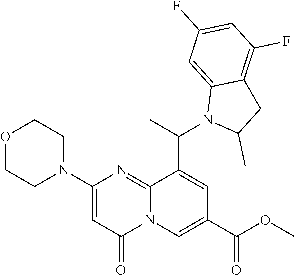 [SMILES: COC(=O)c1cc(C(C)N2c3cc(F)cc(F)c3CC2C)c2nc(N3CCOCC3)cc(=O)n2c1]

A mixture of methyl 9-(1-bromoethyl)-2-morpholino-4-oxo-pyrido[1,2-a]pyrimidine-7-carboxylate (0.5 g, 1.26 mmol, 1 eq) and 4,6-difluoro-2-methyl-indoline (213.47 mg, 1.26 mmol, 1 eq) in DMA (10 mL) was stirred at 55° C. for 12 hours. The mixture was then stirred at 100° C. for 12 hours. LC-MS showed methyl 9-(1-bromoethyl)-2-morpholino-4-oxo-pyrido[1,2-a]pyrimidine-7-carboxylate was consumed completely. The mixture was quenched with water (50 mL) and extracted with ethyl acetate (30 mL×3). The combined organic phase was washed with brine (20 mL×2), dried with anhydrous Na₂SO₄, filtered and concentrated in vacuum. The residue was purified by column chromatography (SiO₂, Petroleum ether/Ethyl acetate˜Ethyl acetate:Methanol=30/1 to 4/1). The solvent was concentrated in vacuum. Compound methyl 9-[1-(4,6-difluoro-2-methyl-indolin-1-yl)ethyl]-2-morpholino-4-oxo-pyrido[1,2-a]pyrimidine-7-carboxylate (0.36 g, 743.04 umol, 58.88% yield) was obtained as yellow solid. ¹H NMR (DMSO-d₆, 400 MHz) δ 9.28 (s, 1H), 8.04 (s, 1H), 6.26˜6.13 (m, 2H), 5.92 (s, 1H), 5.20˜5.07 (m, 1H), 4.31-4.27 (m, 1H), 3.90 (s, 3H), 3.68˜3.65 (m, 6H), 3.48˜3.43 (m, 4H), 1.62 (d, J=7.2 Hz, 3H), 1.17 (d, J=6.0 Hz, 3H).

### Preparation of 9-(1-(4,6-difluoro-2-methylindolin-1-yl)ethyl)-2-morpholino-4-oxo-4H-pyrido[1,2-a]pyrimidine-7-carboxylic acid (Step 2 in Scheme 23)

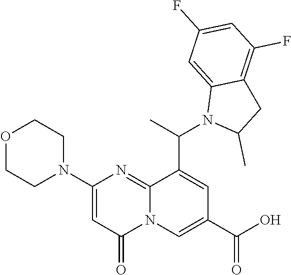 [SMILES: CC1Cc2c(F)cc(F)cc2N1C(C)c1cc(C(=O)O)cn2c(=O)cc(N3CCOCC3)nc12]

To a solution of methyl 9-[1-(4,6-difluoro-2-methyl-indolin-1-yl)ethyl]-2-morpholino-4-oxo-pyrido[1,2-a]pyrimidine-7-carboxylate (80 mg, 165.12 umol, 1 eq) in THF (8 mL) and H₂O (8 mL) was added LiOH·H₂O (20.79 mg, 495.36 umol, 3 eq). The mixture was stirred at 25° C. for an hour. LC-MS showed methyl 9-[1-(4,6-difluoro-2-methyl-indolin-1-yl)ethyl]-2-morpholino-4-oxo-pyrido[1,2-a]pyrimidine-7-carboxylate was consumed completely. The mixture was concentrated under reduced pressure at 45° C. Water (10 mL) was added to the residue and adjusted to pH=4 with 2M HCl. The white precipitate was formed. After filtration, compound 9-[1-(4,6-difluoro-2-methyl-indolin-1-yl)ethyl]-2-morpholino-4-oxo-pyrido[1,2-a] pyrimidine-7-carboxylic acid (50 mg, 106.28 umol, 64.36% yield) was obtained as a off-white solid. ¹H NMR (DMSO-d₆, 400 MHz) δ 13.57 (s, 1H), 9.26 (s, 1H), 8.06 (s, 1H), 6.25˜6.11 (m, 2H), 5.91 (s, 1H), 5.21˜5.07 (m, 1H), 4.32˜4.29 (m, 1H), 3.67˜3.64 (m, 6H), 3.48˜3.42 (m, 4H), 1.63 (d, J=7.2 Hz, 3H), 1.16 (d, J=6.0 Hz, 3H).

### Compound 97

### Preparation of 9-(1-(4,6-difluoro-2-methylindolin-1-yl)ethyl)-N,N-dimethyl-2-morpholino-4-oxo-4H-pyrido[1,2-a]pyrimidine-7-carboxamide (Step 3 in Scheme 23)

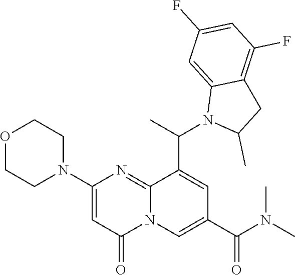 [SMILES: CC1Cc2c(F)cc(F)cc2N1C(C)c1cc(C(=O)N(C)C)cn2c(=O)cc(N3CCOCC3)nc12]

To a mixture of 9-[1-(4,6-difluoro-2-methyl-indolin-1-yl)ethyl]-2-morpholino-4-oxo-pyrido [1,2-a]pyrimidine-7-carboxylic acid (50 mg, 106.28 umol, 1 eq) and N-methylmethanamine (2 M, 192.23 uL, 3.62 eq) in DMF (2 mL) was added DIEA (68.68 mg, 531.39 umol, 92.56 uL, 5 eq) and HATU (60.61 mg, 159.42 umol, 1.5 eq) at 0° C. under N₂. The mixture was stirred at 25° C. for 2 hours. LC-MS showed the reaction was complete. The solution was purified by prep-HPLC (column: Waters Xbridge BEH C18 100*25 mm*5 um; mobile phase: [water (10 mM NH₄HCO₃)-MeCN]; B %: 40%-70%, 10 mins). The solvent was removed under freeze drying. Compound 9-[1-(4,6-difluoro-2-methyl-indolin-1-yl)ethyl]-N,N-dimethyl-2-morpholino-4-oxo-pyrido[1,2-a]pyrimidine-7-carboxamide (15.1 mg, 29.65 umol, 27.90% yield, 97.71% purity) was obtained as yellow solid. ¹H NMR (DMSO-d₆, 400 MHz) δ 8.80 (s, 1H), 7.85 (s, 1H), 6.20˜5.99 (m, 2H), 5.65 (s, 1H), 5.12˜5.10 (m, 1H), 4.30˜4.27 (m, 1H), 3.64˜3.61 (m, 6H), 3.48˜3.39 (m, 4H), 2.98 (s, 6H), 1.65˜1.58 (m, 3H), 1.16˜1.12 (m, 3H). HPLC: 97.71% (220 nm), 98.55% (254 nm). MS (ESI): mass calcd. For C₂₆H₂₉O₃N₅F₂ 497.22 m/z found 498.2 [M+H]⁺.

### Preparation of 9-(1-(7-chloroindolin-1-yl)ethyl)-7-((R)-3-(dimethylamino)pyrrolidine-1-carbonyl)-2-morpholino-4H-pyrido[1,2-a]pyrimidin-4-one (Step 1-3 in Scheme 23)

### Preparation of methyl 9-(1-(7-chloroindolin-1-yl)ethyl)-2-morpholino-4-oxo-4H-pyrido[1,2-a]pyrimidine-7-carboxylate (Step 1 in Scheme 23)

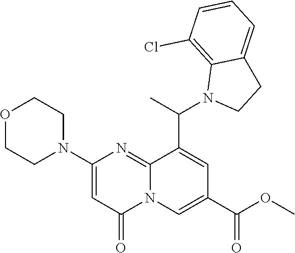 [SMILES: COC(=O)c1cc(C(C)N2CCc3cccc(Cl)c32)c2nc(N3CCOCC3)cc(=O)n2c1]

A solution of methyl 9-(1-bromoethyl)-2-morpholino-4-oxo-pyrido[1,2-a]pyrimidine-7-carboxylate (0.2 g, 504.75 umol, 1 eq) and 7-chloroindoline (100.79 mg, 656.18 umol, 1.3 eq) in DMA (3 mL) was stirred at 55° C. for 10 hours. Then the mixture was stirred at 70° C. for 10 hours. TLC (Petroleum ether:Ethyl acetate=1:1, R_{f}=0.26) and LCMS showed the reaction was nearly complete. The mixture was quenched with ice water (20 mL) and extracted with EtOAc (10 mL×3). The combined organic layer was washed with brine (5 mL×4), dried over Na₂SO₄, filtered and concentrated under reduced pressure. Compound methyl 9-[1-(7-chloroindolin-1-yl)ethyl]-2-morpholino-4-oxo-pyrido[1,2-a]pyrimidine-7-carboxylate (0.2 g, 426.50 umol, 84.50% yield) was obtained as brown solid. ¹H NMR (DMSO-d₆, 400 MHz) δ 9.29 (s, 1H), 7.98 (s, 1H), 6.99˜6.93 (m, 2H), 6.56˜6.51 (m, 1H), 6.32˜6.30 (m, 1H), 5.67 (s, 1H), 3.90 (s, 3H), 3.62˜3.56 (m, 8H), 3.54 (t, J=8.8 Hz, 2H), 3.00 (t, J=8.8 Hz, 2H), 1.55 (d, J=6.4 Hz, 3H).

### Preparation of 9-(1-(7-chloroindolin-1-yl)ethyl)-2-morpholino-4-oxo-4H-pyrido[1,2-a]pyrimidine-7-carboxylic acid (Step 2 in Scheme 23)

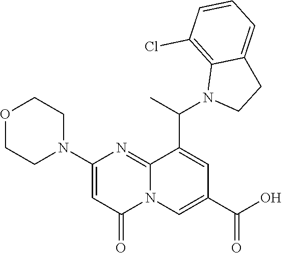 [SMILES: CC(c1cc(C(=O)O)cn2c(=O)cc(N3CCOCC3)nc12)N1CCc2cccc(Cl)c21]

A solution of methyl 9-[1-(7-chloroindolin-1-yl)ethyl]-2-morpholino-4-oxo-pyrido[1,2-a]pyrimidine-7-carboxylate (0.2 g, 426.50 umol, 1 eq) and LiOH·H₂O (53.69 mg, 1.28 mmol, 3 eq) in THF (3 mL) and H₂O (3 mL) was stirred at 20° C. for an hour. TLC (petroleum ether:EtOAc=0:1, R_{f}=0.05) and LCMS showed the reaction was complete. The organic solvent was removed under reduced pressure. 10 mL of water was added and the aqueous was extracted with MTBE (5 mL×3) to remove the impurity. The aqueous was made pH=4 with 1N HCl at 0° C. and there was some solid formed. After filtration, the solid was collected and concentrated under reduced pressure. Compound 9-[1-(7-chloroindolin-1-yl)ethyl]-2-morpholino-4-oxo-pyrido[1,2-a]pyrimidine-7-carboxylic acid (0.13 g, 285.77 umol, 67.00% yield) was obtained as pale yellow solid. ¹H NMR (DMSO-d₆, 400 MHz) δ 13.55 (s, 1H), 9.27 (s, 1H), 7.99 (s, 1H), 7.01-6.93 (m, 2H), 6.54 (t, J=7.6 Hz, 1H), 6.32 (t, J=6.4 Hz, 1H), 5.75 (s, 1H), 3.76˜3.64 (m, 10H), 2.89 (t, J=6.4 Hz, 2H), 1.55 (d, J=6.8 Hz, 3H).

### Preparation of 9-(1-(7-chloroindolin-1-yl)ethyl)-7-((R)-3-(dimethylamino)pyrrolidine-1-carbonyl)-2-morpholino-4H-pyrido[1,2-a]pyrimidin-4-one (Step 3 in Scheme 23)

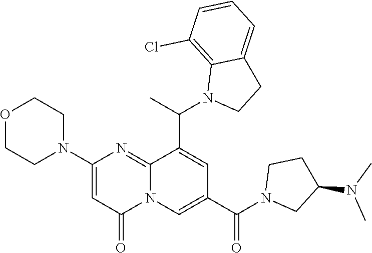 [SMILES: CC(c1cc(C(=O)N2CC[C@@H](N(C)C)C2)cn2c(=O)cc(N3CCOCC3)nc12)N1CCc2cccc(Cl)c21]

To a solution of 9-(1-(7-chloroindolin-1-yl)ethyl)-2-morpholino-4-oxo-4H-pyrido[1,2-a]pyrimidine-7-carboxylic acid (0.13 g, 285.77 umol, 1 eq), (3R)—N,N-dimethylpyrrolidin-3-amine (80.21 mg, 428.66 umol, 89.12 uL, 1.5 eq, 2HCl) and DIEA (184.67 mg, 1.43 mmol, 248.88 uL, 5 eq) in THF (2 mL) was added T3P (272.78 mg, 428.66 umol, 254.94 uL, 50% purity, 1.5 eq) dropwise at 0° C. Then the mixture was stirred at 20° C. for 5 hours. HPLC and LCMS showed the reaction was complete. The mixture was quenched with ice water (3 mL) and the organic solvent was removed under reduced pressure. The aqueous was extracted with DCM and i-PrOH (v:v=3:1, 2 mL×3). The combined organic layer was washed with brine (2 mL), dried over Na₂SO₄, filtered and concentrated under reduced pressure. The residue was purified by prep-HPLC (column: Phenomenex Gemini-NX 80*40 mm*3 um; mobile phase: [water (10 mM NH₄HCO₃)-MeCN]; B %: 22%-52%, 8 mins). The eluent was removed under freeze drying. Compound 9-(1-(7-chloroindolin-1-yl)ethyl)-7-((R)-3-(dimethylamino) pyrrolidine-1-carbonyl)-2-morpholino-4H-pyrido[1,2-a]pyrimidin-4-one (57 mg, 98.09 umol, 34.32% yield, 94.83% purity) was obtained as pale yellow solid. ¹H NMR (DMSO-d₆, 400 MHz) δ 8.90 (s, 1H), 7.88˜7.76 (m, 1H), 6.96˜6.92 (m, 2H), 6.54 (t, J=7.6 Hz, 1H), 6.36˜6.31 (m, 1H), 5.64 (s, 1H), 3.69˜3.55 (m, 12H), 3.30˜3.28 (m, 2H), 2.92˜2.89 (m, 2H), 2.54˜2.48 (m, 1H), 2.18˜1.95 (m, 7H), 1.75-1.68 (m, 1H), 1.55 (d, J=6.8 Hz, 3H). HPLC: 94.83% (220 nm), 97.70% (254 nm). MS (ESI): mass calcd. For C₂₉H₃₅ClN₆O₃ 550.25 m/z found 551.3 [M+H]⁺.

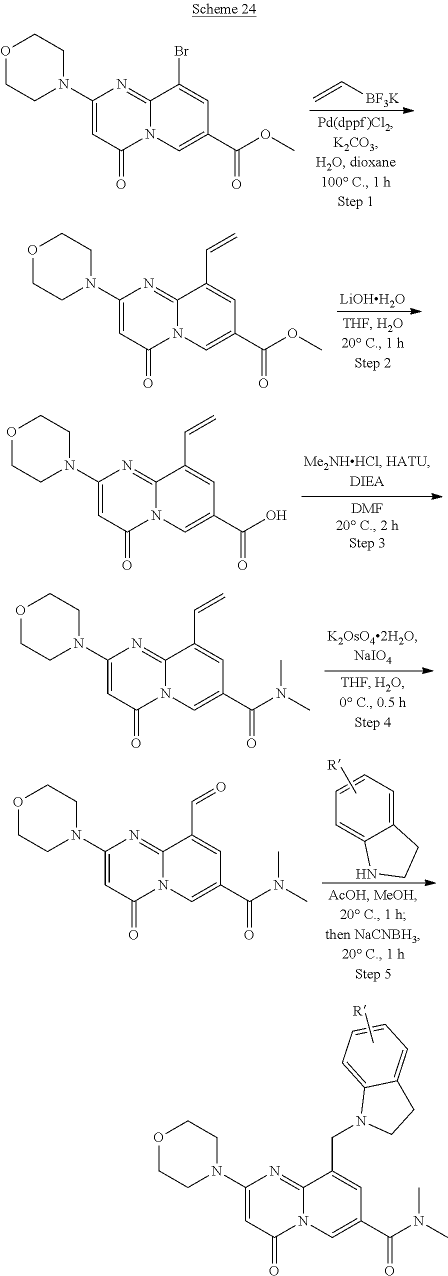

### Compound 98

### General Procedures for Preparing Compounds in Scheme 24

### Preparation of methyl 2-morpholino-4-oxo-9-vinyl-4H-pyrido[1,2-a]pyrimidine-7-carboxylate (Step 1 in Scheme 24)

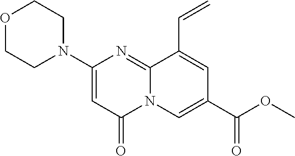 [SMILES: C=Cc1cc(C(=O)OC)cn2c(=O)cc(N3CCOCC3)nc12]

A mixture of methyl 9-bromo-2-morpholino-4-oxo-pyrido[1,2-a]pyrimidine-7-carboxylate (5 g, 13.58 mmol, 1 eq), potassium hydride trifluoro(vinyl)boron (2.73 g, 20.37 mmol, 1.5 eq), Pd(dppf)Cl₂ (496.84 mg, 679.01 umol, 0.05 eq) and K₂CO₃ (3.75 g, 27.16 mmol, 2 eq) in dioxane (50 mL) and H₂O (5 mL) was degassed and purged with N₂ for 3 times and then the mixture was stirred at 100° C. for an hour under N₂ atmosphere. LC-MS showed the reaction was complete. To the mixture was added DCM (80 mL) and water (80 mL), and then filtered through a pad of the Celite. The organic layer was separated and the aqueous was extracted with DCM (60 mL×2). The combined organic layer was washed with brine (100 mL×1), dried over Na₂SO₄, filtered and concentrated under reduced pressure. The residue was triturated with DCM (3 mL) and EtOAc (30 mL), then dried under reduced pressure to give 2.7 g of methyl 2-morpholino-4-oxo-9-vinyl-pyrido[1,2-a]pyrimidine-7-carboxylate. The filtrate was concentrated and the residue was further purified by flash silica gel chromatography (Biotage®; 20 g SepaFlash® Silica Flash Column, Eluent of 0˜100% (Ethyl acetate:DCM=4:1)/Petroleum ether gradient @50 mL/min). The eluent was removed under reduced pressure to give 0.26 g of methyl 2-morpholino-4-oxo-9-vinyl-pyrido[1,2-a]pyrimidine-7-carboxylate. Compound methyl 2-morpholino-4-oxo-9-vinyl-pyrido[1,2-a]pyrimidine-7-carboxylate (2.96 g, 9.37 mmol, 69.03% yield) was obtained as yellow solid. ¹H NMR (DMSO-d₆, 400 MHz) δ 9.24 (s, 1H), 8.21 (s, 1H), 7.28 (dd, J=6.8 Hz, 11.2 Hz, 1H), 6.14 (d, J=18.0 Hz, 1H), 5.68 (s, 1H), 5.55 (d, J=11.2 Hz, 1H), 3.91 (s, 3H), 3.70˜3.56 (m, 8H).

### Preparation of 2-morpholino-4-oxo-9-vinyl-4H-pyrido[1,2-a]pyrimidine-7-carboxylic acid (Step 2 in Scheme 24)

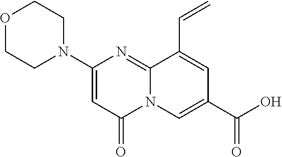 [SMILES: C=Cc1cc(C(=O)O)cn2c(=O)cc(N3CCOCC3)nc12]

To a solution of methyl 2-morpholino-4-oxo-9-vinyl-pyrido[1,2-a]pyrimidine-7-carboxylate (1.9 g, 6.03 mmol, 1 eq) in H₂O (10 mL) and THF (10 mL) was added LiOH·H₂O (1.01 g, 24.10 mmol, 4 eq). The mixture was stirred at 20° C. for an hour. TLC (EtOAc:MeOH=20:1, R_{f}=0.05) and LCMS showed the reaction was complete. The organic solvent was removed under reduced pressure. The aqueous was made pH=4 with 2N HCl at 0° C. and there was some yellow solid formed. After filtration, the solid was collected and concentrated under reduced pressure. Compound 2-morpholino-4-oxo-9-vinyl-pyrido[1,2-a]pyrimidine-7-carboxylic acid (1.7 g, 5.64 mmol, 93.64% yield) was obtained as pale yellow solid. ¹H NMR (DMSO-d₆, 400 MHz) δ 9.25 (s, 1H), 8.23 (s, 1H), 7.31 (dd, J=6.0 Hz, 11.2 Hz, 1H), 6.14 (d, J=17.6 Hz, 1H), 5.69 (s, 1H), 5.55 (d, J=11.2 Hz, 1H), 3.70˜3.66 (m, 8H).

### Preparation of N,N-dimethyl-2-morpholino-4-oxo-9-vinyl-4H-pyrido[1,2-a]pyrimidine-7-carboxamide (Step 3 in Scheme 24)

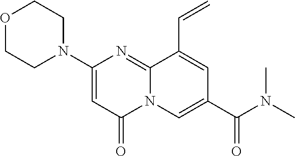 [SMILES: C=Cc1cc(C(=O)N(C)C)cn2c(=O)cc(N3CCOCC3)nc12]

To a solution of 2-morpholino-4-oxo-9-vinyl-pyrido[1,2-a]pyrimidine-7-carboxylic acid (1.7 g, 5.64 mmol, 1 eq), N-methyl methanamine (1.38 g, 16.93 mmol, 1.55 mL, 3 eq, HCl) and DIEA (3.65 g, 28.21 mmol, 4.91 mL, 5 eq) in DMF (15 mL) was added HATU (2.79 g, 7.33 mmol, 1.3 eq) in portions at 0° C. Then the mixture was stirred at 20° C. for 2 hours. TLC (EtOAc:MeOH=20:1, R_{f}=0.1) and LCMS showed the reaction was complete. The mixture was quenched with ice water (50 mL) and the mixture was extracted with DCM and i-PrOH (v:v=3:1, 30 mL×4). The combined organic layer was washed with brine (20 mL×1), dried over Na₂SO₄, filtered and concentrated under reduced pressure. The residue was purified by flash silica gel chromatography (Biotage®; 40 g SepaFlash® Silica Flash Column, Eluent of 0˜23% MeOH/Ethyl acetate gradient @30 mL/min.). The eluent was removed under reduced pressure. Compound N,N-dimethyl-2-morpholino-4-oxo-9-vinyl-pyrido[1,2-a]pyrimidine-7-carboxamide (1.5 g, 4.57 mmol, 80.96% yield) was obtained as pale yellow solid. ¹H NMR (DMSO-d₆, 400 MHz) δ 8.78 (s, 1H), 8.07 (s, 1H), 7.34 (dd, J=6.8 Hz, 10.8 Hz, 1H), 6.15 (d, J=18.0 Hz, 1H), 5.68 (s, 1H), 5.54 (d, J=12.0 Hz, 1H), 3.70˜3.66 (m, 8H), 3.02 (s, 6H).

### Preparation of 9-formyl-N,N-dimethyl-2-morpholino-4-oxo-4H-pyrido[1,2-a]pyrimidine-7-carboxamide (Step 4 in Scheme 24)

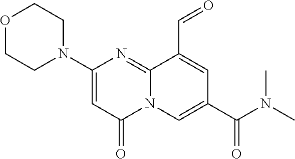 [SMILES: CN(C)C(=O)c1cc(C=O)c2nc(N3CCOCC3)cc(=O)n2c1]

A mixture of N,N-dimethyl-2-morpholino-4-oxo-9-vinyl-pyrido[1,2-a]pyrimidine-7-carboxamide (1.4 g, 4.26 mmol, 1 eq) and K₂OsO₄·2H₂O (157.09 mg, 426.35 umol, 0.1 eq) in THF (20 mL) and H₂O (20 mL) was stirred at 20° C. for half an hour. Then NaIO₄ (2.74 g, 12.79 mmol, 708.75 uL, 3 eq) was added in portions at 0° C. and the mixture was stirred at 0° C. for half an hour. TLC (EtOAc:MeOH=20:1, R_{f}=0.12) and LCMS showed the reaction was complete. The mixture was extracted with EtOAc (20 mL×4). The combined organic layer was washed with brine (15 mL×2), dried over Na₂SO₄, filtered and concentrated under reduced pressure. Then the aqueous was extracted with DCM and i-PrOH (v:v=3:1, 20 mL×4). The combined organic layer was washed with brine (15 mL×1), dried over Na₂SO₄, filtered and concentrated under reduced pressure. Compound 9-formyl-N,N-dimethyl-2-morpholino-4-oxo-pyrido[1,2-a] pyrimidine-7-carboxamide (1 g, 3.03 mmol, 71.00% yield) was obtained as brown solid. ¹H NMR (DMSO-d₆, 400 MHz) δ 10.69 (s, 1H), 9.02 (s, 1H), 8.18 (s, 1H), 5.73 (s, 1H), 3.70˜3.66 (m, 8H), 3.02 (s, 6H).

### Preparation of Compounds in Step 5 Scheme 24

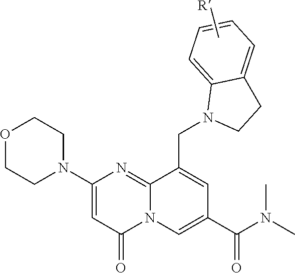

A solution of 9-formyl-N,N-dimethyl-2-morpholino-4-oxo-pyrido[1,2-a]pyrimidine-7-carboxamide (454.08 umol, 1 eq), AcOH (13.62 mmol, 779.07 uL, 30 eq) and indoline (590.30 umol 698.46 umol, 1.3 eq˜2.0 eq) in MeOH (6 mL/mmol˜8 mL/mmol) was stirred at 20° C. for an hour. Then NaBH₃CN (908.16 umol, 2 eq) was added in portions at 0° C. and the mixture was stirred at 20° C. for an hour. The mixture was quenched with ice water at 0° C. and the organic solvent was removed under reduced pressure. The aqueous was made pH=8 with sat. NaHCO₃, then extracted with EtOAc. The combined organic layer was washed with brine, dried over Na₂SO₄, filtered and concentrated under reduced pressure. The residue was purified by prep-HPLC (column: Phenomenex Gemini-NX 80*40 mm*3 um or Phenomenex Gemini—NX C18 75*30 mm*3 um; mobile phase: [water (10 mM NH₄HCO₃)-MeCN]; B %: 20%-55%, 8 mins). The eluent was lyophilized to give desired product.

### Preparation of 9-((4,6-difluoroindolin-1-yl)methyl)-N,N-dimethyl-2-morpholino-4-oxo-4H-pyrido[1,2-a]pyrimidine-7-carboxamide (Step 5 in Scheme 24)

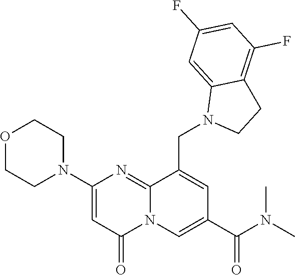 [SMILES: CN(C)C(=O)c1cc(CN2CCc3c(F)cc(F)cc32)c2nc(N3CCOCC3)cc(=O)n2c1]

A solution of 9-formyl-N,N-dimethyl-2-morpholino-4-oxo-pyrido[1,2-a]pyrimidine-7-carboxamide (0.3 g, 454.08 umol, 50% purity, 1 eq), AcOH (818.03 mg, 13.62 mmol, 779.07 uL, 30 eq) and 4,6-difluoroindoline (152.64 mg, 590.30 umol, 60% purity, 1.3 eq) in MeOH (3 mL) was stirred at 20° C. for an hour. Then NaBH₃CN (57.07 mg, 908.16 umol, 2 eq) was added in portions at 0° C. and the mixture was stirred at 20° C. for an hour. HPLC and LCMS showed the reaction was complete. The mixture was quenched with ice water (1 mL) at 0° C. and the organic solvent was removed under reduced pressure. The aqueous was made pH=8 with sat. NaHCO₃, then extracted with EtOAc (2 mL×4). The combined organic layer was washed with brine (5 mL×1), dried over Na₂SO₄, filtered and concentrated under reduced pressure. The residue was purified by prep-HPLC (column: column: Phenomenex Gemini-NX 80*40 mm*3 um; mobile phase: [water (10 mM NH₄HCO₃)-MeCN]; B %: 25%-45%, 8 mins). The eluent was removed under freeze drying. Compound 9-[(4,6-difluoroindolin-1-yl)methyl]-N,N-dimethyl-2-morpholino-4-oxo-pyrido[1,2-a]pyrimidine-7-carboxamide (37 mg, 77.30 umol, 17.02% yield, 98.08% purity) was obtained as off-white solid. ¹H NMR (DMSO-d₆, 400 MHz) δ 8.77 (s, 1H), 7.65 (s, 1H), 6.33˜6.26 (m, 2H), 5.69 (s, 1H), 4.55 (s, 2H), 3.69˜3.62 (m, 8H), 3.60 (t, J=8.4 Hz, 2H), 3.00 (t, J=8.8 Hz, 2H), 2.97 (s, 6H). HPLC: 98.08% (220 nm), 97.97% (254 nm). MS (ESI): mass calcd. For C₂₄H₂₅F₂N₅O₃ 469.19 m/z found 470.2 [M+H]⁺.

### Compound 99

### 9-((4,7-difluoroindolin-1-yl)methyl)-N,N-dimethyl-2-morpholino-4-oxo-4H-pyrido[1,2-a]pyrimidine-7-carboxamide was prepared according to the procedure described herein for Step 5 in Scheme 24

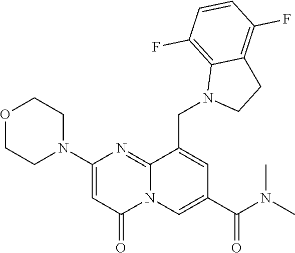 [SMILES: CN(C)C(=O)c1cc(CN2CCc3c(F)ccc(F)c32)c2nc(N3CCOCC3)cc(=O)n2c1]

Compound 99 (14.8 mg, 30.17 umol, 16.61% yield, 95.72% purity) was obtained as off white solid. ¹H NMR (DMSO-d₆, 400 MHz) δ 8.78 (s, 1H), 7.75 (s, 1H), 6.95˜6.88 (m, 1H), 6.45˜6.40 (m, 1H), 5.68 (s, 1H), 4.70 (s, 2H), 3.69˜3.66 (m, 8H), 3.57 (t, J=8.8 Hz, 2H), 3.07 (t, J=8.8 Hz, 2H), 2.97 (s, 6H). HPLC: 95.72% (220 nm), 98.26% (254 nm). MS (ESI): mass calcd. For C₂₄H₂₅F₂N₅O₃ 469.19 m/z found 470.2 [M+H]⁺.

### Compound 100

### 9-((4-chloro-7-fluoroindolin-1-yl)methyl)-N,N-dimethyl-2-morpholino-4-oxo-4H-pyrido[1,2-a]pyrimidine-7-carboxamide was prepared according to the procedure described herein for Step 5 in Scheme 24

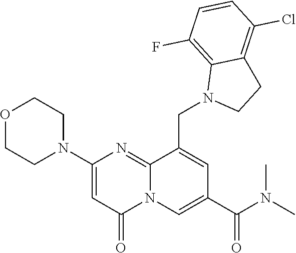 [SMILES: CN(C)C(=O)c1cc(CN2CCc3c(Cl)ccc(F)c32)c2nc(N3CCOCC3)cc(=O)n2c1]

Compound 100 (13.6 mg, 27.27 umol, 9.01% yield, 97.45% purity) was obtained as white solid. ¹H NMR (400 MHz, DMSO-d₆) δ 8.77 (s, 1H), 7.75 (s, 1H), 6.96˜6.91 (m, 1H), 6.65˜6.62 (m, 1H), 5.68 (s, 1H), 4.70 (s, 2H), 3.65 (s, 8H), 3.57 (t, J=8.8 Hz, 2H), 3.07 (t, J=8.8 Hz, 2H), 2.97 (s, 6H). LCMS: 97.45% (220 nm), 98.43% (254 nm). MS (ESI): mass calcd. For C₂₄H₂₅ClFN₅O₃ 485.16 m/z found 486.1 [M+H]⁺.

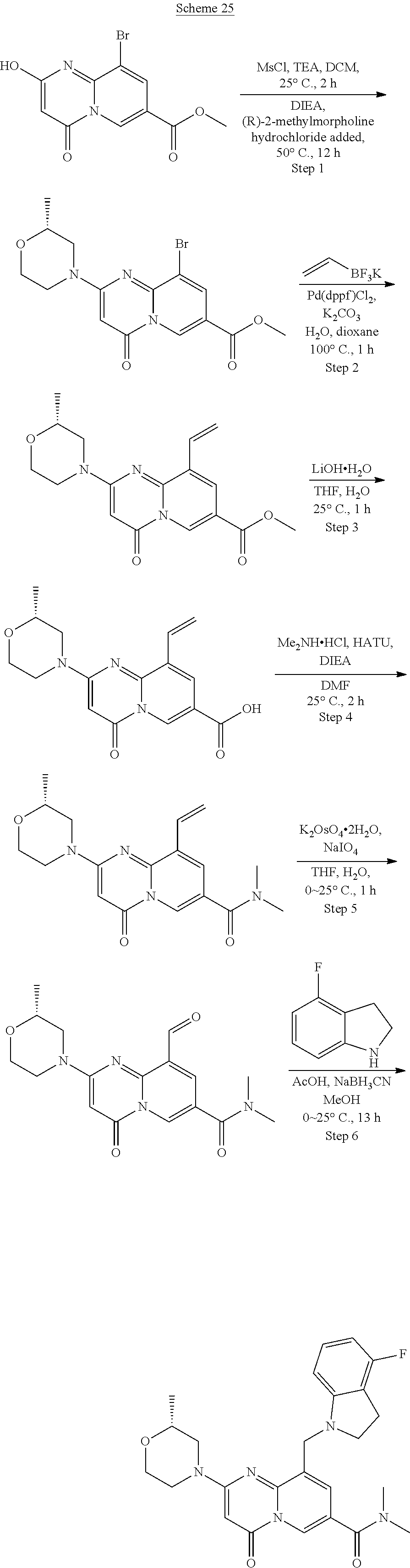

### Compound 101

### Preparation of (R)-9-((4-fluoroindolin-1-yl)methyl)-N,N-dimethyl-2-(2-methylmorpholino)-4-oxo-4H-pyrido[1,2-a]pyrimidine-7-carboxamide (Steps 1-6 in Scheme 25)

### Preparation of (R)-methyl 9-bromo-2-(2-methylmorpholino)-4-oxo-4H-pyrido[1,2-a]pyrimidine-7-carboxylate (Step 1 in Scheme 25)

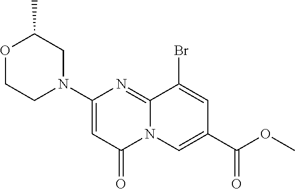 [SMILES: COC(=O)c1cc(Br)c2nc(N3CCO[C@H](C)C3)cc(=O)n2c1]

To a mixture of methyl 9-bromo-2-hydroxy-4-oxo-pyrido[1,2-a]pyrimidine-7-carboxylate (4.3 g, 14.38 mmol, 1 eq) and Et₃N (2.91 g, 28.76 mmol, 4.00 mL, 2 eq) in DCM (60 mL) was added MsCl (3.63 g, 31.69 mmol, 2.45 mL, 2.20 eq) dropwise at 0° C. under N₂. The reaction mixture was stirred at 25° C. for 2 hours. Then (2R)-2-methylmorpholine hydrochloride (2.00 g, 14.53 mmol, 1 eq) and DIEA (3.76 g, 29.06 mmol, 5.06 mL, 2 eq) were added at 25° C. The reaction mixture was stirred at 50° C. for 12 hours. LCMS showed the reaction was nearly complete. The mixture was cooled to 0° C. and then adjusted to pH=7 with 2N HCl. The organic phase was separated and the aqueous phase was extracted with DCM (50 mL×3). The combined organic phase was washed with brine (40 mL), dried with anhydrous Na₂SO₄, filtered and concentrated in vacuum. The crude product was triturated with DCM (15 mL) at 25° C. for half an hour. Compound methyl 9-bromo-2-[(2R)-2-methylmorpholin-4-yl]-4-oxo-pyrido[1,2-a]pyrimidine-7-carboxylate (2.6 g, 6.80 mmol, 46.82% yield) was obtained as yellow solid. ¹H NMR (DMSO-d₆, 400 MHz) δ 9.24 (s, 1H), 8.37 (s, 1H), 5.71 (s, 1H), 3.95˜3.93 (m, 1H), 3.90 (s, 3H), 3.56˜3.46 (m, 2H), 3.36˜3.33 (m, 2H), 3.04˜3.02 (m, 1H), 2.75˜2.66 (m, 1H), 1.16 (d, J=6.0 Hz, 3H).

### Preparation of (R)-methyl 2-(2-methylmorpholino)-4-oxo-9-vinyl-4H-pyrido[1,2-a]pyrimidine-7-carboxylate (Step 2 in Scheme 25)

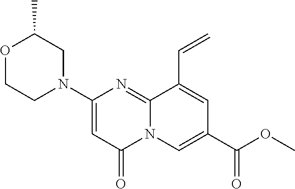 [SMILES: C=Cc1cc(C(=O)OC)cn2c(=O)cc(N3CCO[C@H](C)C3)nc12]

To a mixture of methyl 9-bromo-2-[(2R)-2-methylmorpholin-4-yl]-4-oxo-pyrido[1,2-a]pyrimidine-7-carboxylate (2.6 g, 6.80 mmol, 1 eq) and potassium trifluoro(vinyl)boranuide (1.37 g, 10.20 mmol, 1.5 eq) in dioxane (20 mL) and H₂O (10 mL) was added K₂CO₃ (1.88 g, 13.61 mmol, 2 eq) and Pd(dppf)Cl₂ (248.87 mg, 340.13 umol, 0.05 eq) at 25° C. under N₂. The reaction mixture was stirred at 100° C. for an hour. LCMS showed methyl 9-bromo-2-[(2R)-2-methylmorpholin-4-yl]-4-oxo-pyrido[1,2-a]pyrimidine-7-carboxylate was consumed completely. The reaction mixture was cooled to room temperature and filtered through a pad of the Celite. The filtrate was concentrated under reduced pressure at 45° C. The residue was dissolved in EtOAc (30 mL) and H₂O (30 mL). The organic phase was separated and the aqueous phase was extracted with ethyl acetate (20 mL×2). The combined organic phase was washed with brine (20 mL), dried over anhydrous Na₂SO₄, filtered and concentrated in vacuum. The residue was purified by flash silica gel chromatography (Brotage®; 40 g SepaFlash® Silica Flash Column, Eluent of 0˜100% Ethyl acetate/Petroleum ether gradient @80 mL/min). The solvent was concentrated in vacuum. Compound methyl 2-[(2R)-2-methylmorpholin-4-yl]-4-oxo-9-vinyl-pyrido[1,2-a]pyrimidine-7-carboxylate (0.9 g, 2.73 mmol, 40.17% yield) was obtained as yellow solid. ¹H NMR (DMSO-d₆, 400 MHz) δ 9.25 (s, 1H), 8.22 (s, 1H), 7.30 (dd, J=11.2 Hz, 17.6 Hz, 1H), 6.14 (d, J=17.6 Hz, 1H), 5.71 (s, 1H), 5.56 (d, J=11.2 Hz, 1H), 4.29 (s, 2H), 3.93˜3.89 (m, 4H), 3.58˜3.49 (m, 2H), 3.01 (t, J=9.6 Hz, 1H), 2.69 (t, J=11.6 Hz, 1H), 1.17 (d, J=6.0 Hz, 3H).

### Preparation of (R)-2-(2-methylmorpholino)-4-oxo-9-vinyl-4H-pyrido[1,2-a]pyrimidine-7-carboxylic acid (Step 3 in Scheme 25)

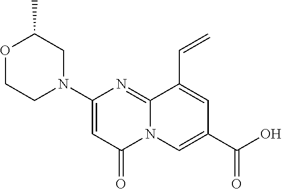 [SMILES: C=Cc1cc(C(=O)O)cn2c(=O)cc(N3CCO[C@H](C)C3)nc12]

To a solution of methyl 2-[(2R)-2-methylmorpholin-4-yl]-4-oxo-9-vinyl-pyrido[1,2-a]pyrimidine-7-carboxylate (0.9 g, 2.73 mmol, 1 eq) in THF (5 mL) was added a solution of LiOH·H₂O (344.02 mg, 8.20 mmol, 3 eq) in H₂O (5 mL) at 25° C. The reaction mixture was stirred at 25° C. for an hour. LCMS the reaction was complete. The mixture was cooled to 0° C. and adjusted to pH=4 with 2N HCl. The yellow precipitate was collected after filtration. Compound 2-[(2R)-2-methylmorpholin-4-yl]-4-oxo-9-vinyl-pyrido[1,2-a]pyrimidine-7-carboxylic acid (0.66 g, 2.09 mmol, 76.60% yield) was obtained as yellow solid. ¹H NMR (DMSO-d₆, 400 MHz) δ 9.25 (s, 1H), 8.23 (s, 1H), 7.31 (dd, J=11.6 Hz, 18.0 Hz, 1H), 6.14 (d, J=17.2 Hz, 1H), 5.70 (s, 1H), 5.56 (d, J=11.6 Hz, 1H), 3.93˜3.90 (m, 2H), 3.55˜3.48 (m, 3H), 3.04˜2.98 (m, 1H), 2.69˜2.50 (m, 1H), 1.17 (d, J=6.0 Hz, 3H).

### Preparation of (R)—N,N-dimethyl-2-(2-methylmorpholino)-4-oxo-9-vinyl-4H-pyrido[1,2-a]pyrimidine-7-carboxamide (Step 4 in Scheme 25)

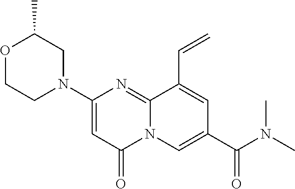 [SMILES: C=Cc1cc(C(=O)N(C)C)cn2c(=O)cc(N3CCO[C@H](C)C3)nc12]

To a mixture of 2-[(2R)-2-methylmorpholin-4-yl]-4-oxo-9-vinyl-pyrido[1,2-a]pyrimidine-7-carboxylic acid (0.66 g, 2.09 mmol, 1 eq), Me₂NH (512.04 mg, 6.28 mmol, 575.33 uL, 100% purity, 3 eq, HCl) and DIEA (1.35 g, 10.47 mmol, 1.82 mL, 5 eq) in DMF (10 mL) was added HATU (1.19 g, 3.14 mmol, 1.5 eq) in portions at 0° C. under N₂. The mixture was stirred at 25° C. for 2 hours. LCMS showed 2-[(2R)-2-methylmorpholin-4-yl]-4-oxo-9-vinyl-pyrido[1,2-a]pyrimidine-7-carboxylic acid was consumed completely. The reaction mixture was quenched with ice water (15 mL) and extracted with ethyl acetate (20 mL×3). The combined organic phase was washed with brine (20 mL), dried with anhydrous Na₂SO₄, filtered and concentrated in vacuum. The residue was purified by flash silica gel chromatography (Biotage®; 8 g SepaFlash® Silica Flash Column, Eluent of 0˜100% Ethyl acetate/Petroleum ether gradient @ 45 mL/min). The solvent was concentrate in vacuum. Compound N,N-dimethyl-2-[(2R)-2-methylmorpholin-4-yl]-4-oxo-9-vinyl-pyrido[1,2-a] pyrimidine-7-carboxamide (0.43 g, 1.26 mmol, 60.00% yield) was obtained as yellow solid. ¹H NMR (DMSO-d₆, 400 MHz) δ 8.77 (s, 1H), 8.06 (s, 1H), 7.34 (dd, J=11.2 Hz, 18.0 Hz, 1H), 6.15 (d, J=16.8 Hz, 1H), 5.70 (s, 1H), 5.54 (d, J=12.0 Hz, 1H), 3.69˜3.59 (m, 1H), 3.55˜3.45 (m, 2H), 3.23˜3.08 (m, 2H), 3.02 (s, 6H), 2.99˜2.93 (m, 1H), 2.70˜2.62 (m, 1H), 1.24 (d, J=6.0 Hz, 3H).

### Preparation of (R)-9-formyl-N,N-dimethyl-2-(2-methylmorpholino)-4-oxo-4H-pyrido[1,2-a]pyrimidine-7-carboxamide (Step 5 in Scheme 25)

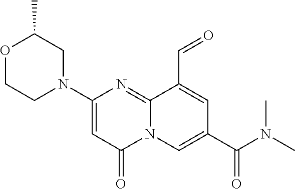 [SMILES: C[C@@H]1CN(c2cc(=O)n3cc(C(=O)N(C)C)cc(C=O)c3n2)CCO1]

To a solution of N,N-dimethyl-2-[(2R)-2-methylmorpholin-4-yl]-4-oxo-9-vinyl-pyrido[1,2-a] pyrimidine-7-carboxamide (0.2 g, 584.13 umol, 1 eq) in THF (5 mL) and H₂O (5 mL) was added K₂OsO₄·2H₂O (21.52 mg, 58.41 umol, 0.1 eq) and the mixture was stirred at 25° C. for half an hour. Then NaIO₄ (374.82 mg, 1.75 mmol, 97.10 uL, 3 eq) was added to the above mixture in portions at 0° C. and the mixture was stirred at 0° C. for half an hour. LCMS showed the reaction was complete. The reaction mixture was quenched with ice water (20 mL) and then extracted with ethyl acetate (20 mL×3). The combined organic phase was washed with brine (15 mL), dried with anhydrous Na₂SO₄, filtered and concentrated in vacuum. Compound 9-formyl-N,N-dimethyl-2-[(2R)-2-methylmorpholin-4-yl]-4-oxo-pyrido[1,2-a]pyrimidine-7-carboxamide (0.1 g, 290.39 umol, 49.71% yield) was obtained as yellow solid. ¹H NMR (DMSO-d₆, 400 MHz) δ 10.70 (s, 1H), 9.01 (s, 1H), 8.17 (s, 1H), 5.76 (s, 1H), 3.92˜3.90 (m, 1H), 3.53˜3.47 (m, 4H), 3.02 (s, 6H), 2.97˜2.95 (m, 1H), 2.73˜2.71 (m, 1H), 1.18 (d, J=6.0 Hz, 3H).

### Preparation of (R)-9-((4-fluoroindolin-1-yl)methyl)-N,N-dimethyl-2-(2-methylmorpholino)-4-oxo-4H-pyrido[1,2-a]pyrimidine-7-carboxamide (Step 6 in Scheme 25)

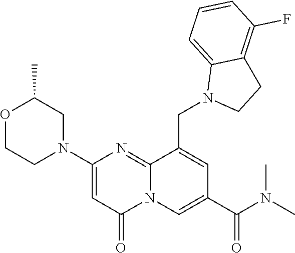 [SMILES: C[C@@H]1CN(c2cc(=O)n3cc(C(=O)N(C)C)cc(CN4CCc5c(F)cccc54)c3n2)CCO1]

To a mixture of 9-formyl-N,N-dimethyl-2-[(2R)-2-methylmorpholin-4-yl]-4-oxo-pyrido[1,2-a] pyrimidine-7-carboxamide (0.1 g, 290.39 umol, 1 eq) and 4-fluoroindoline (39.83 mg, 290.39 umol, 1 eq) in MeOH (10 mL) was added AcOH (523.16 mg, 8.71 mmol, 498.24 uL, 30 eq) at 25° C. and the mixture was stirred at 25° C. for 12 hours. Then the mixture was cooled to 0° C. and NaBH₃CN (36.50 mg, 580.78 umol, 2 eq) was added in portions. The mixture was stirred at 25° C. for an hour. LCMS showed the reaction was complete. The reaction mixture was quenched with ice water (1 mL) and concentrated under reduced pressure at 45° C. The residue was purified by prep-HPLC (column: Phenomenex Gemini-NX 80*30 mm*3 um; mobile phase: [water (10 mM NH₄HCO₃)-MeCN]; B %: 25%-55%, 8 mins). The solvent was removed under freeze drying. Compound 9-[(4-fluoroindolin-1-yl)methyl]-N,N-dimethyl-2-[(2R)-2-methylmorpholin-4-yl]-4-oxo-pyrido[1,2-a]pyrimidine-7-carboxamide (18.0 mg, 37.08 umol, 12.77% yield, 95.89% purity) was obtained as off-white solid. ¹H NMR (DMSO-d₆, 400 MHz) δ 8.77 (s, 1H), 7.68 (s, 1H), 7.02˜6.96 (m, 1H), 6.41-6.34 (m, 2H), 5.70 (s, 1H), 4.51 (s, 2H), 4.42˜4.26 (m, 2H), 3.87 (d, J=11.2 Hz, 1H), 3.57˜3.46 (m, 4H), 3.03 (t, J=8.4 Hz, 2H), 2.96 (s, 6H), 2.95˜2.93 (m, 1H), 2.64˜2.61 (m, 1H), 1.13 (d, J=6.4 Hz, 3H). HPLC: 95.89% (220 nm), 99.55% (254 nm). MS (ESI): mass calcd. For C₂₅H₂₈O₃N₅F 465.22 m/z found 466.2 [M+H]⁺.

### Compound 102 and Compound 103

### Preparation of (S)-8-((4-fluoro-2-methylindolin-1-yl)methyl)-N,N-dimethyl-2-morpholino-4-oxo-4H-chromene-6-carboxamide and (R)-8-((4-fluoro-2-methylindolin-1-yl)methyl)-N,N-dimethyl-2-morpholino-4-oxo-4H-chromene-6-carboxamide

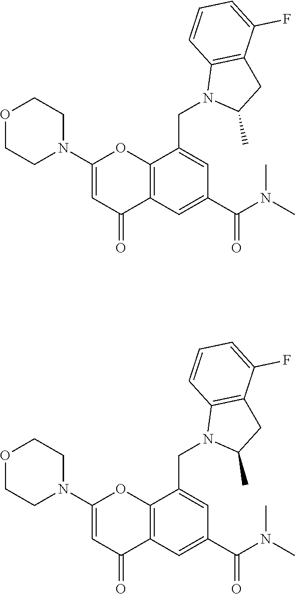 [SMILES: C[C@H]1Cc2c(F)cccc2N1Cc1cc(C(=O)N(C)C)cc2c(=O)cc(N3CCOCC3)oc12 ; C[C@@H]1Cc2c(F)cccc2N1Cc1cc(C(=O)N(C)C)cc2c(=O)cc(N3CCOCC3)oc12]

257 mg of Compound 33 was performed by chiral separation. (Chiral Preparative Instrument: Thar 80 preparative SFC; Column: DAICEL CHIRALPAK AD (250 mm*30 mm, 10 um); mobile phase: [Neu-EtOH]; B %: 55%-55%, 6 mins). Two isomers were obtained and the chiral centers were randomly assigned.

Compound 102 was assigned as 8-[[(2S)-4-fluoro-2-methyl-indolin-1-yl]methyl]-N,N-dimethyl-2-morpholino-4-oxo-chromene-6-carboxamide (white solid, 108.1 mg, 232.22 umol, 42.06% yield, 100% purity, Rt=1.452 minutes on chiral HPLC, 99.72% e.e.). ¹H NMR (DMSO-d₆, 400 MHz) δ 7.81 (s, 1H), 7.52 (s, 1H), 6.96 (d, J=5.2 Hz, 1H), 6.38 (t, J=8.0 Hz, 1H), 6.10 (d, J=7.6 Hz, 1H), 5.59 (s, 1H), 4.63˜4.54 (m, 2H), 3.89 (d, J=5.2 Hz, 1H), 3.70 (s, 4H), 3.53 (s, 4H), 2.94 (s, 3H), 2.84 (s, 3H), 2.68˜2.63 (m, 2H), 1.26 (d, J=5.2 Hz, 3H). HPLC: 100.00% (220 nm), 100.00% (254 nm). MS (ESI): mass calcd. For C₂₆H₂₈FN₃O₄ 465.21 m/z found 466.2 [M+H]⁺.

Compound 103 was assigned as 8-[[(2R)-4-fluoro-2-methyl-indolin-1-yl]methyl]-N,N-dimethyl-2-morpholino-4-oxo-chromene-6-carboxamide (white solid, 97 mg, 208.37 umol, 37.74% yield, 100% purity, Rt=1.800 minutes, on chiral HPLC, 98.52% e.e.).

Chiral Analytical Instrument: Waters UPCC with PDA; Column: Chiralpak AD-3, 50*4.6 mm I.D., 3 um; mobile phase: [CO₂-EtOH (0.05% IPAm)]; B %: 5%-50%, 3 mins; flow rate: 3.4 mL/min; column temp.: 35° C.; ABPR: 1800 psi. ¹H NMR (DMSO-d₆, 400 MHz) δ 7.81 (s, 1H), 7.52 (s, 1H), 6.96 (d, J=6.4 Hz, 1H), 6.38 (d, J=7.6 Hz, 1H), 6.10 (d, J=2.8 Hz, 1H), 5.59 (s, 1H), 4.64˜4.54 (m, 2H), 3.89 (d, J=5.6 Hz, 1H), 3.70 (s, 4H), 3.53 (s, 4H), 2.94 (s, 3H), 2.84 (s, 3H), 2.68˜2.63 (m, 2H), 1.25 (d, J=4.4 Hz, 3H). HPLC: 100.00% (220 nm), 100.00% (254 nm). MS (ESI): mass calcd. For C₂₆H₂₈FN₃O₄ 465.21 m/z found 466.2 [M+H]⁺.

### Cellular Assays

Cellular activity of PI3Kβ inhibitors was determined by quantifying the phosphorylation of AKT in HCC70 cells. AKT phosphorylated at Ser473 and Thr308 were measured using western blotting and specific primary antibodies from Cell Signaling (CST), Danvers, MA. On day 1, HCC70 cells (ATCC #CRL-2315) were seeded into Falcon 12-well cell Culture Plate (Corning #353043) at 100,000 cells per well, in 1 ml complete culture medium (RPMI-1640, GIBCO #11875093) containing 10% heat inactivated FBS (GIBCO #10438026), 1% Pen-Strep (GIBCO #15140122) and incubated at 37° AC, 5% CO₂ overnight. On day 2, compound (500 nM) or DMSO (0.1%, Sigma Aldrich #8418) was added and cells were further incubated for 60 min at 37° AC, 5% CO₂ in a total volume of 1 ml fresh medium.

At the end of the cell treatment period, media were removed by aspiration and adherent cells were lysed in 100 uL RIPA buffer (Sigma Aldrich #R0278). Equal volume (15 uL) of proteins was resolved by SDS-PAGE and transferred to PVDF (Millipore #IPFL00010) membrane for immunoblot analysis. Primary antibodies to the following proteins were used for immunoblotting: phosphor-AKT (Ser473) (CST #4060), phosphor-AKT (Thr308) (CST #2965), total AKT (CST #9272). Secondary goat anti-rabbit IgG fluorophore conjugated antibody (Invitrogen #A21109) was used to visualize the indicated proteins on an Odyssey CLx imaging system (Li-Cor). Quantification of band intensities was performed using Odyssey 3.0 software. Inhibition of Akt activity was calculated by band intensities using the formula:

$$Akt inhibition ( AI ) = ( \frac{p - AKT ( Ser 473 ) or p - AKT ( Thr 308 )}{Total AKT} ) \text{} \text{} Compound \div ( \frac{p - AKT ( Ser 473 ) or p - AKT ( Thr 308 )}{Total AKT} ) DMSO .$$

Index was evaluated by % of AI. Data represent Index+(20˜60%), ++(60%˜80%), +++(80%˜90%), and ++++>90%.

TABLE 1
Biological data - Cellular assay

| | Index of AI (HCC70) | |
|---|---|---|
| Compound ID | p-AKT ser 473 | p-AKT Thr 308 |
| Compound 1 | ++ | ++ |
| Compound 2 | ++ | ++ |
| Compound 3 | ++ | ++ |
| Compound 4 | ++ | ++ |
| Compound 5 | ++ | ++ |
| Compound 6 | ++ | ++ |
| Compound 7 | ++ | ++ |
| Compound 8 | ++ | ++ |
| Compound 9 | +++ | +++ |
| Compound 10 | ++++ | ++++ |
| Compound 11 | ++++ | ++++ |
| Compound 12 | ++++ | ++++ |
| Compound 13 | +++ | +++ |
| Compound 14 | + | + |
| Compound 15 | ++++ | ++++ |
| Compound 16 | ++++ | ++++ |
| Compound 17 | ++ | ++ |
| Compound 18 | − | − |
| Compound 19 | ++++ | ++++ |
| Compound 20 | ++ | ++ |
| Compound 21 | +++ | +++ |
| Compound 22 | ++++ | ++++ |
| Compound 23 | ++ | ++ |
| Compound 24 | ++++ | ++++ |
| Compound 25 | ++++ | ++++ |
| Compound 26 | − | − |
| Compound 27 | ++++ | ++++ |
| Compound 28 | ++ | ++ |
| Compound 29 | ++++ | ++++ |
| Compound 30 | +++ | +++ |
| Compound 31 | ++++ | ++++ |
| Compound 32 | ++++ | ++++ |
| Compound 33 | ++++ | ++++ |
| Compound 34 | +++ | +++ |
| Compound 35 | − | − |
| Compound 36 | ++++ | ++++ |
| Compound 37 | +++ | +++ |
| Compound 38 | ++++ | ++++ |
| Compound 39 | ++++ | ++++ |
| Compound 40 | ++++ | ++++ |
| Compound 41 | +++ | +++ |
| Compound 42 | ++++ | ++++ |
| Compound 43 | +++ | +++ |
| Compound 44 | +++ | +++ |
| Compound 45 | ++++ | ++++ |
| Compound 46 | +++ | +++ |
| Compound 47 | ++++ | ++++ |
| Compound 48 | +++ | +++ |
| Compound 49 | ++ | ++ |
| Compound 50 | ++++ | ++++ |
| Compound 51 | ++++ | ++++ |
| Compound 52 | ++ | ++ |
| Compound 53 | ++ | ++ |
| Compound 54 | + | + |
| Compound 55 | + | + |
| Compound 56 | ++ | ++ |
| Compound 57 | ++ | ++ |
| Compound 58 | +++ | +++ |
| Compound 59 | +++ | +++ |
| Compound 60 | +++ | +++ |
| Compound 61 | + | + |
| Compound 62 | +++ | +++ |
| Compound 63 | ++++ | ++++ |
| Compound 64 | ++++ | ++++ |
| Compound 65 | + | + |
| Compound 66 | +++ | +++ |
| Compound 67 | ++ | ++ |
| Compound 68 | ++++ | ++++ |
| Compound 69 | − | − |
| Compound 70 | +++ | +++ |
| Compound 71 | ++++ | ++++ |
| Compound 72 | + | + |
| Compound 73 | − | − |
| Compound 74 | − | − |
| Compound 75 | − | − |
| Compound 76 | − | − |
| Compound 77 | − | − |
| Compound 78 | +++ | +++ |
| Compound 79 | +++ | +++ |
| Compound 80 | ++ | ++ |
| Compound 81 | ++ | ++ |
| Compound 82 | +++ | +++ |
| Compound 83 | ++ | ++ |
| Compound 84 | +++ | +++ |
| Compound 85 | ++ | ++ |
| Compound 86 | ++++ | ++++ |
| Compound 87 | ++ | ++ |
| Compound 88 | ++ | ++ |
| Compound 89 | + | N/A |
| Compound 90 | ++++ | ++++ |
| Compound 91 | ++++ | ++++ |
| Compound 92 | ++ | ++ |
| Compound 93 | +++ | +++ |
| Compound 94 | ++ | ++ |
| Compound 95 | ++ | ++ |
| Compound 96 | ++++ | ++++ |
| Compound 97 | + | + |
| Compound 98 | +++ | +++ |
| Compound 99 | +++ | +++ |
| Compound 100 | +++ | +++ |
| Compound 101 | ++++ | ++++ |
| Compound 102 | ++++ | ++++ |

### Enzyme Binding Assays (KINOMEscan)

Kinase enzyme binding affinities of compounds disclosed herein were determined using the KINOMEscan technology performed by Eurofins DiscoveRx Corporation, San Diego, California, USA (www.eurofinsdiscoveryservices.com). Table 2 reports the obtained PIK3β Kd values (nM) of, with the PIK3β Kd being the inhibitor binding constant. Table 3 reports the obtained Kd values (nM), with the Kd being the inhibitor binding constant:

TABLE 2
Biological data - PIK3β Binding Activity and Cellular assay

| | Kd (nM) | Index of AI (HCC70) | |
|---|---|---|---|
| Compound | PIK3Cb_h | p-AKT ser 473 | p-AKT Thr 308 |
| Compound 10 | 0.20 | ++++ | ++++ |
| Compound 12 | 0.55 | +++ | +++ |
| Compound 13 | 0.14 | ++++ | ++++ |
| Compound 15 | 0.059 | ++++ | ++++ |
| Compound 16 | 0.19 | ++++ | ++++ |
| Compound 19 | 0.067 | ++++ | ++++ |
| Compound 21 | 0.31 | +++ | +++ |
| Compound 25 | 0.21 | ++++ | ++++ |
| Compound 27 | 0.28 | ++++ | ++++ |
| Compound 29 | 0.17 | ++++ | ++++ |
| Compound 30 | 0.32 | +++ | +++ |
| Compound 32 | 0.18 | ++++ | ++++ |
| Compound 33 | 0.49 | ++++ | ++++ |
| Compound 34 | 0.37 | +++ | +++ |
| Compound 38 | 0.52 | ++++ | ++++ |
| Compound 39 | 0.072 | ++++ | ++++ |
| Compound 42 | 0.26 | ++++ | ++++ |
| Compound 44 | 1.3 | +++ | +++ |
| Compound 48 | 0.26 | +++ | +++ |
| Compound 49 | 0.67 | ++ | ++ |
| Compound 50 | 0.11 | ++++ | ++++ |
| Compound 51 | 0.12 | ++++ | ++++ |
| Compound 58 | 2.0 | +++ | +++ |
| Compound 59 | 0.61 | +++ | +++ |

TABLE 3
Biological data - Binding Activity and Cellular assay

| | | | | | Index of AI (HCC70) | |
|---|---|---|---|---|---|---|
| | Kd(nM) | | | | p-AKT ser | p-AKT |
| Compound | PIK3Ca_h | PIK3Cb_h | PIK3Cd_h | PIK3Cg_h | 473 | Thr 308 |
| Compound 10 | 22 | 0.2 | 8 | 730 | ++++ | ++++ |
| Compound 16 | 36 | 0.19 | 8.1 | 110 | ++++ | ++++ |
| Compound 21 | 45 | 0.31 | 58 | 2200 | +++ | +++ |
| Compound 32 | 54 | 0.18 | 19 | 500 | ++++ | ++++ |
| Compound 33 | 17 | 0.49 | 12 | 320 | ++++ | ++++ |
| Compound 48 | 130 | 0.26 | 40 | 730 | +++ | +++ |
| Compound 48- | >10000 | 400 | >10000 | >10000 | − | − |
| P1 | | | | | | |
| Compound 48- | >10000 | 740 | >10000 | >10000 | − | − |
| P2 | | | | | | |
| Compound 48- | 92 | 0.11 | 8.9 | 1100 | ++++ | ++++ |
| P3 | | | | | | |
| Compound 48- | 190 | 0.12 | 18 | 1600 | ++++ | ++++ |
| P4 | | | | | | |
| Compound 90 | 3.2 | 0.075 | 1.5 | 87 | ++++ | ++++ |
| Compound 91 | 22 | 1 | 7.4 | 230 | ++++ | ++++ |

Applicant's disclosure is described herein in preferred embodiments with reference to the FIGURES, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the description, herein, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

1. A compound having the structural formula (I): 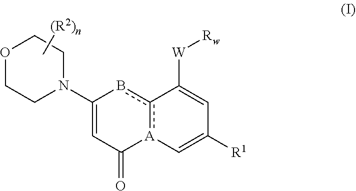
wherein
A represents carbon and B represents oxygen, or each of A and B represent nitrogen;
W represents —CHR-Q- or -Q-CHR—; wherein Q represents a bond, O, S, CH₂ or NR′; R and R′ is independently selected from hydrogen, C₁₋₄alkyl or C₁₋₄alkyl substituted with —OH or halo;
R_{w} represents Ar or a substituted or unsubstituted indoline group, wherein Ar is a group comprising a substituted or unsubstituted 6-membered aromatic or hetero-aromatic ring;
R² represents C₁₋₃ alkyl, CN, CH₂OH, CH₂F or CONH₂;
R¹ represents H, NH₂, OH, CN, CF₃, CHO, COC₁₋₄alkyl, CH₂C(═O)—NR^{1a}R^{1b}, C(═O)—NR^{1a}R^{1b}, CH₂COOR^{1c}, NR^{1d}R^{1e}, C₁₋₄ alkyl, O—C₁₋₄ alkyl, Het, Ar, NR^{1h}(C═O)R¹ⁱ, NR^{1j}(C═O)NR^{1k}R^{1l}, NR^{1j}(SO₂)R^{1m}, SO₂NR¹ⁿR^{1o}, NR^{1j}(C═O)C(CN)C(OH)R^{1p} 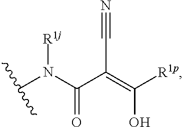
P(O)MeMe, P(O)OMeOMe; provided that, when A is carbon, B is oxygen and R_{w} is Ar, R¹ represents NH₂, CN, CHO, COC₁₋₄alkyl, CH₂C(═O)—NR^{1a}R^{1b}, CH₂COOR^{1c}, NR^{1d}R^{1e}, O—C₂₋₄ alkyl, Het, NR^{1h}(C═O)R¹ⁱ, NR^{1j}(C═O)NR^{1k}R^{1l}, NR^{1j}(SO₂)R^{1m}, SO₂NR¹ⁿR^{1o}, NR^{1j}(C═O)C(CN)C(OH)R^{1p}, P(O)MeMe, P(O)OMeOMe; when A and B is nitrogen, R_{w} is Ar, R¹ represents NH₂, CN, CHO, COC₁₋₄alkyl, CH₂C(═O)—NR^{1a}R^{1b}, CH₂COOR^{1c}, NR^{1d}R^{1e}, O—C₂₋₄ alkyl, Het, NR^{1h}(C═O)R¹ⁱ, NR^{1j}(C═O)NR^{1k}R^{1l}, NR^{1j}(SO₂)R^{1m}, SO₂NR¹ⁿR^{1o}, NR^{1j}(C═O)C(CN)C(OH)R^{1p}, P(O)MeMe, P(O)OMeOMe, C(═O)—NR^{1q}R^{1r}, wherein R^{1q} and R^{1r} together form a 3- to 8-membered nitrogen-containing heterocyclyl ring with one or more substituent selected from the group consisting of C₁₋₃ alkyl and NR^{3a}R^{3b};
wherein each of R^{1a}, R^{1b}, R^{1c}, R^{1d}, R^{1e}, R^{1h}, R¹ⁱ, R^{1j}, R^{1k}, R^{1l}, R^{1m}, R¹ⁿ, R^{1o}, R^{1p} is independently selected from H, C₁₋₄ alkyl, C₃₋₈cycloalkyl, C₃₋₈heterocycloakyl or C₁₋₄ alkyl substituted with one or more substituent selected from the group consisting of hydroxyl, OMe, CN, fluoro, NR^{3a}R^{3b}, C₃₋₈cycloalkyl, C₃₋₈ heterocycloakyl; R^{1a} and R^{1b}, R^{1d} and R^{1e}, R¹ⁿ and R^{1o} are optionally together form a 3- to 8-membered nitrogen-containing heterocyclyl ring with one or more substituent selected from the group consisting of C₁₋₃ alkyl and NR^{3a}R^{3b};
C₁₋₄ alkyl is unsubstituted or substituted with one or more substituent selected from the group consisting of OH, F, NR^{1d}R^{1e};
R^{3a} and R^{3b} each independently are selected from the group consisting of H, and C₁₋₄ alkyl;
Het represents a 3- to 8-membered saturated or partially saturated monocyclic, bridged or spiro heterocyclyl containing at least one heteroatom each independently selected from O, S, S(═O)ₚ and N; which substituted wish one or two substituents each independently selected from the group consisting of halo, NR^{3a}R^{3b}, C₁₋₄ alkyl, OH, OMe and CN;
n represents 0, 1, 2, 3 or 4; and
p represents 1 or 2;
or a pharmaceutically acceptable form or an isotope derivative thereof.

2. The compound of claim 1, wherein A is carbon and B is oxygen, having the structural formula (Ia):

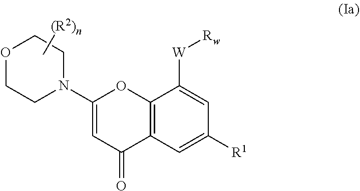

3. The compound of claim 2, wherein R_{w} is Ar.

4. The compound of claim 3, wherein W is —CHR⁴—NR⁵—, wherein each of R⁴ and R⁵ is independently selected from H, methyl or ethyl.

5. The compound of claim 2, wherein R_{w} is a substituted or unsubstituted indoline group.

6. The compound of claim 5, wherein W is —CHR⁴, wherein R⁴ is selected from H, methyl or ethyl.

7. The compound of claim 1, wherein each of A and B is nitrogen, having the structural formula (Ib):

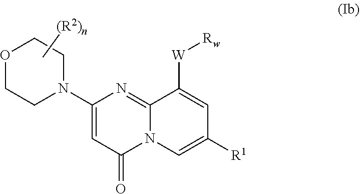

8. The compound of claim 7, wherein R_{w} is Ar.

9. The compound of claim 8, wherein W is —CHR⁴—NR⁵—, wherein each of R⁴ and R⁵ is independently selected from H, methyl or ethyl.

10. The compound of claim 7, wherein R_{w} is a substituted or unsubstituted indoline group.

11. The compound of claim 10, wherein W is —CHR⁴, wherein R⁴ is selected from H, methyl or ethyl.

12. (canceled)

13. (canceled)

14. The compound of claim 1, wherein Ar or the indoline group is substituted with one or more substitution groups selected from the group consisting of halogen, C₁₋₄ alkyl, OC₁₋₄ alkyl, CN, CHF₂, CF₃ and CH₂OH.

15. (canceled)

16. (canceled)

17. The compound of claim 1, wherein W—R_{w}, is selected from: 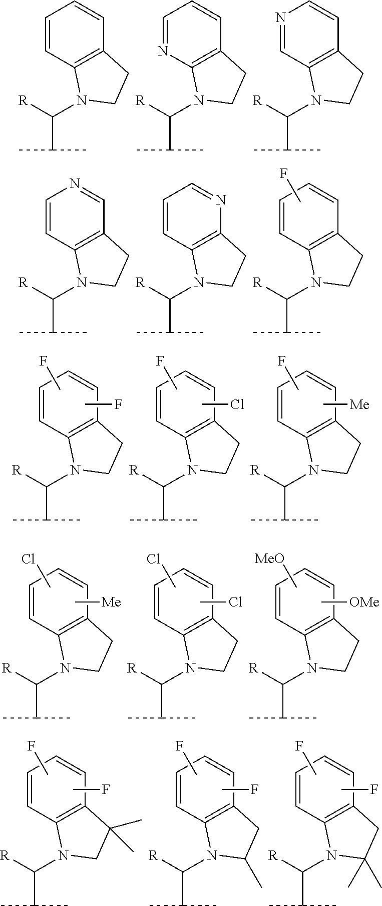
wherein R is independently selected from hydrogen, C₁₋₄alkyl or C₁₋₄alkyl substituted with —OH or halo.

18. The compound of claim 1, wherein n is 0.

19. The compound of claim 1, wherein n is 1.

20. (canceled)

21. (canceled)

22. The compound of claim 1, wherein R¹ is C(═O)—NR^{1a}R^{1b}, wherein each of R^{1a} and R^{1b} is independently selected from H and C₁₋₄ alkyl.

23. The compound of claim 1, wherein R¹ is C(═O)NR^{1q}R^{1r}, wherein R^{1q} and R^{1r} together form a 3- to 8-membered nitrogen-containing heterocyclyl ring optionally substituted with one of more of C₁₋₃ alkyl and NR^{3a}R^{3b}.

24. (canceled)

25. (canceled)

26. (canceled)

27. The compound of claim 1, wherein R¹ is C₁₋₄ alkyl, optionally substituted with hydroxy, —O—C₁₋₄ alkyl and NR^{1c}R^{1d}.

28. A compound selected from the group consisting of:

| Entry | Compd | Structure | Name |
|---|---|---|---|
| 1 | Compound 9 | 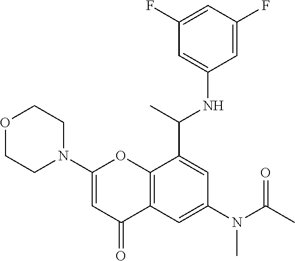 [SMILES: CC(=O)N(C)c1cc(C(C)Nc2cc(F)cc(F)c2)c2oc(N3CCOCC3)cc(=O)c2c1] | N-(8-(1-((3,5- difluorophenyl)amino)ethyl)-2- morpholino-4-oxo-4H-chromen-6- yl)-N-methylacetamide |
| 2 | Compound 10 | 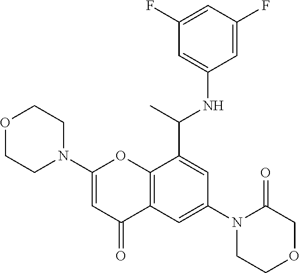 [SMILES: CC(Nc1cc(F)cc(F)c1)c1cc(N2CCOCC2=O)cc2c(=O)cc(N3CCOCC3)oc12] | 4-(8-(1-((3,5- difluorophenyl)amino)ethyl)-2- morpholino-4-oxo-4H-chromen-6- yl)morpholin-3-one |
| 3 | Compound 11 | 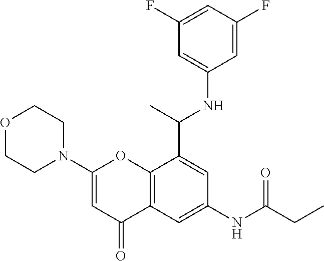 [SMILES: CCC(=O)Nc1cc(C(C)Nc2cc(F)cc(F)c2)c2oc(N3CCOCC3)cc(=O)c2c1] | N-(8-(1-((3,5- difluorophenyl)amino)ethyl)-2- morpholino-4-oxo-4H-chromen-6- yl)propionamide |
| 4 | Compound 12 | 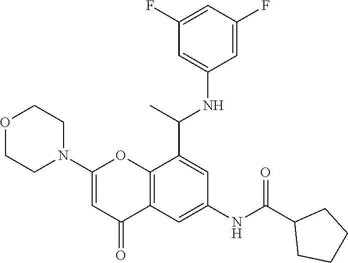 [SMILES: CC(Nc1cc(F)cc(F)c1)c1cc(NC(=O)C2CCCC2)cc2c(=O)cc(N3CCOCC3)oc12] | N-(8-(1-((3,5- difluorophenyl)amino)ethyl)-2- morpholino-4-oxo-4H-chromen-6- yl)cyclopentanecarboxamide |
| 5 | Compound 13 | 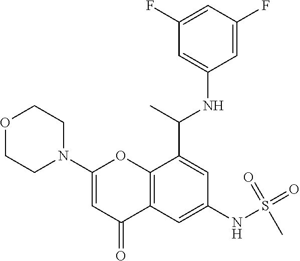 [SMILES: CC(Nc1cc(F)cc(F)c1)c1cc(NS(C)(=O)=O)cc2c(=O)cc(N3CCOCC3)oc12] | N-(8-(1-((3,5- difluorophenyl)amino)ethyl)-2- morpholino-4-oxo-4H-chromen-6- yl)methanesulfonamide |
| 6 | Compound 15 | 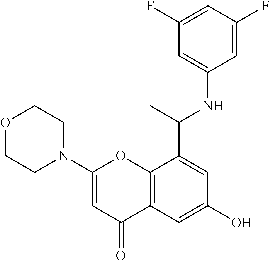 [SMILES: CC(Nc1cc(F)cc(F)c1)c1cc(O)cc2c(=O)cc(N3CCOCC3)oc12] | 8-(1-((3,5- difluorophenyl)amino)ethyl)-6- hydroxy-2-morpholino-4H- chromen-4-one |
| 7 | Compound 16 | 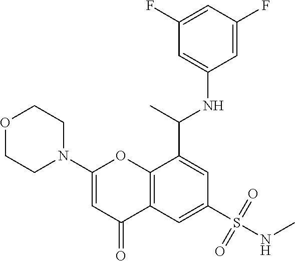 [SMILES: CNS(=O)(=O)c1cc(C(C)Nc2cc(F)cc(F)c2)c2oc(N3CCOCC3)cc(=O)c2c1] | 8-(1-((3,5- difluorophenyl)amino)ethyl)-N- methyl-2-morpholino-4-oxo-4H- chromene-6-sulfonamide |
| 8 | Compound 19 | 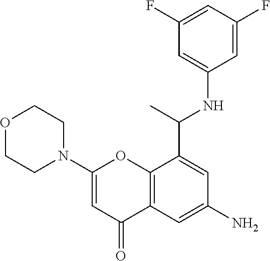 [SMILES: CC(Nc1cc(F)cc(F)c1)c1cc(N)cc2c(=O)cc(N3CCOCC3)oc12] | 8-(1-((3,5- difluorophenyl)amino)ethyl)-6- hydroxy-2-morpholino-4H- chromen-4-one |
| 9 | Compound 21 | 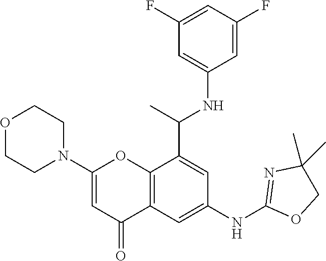 [SMILES: CC(Nc1cc(F)cc(F)c1)c1cc(NC2=NC(C)(C)CO2)cc2c(=O)cc(N3CCOCC3)oc12] | 8-(1-((3,5- difluorophenyl)amino)ethyl)-6- ((4,4-dimethyl-4,5-dihydrooxazol- 2-yl)amino)-2-morpholino-4H- chromen-4-one |
| 10 | Compound 22 | 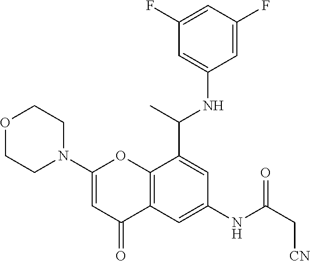 [SMILES: CC(Nc1cc(F)cc(F)c1)c1cc(NC(=O)CC#N)cc2c(=O)cc(N3CCOCC3)oc12] | 2-cyano-N-(8-(1-((3,5- difluorophenyl)amino)ethyl)-2- morpholino-4-oxo-4H-chromen-6- yl)acetamide |
| 11 | Compound 24 | 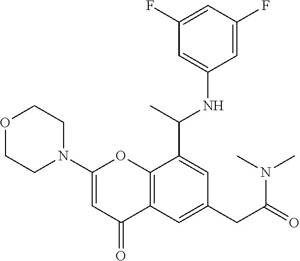 [SMILES: CC(Nc1cc(F)cc(F)c1)c1cc(CC(=O)N(C)C)cc2c(=O)cc(N3CCOCC3)oc12] | 2-(8-(1-((3,5- difluorophenyl)amino)ethyl)-2- morpholino-4-oxo-4H-chromen-6- yl)-N,N-dimethylacetamide |
| 12 | Compound 25 | 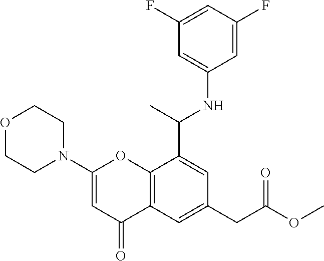 [SMILES: COC(=O)Cc1cc(C(C)Nc2cc(F)cc(F)c2)c2oc(N3CCOCC3)cc(=O)c2c1] | methyl 2-(8-(1-((3,5- difluorophenyl)amino)ethyl)-2- morpholino-4-oxo-4H-chromen-6- yl)acetate |
| 13 | Compound 27 | 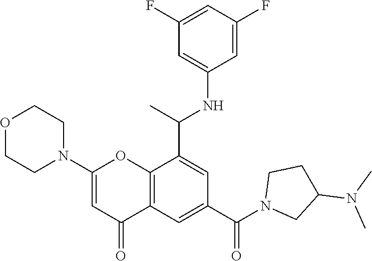 [SMILES: CC(Nc1cc(F)cc(F)c1)c1cc(C(=O)N2CCC(N(C)C)C2)cc2c(=O)cc(N3CCOCC3)oc12] | 8-(1-((3,5- difluorophenyl)amino)ethyl)-6-(3- (dimethylamino)pyrrolidine-1- carbonyl)-2-morpholino-4H- chromen-4-one |
| 14 | Compound 29 | 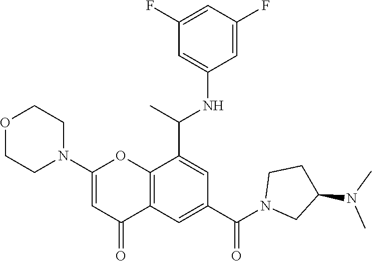 [SMILES: CC(Nc1cc(F)cc(F)c1)c1cc(C(=O)N2CC[C@@H](N(C)C)C2)cc2c(=O)cc(N3CCOCC3)oc12] | 8-(1-((3,5- difluorophenyl)amino)ethyl)-6- ((R)-3- (dimethylamino)pyrrolidine-1- carbonyl)-2-morpholino-4H- chromen-4-one |
| 15 | Compound 30 | 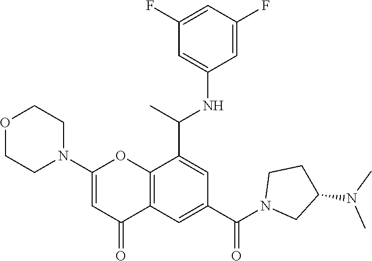 [SMILES: CC(Nc1cc(F)cc(F)c1)c1cc(C(=O)N2CC[C@H](N(C)C)C2)cc2c(=O)cc(N3CCOCC3)oc12] | 8-(1-((3,5- difluorophenyl)amino)ethyl)-6- ((S)-3- (dimethylamino)pyrrolidine-1- carbonyl)-2-morpholino-4H- chromen-4-one |
| 16 | Compound 31 | 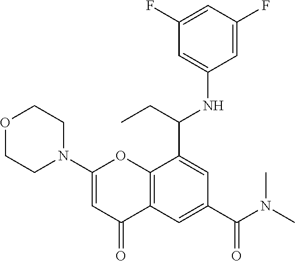 [SMILES: CCC(Nc1cc(F)cc(F)c1)c1cc(C(=O)N(C)C)cc2c(=O)cc(N3CCOCC3)oc12] | 8-(1-((3,5- difluorophenyl)amino)propyl)- N,N-dimethyl-2-morpholino-4- oxo-4H-chromene-6-carboxamide |
| 17 | Compound 32 | 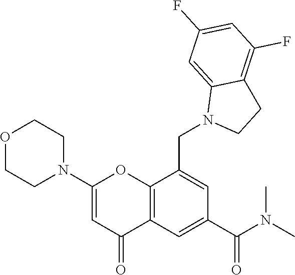 [SMILES: CN(C)C(=O)c1cc(CN2CCc3c(F)cc(F)cc32)c2oc(N3CCOCC3)cc(=O)c2c1] | 8-((4,6-difluoroindolin-1- yl)methyl)-N,N-dimethyl-2- morpholino-4-oxo-4H-chromene- 6-carboxamide |
| 18 | Compound 33 | 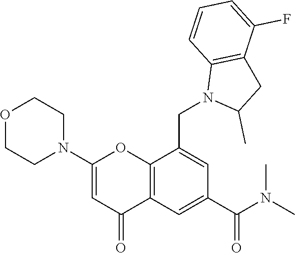 [SMILES: CC1Cc2c(F)cccc2N1Cc1cc(C(=O)N(C)C)cc2c(=O)cc(N3CCOCC3)oc12] | 8-((4-fluoro-2-methylindolin-1- yl)methyl)-N,N-dimethyl-2- morpholino-4-oxo-4H-chromene- 6-carboxamide |
| 19 | Compound 34 | 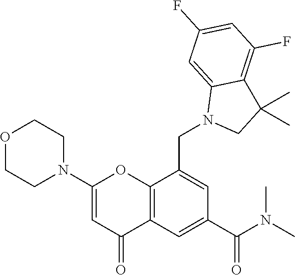 [SMILES: CN(C)C(=O)c1cc(CN2CC(C)(C)c3c(F)cc(F)cc32)c2oc(N3CCOCC3)cc(=O)c2c1] | 8-((4,6-difluoro-3,3- dimethylindolin-1-yl)methyl)- N,N-dimethyl-2-morpholino-4- oxo-4H-chromene-6-carboxamide |
| 20 | Compound 36 | 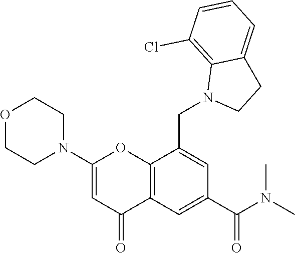 [SMILES: CN(C)C(=O)c1cc(CN2CCc3cccc(Cl)c32)c2oc(N3CCOCC3)cc(=O)c2c1] | 8-((7-chloroindolin-1-yl)methyl)- N,N-dimethyl-2-morpholino-4- oxo-4H-chromene-6-carboxamide |
| 21 | Compound 37 | 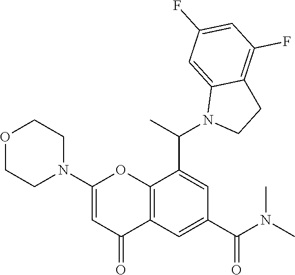 [SMILES: CC(c1cc(C(=O)N(C)C)cc2c(=O)cc(N3CCOCC3)oc12)N1CCc2c(F)cc(F)cc21] | 8-(1-(4,6-difluoroindolin-1- yl)ethyl)-N,N-dimethyl-2- morpholino-4-oxo-4H-chromene- 6-carboxamide |
| 22 | Compound 38 | 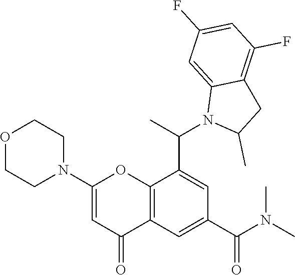 [SMILES: CC1Cc2c(F)cc(F)cc2N1C(C)c1cc(C(=O)N(C)C)cc2c(=O)cc(N3CCOCC3)oc12] | 8-(1-(4,6-difluoro-2- methylindolin-1-yl)ethyl)-N,N- dimethyl-2-morpholino-4-oxo-4H- chromene-6-carboxamide |
| 23 | Compound 39 | 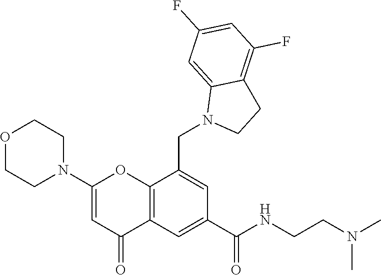 [SMILES: CN(C)CCNC(=O)c1cc(CN2CCc3c(F)cc(F)cc32)c2oc(N3CCOCC3)cc(=O)c2c1] | 8-((4,6-difluoroindolin-1- yl)methyl)-N-(2- (dimethylamino)ethyl)-2- morpholino-4-oxo-4H-chromene- 6-carboxamide |
| 24 | Compound 40 | 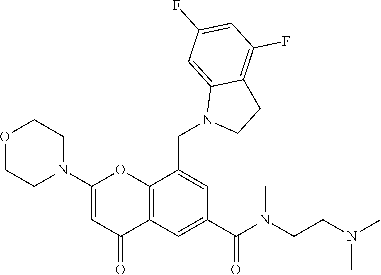 [SMILES: CN(C)CCN(C)C(=O)c1cc(CN2CCc3c(F)cc(F)cc32)c2oc(N3CCOCC3)cc(=O)c2c1] | 8-((4,6-difluoroindolin-1- yl)methyl)-N-(2- (dimethylamino)ethyl)-N-methyl- 2-morpholino-4-oxo-4H- chromene-6-carboxamide |
| 25 | Compound 41 | 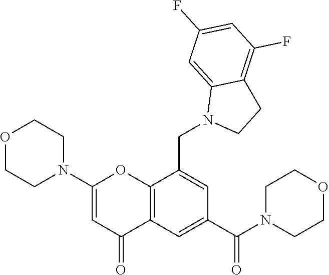 [SMILES: O=C(c1cc(CN2CCc3c(F)cc(F)cc32)c2oc(N3CCOCC3)cc(=O)c2c1)N1CCOCC1] | 8-((4,6-difluoroindolin-1- yl)methyl)-6-(morpholine-4- carbonyl)-2-morpholino-4H- chromen-4-one |
| 26 | Compound 42 | 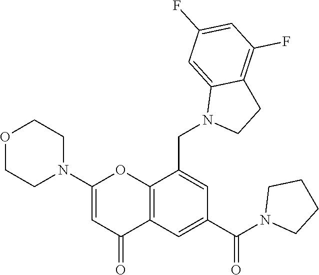 [SMILES: O=C(c1cc(CN2CCc3c(F)cc(F)cc32)c2oc(N3CCOCC3)cc(=O)c2c1)N1CCCC1] | 8-((4,6-difluoroindolin-1- yl)methyl)-2-morpholino-6- (pyrrolidine-1-carbonyl)-4H- chromen-4-one |
| 27 | Compound 43 | 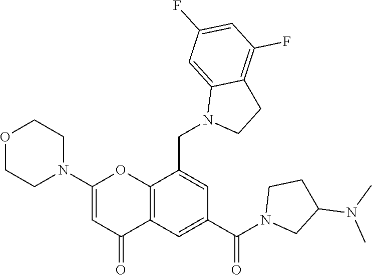 [SMILES: CN(C)C1CCN(C(=O)c2cc(CN3CCc4c(F)cc(F)cc43)c3oc(N4CCOCC4)cc(=O)c3c2)C1] | 8-((4,6-difluoroindolin-1- yl)methyl)-6-(3- (dimethylamino)pyrrolidine-1- carbonyl)-2-morpholino-4H- chromen-4-one |
| 28 | Compound 44 | 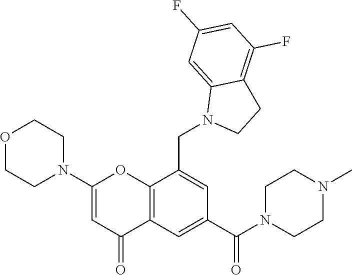 [SMILES: CN1CCN(C(=O)c2cc(CN3CCc4c(F)cc(F)cc43)c3oc(N4CCOCC4)cc(=O)c3c2)CC1] | 8-((4,6-difluoroindolin-1- yl)methyl)-6-(4-methylpiperazine- 1-carbonyl)-2-morpholino-4H- chromen-4-one |
| 29 | Compound 45 | 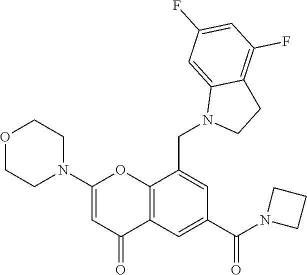 [SMILES: O=C(c1cc(CN2CCc3c(F)cc(F)cc32)c2oc(N3CCOCC3)cc(=O)c2c1)N1CCC1] | 6-(azetidine-1-carbonyl)-8-((4,6- difluoroindolin-1-yl)methyl)-2- morpholino-4H-chromen-4-one |
| 30 | Compound 46 | 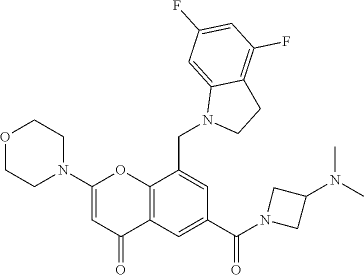 [SMILES: CN(C)C1CN(C(=O)c2cc(CN3CCc4c(F)cc(F)cc43)c3oc(N4CCOCC4)cc(=O)c3c2)C1] | 8-((4,6-difluoroindolin-1- yl)methyl)-6-(3- (dimethylamino)azetidine-1- carbonyl)-2-morpholino-4H- chromen-4-one |
| 31 | Compound 47 | 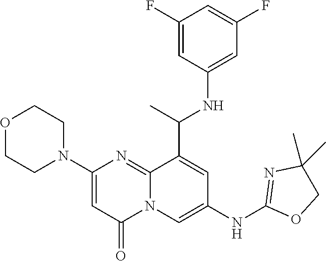 [SMILES: CC(Nc1cc(F)cc(F)c1)c1cc(NC2=NC(C)(C)CO2)cn2c(=O)cc(N3CCOCC3)nc12] | 9-(1-((3,5- difluorophenyl)amino)ethyl)-7- ((4,4-dimethyl-4,5-dihydrooxazol- 2-yl)amino)-2-morpholino-4H- pyrido[1,2-a]pyrimidin-4-one |
| 32 | Compound 48 | 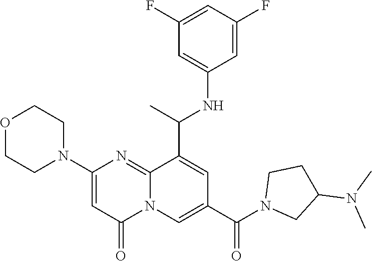 [SMILES: CC(Nc1cc(F)cc(F)c1)c1cc(C(=O)N2CCC(N(C)C)C2)cn2c(=O)cc(N3CCOCC3)nc12] | 9-(1-((3,5- difluorophenyl)amino)ethyl)-7-(3- (dimethylamino)pyrrolidine-1- carbonyl)-2-morpholino-4H- pyrido[1,2-a]pyrimidin-4-one |
| 33 | Compound 50 | 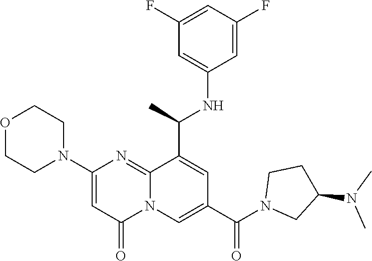 [SMILES: C[C@@H](Nc1cc(F)cc(F)c1)c1cc(C(=O)N2CC[C@@H](N(C)C)C2)cn2c(=O)cc(N3CCOCC3)nc12] | 9-((R)-1-((3,5- difluorophenyl)amino)ethyl)-7- ((R)-3- (dimethylamino)pyrrolidine-1- carbonyl)-2-morpholino-4H- pyrido[1,2-a]pyrimidin-4-one |
| 34 | Compound 51 | 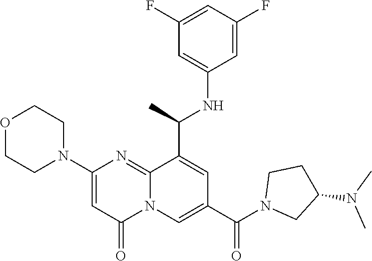 [SMILES: C[C@@H](Nc1cc(F)cc(F)c1)c1cc(C(=O)N2CC[C@H](N(C)C)C2)cn2c(=O)cc(N3CCOCC3)nc12] | 9-((R)-1-((3,5- difluorophenyl)amino)ethyl)-7- ((S)-3- (dimethylamino)pyrrolidine-1- carbonyl)-2-morpholino-4H- pyrido[1,2-a]pyrimidin-4-one |
| 35 | Compound 58 | 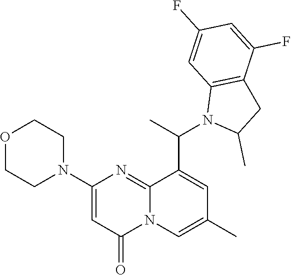 [SMILES: Cc1cc(C(C)N2c3cc(F)cc(F)c3CC2C)c2nc(N3CCOCC3)cc(=O)n2c1] | 9-(1-(4,6-difluoro-2- methylindolin-1-yl)ethyl)-7- methyl-2-morpholino-4H- pyrido[1,2-a]pyrimidin-4-one |
| 36 | Compound 59 | 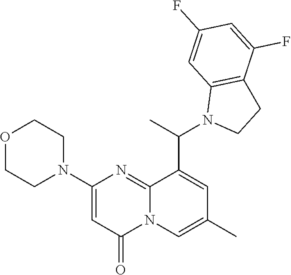 [SMILES: Cc1cc(C(C)N2CCc3c(F)cc(F)cc32)c2nc(N3CCOCC3)cc(=O)n2c1] | 9-(1-(4,6-difluoroindolin-1- yl)ethyl)-7-methyl-2-morpholino- 4H-pyrido[1,2-a]pyrimidin-4-one |
| 37 | Compound 60 | 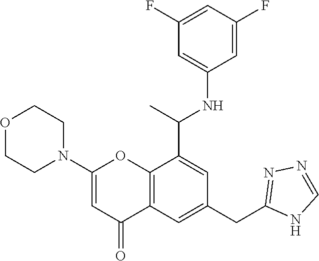 [SMILES: CC(Nc1cc(F)cc(F)c1)c1cc(Cc2nnc[nH]2)cc2c(=O)cc(N3CCOCC3)oc12] | 6-((4H-1,2,4-triazol-3-yl)methyl)- 8-(1-((3,5- difluorophenyl)amino)ethyl)-2- morpholino-4H-chromen-4-one |
| 38 | Compound 62 | 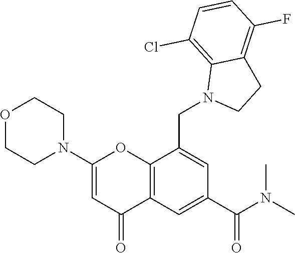 [SMILES: CN(C)C(=O)c1cc(CN2CCc3c(F)ccc(Cl)c32)c2oc(N3CCOCC3)cc(=O)c2c1] | 8-((7-chloro-4-fluoroindolin-1- yl)methyl)-N,N-dimethyl-2- morpholino-4-oxo-4H-chromene- 6-carboxamide |
| 39 | Compound 63 | 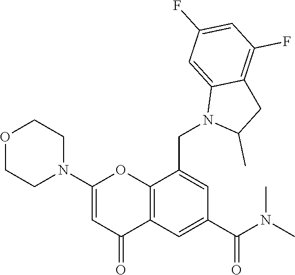 [SMILES: CC1Cc2c(F)cc(F)cc2N1Cc1cc(C(=O)N(C)C)cc2c(=O)cc(N3CCOCC3)oc12] | 8-((4,6-difluoro-2-methylindolin- 1-yl)methyl)-N,N-dimethyl-2- morpholino-4-oxo-4H-chromene- 6-carboxamide |
| 40 | Compound 64 | 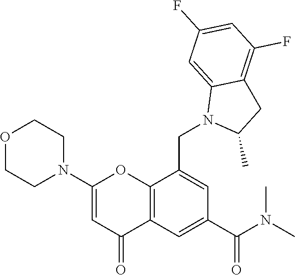 [SMILES: C[C@H]1Cc2c(F)cc(F)cc2N1Cc1cc(C(=O)N(C)C)cc2c(=O)cc(N3CCOCC3)oc12] | (S)-8-((4,6-difluoro-2- methylindolin-1-yl)methyl)-N,N- dimethyl-2-morpholino-4-oxo-4H- chromene-6-carboxamide |
| 41 | Compound 66 | 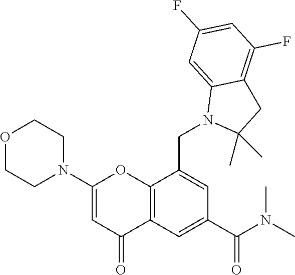 [SMILES: CN(C)C(=O)c1cc(CN2c3cc(F)cc(F)c3CC2(C)C)c2oc(N3CCOCC3)cc(=O)c2c1] | 8-((4,6-difluoro-2,2- dimethylindolin-1-yl)methyl)- N,N-dimethyl-2-morpholino-4- oxo-4H-chromene-6-carboxamide |
| 42 | Compound 68 | 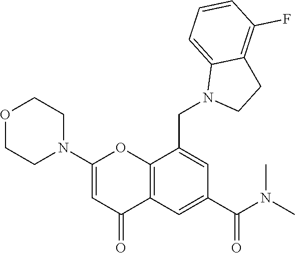 [SMILES: CN(C)C(=O)c1cc(CN2CCc3c(F)cccc32)c2oc(N3CCOCC3)cc(=O)c2c1] | 8-((4-fluoroindolin-1-yl)methyl)- N,N-dimethyl-2-morpholino-4- oxo-4H-chromene-6-carboxamide |
| 43 | Compound 70 | 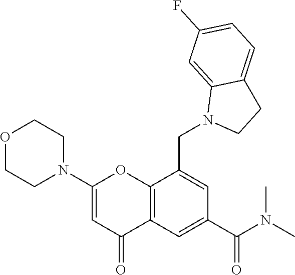 [SMILES: CN(C)C(=O)c1cc(CN2CCc3ccc(F)cc32)c2oc(N3CCOCC3)cc(=O)c2c1] | 8-((6-fluoroindolin-1-yl)methyl)- N,N-dimethyl-2-morpholino-4- oxo-4H-chromene-6-carboxamide |
| 44 | Compound 71 | 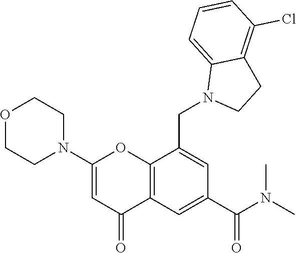 [SMILES: CN(C)C(=O)c1cc(CN2CCc3c(Cl)cccc32)c2oc(N3CCOCC3)cc(=O)c2c1] | 8-((4-chloroindolin-1-yl)methyl)- N,N-dimethyl-2-morpholino-4- oxo-4H-chromene-6-carboxamide |
| 45 | Compound 78 | 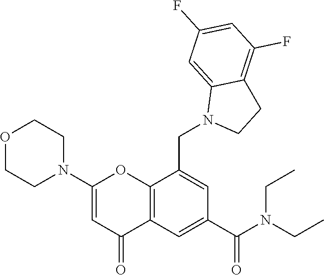 [SMILES: CCN(CC)C(=O)c1cc(CN2CCc3c(F)cc(F)cc32)c2oc(N3CCOCC3)cc(=O)c2c1] | 8-((4,6-difluoroindolin-1- yl)methyl)-N,N-diethyl-2- morpholino-4-oxo-4H-chromene- 6-carboxamide |
| 46 | Compound 79 | 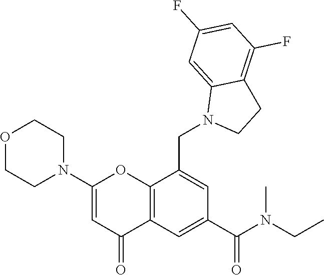 [SMILES: CCN(C)C(=O)c1cc(CN2CCc3c(F)cc(F)cc32)c2oc(N3CCOCC3)cc(=O)c2c1] | 8-((4,6-difluoroindolin-1- yl)methyl)-N-ethyl-N-methyl-2- morpholino-4-oxo-4H-chromene- 6-carboxamide |
| 47 | Compound 82 | 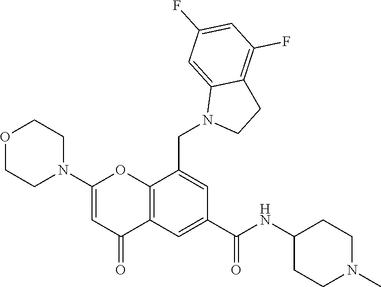 [SMILES: CN1CCC(NC(=O)c2cc(CN3CCc4c(F)cc(F)cc43)c3oc(N4CCOCC4)cc(=O)c3c2)CC1] | 8-((4,6-difluoroindolin-1- yl)methyl)-N-(1-methylpiperidin- 4-yl)-2-morpholino-4-oxo-4H- chromene-6-carboxamide |
| 48 | Compound 84 | 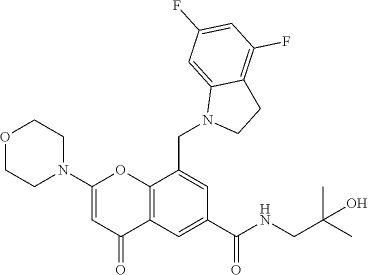 [SMILES: CC(C)(O)CNC(=O)c1cc(CN2CCc3c(F)cc(F)cc32)c2oc(N3CCOCC3)cc(=O)c2c1] | 8-((4,6-difluoroindolin-1- yl)methyl)-N-(2-hydroxy-2- methylpropyl)-2-morpholino-4- oxo-4H-chromene-6-carboxamide |
| 49 | Compound 86 | 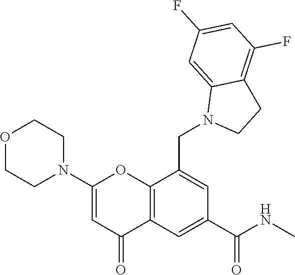 [SMILES: CNC(=O)c1cc(CN2CCc3c(F)cc(F)cc32)c2oc(N3CCOCC3)cc(=O)c2c1] | 8-((4,6-difluoroindolin-1- yl)methyl)-N-methyl-2- morpholino-4-oxo-4H-chromene- 6-carboxamide |
| 50 | Compound 90 | 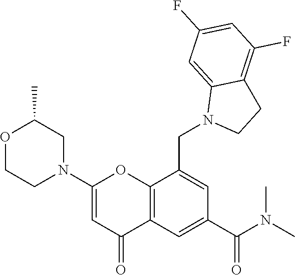 [SMILES: C[C@@H]1CN(c2cc(=O)c3cc(C(=O)N(C)C)cc(CN4CCc5c(F)cc(F)cc54)c3o2)CCO1] | (R)-8-((4,6-difluoroindolin-1- yl)methyl)-N,N-dimethyl-2-(2- methylmorpholino)-4-oxo-4H- chromene-6-carboxamide |
| 51 | Compound 91 | 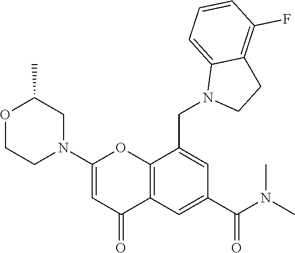 [SMILES: C[C@@H]1CN(c2cc(=O)c3cc(C(=O)N(C)C)cc(CN4CCc5c(F)cccc54)c3o2)CCO1] | (R)-8-((4-fluoroindolin-1- yl)methyl)-N,N-dimethyl-2-(2- methylmorpholino)-4-oxo-4H- chromene-6-carboxamide |
| 52 | Compound 93 | 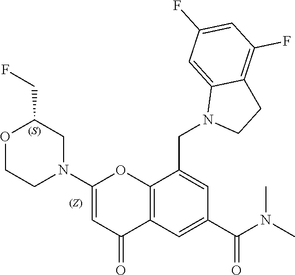 | (S)-8-((4,6-difluoroindolin-1- yl)methyl)-2-(2- (fluoromethyl)morpholino)-N,N- dimethyl-4-oxo-4H-chromene-6- carboxamide |
| 53 | Compound 96 | 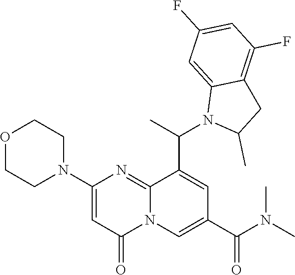 [SMILES: CC1Cc2c(F)cc(F)cc2N1C(C)c1cc(C(=O)N(C)C)cn2c(=O)cc(N3CCOCC3)nc12] | 9-(1-(4,6-difluoro-2- methylindolin-1-yl)ethyl)-N,N- dimethyl-2-morpholino-4-oxo-4H- pyrido[1,2-a]pyrimidine-7- carboxamide |
| 54 | Compound 98 | 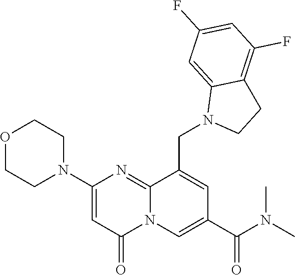 [SMILES: CN(C)C(=O)c1cc(CN2CCc3c(F)cc(F)cc32)c2nc(N3CCOCC3)cc(=O)n2c1] | 9-((4,6-difluoroindolin-1- yl)methyl)-N,N-dimethyl-2- morpholino-4-oxo-4H-pyrido[1,2- a]pyrimidine-7-carboxamide |
| 55 | Compound 99 | 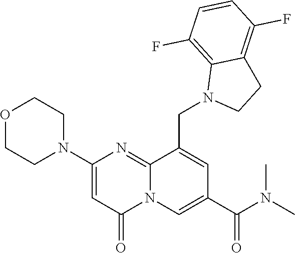 [SMILES: CN(C)C(=O)c1cc(CN2CCc3c(F)ccc(F)c32)c2nc(N3CCOCC3)cc(=O)n2c1] | 9-((4,7-difluoroindolin-1- yl)methyl)-N,N-dimethyl-2- morpholino-4-oxo-4H-pyrido[1,2- a]pyrimidine-7-carboxamide |
| 56 | Compound 100 | 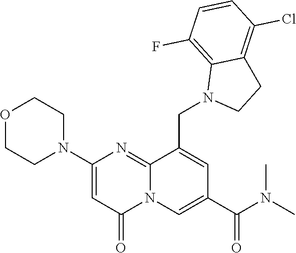 [SMILES: CN(C)C(=O)c1cc(CN2CCc3c(Cl)ccc(F)c32)c2nc(N3CCOCC3)cc(=O)n2c1] | 9-((4-chloro-7-fluoroindolin-1- yl)methyl)-N,N-dimethyl-2- morpholino-4-oxo-4H-pyrido[1,2- a]pyrimidine-7-carboxamide |
| 57 | Compound 101 | 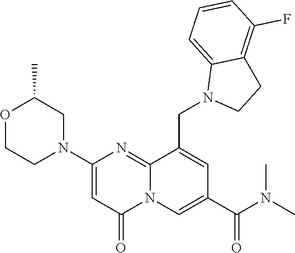 [SMILES: C[C@@H]1CN(c2cc(=O)n3cc(C(=O)N(C)C)cc(CN4CCc5c(F)cccc54)c3n2)CCO1] | (R)-9-((4-fluoroindolin-1- yl)methyl)-N,N-dimethyl-2-(2- methylmorpholino)-4-oxo-4H- pyrido[1,2-a]pyrimidine-7- carboxamide |
| 58 | Compound 102 | 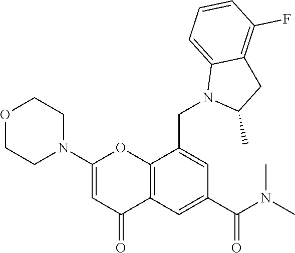 [SMILES: C[C@H]1Cc2c(F)cccc2N1Cc1cc(C(=O)N(C)C)cc2c(=O)cc(N3CCOCC3)oc12] | (S)-8-((4-fluoro-2-methylindolin- 1-yl)methyl)-N,N-dimethyl-2- morpholino-4-oxo-4H-chromene- 6-carboxamide |

or a pharmaceutically acceptable form or an isotope derivative thereof.

29. A pharmaceutical composition comprising a compound according to claim 1 and a pharmaceutically acceptable excipient, carrier, or diluent.

30. (canceled)

31. (canceled)

32. (canceled)

33. (canceled)

34. A unit dosage form comprising a pharmaceutical composition of claim 29.

35. (canceled)

36. (canceled)

37. (canceled)

38. (canceled)

39. A method for treating cancer, comprising administering to a subject in need thereof a therapeutically effective amount of a compound according to claim 1.

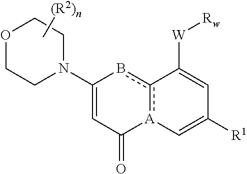

40. (canceled)

41. (canceled)

42. (canceled)

43. (canceled)

44. (canceled)

45. (canceled)
